SEQUENCE LISTING (131 sequences)

SEQ ID NO: 1 - protein, 93 aa, Homo sapiens
  REGION 1..93: note = misc_feature - DAP10 polypeptide
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 2 - protein, 42 aa, synthetic construct
  REGION 1..42: note = Synthetic: 4-1BB costimulation endodomain polypeptide
KRGRKKLLYIFKQPFMRPVQTTQEEDGCSCRFPEEEEGGCEL

SEQ ID NO: 3 - protein, 113 aa, synthetic construct
  REGION 1..113: note = Synthetic: CD3z signaling domain polypeptide 1
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 112 aa, synthetic construct
  REGION 1..112: note = Synthetic: CD3z signaling domain polypeptide 2
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 48 aa, synthetic construct
  REGION 1..48: note = Synthetic: CD27 costimulatory domain polypeptide
QRRKYRSNKGESPVEPAEPCHYSCPREEEGSTIPIQEDYRKPEPACSP

SEQ ID NO: 6 - protein, 21 aa, synthetic construct
  REGION 1..21: note = Synthetic: secretion signal 1 polypeptide
MALPVTALLLPLALLLHAARP

SEQ ID NO: 7 - protein, 48 aa, synthetic construct
  REGION 1..48: note = Synthetic: secretion signal 2 polypeptide
MRISKPHLRSISIQCYLCLLLNSHFLTEAGIHVFILGCFSAGLPKTEA

SEQ ID NO: 8 - protein, 114 aa, synthetic construct
  REGION 1..114: note = Synthetic: IL-5 polypeptide
NWVNVISDLKKIEDLIQSMHIDATLYTESDVHPSCKVTAMKCELLELQVISLESGDASIHDTVENLIILANNSLSSNGNVTESGCKECEELEEKNIKEFLQSFVHIVQMFINTS

SEQ ID NO: 9 - protein, 23 aa, synthetic construct
  REGION 1..23: note = Synthetic: P2A cleavage motif 1 polypeptide
SGSGATNFSLLKQAGDVEENPGP

SEQ ID NO: 10 - protein, 4 aa, synthetic construct
  REGION 1..4: note = Synthetic: furin cleavage site polypeptide
RAKR

SEQ ID NO: 11 - protein, 27 aa, synthetic construct
  REGION 1..27: note = Synthetic: cleavable linker polypeptide
RAKRSGSGATNFSLLKQAGDVEENPGP

SEQ ID NO: 12 - protein, 19 aa, synthetic construct
  REGION 1..19: note = Synthetic: P2A cleavage motif 2 polypeptide
ATNFSLLKQAGDVEENPGP

SEQ ID NO: 13 - protein, 23 aa, synthetic construct
  REGION 1..23: note = Synthetic: F2A cleavage motif polypeptide
VKQTLNNFDLLKLAGDVESNPGP

SEQ ID NO: 14 - protein, 20 aa, synthetic construct
  REGION 1..20: note = Synthetic: E2A cleavage motif polypeptide
QCTNYALLKLAGDVESNPGP

SEQ ID NO: 15 - protein, 17 aa, synthetic construct
  REGION 1..17: note = Synthetic: T2A cleavage motif polypeptide
EGRSLLTCGDVEENPGP

SEQ ID NO: 16 - DNA, 553 bp, synthetic construct
  misc_feature 1..553: note = Synthetic: IRES 1 nucleotide
  source 1..553: mol_type = other DNA
ctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgata

SEQ ID NO: 17 - DNA, 461 bp, synthetic construct
  misc_feature 1..461: note = Synthetic: IRES 2 nucleotide
  source 1..461: mol_type = other DNA
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccac

SEQ ID NO: 18 - protein, 93 aa, synthetic construct
  REGION 1..93: note = Synthetic: DAP10-K84R polypeptide
MIHLGHILFLLLLPVAAAQTTPGERSSLPAFYPGTSGSCSGCGSLSLPLLAGLVAADAVASLLIVGAVFLCARPRRSPAQEDGRVYINMPGRG

SEQ ID NO: 19 - protein, 93 aa, synthetic construct
  REGION 1..93: note = Synthetic: DAP10-Y86F polypeptide
MIHLGHILFLLLLPVAAAQTTPGERSSLPAFYPGTSGSCSGCGSLSLPLLAGLVAADAVASLLIVGAVFLCARPRRSPAQEDGKVFINMPGRG

SEQ ID NO: 20 - protein, 93 aa, synthetic construct
  REGION 1..93: note = Synthetic: DAP10-K84R-Y86F polypeptide
MIHLGHILFLLLLPVAAAQTTPGERSSLPAFYPGTSGSCSGCGSLSLPLLAGLVAADAVASLLIVGAVFLCARPRRSPAQEDGRVFINMPGRG

SEQ ID NO: 21 - protein, 473 aa, synthetic construct
  REGION 1..473: note = Synthetic: DAP10.0 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVYINMPGRGRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 22 - DNA, 1422 bp, synthetic construct
  misc_feature 1..1422: note = Synthetic: DAP10.0 Nucleic Acid
  source 1..1422: mol_type = other DNA
atgtccgtgcctacccaggtgctgggcctgctgctgctgtggctgaccgacgccagatgcatgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcaaggtgtacatcaacatgcccggcagaggacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 23 - protein, 586 aa, synthetic construct
  REGION 1..586: note = Synthetic: DAP10.3 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 24 - DNA, 1761 bp, synthetic construct
  misc_feature 1..1761: note = Synthetic: DAP10.3 Nucleic Acid
  source 1..1761: mol_type = other DNA
atgtccgtgcctacccaggtgctgggcctgctgctgctgtggctgaccgacgccagatgcatgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcaaggtgtacatcaacatgcccggcagaggaagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 25 - protein, 628 aa, synthetic construct
  REGION 1..628: note = Synthetic: DAP10.4 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 26 - DNA, 1887 bp, synthetic construct
  misc_feature 1..1887: note = Synthetic: DAP10.4 Nucleic Acid
  source 1..1887: mol_type = other DNA
atgtccgtgcctacccaggtgctgggcctgctgctgctgtggctgaccgacgccagatgcatgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaaaggtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 27 - protein, 628 aa, synthetic construct
  REGION 1..628: note = Synthetic: DAP10.5 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 28 - DNA, 1887 bp, synthetic construct
  misc_feature 1..1887: note = Synthetic: DAP10.5 Nucleic Acid
  source 1..1887: mol_type = other DNA
atgtccgtgcctacccaggtgctgggcctgctgctgctgtggctgaccgacgccagatgcatgatccacctgggacacatcctgttcctgctgctgcttccagtggccgccgctcaaacaacccctggcgagagaagcagcctgcccgccttctaccccggtacatctggcagctgcagcggctgcggcagcttgtctctgcctctgctggccggactggtggctgctgatgccgtggccagcctgctgatcgtgggcgccgtgttcctgtgcgccagacccagaaggtcccctgcccaggaggacggcaaggtgttcatcaacatgcctggcagaggcaaaagaggcagaaagaagctgctgtacatcttcaagcagcctttcatgcggcctgtgcagaccacccaggaggaagatggctgctcttgtcgatttccagaagaggaagaaggcggctgtgaactgagagtgaagttcagcagatccgccgacgcccctgcttatcagcagggccagaaccagctgtataatgagctgaacctgggccggcgggaagagtacgacgtgctggacaagcggcggggcagagatcctgagatgggcggaaaacctcaaagaaggaagaacccccaggagggactgtacaacgagctgcagaaggataagatggccgaggcctacagcgagatcggcatgaaaggagaaagaagaagaggaaaaggccacgacggcctgtaccagggcctgagcaccgccaccaaggacacctacgacgccctgcacatgcaggccctgccacctagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 29 - protein, 628 aa, synthetic construct
  REGION 1..628: note = Synthetic: DAP10.6 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 30 - DNA, 1887 bp, synthetic construct
  misc_feature 1..1887: note = Synthetic: DAP10.6 Nucleic Acid
  source 1..1887: mol_type = other DNA
atgtccgtgcctacccaggtgctgggcctgctgctgctgtggctgaccgacgccagatgcatgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttagagtgaagttcagcagaagcgccgacgcccctgcttaccagcagggccagaaccagctgtataatgagctgaacctgggaagaagggaagagtacgacgtgctggacaagcggcggggcagagatcctgagatgggcggaaaacctcaaagaaggaagaaccctcaggagggcctgtacaacgagctgcagaaagacaagatggccgaggcctacagcgagatcggcatgaaaggcgagagacggcggggcaagggccacgacggcctgtaccaaggcctgtcaacagccaccaaggacacctacgacgccctgcacatgcaggccctgccacctagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 31 - protein, 473 aa, synthetic construct
  REGION 1..473: note = Synthetic: DAP10.13 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVYINMPGRGRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 32 - DNA, 1422 bp, synthetic construct
  misc_feature 1..1422: note = Synthetic: DAP10.13 Nucleic Acid
  source 1..1422: mol_type = other DNA
atgtccgtgcctacccaggtgctgggcctgctgctgctgtggctgaccgacgccagatgcatgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcagagtgtacatcaacatgcccggcagaggacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 33 - protein, 586 aa, synthetic construct
  REGION 1..586: note = Synthetic: DAP10.14 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 34 - DNA, 1761 bp, synthetic construct
  misc_feature 1..1761: note = Synthetic: DAP10.14 Nucleic Acid
  source 1..1761: mol_type = other DNA
atgtccgtgcctacccaggtgctgggcctgctgctgctgtggctgaccgacgccagatgcatgatccacctgggccacatcctgttcctgctgctgctgcctgtggccgctgcccagaccacccctggagaaagatctagccttccagccttctaccccgggaccagcggaagctgcagcggctgcggcagcttgtctctgcctctgctggccggcctggtggccgccgacgctgttgcatctctgctgatcgtgggcgccgtctttctgtgcgccagacccagacggagccctgctcaggaagatggcagagtgtacatcaacatgcctggcagaggcagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 35 - protein, 628 aa, synthetic construct
  REGION 1..628: note = Synthetic: DAP10.15 Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 36 - DNA, 1887 bp, synthetic construct
  misc_feature 1..1887: note = Synthetic: DAP10.15 Nucleic Acid
  source 1..1887: mol_type = other DNA
atgtccgtgcctacccaggtgctgggcctgctgctgctgtggctgaccgacgccagatgcatgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaagagtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctga

SEQ ID NO: 37 - protein, 93 aa, synthetic construct
  REGION 1..93: note = Synthetic: DAP10-D57A
MIHLGHILFLLLLPVAAAQTTPGERSSLPAFYPGTSGSCSGCGSLSLPLLAGLVAAAAVASLLIVGAVFLCARPRRSPAQEDGKVYINMPGRG

SEQ ID NO: 38 - protein, 93 aa, synthetic construct
  REGION 1..93: note = Synthetic: DAP10-N88Q
MIHLGHILFLLLLPVAAAQTTPGERSSLPAFYPGTSGSCSGCGSLSLPLLAGLVAADAVASLLIVGAVFLCARPRRSPAQEDGKVYIQMPGRG

SEQ ID NO: 39 - protein, 93 aa, synthetic construct
  REGION 1..93: note = Synthetic: DAP10-M89Q
MIHLGHILFLLLLPVAAAQTTPGERSSLPAFYPGTSGSCSGCGSLSLPLLAGLVAADAVASLLIVGAVFLCARPRRSPAQEDGKVYINQPGRG

SEQ ID NO: 40 - protein, 68 aa, synthetic construct
  REGION 1..68: note = Synthetic: CD28 costimulatory domain
FWVLVVVGGVLACYSLLVTVAFIIFWVRSKRSRLLHSDYMNMTPRRPGPTRKHYQPYAPPRDFAAYRS

SEQ ID NO: 41 - protein, 44 aa, synthetic construct
  REGION 1..44: note = Synthetic: CD28 costimulatory domain
FWVRSKRSRLLHSDYMNMTPRRPGPTRKHYQPYAPPRDFAAYRS

SEQ ID NO: 42 - protein, 35 aa, synthetic construct
  REGION 1..35: note = Synthetic: ICOS costimulatory domain
TKKKYSSSVHDPNGEYMFMRAVNTAKKSRLTDVTL

SEQ ID NO: 43 - protein, 37 aa, synthetic construct
  REGION 1..37: note = Synthetic: OX40
RRDQRLPPDAHKPPGGGSFRTPIQEEQADAHSTLAKI

SEQ ID NO: 44 - protein, 22 aa, synthetic construct
  REGION 1..22: note = Synthetic: P2A cleavage motif 2 polypeptide
GSGATNFSLLKQAGDVEENPGP

SEQ ID NO: 45 - protein, 20 aa, synthetic construct
  REGION 1..20: note = Synthetic: Signal peptide
MSVPTQVLGLLLLWLTDARC

SEQ ID NO: 46 - DNA, 678 bp, synthetic construct
  misc_feature 1..678: note = Synthetic: DAP10.3.1 (+sig domain, -FP2A, -CD19t) Nucleic Acid
  source 1..678: mol_type = other DNA
atgtccgtgcctacccaggtgctgggcctgctgctgctgtggctgaccgacgccagatgcatgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcaaggtgtacatcaacatgcccggcagaggaagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctaga

SEQ ID NO: 47 - protein, 226 aa, synthetic construct
  REGION 1..226: note = Synthetic: DAP10.3.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 48 - DNA, 804 bp, synthetic construct
  misc_feature 1..804: note = Synthetic: DAP10.4.1 (+sig domain, -FP2A, -CD19t) Nucleic Acid
  source 1..804: mol_type = other DNA
atgtccgtgcctacccaggtgctgggcctgctgctgctgtggctgaccgacgccagatgcatgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaaaggtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccaga

SEQ ID NO: 49 - protein, 268 aa, synthetic construct
  REGION 1..268: note = Synthetic: DAP10.4.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 50 - DNA, 804 bp, synthetic construct
  misc_feature 1..804: note = Synthetic: DAP10.5.1 (+sig domain, -FP2A, -CD19t) Nucleic Acid
  source 1..804: mol_type = other DNA
atgtccgtgcctacccaggtgctgggcctgctgctgctgtggctgaccgacgccagatgcatgatccacctgggacacatcctgttcctgctgctgcttccagtggccgccgctcaaacaacccctggcgagagaagcagcctgcccgccttctaccccggtacatctggcagctgcagcggctgcggcagcttgtctctgcctctgctggccggactggtggctgctgatgccgtggccagcctgctgatcgtgggcgccgtgttcctgtgcgccagacccagaaggtcccctgcccaggaggacggcaaggtgttcatcaacatgcctggcagaggcaaaagaggcagaaagaagctgctgtacatcttcaagcagcctttcatgcggcctgtgcagaccacccaggaggaagatggctgctcttgtcgatttccagaagaggaagaaggcggctgtgaactgagagtgaagttcagcagatccgccgacgcccctgcttatcagcagggccagaaccagctgtataatgagctgaacctgggccggcgggaagagtacgacgtgctggacaagcggcggggcagagatcctgagatgggcggaaaacctcaaagaaggaagaacccccaggagggactgtacaacgagctgcagaaggataagatggccgaggcctacagcgagatcggcatgaaaggagaaagaagaagaggaaaaggccacgacggcctgtaccagggcctgagcaccgccaccaaggacacctacgacgccctgcacatgcaggccctgccacctaga

SEQ ID NO: 51 - protein, 268 aa, synthetic construct
  REGION 1..268: note = Synthetic: DAP10.5.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGKVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 52 - DNA, 804 bp, synthetic construct
  misc_feature 1..804: note = Synthetic: DAP10.6.1 (+sig domain, -FP2A, -CD19t) Nucleic Acid
  source 1..804: mol_type = other DNA
atgtccgtgcctacccaggtgctgggcctgctgctgctgtggctgaccgacgccagatgcatgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttagagtgaagttcagcagaagcgccgacgcccctgcttaccagcagggccagaaccagctgtataatgagctgaacctgggaagaagggaagagtacgacgtgctggacaagcggcggggcagagatcctgagatgggcggaaaacctcaaagaaggaagaaccctcaggagggcctgtacaacgagctgcagaaagacaagatggccgaggcctacagcgagatcggcatgaaaggcgagagacggcggggcaagggccacgacggcctgtaccaaggcctgtcaacagccaccaaggacacctacgacgccctgcacatgcaggccctgccacctaga

SEQ ID NO: 53 - protein, 268 aa, synthetic construct
  REGION 1..268: note = Synthetic: DAP10.6.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - DNA, 339 bp, synthetic construct
  misc_feature 1..339: note = Synthetic: DAP10.13.1 (+sig domain, -FP2A, -CD19t) Nucleic Acid
  source 1..339: mol_type = other DNA
atgtccgtgcctacccaggtgctgggcctgctgctgctgtggctgaccgacgccagatgcatgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcagagtgtacatcaacatgcccggcagagga

SEQ ID NO: 55 - protein, 113 aa, synthetic construct
  REGION 1..113: note = Synthetic: DAP10.13.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVYINMPGRG

SEQ ID NO: 56 - DNA, 678 bp, synthetic construct
  misc_feature 1..678: note = Synthetic: DAP10.14.1 (+sig domain, -FP2A, -CD19t) Nucleic Acid
  source 1..678: mol_type = other DNA
atgtccgtgcctacccaggtgctgggcctgctgctgctgtggctgaccgacgccagatgcatgatccacctgggccacatcctgttcctgctgctgctgcctgtggccgctgcccagaccacccctggagaaagatctagccttccagccttctaccccgggaccagcggaagctgcagcggctgcggcagcttgtctctgcctctgctggccggcctggtggccgccgacgctgttgcatctctgctgatcgtgggcgccgtctttctgtgcgccagacccagacggagccctgctcaggaagatggcagagtgtacatcaacatgcctggcagaggcagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctaga

SEQ ID NO: 57 - protein, 226 aa, synthetic construct
  REGION 1..226: note = Synthetic: DAP10.14.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 58 - DNA, 804 bp, synthetic construct
  misc_feature 1..804: note = Synthetic: DAP10.15.1 (+sig domain, -FP2A, -CD19t) Nucleic Acid
  source 1..804: mol_type = other DNA
atgtccgtgcctacccaggtgctgggcctgctgctgctgtggctgaccgacgccagatgcatgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaagagtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccaga

SEQ ID NO: 59 - protein, 268 aa, synthetic construct
  REGION 1..268: note = Synthetic: DAP10.15.1 (+sig domain, -FP2A, -CD19t) Amino Acid
MSVPTQVLGLLLLWLTDARCMIHLGHILFLLLLPVAAAQTTPGERSSLPAFYPGTSGSCSGCGSLSLPLLAGLVAADAVASLLIVGAVFLCARPRRSPAQEDGRVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 60 - DNA, 618 bp, synthetic construct
  misc_feature 1..618: note = Synthetic: DAP10.3.2 (-sig domain, -FP2A, -CD19t) Nucleic Acid
  source 1..618: mol_type = other DNA
atgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcaaggtgtacatcaacatgcccggcagaggaagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctaga

SEQ ID NO: 61 - protein, 206 aa, synthetic construct
  REGION 1..206: note = Synthetic: DAP10.3.2 (-sig domain, -FP2A, -CD19t) Amino Acid
MIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - DNA, 744 bp, synthetic construct
  misc_feature 1..744: note = Synthetic: DAP10.4.2 (-sig domain, -FP2A, -CD19t) Nucleic Acid
  source 1..744: mol_type = other DNA
atgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaaaggtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccaga

SEQ ID NO: 63 - protein, 248 aa, synthetic construct
  REGION 1..248: note = Synthetic: DAP10.4.2 (-sig domain, -FP2A, -CD19t) Amino Acid
MIHLGHILFLLLLPVAAAQTTPGERSSLPAFYPGTSGSCSGCGSLSLPLLAGLVAADAVASLLIVGAVFLCARPRRSPAQEDGKVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - DNA, 744 bp, synthetic construct
  misc_feature 1..744: note = Synthetic: DAP10.5.2 (-sig domain, -FP2A, -CD19t) Nucleic Acid
  source 1..744: mol_type = other DNA
atgatccacctgggacacatcctgttcctgctgctgcttccagtggccgccgctcaaacaacccctggcgagagaagcagcctgcccgccttctaccccggtacatctggcagctgcagcggctgcggcagcttgtctctgcctctgctggccggactggtggctgctgatgccgtggccagcctgctgatcgtgggcgccgtgttcctgtgcgccagacccagaaggtcccctgcccaggaggacggcaaggtgttcatcaacatgcctggcagaggcaaaagaggcagaaagaagctgctgtacatcttcaagcagcctttcatgcggcctgtgcagaccacccaggaggaagatggctgctcttgtcgatttccagaagaggaagaaggcggctgtgaactgagagtgaagttcagcagatccgccgacgcccctgcttatcagcagggccagaaccagctgtataatgagctgaacctgggccggcgggaagagtacgacgtgctggacaagcggcggggcagagatcctgagatgggcggaaaacctcaaagaaggaagaacccccaggagggactgtacaacgagctgcagaaggataagatggccgaggcctacagcgagatcggcatgaaaggagaaagaagaagaggaaaaggccacgacggcctgtaccagggcctgagcaccgccaccaaggacacctacgacgccctgcacatgcaggccctgccacctaga

SEQ ID NO: 65 - protein, 248 aa, synthetic construct
  REGION 1..248: note = Synthetic: DAP10.5.2 (-sig domain, -FP2A, -CD19t) Amino Acid
MIHLGHILFLLLLPVAAAQTTPGERSSLPAFYPGTSGSCSGCGSLSLPLLAGLVAADAVASLLIVGAVFLCARPRRSPAQEDGKVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 66 - DNA, 744 bp, synthetic construct
  misc_feature 1..744: note = Synthetic: DAP10.6.2 (-sig domain, -FP2A, -CD19t) Nucleic Acid
  source 1..744: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttagagtgaagttcagcagaagcgccgacgcccctgcttaccagcagggccagaaccagctgtataatgagctgaacctgggaagaagggaagagtacgacgtgctggacaagcggcggggcagagatcctgagatgggcggaaaacctcaaagaaggaagaaccctcaggagggcctgtacaacgagctgcagaaagacaagatggccgaggcctacagcgagatcggcatgaaaggcgagagacggcggggcaagggccacgacggcctgtaccaaggcctgtcaacagccaccaaggacacctacgacgccctgcacatgcaggccctgccacctaga

SEQ ID NO: 67 - protein, 248 aa, synthetic construct
  REGION 1..248: note = Synthetic: DAP10.6.2 (-sig domain, -FP2A, -CD19t) Amino Acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 68 - DNA, 279 bp, synthetic construct
  misc_feature 1..279: note = Synthetic: DAP10.13.2 (-sig domain, -FP2A, -CD19t) Nucleic Acid
  source 1..279: mol_type = other DNA
atgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcagagtgtacatcaacatgcccggcagagga

SEQ ID NO: 69 - protein, 93 aa, synthetic construct; identical to SEQ ID NO: 18
  REGION 1..93: note = Synthetic: DAP10.13.2 (-sig domain, -FP2A, -CD19t) Amino Acid

SEQ ID NO: 70 - DNA, 618 bp, synthetic construct
  misc_feature 1..618: note = Synthetic: DAP10.14.2 (-sig domain, -FP2A, -CD19t) Nucleic Acid
  source 1..618: mol_type = other DNA
atgatccacctgggccacatcctgttcctgctgctgctgcctgtggccgctgcccagaccacccctggagaaagatctagccttccagccttctaccccgggaccagcggaagctgcagcggctgcggcagcttgtctctgcctctgctggccggcctggtggccgccgacgctgttgcatctctgctgatcgtgggcgccgtctttctgtgcgccagacccagacggagccctgctcaggaagatggcagagtgtacatcaacatgcctggcagaggcagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctaga

SEQ ID NO: 71 - protein, 206 aa, synthetic construct
  REGION 1..206: note = Synthetic: DAP10.14.2 (-sig domain, -FP2A, -CD19t) Amino Acid
MIHLGHILFLLLLPVAAAQTTPGERSSLPAFYPGTSGSCSGCGSLSLPLLAGLVAADAVASLLIVGAVFLCARPRRSPAQEDGRVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 72 - DNA, 744 bp, synthetic construct
  misc_feature 1..744: note = Synthetic: DAP10.15.2 (-sig domain, -FP2A, -CD19t) Nucleic Acid
  source 1..744: mol_type = other DNA
atgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaagagtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccaga

SEQ ID NO: 73 - protein, 248 aa, synthetic construct
  REGION 1..248: note = Synthetic: DAP10.15.2 (-sig domain, -FP2A, -CD19t) Amino Acid
MIHLGHILFLLLLPVAAAQTTPGERSSLPAFYPGTSGSCSGCGSLSLPLLAGLVAADAVASLLIVGAVFLCARPRRSPAQEDGRVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 74 - protein, 216 aa, synthetic construct
  REGION 1..216: note = Synthetic: NKG2D
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 75 - protein, 21 aa, synthetic construct
  REGION 1..21: note = Synthetic: NKG2D transmembrane domain
PFFFCCFIAVAMGIRFIIMVA

SEQ ID NO: 76 - protein, 165 aa, synthetic construct
  REGION 1..165: note = Synthetic: NKG2D ligand-binding domain and transmembrane domain
PFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 77 - protein, 18 aa, synthetic construct
  REGION 1..18: note = Synthetic: Linker 1 polypeptide
GSTSGSGKPGSGEGSTKG

SEQ ID NO: 78 - protein, 75 aa, synthetic construct
  REGION 1..75: note = Synthetic: DAP10 mature form
QTTPGERSSLPAFYPGTSGSCSGCGSLSLPLLAGLVAADAVASLLIVGAVFLCARPRRSPAQEDGKVYINMPGRG

SEQ ID NO: 79 - protein, 21 aa, synthetic construct
  REGION 1..21: note = Synthetic: DAP10 transmembrane domain
LLAGLVAADAVASLLIVGAVF

SEQ ID NO: 80 - protein, 30 aa, synthetic construct
  REGION 1..30: note = Synthetic: DAP10 extracellular domain
QTTPGERSSLPAFYPGTSGSCSGCGSLSLP

SEQ ID NO: 81 - protein, 45 aa, synthetic construct
  REGION 1..45: note = Synthetic: DAP10 transmembrane and cytoplasmic domain
LLAGLVAADAVASLLIVGAVFLCARPRRSPAQEDGKVYINMPGRG

SEQ ID NO: 82 - protein, 113 aa, synthetic construct
  REGION 1..113: note = Synthetic: CD3z signaling peptide 3 (1XX)
RVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 83 - protein, 632 aa, synthetic construct
  REGION 1..632: note = Synthetic: DAP10.21-FP2A-EGFRt Amino Acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 84 - DNA, 1902 bp, synthetic construct
  misc_feature 1..1902: note = Synthetic: DAP10.21-FP2A-EGFRt Nucleotide
  source 1..1902: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttagagtgaagttcagcagaagcgccgacgcccctgcttaccagcagggccagaaccagctgtataatgagctgaacctgggaagaagggaagagtacgacgtgctggacaagcggcggggcagagatcctgagatgggcggaaaacctcaaagaaggaagaaccctcaggagggcctgtacaacgagctgcagaaagacaagatggccgaggcctacagcgagatcggcatgaaaggcgagagacggcggggcaagggccacgacggcctgtaccaaggcctgtcaacagccaccaaggacacctacgacgccctgcacatgcaggccctgccacctagacgcgcgaagcgatcaggcagcggggcgacaaatttcagccttctgaaacaagcaggcgacgtggaagaaaaccccggtccaatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgataa

SEQ ID NO: 85 - protein, 632 aa, synthetic construct
  REGION 1..632: note = Synthetic: DAP10.28-FP2A-EGFRt Amino Acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPRRAKRSGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 86 - DNA, 1902 bp, synthetic construct
  misc_feature 1..1902: note = Synthetic: DAP10.28-FP2A-EGFRt Nucleotide
  source 1..1902: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttcgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccagacgcgcgaagcgatcaggcagcggggcgacaaatttcagccttctgaaacaagcaggcgacgtggaagaaaaccccggtccaatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgataa

SEQ ID NO: 87 - protein, 590 aa, synthetic construct
  REGION 1..590: note = Synthetic: DAP10.29-FP2A-EGFRt Amino Acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPRRAKRSGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 88 - DNA, 1776 bp, synthetic construct
  misc_feature 1..1776: note = Synthetic: DAP10.29-FP2A-EGFRt Nucleotide
  source 1..1776: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggccgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccagacgcgcgaagcgatcaggcagcggggcgacaaatttcagccttctgaaacaagcaggcgacgtggaagaaaaccccggtccaatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgataa

SEQ ID NO: 89 - protein, 246 aa, synthetic construct
  REGION 1..246: note = Synthetic: Truncated LNGFR Amino Acid
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 90 - DNA, 744 bp, synthetic construct
  misc_feature 1..744: note = Synthetic: Truncated LNGFR Nucleic Acid
  source 1..744: mol_type = other DNA
aaagaggcctgtcctaccggcctgtatacacactctggcgagtgctgcaaggcctgcaatcttggagaaggcgtggcacagccttgcggcgctaatcagacagtgtgcgagccttgcctggacagcgtgacctttagcgacgtggtgtctgccaccgagccatgcaagccttgtaccgagtgtgtgggcctgcagagcatgtctgccccttgtgtggaagccgacgatgccgtgtgtagatgcgcctacggctactaccaggacgagacaacaggcagatgcgaggcctgtagagtgtgtgaagccggctctggactggtgttcagctgccaagacaagcagaacaccgtgtgcgaggaatgccccgatggcacctatagcgacgaggccaaccatgtggatccctgcctgccttgtactgtgtgcgaagataccgagcggcagctgcgcgagtgtacaagatgggctgatgccgagtgcgaagagatccccggcagatggatcaccagaagcacacctccagagggcagcgatagcacagccccttctacacaagagcccgaggctcctcctgagcaggatctgattgcctctacagtggccggcgtggtcacaacagtgatgggatcttctcagcccgtggtcaccagaggcaccaccgacaatctgatccccgtgtactgtagcatcctggccgccgtggttgtgggactcgtggcctatatcgccttcaagagatgataa

SEQ ID NO: 91 - protein, 335 aa, synthetic construct
  REGION 1..335: note = Synthetic: Truncated EGFR (EGFRt) Amino Acid
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 92 - DNA, 1011 bp, synthetic construct
  misc_feature 1..1011: note = Synthetic: Truncated EGFR (EGFRt) Nucleic acid
  source 1..1011: mol_type = other DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgataa

SEQ ID NO: 93 - protein, 18 aa, synthetic construct
  REGION 1..18: note = Synthetic: Dap10 signal peptide
MIHLGHILFLLLLPVAAA

SEQ ID NO: 94 - protein, 632 aa, synthetic construct; identical to SEQ ID NO: 83
  REGION 1..632: note = Synthetic: DAP10.6.3 (+sig domain, +FP2A, +EGFRt) Amino Acid

SEQ ID NO: 95 - DNA, 1902 bp, synthetic construct; identical to SEQ ID NO: 84
  misc_feature 1..1902: note = Synthetic: DAP10.6.3 (+sig domain, +FP2A, +EGFRt) Nucleic Acid
  source 1..1902: mol_type = other DNA

SEQ ID NO: 96 - protein, 608 aa, synthetic construct
  REGION 1..608: note = Synthetic: DAP10.16 Amino acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 97 - DNA, 1830 bp, synthetic construct
  misc_feature 1..1830: note = Synthetic: DAP10.16 Nucleic acid
  source 1..1830: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttcgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctgataa

SEQ ID NO: 98 - protein, 248 aa, synthetic construct
  REGION 1..248: note = Synthetic: DAP10.16.1 (+sig domain, -FP2A, -CD19) Amino acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 99 - DNA, 744 bp, synthetic construct
  misc_feature 1..744: note = Synthetic: DAP10.16.1 (+sig domain, -FP2A, -CD19) Nucleic acid
  source 1..744: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttcgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccaga

SEQ ID NO: 100 - protein, 230 aa, synthetic construct
  REGION 1..230: note = Synthetic: DAP10.16.2 (-sig domain, -FP2A, -CD19) Amino acid
QTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 101 - DNA, 690 bp, synthetic construct
  misc_feature 1..690: note = Synthetic: DAP10.16.2 (-sig domain, -FP2A, -CD19) Nucleic acid
  source 1..690: mol_type = other DNA
cagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttcgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccaga

SEQ ID NO: 102 - protein, 632 aa, synthetic construct; identical to SEQ ID NO: 85
  REGION 1..632: note = Synthetic: DAP10.16.3 (+sig domain, +FP2A, +EGFRt) Amino Acid

SEQ ID NO: 103 - DNA, 1902 bp, synthetic construct; identical to SEQ ID NO: 86
  misc_feature 1..1902: note = Synthetic: DAP10.16.3 (+sig domain, +FP2A, +EGFRt) Nucleic Acid
  source 1..1902: mol_type = other DNA

SEQ ID NO: 104 - protein, 566 aa, synthetic construct
  REGION 1..566: note = Synthetic: DAP10.17 Amino acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 105 - DNA, 1704 bp, synthetic construct
  misc_feature 1..1704: note = Synthetic: DAP10.17 Nucleic acid
  source 1..1704: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggccgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccagacgcgcgaagcgatcaggcagcggggcgacaaatttcagccttctgaaacaagcaggcgacgtggaagaaaaccccggtccaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctgataa

SEQ ID NO: 106 - protein, 206 aa, synthetic construct
  REGION 1..206: note = Synthetic: DAP10.17.1 (+sig domain, -FP2A, -CD19) Amino acid
MIHLGHILFLLLLPVAAAQTTPGERSSLPAFYPGTSGSCSGCGSLSLPLLAGLVAADAVASLLIVGAVFLCARPRRSPAQEDGRVFINMPGRGRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 107 - DNA, 618 bp, synthetic construct
  misc_feature 1..618: note = Synthetic: DAP10.17.1 (+sig domain, -FP2A, -CD19) Nucleic acid
  source 1..618: mol_type = other DNA
atgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggccgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccaga

SEQ ID NO: 108 - protein, 188 aa, synthetic construct
  REGION 1..188: note = Synthetic: DAP10.17.2 (-sig domain, -FP2A, -CD19) Amino acid
QTTPGERSSLPAFYPGTSGSCSGCGSLSLPLLAGLVAADAVASLLIVGAVFLCARPRRSPAQEDGRVFINMPGRGRVKFSRSADAPAYQQGQNQLYNELNLGRREEYDVLDKRRGRDPEMGGKPQRRKNPQEGLFNELQKDKMAEAFSEIGMKGERRRGKGHDGLFQGLSTATKDTFDALHMQALPPR

SEQ ID NO: 109 - DNA, 564 bp, synthetic construct
  misc_feature 1..564: note = Synthetic: DAP10.17.2 (-sig domain, -FP2A, -CD19) Nucleic acid
  source 1..564: mol_type = other DNA
cagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggccgagtgaagttcagtagaagcgccgacgcccctgcctaccagcagggccagaaccagctgtacaacgagctgaacctgggcagacgcgaggaatacgacgtgctggacaagcggcggggcagagatcctgagatgggcggaaaacctcaaagaagaaagaacccccaggagggcctgttcaacgagctgcagaaagataagatggccgaggccttcagcgagatcggcatgaaaggcgagagacgacgtggaaagggccacgacggcttgtttcagggcctgagcaccgccacaaaggacaccttcgacgccctgcacatgcaggccctcccccccaga

SEQ ID NO: 110 - protein, 590 aa, synthetic construct; identical to SEQ ID NO: 87
  REGION 1..590: note = Synthetic: DAP10.17.3 (+sig domain, +FP2A, +EGFRt) Amino Acid

SEQ ID NO: 111 - DNA, 1776 bp, synthetic construct; identical to SEQ ID NO: 88
  misc_feature 1..1776: note = Synthetic: DAP10.17.3 (+sig domain, +FP2A, +EGFRt) Nucleic Acid
  source 1..1776: mol_type = other DNA

SEQ ID NO: 112 - DNA, 1809 bp, synthetic construct
  misc_feature 1..1809: note = Synthetic: 3H7-5.1-P2A-DAP10.1 Nucleic Acid
  source 1..1809: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctgctgcctgtggccgccgctcagaccacccctggcgagagaagcagcctgcccgccttctaccccgggaccagcggatcttgtagcggctgcggcagcttgtctctgcctctgctcgccggcctggtggctgctgatgccgtggccagcctgctgatcgtgggcgccgtctttctgtgcgccagacctagaaggtcccctgcccaggaggacggcaaggtgttcatcaacatgcctggaagaggctga

SEQ ID NO: 113 - protein, 602 aa, synthetic construct
  REGION 1..602: note = Synthetic: 3H7-5.1-P2A-DAP10.1 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVFINMPGRG

SEQ ID NO: 114 - DNA, 1809 bp, synthetic construct
  misc_feature 1..1809: note = Synthetic: 3H7-5.1-P2A-DAP10.2 Nucleic Acid
  source 1..1809: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctgctccccgtggccgccgctcagaccacccctggagaaagaagcagcctgcctgccttctaccccgggaccagcggctcatgcagcggttgtggcagcctgagcctgcctctgctggccggcctggtggctgctgatgccgtggcatctctgctgatcgtgggcgccgtctttctgtgcgccagacctagacggagccccgcccaggaggacggcagagtgttcatcaacatgcctggcagaggctga

SEQ ID NO: 115 - protein, 602 aa, synthetic construct
  REGION 1..602: note = Synthetic: 3H7-5.1-P2A-DAP10.2 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGRVFINMPGRG

SEQ ID NO: 116 - DNA, 2148 bp, synthetic construct
  misc_feature 1..2148: note = Synthetic: 3H7-5.1-P2A-DAP10.3 Nucleic Acid
  source 1..2148: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctgctgcctgtggccgctgcccagaccacccctggagaaagatctagccttccagccttctaccccgggaccagcggaagctgcagcggctgcggcagcttgtctctgcctctgctggccggcctggtggccgccgacgctgttgcatctctgctgatcgtgggcgccgtctttctgtgcgccagacccagacggagccctgctcaggaagatggcaaagtgtacatcaacatgcctggcagaggcagagtgaagttcagcagaagcgccgatgcccctgcctatcagcagggccagaaccagctgtacaacgagctgaacctgggcagacgcgaggaatacgacgtgctggacaagcggcggggacgggaccccgagatgggcggcaagcctcaaagaaggaagaacccccaggagggcctgtataatgagctgcagaaagataagatggccgaggcctacagcgagatcggcatgaaaggcgagagacggcggggcaagggccacgacggcctgtaccagggcctgagcaccgccacaaaggacacctacgacgccctgcacatgcaggccctgccacctagatga

SEQ ID NO: 117 - protein, 715 aa, synthetic construct
  REGION 1..715: note = Synthetic: 3H7-5.1-P2A-DAP10.3 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 118 - DNA, 2274 bp, synthetic construct
  misc_feature 1..2274: note = Synthetic: 3H7-5.1-P2A-DAP10.4 Nucleic Acid
  source 1..2274: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctgctgcctgtggccgccgctcaaacaacccctggcgagagatctagcctgccagccttctaccccggtacaagcggatcttgcagcggctgcggcagcctgtctctgcctctgctggccggcctggtggctgctgatgctgtggccagcctgctgatcgtgggcgccgtgttcctgtgcgccagacctagaaggtcccctgcccaggaggatggaaaggtgtacatcaacatgcctggcagaggcaagagaggcagaaagaagctgctgtacatcttcaagcagcctttcatgagaccagtgcagaccacccaggaggaggacggatgtagctgcagattccccgaggaagaagaaggcggctgtgaactgagagtgaagttcagcagaagcgccgacgcccctgcttatcagcagggccagaaccagctgtacaacgagctgaacctgggcagaagggaagagtacgacgtgctggacaagcggcggggacgggaccccgagatgggcggaaaacctcaaagacggaagaacccccaggagggcctttataatgagctgcagaaagataagatggccgaggcctacagcgagatcggcatgaaaggagaaagacggcggggcaaaggccacgacggcctgtaccagggcctgagcaccgccacaaaggacacctacgacgccctgcacatgcaggccctgccccccagatga

SEQ ID NO: 119 - protein, 757 aa, synthetic construct
  REGION 1..757: note = Synthetic: 3H7-5.1-P2A-DAP10.4 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 120 - DNA, 2274 bp, synthetic construct
  misc_feature 1..2274: note = Synthetic: 3H7-5.1-P2A-DAP10.5 Nucleic Acid
  source 1..2274: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggacacatcctgttcctgctgctgcttccagtggccgccgctcaaacaacccctggcgagagaagcagcctgcccgccttctaccccggtacatctggcagctgcagcggctgcggcagcttgtctctgcctctgctggccggactggtggctgctgatgccgtggccagcctgctgatcgtgggcgccgtgttcctgtgcgccagacccagaaggtcccctgcccaggaggacggcaaggtgttcatcaacatgcctggcagaggcaaaagaggcagaaagaagctgctgtacatcttcaagcagcctttcatgcggcctgtgcagaccacccaggaggaagatggctgctcttgtcgatttccagaagaggaagaaggcggctgtgaactgagagtgaagttcagcagatccgccgacgcccctgcttatcagcagggccagaaccagctgtataatgagctgaacctgggccggcgggaagagtacgacgtgctggacaagcggcggggcagagatcctgagatgggcggaaaacctcaaagaaggaagaacccccaggagggactgtacaacgagctgcagaaggataagatggccgaggcctacagcgagatcggcatgaaaggagaaagaagaagaggaaaaggccacgacggcctgtaccagggcctgagcaccgccaccaaggacacctacgacgccctgcacatgcaggccctgccacctagatga

SEQ ID NO: 121 - protein, 757 aa, synthetic construct
  REGION 1..757: note = Synthetic: 3H7-5.1-P2A-DAP10.5 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 122 - DNA, 2274 bp, synthetic construct
  misc_feature 1..2274: note = Synthetic: 3H7-5.1-P2A-DAP10.6 Nucleic Acid
  source 1..2274: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctcctgcctgtggccgctgctcagaccacccctggagaaagatctagcctgcccgctttctaccccgggaccagcggcagctgcagcggatgcggctctctgagcctgcctctgctggccggcctcgtggccgccgatgctgtggccagcctgctgatcgtgggcgccgtgttcctgtgcgccagacccagaagaagccctgcccaggaggatggcagagtgttcatcaacatgcctggcagaggcaaaagaggcagaaagaagctgctgtacatcttcaagcagcctttcatgcggcccgtgcagacaacccaggaggaggacggctgtagctgtagattccccgaggaagaagaaggcggctgcgagcttagagtgaagttcagcagaagcgccgacgcccctgcttaccagcagggccagaaccagctgtataatgagctgaacctgggaagaagggaagagtacgacgtgctggacaagcggcggggcagagatcctgagatgggcggaaaacctcaaagaaggaagaaccctcaggagggcctgtacaacgagctgcagaaagacaagatggccgaggcctacagcgagatcggcatgaaaggcgagagacggcggggcaagggccacgacggcctgtaccaaggcctgtcaacagccaccaaggacacctacgacgccctgcacatgcaggccctgccacctagatga

SEQ ID NO: 123 - protein, 757 aa, synthetic construct
  REGION 1..757: note = Synthetic: 3H7-5.1-P2A-DAP10.6 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 124 - DNA, 1935 bp, synthetic construct
  misc_feature 1..1935: note = Synthetic: 3H7-5.1-P2A-DAP10.7 Nucleic Acid
  source 1..1935: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctgctgcctgtggccgctgctcagaccacacctggcgagagaagcagcctgcccgccttctaccccggtacaagcggctcttgttctggctgcggcagcctgagcctgcctctgctcgccggcctggtggccgccgacgccgtggcatctctgctgatcgtgggcgccgtgttcctgtgcgccagacctagacggagccctgcccaggaggacggcaaagtgtacatcaacatgcctggaagaggcaagcggggcagaaagaagctgctgtacatcttcaagcagcctttcatgagacccgtgcagaccacccaggaggaagatggctgtagctgcagattccccgaggaagaagagggcggatgcgagctgtga

SEQ ID NO: 125 - protein, 644 aa, synthetic construct
  REGION 1..644: note = Synthetic: 3H7-5.1-P2A-DAP10.7 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRGKRGRKKLLYIFKQPFMRPVQTTQEEDGCSCRFPEEEEGGCEL

SEQ ID NO: 126 - DNA, 1935 bp, synthetic construct
  misc_feature 1..1935: note = Synthetic: 3H7-5.1-P2A-DAP10.8 Nucleic Acid
  source 1..1935: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggacacatcctgttcctgctgctgctgcctgtggccgctgctcagaccacccctggcgagagaagcagcctgcccgccttctaccccggcaccagcggatcttgtagcggctgcggcagcctgagcctgcctctgctcgccggcctggtggccgccgacgccgtggcatctctgctgatcgtgggcgccgtctttctgtgcgccagacctagacggagccctgcccaggaggacggcaaagtgttcatcaacatgcctggaagaggcaagcggggcagaaagaagctgctgtacatcttcaagcagcctttcatgagacccgtgcagacaacccaggaggaagatggctgcagctgcagattccccgaggaagaggaaggcggctgtgaactgtga

SEQ ID NO: 127 - protein, 644 aa, synthetic construct
  REGION 1..644: note = Synthetic: 3H7-5.1-P2A-DAP10.8 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVFINMPGRGKRGRKKLLYIFKQPFMRPVQTTQEEDGCSCRFPEEEEGGCEL

SEQ ID NO: 128 - DNA, 1935 bp, synthetic construct
  misc_feature 1..1935: note = Synthetic: 3H7-5.1-P2A-DAP10.9 Nucleic Acid
  source 1..1935: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgttcctgctgctgctgcctgtggctgccgctcagacaacccctggcgagagaagcagcctgcccgccttctaccccgggaccagcggctcttgttctggctgcggcagcctgagcctgcctctgctcgccggcctggtggccgccgacgccgtggccagcctgctgatcgtgggcgccgtgttcctgtgcgccagacctagaaggtcccctgcccaggaggacggcagagtgttcatcaacatgcctggcagaggcaagcggggcagaaagaagctgctgtacatcttcaagcagcctttcatgcggcctgtgcagaccacacaggaggaagatggctgcagctgcagattccccgaggaagaggaaggcggatgtgaactgtga

SEQ ID NO: 129 - protein, 644 aa, synthetic construct
  REGION 1..644: note = Synthetic: 3H7-5.1-P2A-DAP10.9 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGRVFINMPGRGKRGRKKLLYIFKQPFMRPVQTTQEEDGCSCRFPEEEEGGCEL

SEQ ID NO: 130 - DNA, 1809 bp, synthetic construct
  misc_feature 1..1809: note = Synthetic: 3H7-5.1-P2A-DAP10.0 Nucleic Acid
  source 1..1809: mol_type = other DNA
atgtccgtgcctacccaggtgctgggcctgctgctgctgtggctgaccgacgccagatgcgaaatagtgatgacgcagtctccagccaccctgtctgtgtctccaggggaaagaaccaccctctcctgcagggccagtcagagtgttagcagcaacttagcctggtaccttcagaaacctggccaggctcccaggctcctcatctatggtgcatccaccagggccactggtatcccagccaggttcagtggcagtgggtctgggacagagttcattctcaccatcagcagcctgcagtctgaagattttgcagtttattactgtcagcagtataataactggccgatcaccttcggccaagggacacggctggagattaaaggtggaggtggatctggaggaggaggatccggtggaggaggtgaagtgcaactggtggagtctgggggaggcttggtacagcctggcaggtccctgagactctcctgtgcagcctctggattcaccttttatgattatgccatgcactgggtccggcaagctccagggaagggcctggagtgggtctcaggtattagttggaatagtggttacataggctatgcggactctgtgaagggccgattcaccatctccagagacaacgccaagaactccctgtatctgcaaatgaacagtctgagagctgaggacacggccttgtattactgtgcaaaagataacagctatggaaagttctactacggtttggacgtctggggccaagggaccacggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggggctacgaacttctcccttcttaaacaagcgggagacgtggaagaaaatcccggacctatgatccacctgggccacatcctgtttctgctgctgcttcctgtggccgctgctcagacaacacctggcgagagatctagcctgcctgccttctatcctggcaccagcggctcttgttctggctgtggatctctgagcctgcctctgctggctggactggttgctgctgatgctgtggcctctctgctgattgtgggcgccgtgttcctgtgtgcccggcctagaagatctcccgctcaagaggatggcaaggtgtacatcaacatgcccggcagaggatga

SEQ ID NO: 131 - protein, 602 aa, synthetic construct
  REGION 1..602: note = Synthetic: 3H7-5.1-P2A-DAP10.0 Protein
MSVPTQVLGLLLLWLTDARCEIVMTQSPATLSVSPGERTTLSCRASQSVSSNLAWYLQKPGQAPRLLIYGASTRATGIPARFSGSGSGTEFILTISSLQSEDFAVYYCQQYNNWPITFGQGTRLEIKGGGGSGGGGSGGGGEVQLVESGGGLVQPGRSLRLSCAASGFTFYDYAMHWVRQAPGKGLEWVSGISWNSGYIGYADSVKGRFTISRDNAKNSLYLQMNSLRAEDTALYYCAKDNSYGKFYYGLDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMIHLGHILFLLLLPVAAAQTTPGERSSLPAFYPGTSGSCSGCGSLSLPLLAGLVAADAVASLLIVGAVFLCARPRRSPAQEDGKVYINMPGRG

(19) United States
(12) Patent Application Publication
(10) Pub. No.: US 2025/0345429 A1
(43) Pub. Date: Nov. 13, 2025

(54) COMPOSITIONS AND METHODS COMPRISING CHIMERIC ADAPTOR POLYPEPTIDES

(71) Applicant: ADICET THERAPEUTICS, INC., Redwood City, CA (US)

(72) Inventors: Blake T. Aftab, Thousand Oaks, CA (US); Marissa Herrman, Newark, CA (US); Jason Romero, East Palo Alto, CA (US); Daulet Satpayev, Redwood City, CA (US); Stewart Abbot, San Diego, CA (US); Arun Bhat, Seattle, WA (US); Jonathan Wong, San Francisco, CA (US)

(21) Appl. No.: 18/870,353

(22) PCT Filed: May 31, 2023
(86) PCT No.: PCT/US2023/024073

§ 371 Date: Nov. 27, 2024

Related U.S. Application Data
(60) Provisional application No. 63/347,194, filed on May 31, 2022.

(51) Int. Cl. A61K 40/30; A61K 40/11; A61K 40/31; A61K 40/35; C07K 14/705; C07K 14/725; C12N 5/0783

(52) U.S. Cl. CPC A61K 40/30; A61K 40/11; A61K 40/31; A61K 40/35; C07K 14/7051; C07K 14/70521; C07K 14/70578; C12N 5/0636; A61K 2239/22; A61K 2239/24; C07K 2319/02; C07K 2319/03; C12N 2510/00

(57) ABSTRACT

Aspects of the disclosure include compositions and methods for treatment of a wide variety of diseases/conditions with engineered host cells, where the engineered host cells comprise a chimeric adaptor (CAD) polypeptide comprising DAP10 and at least one chimeric receptor. The CAD polypeptide may comprise substitution mutations and/or additional protein domains that function in conjunction with associated receptors to enhance cell survival and proliferation of the host cells, and to enhance cell killing activities of non-host cells.

### CROSS-REFERENCE TO RELATED APPLICATION

This application claims the benefit of priority to U.S. Provisional Application Ser. No. 63/347,194 filed on May 31, 2022, the disclosure of which is expressly incorporated by reference herein in its entirety.

### FIELD OF DISCLOSURE

The present disclosure relates generally to cellular immunotherapy, and particularly to compositions and methods comprising mammalian cells including at least one chimeric adaptor polypeptide and one or more chimeric receptors, for improved cell survival, proliferation, signaling, and the like.

### BACKGROUND OF THE DISCLOSURE

Adoptive cellular therapy has undergone near constant iteration for more than thirty years, from early days focusing on basic lymphokine activation and/or tumor infiltration to more recent strategies engineering immune cells to express genetically engineered antigen receptors, such as chimeric antigen receptors (CARs). However, while there have been some hints and indications of curative potential of various approaches along the way, there are a myriad of issues that remain.

One issue relates to the issue of low or lost (i.e., antigen escape) expression of a target of an adoptive cellular therapy. Specifically, a common mechanism of resistance to adoptive (or even innate) cellular therapies is the emergence of cell types (e.g., tumor) with loss or downregulation of the target antigen. Such loss or downregulation can lead to a reduction in efficacy of an adoptive (or innate) cellular response (Majzner R G and Mackall, CL. (2018) Cancer Discovery, 8(10): 1219-26).

Another related issue is the downregulation of naturally-occurring receptor(s) capable to recognize ligands specifically present on cells associated with a particular disease, which also can lead to ineffective cellular responses to various disease conditions. As a representative example, NKG2D is an activating immune receptor found on natural killer (NK) cells, CD8+αβ T cells, and γδ T cells in humans that regulates both innate and adoptive immune responses. The natural ligands of NKG2D include MICA and MICB and several UL16-binding proteins (Bauer S, et al. (1999) Science, vol. 285 5428: 727-729; Burgess S. J., et al. (2008) Immunol Res, 2008, 40(1):18-34). In humans, NKG2D ligands are not expressed on normal cells, but are widely expressed at varying levels on transformed or virally infected cancer cells (see e.g., Bauer S, et al. (1999) Science, vol. 285 5428: 727-729; Burgess S. J., et al. (2008) Immunol Res, 40(1):18-34; Baugh R, et al. (2020) Cancers 12(12): 3827). Expression of NKG2D ligands on a tumor cell surface sensitizes tumor cells to immune cell-mediated destruction by engaging NKG2D to activate NK cells and costimulate effector T cells. Therefore, NKG2D receptor and its ligands are a target of interest for cancer immunotherapy.

Unfortunately, however, NKG2D can be downregulated at times when it is most needed. For example, tumor-derived tumor growth factor-β (TGF-β) can downregulate NKG2D thereby reducing tumor cell killing by NK and CD8⁺ cells (see e.g., Crane, C., et al. (2010) Neuro-Oncology, 12(1): 7-13, and Dasgupta, S., et al. (2005) Journal of Immunol, 175: 5541-50). This, in turn, is associated with poor prognosis for the treatment of tumors.

NKG2D is mentioned as an example to demonstrate that there is a need in the art for compositions and methods that can inter alia improve, e.g., immune cell survival and proliferation, prevent receptor downregulation, and compensate for immune escape of antigens that are, for example, the target of an adoptive immunotherapy approach. Such compositions and methods would improve the prognosis for patients undergoing adoptive immunotherapy.

### SUMMARY OF DISCLOSURE

The present invention addresses the foregoing shortcomings in the prior art with mammalian cells comprising both a chimeric adaptor (CAD) polypeptide comprising human DAP10 and at least one chimeric receptor, optionally wherein the CAD polypeptide associates with the at least one chimeric receptor, and methods of using same. As articulated and demonstrated herein for the first time, the subject CAD constructs and polypeptides can improve the stability of receptors (e.g., chimeric and/or non-chimeric) capable of recognizing target antigens on various cell surfaces, promote a favorable balance of cell signaling pathway(s) upon receptor-target engagement, and/or improve functional properties (e.g., enhanced cytolytic, proliferative, survival and/or costimulatory properties) elicited upon engagement with various ligands of the at least one chimeric receptor, and optionally, engagement of various ligands of non-chimeric receptor(s) that associate with the CAD polypeptide (e.g., NKG2D).

In one aspect, the invention provides a mammalian cell comprising a chimeric adapter (CAD) polypeptide comprising a DAP10 domain, at least one of a costimulatory domain and/or an intracellular signaling domain, and specifically lacking an ectodomain comprising a functional extracellular receptor and/or ligand-binding domain, wherein the mammalian cell further comprises at least one chimeric receptor comprising an extracellular targeting domain that specifically binds to target antigens on a target cell. In preferred embodiments, the DAP10 domain comprises a human DAP10 amino acid sequence.

In embodiments, the chimeric receptor comprises at least one of an intracellular signaling domain and/or a costimulatory domain.

In embodiments, the chimeric receptor associates with DAP10, and comprises at least one DAP10-interacting domain. In embodiments, the DAP10-interacting domain comprises the amino acid sequence set forth in SEQ ID NO: 75, or an amino acid sequence comprising at least 80%, 90%, or 95% sequence identity to SEQ ID NO: 75.

In embodiments, the target antigen on the target cell is selected from the group consisting of CD20, TyrD, B7H6, CD3, CD19; CD123; CD22; CD30; CD70, CD171; CD6, CS-1 (also referred to as CD2 subset 1, Claudin 18.2, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44, CD44v6; CD44v7/8; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); IL,-11Ralpha; Mesothelin; Interleukin 11 receptor alpha (IL-URa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); ErbB3, ErbB4, Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor); IGF-II receptor; carbonic anhydrase IX (CAIX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl G M1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); melanoma-associated antigen; o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD 179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); MAGE-A4; M-AGE-A9; ETS translocation-variant gene 6, located on chromosome 12p (ETV6-AML); sperm protein 17 (SPA17); X Antigen Family, Member IA (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); 5T4, 8H9, ALCAM, B7-1 (CD80), B7-2 (CD86), B7-H4, B7-H6, B-human chorionic gonadotropin, CA-9, CA-125, CD133, CD138, CD23, CD25, CD34, CD4, CD40, CD56, CD8, c-Met, CSPG4, CMV-specific antigen, cytotoxic T-lymphocyte-associated antigen 4 (CTLA-4), DLL3, disialoganglioside GD2, GD3, ductal-epithelial mucine, EBV-specific antigen, EGP-2, EGP-40, endoglin, epithelial tumor antigen, fetal acetylcholine receptor, FBP, folate binding protein, folate receptor-alpha, glioma-associated antigen, glycosphingolipids, gp36, G250/CAIX, HBV-specific antigen, HCV-specific antigen, HER1-HER2, HER2-HER3 in combination, HERV-K, HIV-1 envelope glycoprotein gp41, HPV-specific antigen, KDR, kappa chain, insulin growth factor (IGF1)-l, ligands belonging to the MIC (MICA and MICB) and ULBP (ULBP1-ULBP6) families in humans, LAGA-la, Lewis Y, lambda chain, lectin-reactive AFP, LI-cell adhesion molecule, MAGE, major histocompatibility complex (MHC) molecule, major histocompatibility complex (MHC) molecule presenting a tumor-specific peptide epitope, M-CSF, MN-CA IX, MUC-1, MUC-16, NKG2D, NKG2D ligands, neutrophil elastase, Nkp30, oncofetal antigen (hST4) p53, prostate specific antigen (PSA), PSC1, prostate-specific antigen protein, STEAP1, STEAP2, surface adhesion molecule, surviving and telomerase, TA-72, the extra domain A (FDA) and extra domain B (EDB) of fibronectin and the Al domain of tenascin-C(TnC Al), thyroglobulin, Tem8, tumor stromal antigens, VEGF-A, and immunoglobulin lambda-like polypeptide 1 (IGLL1).

In embodiments, the at least one costimulatory domain of the CAD polypeptide, and optionally, the at least one costimulatory domain of the chimeric receptor, is selected from TLR1, TLR2, TLR3, TLR4, TLR5, TLR6, TLR7, TLR8, TLR9, TLR10, CARD 1l, CD2, CD3C, CD7, CD27, CD28, CD30, CD40, CD54 (ICAM), CD70, CD80, CD83, CD86, CD134 (OX40), CD137 (4-1BB), CD278 (ICOS), FcR, LAT, NKD2C, SLP76, TRIM, and ZAP70, or combinations thereof. In preferred embodiments, the at least one costimulatory domain of the CAD polypeptide is 4-1BB, or CD28.

In embodiments, the CAD polypeptide comprises at least one intracellular signaling domain. In embodiments, the at least one intracellular signaling domain of the CAD polypeptide, and optionally, the at least one intracellular signaling domain of the chimeric receptor, is selected from CD3ζ, DAP12, LFA-1, and repeat (2-5) DAP10 YINM motifs. In preferred embodiments, the at least one intracellular signaling domain of the CAD polypeptide is CD3ζ.

In embodiments, the at least one costimulatory domain of the CAD polypeptide is 4-1BB, and the at least one intracellular signaling domain of the CAD polypeptide is CD3ζ. In preferred embodiments, the CAD polypeptide comprises, from N-terminus to C-terminus, the DAP10 domain, the 4-1BB costimulatory domain followed by the CD3ζ intracellular signaling domain.

In embodiments, the least one costimulatory domain of the CAD polypeptide is CD28, and the at least one intracellular signaling domain of the CAD polypeptide is CD3ζ. In preferred embodiments, the CAD polypeptide comprises, from N-terminus to C-terminus, the DAP10 domain, the CD28 costimulatory domain followed by the CD3ζ intracellular signaling domain.

In embodiments, the CAD polypeptide comprises a 4-1BB costimulatory domain and a CD28 costimulatory domain. In embodiments, the CAD polypeptide comprises, from N-terminus to C-terminus, the DAP10 domain, the 4-1BB costimulatory domain followed by the CD28 costimulatory domain, followed in turn by a CD3ζ intracellular signaling domain. In another embodiment, the CAD polypeptide comprises, from N-terminus to C-terminus, the DAP10 domain, the CD28 costimulatory domain followed by the 4-1BB costimulatory domain, followed in turn by a CD3ζ signaling domain.

In embodiments, the human DAP10 amino acid sequence comprises an amino acid sequence having at least 90%, 95%, 97%, or 99% sequence identity to SEQ ID NO: 1. In embodiments, the human DAP10 amino acid sequence comprises an amino acid sequence having at least 90%, 95%, 97%, or 99% sequence identity to SEQ ID NO: 78. In embodiments, the human DAP10 amino acid sequence comprises an amino acid sequence having at least 90%, 95%, 97%, or 99% sequence identity to SEQ ID NO: 81.

In embodiments, the human DAP10 amino acid sequence comprises a mutated human DAP10 amino acid sequence. In some examples, the mutated human DAP10 amino acid sequence comprises amino acid substitutions at positions corresponding to K84 and/or Y86. In an embodiment, the amino acid substitution at position K84 comprises a K84R substitution. In an embodiment, the amino acid substitution at Y86 comprises a Y86F substitution.

In embodiments, the CAD polypeptide is encoded by an isolated nucleic acid that is operably linked to a regulatable promoter, and the chimeric receptor is encoded by an isolated nucleic acid that is operably linked to a regulatable promoter. In one embodiment, the regulatable promoter operably linked to the nucleic acid that encodes the CAD polypeptide is different than the regulatable promoter that is operably linked to the nucleic acid that encodes the chimeric receptor. In one embodiment, the regulatable promoter operably linked to the nucleic acid that encodes the CAD polypeptide is the same as the regulatable promoter that is operably linked to the nucleic acid that encodes the chimeric receptor.

In embodiments, the isolated nucleic acid that encodes the CAD polypeptide comprises a nucleic acid sequence set forth in SEQ ID NO: 60, or SEQ ID NO: 62, or SEQ ID NO: 64, or SEQ ID NO: 66, or SEQ ID NO: 68, or SEQ ID NO: 70, or SEQ ID NO: 72.

In embodiments, the isolated nucleic acid that encodes the CAD polypeptide comprises a signal peptide SEQ ID NO: 45 at its N-terminus. In embodiments, the isolated nucleic acid that encodes the CAD polypeptide comprises a signal peptide SEQ ID NO: 93 at its N-terminus.

In embodiments, the isolated nucleic acid that encodes the CAD polypeptide encodes for a cytokine. In additional or alternative embodiments, the isolated nucleic acid that encodes the chimeric receptor encodes for a cytokine. In embodiments where both the isolated nucleic acid that encodes the CAD polypeptide and the isolated nucleic acid that encodes the chimeric receptor encode cytokines, the cytokines can be different. In embodiments, the cytokine(s) are selected from the group consisting of IL-2, IL-4, IL-7, IL-15, IL-21, and IL-23. In embodiments, the isolated nucleic acid further encodes a marker protein. In embodiments, the isolated nucleic acid further encodes a marker protein. In embodiments, the marker protein is selected from the group consisting of truncated CD19, CD20 (Rituxumab recognition domain), truncated EGFR, and LNGFR.

In embodiments, wherein when the chimeric receptor comprises an intracellular signaling domain, said CAD polypeptide comprises a costimulatory domain, and vice-versa.

In embodiments, the mammalian cell further comprises at least one receptor that associates with DAP10, wherein said at least one receptor is not the at least one chimeric receptor. In embodiments, the at least one receptor that associates with DAP10 is endogenous. In embodiments, the at least one receptor that associates with DAP10 is exogenous. In embodiments, that at least one receptor that associates with DAP10 is over-expressed. In particular embodiments, the at least one receptor is selected from NKG2D, Ly49H, Ly49D, Sirp-b1, Siglec-15, and Cd3001b. In a preferred embodiment, the at least one receptor is NKG2D.

In embodiments, the mammalian cell is an immune cell, preferably wherein said immune cell is a cytotoxic cell.

In embodiments, a mammalian cell comprising both a CAD polypeptide and at least one chimeric receptor as herein disclosed exhibits in vitro and/or in vivo killing activity against a target cell that exhibits cell surface expression of target antigens recognized by at least one chimeric receptor.

In embodiments, the target cell is a hematological tumor cell.

In embodiments, the target cell is a solid tumor cell.

In embodiments, the in vitro and/or in vivo killing activity is greater than an innate level of in vitro and/or in vivo killing activity in a control mammalian cell that lacks expression of one or both of the at least one chimeric receptor and/or the CAD polypeptide.

In embodiments, the mammalian cell proliferates in response to contact with the target cell. In embodiments, the mammalian cell exhibits increased proliferation in response to contact with the target cell as compared to a control mammalian cell that lacks expression of one or both of the chimeric receptor and/or the CAD polypeptide. In embodiments, the mammalian cell proliferates in a host organism that comprises the target cell.

In embodiments, the mammalian cell expresses pro-inflammatory cytokines in response to contact with the target cell. In embodiments the pro-inflammatory cytokines comprise tumor necrosis factor alpha or interferon gamma.

In one aspect, the invention provides for a plurality of mammalian cells comprising a CAD polypeptide and at least one chimeric receptor, as herein disclosed. In embodiments, the plurality of mammalian cells comprises at least about 10⁶ cells, at least 10⁷ cells, or at least 10⁸ cells, preferably from about 10⁸ to 10¹¹ cells.

In one aspect, the invention provides a method of making a mammalian cell as herein disclosed comprising transfecting the mammalian cell(s) with a construct comprising an isolated nucleic acid that encodes for a CAD polypeptide, and at least one isolated nucleic acid construct that encodes for at least one chimeric receptor.

In embodiments, the method comprises retroviral transduction.

In embodiments, the method comprises ex vivo expansion of the mammalian cell(s), wherein the ex vivo expansion is performed before transfection and/or after transfection of the isolated nucleic acid that encodes for the CAD polypeptide and at least one construct that encodes for the at least one chimeric receptor.

In one aspect, the invention provides a pharmaceutical composition comprising a pharmaceutically acceptable excipient and a mammalian cell comprising a CAD polypeptide and at least one chimeric receptor as herein disclosed, preferably a plurality of the mammalian cells, more preferably at least about 10⁶ cells, at least 10⁷ cells, or at least 10⁸ cells, still more preferably from about 10⁸ to 10¹¹ cells.

In one aspect, the invention provides a method of activating a mammalian cell comprising a CAD polypeptide and at least one chimeric receptor as herein disclosed, or a plurality of said mammalian cells, comprising contacting one or more target cell(s) with the mammalian cell(s). In embodiments, said mammalian cell(s) are introduced into a subject in need thereof, wherein said activating occurs in the subject.

In one aspect, the invention provides a use of a mammalian cell comprising a CAD polypeptide and at least one chimeric receptor as herein disclosed, or a plurality of said mammalian cells, or a pharmaceutical composition comprising said mammalian cell(s), in the preparation of a medicament for treating a subject with a condition for which the mammalian cell(s) reduces at least one symptom or sign of said condition in the subject.

In one aspect, the invention provides a use of a tumor cell killing effective amount of a mammalian cell comprising a CAD polypeptide and at least one chimeric receptor as herein disclosed, or a plurality of said mammalian cells, or a pharmaceutical composition comprising said mammalian cell(s), in the preparation of a medicament for the treatment of cancer in a subject in need thereof.

In one aspect, the invention provides a method of killing a tumor cell, the method comprising contacting the tumor cell with a tumor cell killing effective amount of a mammalian cell comprising a CAD polypeptide and at least one chimeric receptor as herein disclosed, or a plurality of said mammalian cells, or a pharmaceutical composition comprising said mammalian cell(s). In embodiments, the method comprises introducing a therapeutically effective amount of the mammalian cell(s) or the pharmaceutical composition into a host organism comprising the tumor cell. In embodiments, the method comprises introducing into the host organism comprising the tumor cell the therapeutically effective amount of the mammalian cell(s) or the pharmaceutical composition and simultaneously or sequentially administering one or more methods to elevate common chain gamma chain cytokine(s).

In embodiments, administering one or more methods to elevate common gamma chain cytokine(s) comprises administering simultaneously with introducing the mammalian cell(s) or sequentially an amount of common gamma chain cytokine(s) effective to increase proliferation, cytotoxic activity, persistence, or the combination thereof of the introduced mammalian cell(s), preferably wherein the method comprises administering TL-2, more preferably wherein the method comprises administering IW-15.

In embodiments, the one or more methods to elevate common gamma chain cytokine(s) comprise administering an amount of common gamma chain cytokine(s) effective to increase proliferation, cytotoxic activity, persistence, or the combination thereof of the introduced mammalian cell(s) before and/or after introducing the mammalian cell(s).

In embodiments, the one or more methods to elevate common gamma chain cytokine(s) comprises lymphodepletion before introducing the mammalian cell(s).

In embodiments, the one or more methods to elevate common gamma chain cytokine(s) comprises secretion of one or more common gamma chain cytokine(s) from the introduced mammalian cell(s).

In embodiments, the method reduces the in vivo tumor burden in the host organism, and/or increases the mean survival time of the host organism as compared to a control organism, wherein the control organism is not treated with the mammalian cell(s) or the pharmaceutical composition.

Other features, objects, and advantages will be apparent from the disclosure that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1D are schematic depictions of exemplary chimeric DAP10 adaptor polypeptides of the present disclosure, in conjunction with a receptor with which they associate. In the depicted illustrations, the receptor is NKG2D. FIG. 1A depicts a chimeric DAP10 adaptor polypeptide with K84R and Y86F modifications; FIG. 1B depicts a chimeric DAP10 adaptor polypeptide with K84R and Y86F modifications, as well as a C-terminal fusion comprising CD3ζ signaling domain; FIG. 1C depicts a chimeric DAP10 adaptor polypeptide with K84R and Y86F modifications, as well as a C-terminal fusion comprising a 4-1BB costimulatory domain; and FIG. 1D depicts a chimeric DAP10 adaptor polypeptide with K84R and Y86F modifications, as well as a C-terminal fusion comprising both a 4-1BB costimulatory domain and a CD3ζ signaling domain.

FIGS. 2A-2C are graphs showing a cytotoxicity index of various chimeric DAP10 adaptor polypeptides of the present disclosure expressed in Vδ1 cells and tested against PLC/PRF/5 cells, as compared to tumor cells alone and a control chimeric antigen receptor (CAR) construct also expressed in Vδ1 cells.

FIGS. 2D-2F are plots showing extent of Vδ1 cell proliferation seen in the assays of FIGS. 2A-2C.

FIGS. 3A-3C are graphs showing a cytotoxicity index of various chimeric DAP10 adaptor polypeptides of the present disclosure expressed in Vδ1 cells and tested against HepG2 cells, as compared to tumor cells alone and a control CAR construct also expressed in Vδ1 cells.

FIGS. 3D-3F are plots showing extent of Vδ1 cell proliferation seen in the assays of FIGS. 3A-3C.

FIG. 4 is a graph showing survival of Vδ1 cells transduced with various chimeric DAP10 adaptor constructs of the present disclosure, following 5 days of co-culture with PLC/PRF/5 cells.

FIGS. 5A-5B are graphs illustrating robust in vivo tumor growth via Vδ1 cells transduced with select chimeric DAP10 adaptor polypeptides of the present disclosure.

FIG. 6 is a graph illustrating NKG2D expression level on Vδ1 cells transduced with various DAP10 adaptor polypeptides of the present disclosure and following co-culture of the transduced cells with PLC cells. The greatest NKG2D expression level was observed in cells transduced with a DAP10 adaptor polypeptide that included each of K84R, Y86F, a 4-1BB costimulatory domain and a CD3ζ signaling domain.

FIG. 7 is a western blot for CAD protein visualized by anti-DAP10 and anti-CD3ζ antibodies that illustrates that DAP10 CAD expression is similar across different lots of Vδ1 cells.

FIGS. 8A-8D are graphs illustrating that cytotoxic activity of DAP10 CADs is mediated by NKG2D. CAD+(FIGS. 8A-8B) or chimeric antigen receptor (CAR)+Vδ1 cells (FIGS. 8C-8D) were preincubated with various dilutions of either anti-NKG2D antibody (clone 1D11) or isotype control (1p g/ml-0.01 ng/ml) prior to co-culture with luciferase-labeled target cells (PLC/PRF/5 or HL60). Target cell killing was assessed after 18 hours by measuring luciferase signal. NKG2D-mediated cytotoxicity can be assessed by comparing % cytotoxicity with isotype pre-incubation to % cytotoxicity with NKG2D antibody pre-incubation.

FIGS. 9A-9C illustrate that DAP10 CADs of the present disclosure have consistent molecular activation signature. The data was obtained from Nanostring analysis post stimulation from multiple donors and cell lines.

FIGS. 10A-10G illustrate that Vδ1 cells transduced with DAP10 CADs of the present disclosure exhibit anti-cancer activity against various cancer types having a broad range of NKG2D ligand expression levels/patterns in an 18-hour assay. The graphs shown at FIGS. 10A-10G represent % cytotoxic activity of Vδ1 cells transduced with DAP10 CADs. DAP10 CAD+Vδ1 cells or controls were co-cultured with a variety of luciferase-expressing target cell lines across varying E:T ratios (1:6-10:1) in a short term 18-hour cytotoxicity assay.

FIG. 10H is a table showing that tested target cell lines comprise a broad range of NKG2D ligand (MICA/B, ULBP1, ULBP2/5/6, ULBP3, ULBP4) expression levels/patterns. Data is presented as fold change mean fluorescence intensity (MFI) of NKG2D ligand over relevant isotype control.

FIGS. 10I-10J are a series of graphs showing the raw data used to populate the table shown at FIG. 10H.

FIGS. 11A-11G illustrate that Vδ1 cells transduced with DAP10 CADs of the present disclosure exhibit anti-cancer activity against various cancer types having a broad range of NKG2D ligand expression levels/patterns in an 120-hour assay. FIGS. 11A-11G are graphs illustrating cytotoxicity index of Vδ1 cells transduced with DAP10 CADs, at different effector:target ratios, compared to controls. Tested target cell lines comprise a broad range of NKG2D ligand (MICA/B, ULBP1, ULBP2/5/6, ULBP3, ULBP4) expression levels/patterns (see FIG. 10H-10J).

FIGS. 12A-12B are graphs showing that cytotoxic activity of Vδ1 cells transduced with DAP10 CADs is comparable for different lots of Vδ1 cells and DAP10 CADs. A 120 hour cytotoxicity assay is shown at FIG. 12A, where target cells were PLC/PRF/5 cells. % reduction in cytotoxicity of tumor alone relative to treated using the final time point of the assay of FIG. 12A, is shown at FIG. 12B.

FIG. 12C is a graph showing a comparison of cytoxicity of Vδ1 cells transduced with either DAP10.6, DAP10.16, or DAP10.17 constructs. Data obtained for each construct is an aggregate of 3 donors. PLC/PRF/5 cells were used as target cells in a 120-hour cytotoxicity assay. Controls included DAP10.0, and PLC/PRF/5 alone. Using a stringent E:T ratio, in this particular assay DAP10.6 showed improved cytotoxicity as copared to DAP10.16 and DAP10.17.

FIGS. 12D-12F are graphs illustrating donor dependence of three different DAP10 constructs (DAP10.6, FIG. 12D; DAP10.16, FIG. 12E; DAP10.17, FIG. 12F) as measured by cytotoxicity index in a 120-hour co-culture cytotoxicity assay. The DAP10 constructs were transduced into Vδ1 cells, target cells were PLC/PRF/5 cells. Controls included untransduced Vδ1 cells from the same three different donors.

FIGS. 13A-13B illustrate that DAP10 CAD stimulation results in a polyfunctional cytokine profile, that is a function of different DAP10 CAD constructs and target cell type. A cytokine profile as a function of DAP10 CAD and target cell is depicted at FIG. 13A. FIG. 13B is a graph showing interferon gamma induction as a function of DAP10 CAD and target cell type.

FIGS. 13C-13F are plots showing levels of interferon gamma secretion from DAP10 CAD+Vδ1 cells alone (effector) and after co-culture with various target cells.

FIG. 14 illustrates cytokine profile of Vδ1 cells transduced with DAP10 CAD in presence or absence of target cell, as compared to cytokine profile of Vδ1 cells transduced with a chimeric antigen receptor (CAR).

FIG. 15 illustrates DAP10 CADs of the present disclosure drive proliferation of Vδ1 cells from multiple donors. FIG. 15 shows plots illustrating donor dependence on proliferation of Vδ1 cells obtained from two different donors (SCT29 and SCT46), transduced with either DAP10.6, DAP10.16, or DAP10.17, relying on a co-culture experiment with PLC/PRF/5 cells. Controls included prior tested Vδ1 cells obtained from a different donor (SCT06) and transduced with DAP10.6 (positive control), and Vδ1 cells from the two different donors (SCT29 and SCT46) transduced with DAP10.0 (negative control).

FIGS. 16A-16B illustrate in vivo tumor control in a mouse model for Vδ1 cells transduced with a DAP10 CAD. FIG. 16A is a graph comparing in vivo tumor control of DAP10.6 and a DAP10 CAD including a modified (“1XX”) CD3ζ intracellular signaling domain (DAP10.16). A schematic of the experimental procedure is depicted at FIG. 16B.

FIGS. 17A-C illustrate that anti-tumor activity of Vδ1 cells transduced with a DAP10 CAD of the present disclosure exhibits anti-tumor activity with kinetics similar to CAR Vδ1 cells. FIG. 17A is a graph showing comparison of in vivo tumor growth kinetics for DAP10 CAD+Vδ1 cells compared to CAR Vδ1 cells in an HCT-15 mouse xenograft model. FIG. 17B is a graph quantifying tumor volume at day 27. FIG. 17C is a schematic illustration of the experimental procedure used to obtain the data depicted at FIGS. 17A-17B.

FIGS. 18A-18D illustrate that Vδ1 cells transduced with a DAP10 CAD of the present disclosure proliferate in tumor tissue in vivo in a mouse model. FIG. 18A are flow cytometry plots illustrating that proliferation of Vδ1 cells transduced with a DAP10 CAD is specific to tumor tissue, and FIG. 18B is a graph showing that proliferation in tumor tissue progresses over 14 days. FIG. 18C is a graph showing quantification of Vδ1 cells in tumor tissue or other tissues taken 4, 7, and 14 days after treatment. The experimental procedure used to obtain the data depicted in FIGS. 18A-18C is schematically shown at FIG. 18D.

FIG. 19 illustrates that treatment of mice with Vδ1 cells transduced with a DAP10 CAD of the present disclosure is not associated with significant changes in body weight.

FIGS. 20A-20B illustrate that Vδ1 cells transduced with a DAP10 CAD of the present disclosure target tumor cells while sparing non-tumor cells. Vδ1 cells transduced with a DAP10 CAD of the present disclosure significantly reduced THP1 cell viability, compared to control as depicted in the graph of FIG. 20A, and do not target healthy PBMCs, as shown in the graph of FIG. 20B.

FIG. 21 is a graph showing Vδ1 cell fold-expansion in small scale shake flask expansions for six different donors. Fold-expansion was measured on day 14. The data compares Vδ1 cells transduced with DAP10.6 and DAP10.16 CADs.

FIGS. 22A-22C are graphs illustrating expansion kinetics of VS1 cells transduced with lead DAP10 CADs of the present disclosure (DAP10.6, DAP10.16, DAP10.17), as compared to controls. Data at FIG. 22A corresponds to Vδ1 cells obtained from a first donor (SCT06), data at FIG. 22B corresponds to Vδ1 cells obtained from a second donor (SCT29), and data at FIG. 22C corresponds to Vδ1 cells obtained from a third donor (SCT45). For each of FIGS. 22A-22C, % VS1 cells was measured as a function of expansion time (days).

FIG. 23A depicts a schematic representing a process for generating “off-the-shelf” allogeneic DAP10 CAD Vδ1 cells.

FIGS. 23B-23D are graphs showing independent expansions of VS1 cells transduced with preferred DAP10 CAD constructs of the present disclosure. Vδ1 cells used for the experiments depicted were obtained from three different donors (SCT06, SCT29, SCT45), respectively.

FIGS. 23E-23F are graphs illustrating that ex vivo culture of Vδ1 cells results in substantial fold expansion (FIG. 23E), and robust DAP10 CAD transduction (FIG. 23F). The data at FIG. 23E is represented as Vδ1 fold-expansion, and the data at FIG. 23F is represented as % DAP10 CAD of Vδ1 cells.

FIG. 23G depicts plots illustrating cellular composition of Vδ1 cells, Vδ2 cells, ap cells, and NK cells over time, expressed as % of culture.

FIGS. 24A-24C are graphs illustrating the results of re-stimulation assay of Vδ1 γδ cells co-expressing a CAR construct and various DAP10 CAD constructs using Raji cells (FIG. 24A), Mino cells (FIG. 24B), and MOLP-8 cells (FIG. 24C), respectively.

### DETAILED DESCRIPTION

The present invention provides mammalian cell(s), and compositions thereof, that comprise a chimeric adaptor (CAD) polypeptide comprising a DAP10 domain, at least one of a costimulatory domain and/or an intracellular signaling domain, and specifically lacking an ectodomain comprising a ligand-binding domain, wherein the mammalian cell(s) further express at least one chimeric receptor comprising an extracellular targeting domain that specifically binds to target antigens on a target cell. In embodiments, the at least one chimeric receptor comprises an extracellular targeting domain and a DAP10-interacting domain, optionally further comprising at least one costimulatory domain and/or at least one intracellular signaling domain. In embodiments, the CAD polypeptides of the subject invention may further comprise a transmembrane domain (e.g., SEQ ID NO: 79) and/or an extracellular domain (e.g., SEQ ID NO: 80 or other extracellular spacer domain), but will specifically lack a functional extracellular receptor and/or ligand-binding domain. In contrast, the prior art has typically employed DAP10 as a component of a CAR or NKG2D fusion chimera. See, e.g., Zhao et al., Oncolmmunology. 2019; 8(1): e1509173; Lynch et al., 2017, Immunol 152:472; US2020/0308248; WO/2018/183385; CN109096404; CN111995689. The CAD polypeptides and chimeric receptors of the subject invention are clearly different from that found in nature, generally comprising at least two polypeptide domains that are not naturally linked together, and optionally further including additional advantageous signaling domains and mutations as detailed herein.

The CAD polypeptides of the subject invention preferably include a DAP10 domain comprising human DAP10, optionally including one or more substitution mutations, deletion mutations, and/or addition mutations. For example, the DAP10 domain may have a Y86F mutation and/or a K84R mutation.

The “costimulatory domain” in the context of a CAD polypeptide, and in embodiments, a chimeric receptor, of the present disclosure enhances cell proliferation, cell survival and development of memory cells for cytotoxic cells that express the CAD polypeptide and/or the chimeric receptor. The CAD polypeptides and/or chimeric receptors of the invention may include one or more costimulatory domains selected from the costimulatory domains of proteins in the TNFR superfamily, CD28, CD137 (4-1BB), CD134 (OX40), Dap10, CD27, CD2, CD7, CD5, ICAM-1, LFA-1 (CD1 la/CD18), Lck, TNFR-I, PD-1, TNFR-II, Fas, CD30, CD40, ICOS LIGHT, NKG2C, B7-H3, or combinations thereof. If the CAD or chimeric receptor includes more than one costimulatory domain, these domains may be arranged in tandem, optionally separated by a linker. The costimulatory domain is an intracellular domain that may locate between the DAP10 domain and the optional intracellular signaling domain in the CAD.

In embodiments, the costimulatory domain includes a costimulatory domain of CD28, CD27, ICOS, 4-1BB, OX40, and CD40L. The term “costimulatory domain” as used herein also encompasses any modifications thereof, examples of which are described in US Patent Application No. 20200129554; US Patent Application No. 20200317777; WO2019010383; Li, W., et al., (2020) Immunity 53: 456-470; and Li, G., et al., (2017) J Immunol 198(1 Supplement): 198.4, the contents of each of which are incorporated herein in their entirety.

The “intracellular signaling domain” in the context of a CAD polypeptide, and in embodiments, a chimeric receptor, of the present disclosure transduces the effector function signal and directs the cytotoxic cell to perform its specialized function, i.e., harming and/or destroying the target cells. Examples of suitable intracellular signaling domains include, e.g., the ζ chain of the T cell receptor complex or any of its homologs, e.g., η chain, FcsRly and β chains, MB 1 (Iga) chain, B29 (Ig) chain, etc., human CD3 (chain, CD3 polypeptides (A, 6 and F), syk family tyrosine kinases (Syk, ZAP 70, etc.), src family tyrosine kinases (Lck, Fyn, Lyn, etc.) and other molecules involved in T cell transduction, such as CD2, CD5 and CD28. In embodiments, the intracellular signaling domain of a CAD polypeptide and/or chimeric receptor may be human CD³ζ chain, FcyRIII, FcsRI, cytoplasmic tails of Fc receptors, an immunoreceptor tyrosine-based activation motif (ITAM) bearing cytoplasmic receptors and combinations thereof.

The intracellular signaling domains may include intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (Park et al., “Are all chimeric antigen receptors created equal?” J Clin Oncol., vol. 33, pp. 651-653, 2015). Additional intracellular signaling domains include signaling domains used by NK and NKT cells (Hermanson, et al., “Utilizing chimeric antigen receptors to direct natural killer cell activity,” Front Immunol., vol. 6, p. 195, 2015) such as signaling domains of NKp30 (B7-H6) (Zhang et al., “An NKp30-based chimeric antigen receptor promotes T cell effector functions and antitumor efficacy in vivo,” J Immunol., vol. 189, pp. 2290-2299, 2012), and DAP12 (Topfer et al., “DAP12-based activating chimeric antigen receptor for NK cell tumor immunotherapy,” J Immunol., vol. 194, pp. 3201-3212, 2015), NKG2D, NKp44, NKp46, DAP10, and CD3z. Additionally intracellular signaling domains also includes signaling domains of human Immunoglobulin receptors that contain immunoreceptor tyrosine based activation motif (ITAM) such as FcgammaRI, FcgammaRIIA, FcgammaRlIC, FcgammaRIIIA, FcRL5 (Gillis et al., “Contribution of Human Fc.gamma.Rs to Disease with Evidence from Human Polymorphisms and Transgenic Animal Studies,” Front Immunol., vol. 5, p. 254, 2014).

In embodiments, the intracellular signaling domain includes a cytoplasmic signaling domain of TCR ζ, FcR γ, FcR β, CD3 γ, CD3 δ, CD3 ε, CD5, CD22, CD79a, CD79b, or CD66d. In exemplary embodiments the intracellular signaling domain in the CAD and/or chimeric receptor includes a cytoplasmic signaling domain of human CD3ζ. The term “intracellular signaling domain” as used herein also encompasses any modifications thereof, examples of which are described in US Patent Application No. 2020/0317777, as well as Combadiere, B., et al., (1996) J Exp Med 183(5): 2109-17; Lowin-Kropf B., et al., (1998) J Cell Biol 140(4): 861-871; Ardouin L., et al., (1999) Immunity 10(4): 409-20; Liu H. and Vignali D A A., (1999) J Immunol 163: 599-602; Kersh E N., et al., J Exp Med (1999) 190(11): 1627-36; Chae W J., et al., (2004) Int Immunol 16(9): 1225-36; Becker, A M., et al., (2007) J Immunol 178(7): 4120-8; Methi T., et al., (2007) Eur J Immunol 37(9): 2539-48; Baudouin S J., et al., (2008) Mol Biol Cell 19(6): 2444-56; Zhao Y., et al., (2009) J Immunol 183(9): 5563-74; Kochenderfer J N., et al., (2010) Blood 116(19): 3875-86; Bridgeman J S., et al., (2014) Clin Exp Immunol 175(2): 258-67; Long A H., et al., (2015) Nat Med 21(6): 581-90; Hwang S., et al., (2015) Nat Commun 6: 6982; WO2019126748; Feucht J., et al., (2019) Nat Med 25(1): 82-88; Roda-Navarro, P., and Reyburn, HT., (2009) J Biol Chem 284(24): 16463-16472; Giurisato, E., et al., (2007) Mol Cell Biol 27(24): 8583-8599; and Wu, J., et al., (2000) J Exp Med 192(7): 1059-1068, the contents of each of which are incorporated herein in their entirety.

In embodiments, two or more components of a CAD polypeptide and/or a chimeric receptor of the invention may be separated by one or more linkers. Linkers are oligo- or polypeptide regions of from about 1 to 100 amino acids in length. In some embodiments, the linkers may be, for example, 5-12 amino acids in length, 5-15 amino acids in length or 5 to 20 amino acids in length. Linkers may be composed of flexible residues like glycine and serine so that the adjacent protein domains are free to move relative to one another. Longer linkers, for example those longer than 100 amino acids, may be used in connection with alternate embodiments of the invention, and may be selected to, for example, ensure that two adjacent domains do not sterically interfere with one another. Examples of linkers which may be used in the instant invention include but are not limited to 2A linkers (for example T2A), 2A-like linkers or functional equivalents thereof.

In an exemplary embodiment, a chimeric DAP10-4-1BB adaptor polypeptide is provided comprising a DAP10 domain and a 4-1BB costimulatory domain. In another exemplary embodiment, a chimeric DAP10-CD28 adaptor polypeptide is provided comprising a DAP10 domain and a CD28 costimulatory domain. In another exemplary embodiment, a chimeric DAP10-4-1BB-CD3ζ adaptor polypeptide is provided comprising a DAP10 domain, a 4-1BB costimulatory domain, and a CD3 intracellular signaling domain. In another exemplary embodiment, a chimeric DAP10-CD28-CD3ζ adaptor polypeptide is provided comprising a DAP10 domain, a CD28 costimulatory domain, and a CD3ζ intracellular signaling domain. In yet another exemplary embodiment, a chimeric DAP10-4-1BB-CD28-CD3ζ adaptor polypeptide is provided comprising a DAP10 domain, a 4-1BB costimulatory domain, a CD28 costimulatory domain, and a CD3ζ intracellular signaling domain.

The chimeric adaptor polypeptides of the subject invention may optionally further comprise a transmembrane domain. The transmembrane domain of the CAD is a region that is capable of spanning the plasma membrane of the cytotoxic cells. The transmembrane domain is selected from a transmembrane region of a transmembrane protein such as, for example, Type I transmembrane proteins, an artificial hydrophobic sequence or a combination thereof. Suitable examples of the transmembrane domain include the transmembrane regions of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Synthetic transmembrane domains may include a triplet of phenylalanine, tryptophan and valine. Optionally, a short oligo- or polypeptide linker, preferably between 2 and 10 amino acids in length, may form the linkage between the transmembrane domain and the intracellular signaling domain of the CAD. A glycine-serine doublet provides a particularly suitable linker between the transmembrane domain and the intracellular signaling domain.

The chimeric adaptor polypeptides of the subject invention may optionally further comprise an extracellular spacer domain. The extracellular spacer domain of the CAD is a hydrophilic region which is typically located between a ligand-binding domain (which is absent in the CAD polypeptides of the present invention) and the transmembrane domain. In some embodiments, this domain facilitates proper protein folding for the CAD. The extracellular spacer domain may include a domain selected from Fc fragments of antibodies, hinge regions of antibodies, CH2 regions of antibodies, CH3 regions of antibodies, artificial spacer sequences or combinations thereof. Examples of extracellular spacer domains include CD8a hinge, artificial spacers made of polypeptides which may be as small as, three glycines (Gly), as well as CH1 and CH3 domains of IgGs (such as human IgG4).

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Optional” or “optionally” as used herein means that the particular limitation, event, circumstance, and the like can but need not occur, and that the description includes instances where said limitation, event, or circumstance occurs and instances where it does not.

As used herein, the term “DAP10” refers to the transmembrane adaptor protein present in lymphoid and myeloid cells of mammals whose exact sequence might vary slightly based on the species, isoform and from individual to individual. Alternative names for DAP10, as recognized in the art, include Hematopoietic cell signal transducer (HCST), DNAX-activation protein 10, membrane protein DAP10, transmembrane adaptor protein KAP10 (KAP10) and PIK3AP. For example, in humans DAP10 refers to the protein represented by the predominant polypeptide sequence UnitProt Q9UBK5 and NCBI accession NP_055081.1 and AF072845; however, different isoforms and variants may exist. While the name DAP10 might refer to multiple proteins with related structures and polypeptide sequences from various species, to protein members of the DAP10 protein family having high sequence identity to human DAP10 (SEQ ID NO: 1), a skilled worker will be able to identify a human DAP10 related protein in mammals, even if it differs from the sequences referenced herein. Full-length DAP10 (SEQ ID NO: 1) may be processed in a cell to a mature form (SEQ ID NO: 78) that does not have the signal peptide SEQ ID NO: 93.

The term “host cell” as used herein refers to a cell type selected to express both a CAD polypeptide and at least one chimeric receptor of the present disclosure. A host cell may endogenously expresses at least one receptor that associates with DAP10, and by extension, the chimeric adaptor polypeptides of the present disclosure. A host cell may be engineered to express at least one chimeric receptor that associates with DAP10, and by extension, the chimeric adaptor polypeptides of the present disclosure. Exemplary host cells can include, without limitation, a wide variety of immune cells, including in particular cytotoxic cells, preferable examples of which are herein disclosed (e.g., γδ T-cells, ap T cells, NK cells, NKT cells, B-cells, neutrophils, monocytes/macrophages). It is also within the scope of this disclosure that “host cells” can include non-immune cells, for example and without limitation, stem cells (e.g., embryonic stem cells, hematopoietic stem cells, stromal stem cells, induced pluripotent stem cells, and the like).

As used herein, the term “T lymphocyte” or “T cell” refers to an immune cell that expresses or has expressed CD3 (CD3+) and a T Cell Receptor (TCR+). T cells play a central role in cell-mediated immunity. A T cell that “has expressed” CD3 and a TCR has been engineered to eliminate CD3 and/or TCR cell surface expression.

The term “γδ T-cells (gamma delta T-cells)” as used herein refers to a subset of T-cells that express a distinct T-cell receptor (TCR), namely γδ TCR, on their surface, composed of one γ-chain and one δ-chain. The term “γδ T-cells” specifically includes all subsets of γδ T-cells, including, without limitation, Vδ1, Vδ2, and Vδ3 γδ T cells, as well as naive, effector memory, central memory, and terminally differentiated γδ T-cells. As a further example, the term “γδ T-cells” includes Vδ4, Vδ5, Vδ7, and Vδ8 γδ T cells, as well as Vγ2, Vγ3, Vγ5, Vγ8, Vγ9, Vγ10, and Vγ11 γδ T cells. In some embodiments, the γδ T-cells are Vδ1⁻, Vδ2⁻, or Vδ1⁻ and Vδ2⁻. Compositions and methods for making and using engineered and non-engineered γδ T cells and/or sub-types thereof include, without limitation, those described in US 2016/0175358; WO 2017/197347; U.S. Pat. No. 9,499,788; US 2018/0169147; U.S. Pat. No. 9,907,820; US 2018/0125889 and US 2017/0196910, the contents of each of which are incorporated by reference for all purposes, including the said compositions and methods for making and using engineered and non-engineered γδ T cells and/or sub-types thereof. The present application further contemplates T cells, or other engineered leukocytes or lymphocytes, that express one γ-chain or one δ-chain, optionally in combination with a second polypeptide to form a functional TCR. Such engineered leukocytes or lymphocytes, that express one γ-chain or one δ-chain may be used in the methods or present in the compositions described herein.

The γδ T cells described herein can be 61, 62, 63, or 64 γδ T cells, or combinations thereof. In some cases, the γδ T cells are mostly (>50%), substantially (>90%), essentially all, or entirely 62 γδ T cells. In some cases, the γδ T cells are mostly (>50%), substantially (>90%), essentially all, or entirely δ1 γδ T cells. In some cases, the γδ T cells are mostly (>50%), substantially (>90%), essentially all, or entirely 63 γδ T cells.

γδ T cells for use as described herein can be obtained from an allogeneic or an autologous donor. The γδ T cells can be, partially or entirely purified, or not purified, and expanded ex vivo. Methods and compositions for ex vivo expansion include, without limitation, those described in WO 2017/197347. The expansion may be performed before or after, or before and after, a chimeric adaptor polypeptide of the present disclosure is introduced into the γδ T cell(s). Other additional or alternative methods of expansion include the use of, e.g., artificial antigen-presenting cells (aAPCs), aminobisphosphonates, cytokine cocktails, and feeder cells (Cortes-Selva, D et al., (2021) Trends Pharmacol Sci. 42(1): 45-59).

As used herein, the term “up T cell” refers to T cells expressing a and R chains of the TCR as part of a complex with CD3 chain molecules. Each a and R chain contains one variable and one constant domain. αβ T cells primarily recognize peptide antigens presented by major histocompatibility complex (MHC) class I and class II molecules, where most of the receptor diversity is contained within the third complementarity determining region (CDR3) of the TCR α and β chains.

As used herein, the term “Natural killer (NK) cell” refers to CD56⁺CD3⁻ granular lymphocytes that play important roles in immunity against viruses and in the immune surveillance of tumors, and constitute a critical cellular subset of the innate immune system (Godfrey J, et al. Leuk Lymphoma 2012 53:1666-1676). NK cells express a remarkably diverse repertoire of inhibitory and activating receptors on their cell surface, which regulates their immune responses. NK cells can kill transformed or infected cells by the release of perforin and granzymes or by using effector molecules of the tumor necrosis factor (TNF) family, such as TNF, TNF-related apoptosis inducing ligand (TRAIL), and Fas ligand, which induce apoptosis in the target cells. Additionally, upon activation NK cells rapidly produce chemokines and cytokines, including interferon (IFN)-7, GM-CSF, and IL-10, that recruit and affect the function of hematopoietic and nonhematopoietic cells in the host. Unlike cytotoxic CD8 T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are considered fairly safe effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

NK cells can be obtained from an allogeneic or an autologous donor. The NK cells can be partially or entirely purified, or not purified, and expanded ex vivo. Methods and compositions for ex vivo expansion include, without limitation, those described in Becker et al., (2016) Cancer Immunol. Immunother. 65(4): 477-84). The expansion may be performed before or after, or before and after, a chimeric DAP10 adaptor polypeptide and/or chimeric receptor is introduced into the NK cell(s). Briefly, and without limitation, expansion of NK cells can include the use of engineered feeder cells, cytokine cocktails (e.g., IL-2, IL-15), and/or aAPCs (Cortes-Selva, D et al., (2021) Trends Pharmacol Sci. 42(1): 45-59).

In some examples, placental hematopoietic stem-cell derived natural killer (PNK) cells or immortalized cell lines (e.g., NK-92) may be engineered to express chimeric adaptor polypeptides of the present disclosure. In other examples, NK cells that can be used for engineering the expression of chimeric adaptor polypeptides herein can be differentiated from human embryonic stem cells (hESCs) and induced pluripotent stem cells (iPSCs). As used herein, the term “Natural killer T (NKT) cells” are T lineage cells that share morphological and functional characteristics with both T cells and NK cells. NKT cells are rapid responders of the innate immune system and mediate potent immunoregulatory and effector functions in a variety of disease settings. Ligand recognition in NKT cells leads to rapid secretion of proinflammatory cytokines (such as IFN-γ and TNF-α) and anti-inflammatory cytokines (such as IL-4, IL-10, and IL-13) that enhance the immune response to e.g., cancer by directly targeting tumor cells and by indirectly modulating the antitumor response through the release of diverse cytokines or by altering the TME. Following activation, NKT cells can immediately commence cytokine secretion without first having to differentiate into effector cells. The rapidity of their response makes NKT cells important players in the very first lines of innate defense against some types of bacterial and viral infections. In addition, many of the cytokines secreted by NKT cells have powerful effects on up T cell differentiation and function, linking NKT cells to adaptive defense. NKT cells bridge the adaptive immune system with the innate immune system. Unlike conventional T cells that recognize peptide antigens presented by major histocompatibility complex (MHC) molecules, NKT cells recognize glycolipid antigen presented by a molecule called CD1d. NKT cells can be obtained from an allogeneic or an autologous donor. The NKT cells can be partially or entirely purified, or not purified, and expanded ex vivo. Briefly and without limitation, NKT cells can be expanded via the use of ex vivo IL-2, and/or monoclonal antibodies specific for the TCR α-chain CDR3 loop (Cortes-Selva, D et al., (2021) Trends Pharmacol Sci. 42(1): 45-59).

As used herein, the term “γδ natural killer T cells” or “γδ NKT cells” refers to iPSC-derived cells that express γδ TCRs and NK receptors, but lack the expression of hallmark γδ T cell markers (Cortés-Selva, D et al., (2021) Trends Pharmacol Sci. 42(1): 45-59). These cells have been shown to have anti-tumor activity against a broad number of cancer cell lines, but not against normal cells, and showed more potent killing than donor-derived γδ T cells or donor-derived NK cells (Zeng J et al., (2019) PLoS ONE 14(5): e0216815). Chimeric adaptor polypeptides and chimeric receptors can be expressed in γδ NKT cells, in embodiments herein, for use in accordance with the methods disclosed herein.

As used herein, the term “myeloid cells” refers to a subgroup of leukocytes represented by granulocytes, monocytes, macrophages, and dendritic cells (DCs). They circulate through the blood and lymphatic system and are rapidly recruited to sites of tissue damage and infection via various chemokine receptors. Within the tissues they are activated for phagocytosis as well as secretion of inflammatory cytokines, thereby playing major roles in protective immunity. Myeloid cells can also be found in tissues under steady-state condition, where they control development, homeostasis, and tissue repair.

As used herein, the term “macrophages” refers to highly plastic innate cells with functional and phenotypic signatures that can be shaped in response to various stimuli. Macrophage polarization is broadly simplified into two different states, either a M1 phenotype (classically activated) in response to factors such as lipopolysaccharide (LPS) or IFN-γ, or a M2 phenotype in response to cytokines such as IL-4, IL-5, and IL-13. An example of M1-like macrophages express iNOS and proinflammatory cytokines such as TNF-α, IL1-β, IL-6, IL-12, and IL-23. An example of M2 macrophages exhibit increased expression of CD209, CD200R, CD1a, and CD1b in humans, and have been implicated in wound healing and antitumor responses. The ability of macrophages to infiltrate solid tumors and be reprogrammed, as well as the antitumor effects associated with a switch to the M1 phenotype, render macrophages relevant to the present disclosure in terms of engineered macrophages that express a chimeric adaptor polypeptide described herein. For example, it has been shown that macrophages can be reprogrammed towards antitumor M1 phenotype cells that are capable of producing nitric oxide and inducing IL-12-dependent NK-mediated antitumor effects by inhibiting NK-κB signaling in a murine model of ovarian cancer (Zhang F et al., (2019) Nat Commun 10: 3974).

Macrophages can be obtained/derived from an allogeneic or an autologous donor. The macrophages can be partially or entirely purified, or not purified, and cultured ex vivo (see, e.g., Davies J Q and Gordon A (2005) Methods Mol Biol 290:105016). In some embodiments, the present disclosure encompasses macrophages derived from hESCs (Karlsson, K R et al., (2008) Exp Hematol 36: 1167-1175), or iPSC-derived macrophages (Takata K. et al., (2017) Immunity 47: 183-198).

As used herein, the term “NKG2D receptor” refers to a transmembrane protein belonging to the NKG2 family of C-type lectin-like receptors. NKG2D serves as a primary activating receptor wherein ligand binding triggers cytotoxicity and cytokine production. NKG2D provides costimulation through an associated adaptor molecule, DAP10, which recruits phosphatidylinositol-3 kinase. In mice, NKG2D also associates with DAP12, which recruits protein tyrosine kinases. NKG2D is encoded by KLRK1 gene which is located in the NK-gene complex (NKC) situated on chromosome 6 in mice and chromosome 12 in humans. In humans, NKG2D is expressed by NK cells, γδ T cells and CD8+αβ T cells, and CD4+ T cells under certain pathological conditions (Stanjanovic A., et al. (2018) Front. Immunol. 23: 1-15). In mice, NKG2D is expressed by NK cells, NK1.1+ T cells, γδ T cells, activated CD8+αβ T cells and activated macrophages. The full length human NKG2G amino acid sequence is set forth herein as SEQ ID NO: 74, the amino acid sequence of the transmembrane domain of human NKG2D is set forth herein as SEQ ID NO: 75, and the amino acid sequence of the transmembrane and extracellular ligand binding domain of NKG2D is set forth herein as SEQ ID NO: 76.

The term “recombinant mammalian cell” as used herein refers to cell or cell line derived from a mammal comprising at least one alteration brought about using genetic engineering technology. In some embodiments, a “recombinant mammalian cell” is a γδ T cell, or an NK cell, or an NKT cell, or an ap T cell, etc., that comprises a nucleic acid construct that encodes a chimeric DAP10 adaptor polypeptide and/or a chimeric receptor, preferably both a chimeric DAP10 adaptor polypeptide and a chimeric receptor. A “recombinant mammalian cell” can be derived from any mammal such as e.g., a human, a rodent, etc.

As used herein, the term “TCR” or “T cell receptor” refers to a dimeric heterologous cell surface signaling protein forming an alpha-beta or gamma-delta receptor or combinations thereof. αβ TCRs recognize an antigen presented by an MHC molecule, whereas γδ TCR can recognize an antigen independently of MHC presentation.

The term “MHC” (major histocompatibility complex) refers to a subset of genes that encodes cell-surface antigen-presenting proteins. In humans, these genes are referred to as human leukocyte antigen (HLA) genes. Herein, the abbreviations MHC or HLA are used interchangeably.

The term “antigen” or “Ag” as used herein is defined as a molecule that provokes an immune response. This immune response may involve either antibody production, or the activation of specific immunologically-competent cells, or both. The skilled artisan will understand that any macromolecule, including proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized, or can be derived from a biological sample. Such a biological sample can include, but is not limited to a tissue sample, a tumor sample, a cell or a biological fluid.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies (including agonist, antagonist, neutralizing antibodies, full length or intact monoclonal antibodies), antibody compositions with polyepitopic specificity, multivalent antibodies, multispecific antibodies (e.g., bispecific antibodies so long as they exhibit the desired biological activity), formed from at least two intact antibodies, diabodies, single domain antibodies (sdAbs), as long as they exhibit the desired biological or immunological activity, single chain fragment variable (scFv), F(ab) and F(ab′)₂, as well as single chain antibodies and humanized antibodies (Harlow et ah, 1999, In: Using Antibodies: A Laboratory Manual, Cold Spring Harbor Laboratory Press, NY: Harlow et ah, 1989, In; Antibodies: A Laboratory Manual, Cold Spring Harbor, N.Y.; Houston et ah, 1988, Proc. Nat Acad. Sci. USA 85:5879-5883: Bird et ah, 1988, Science 242:423-426).

The term “epitope” includes any protein determinant, lipid or carbohydrate determinant capable of specific binding to an immunoglobulin or receptor, for example a T-cell receptor. Epitopic determinants usually consist of active surface groupings of molecules such as amino acids, lipids or sugar side chains and usually have specific three-dimensional structural characteristics, as well as specific charge characteristics.

A “modification” of an amino acid residue/position, as used herein, refers to a change of a primary amino acid sequence as compared to a starting amino acid sequence, wherein the change results from a sequence alteration involving said amino acid residue/positions. A “modification” of an amino acid residue/position is synonymous with “mutation” of an amino acid residue/position. For example, typical modifications include substitution of the residue (or at said position) with another amino acid (e.g., a conservative or non-conservative substitution), insertion of one or more amino acids, and deletion of one or more amino acids. An “amino acid substitution”, or variation thereof, refers to the replacement of an existing amino acid residue in a predetermined (starting) amino acid sequence with a different amino acid residue. Generally and preferably, the modification results in alteration in at least one physicobiochemical activity of the variant polypeptide compared to a polypeptide comprising the starting (or “wild type”) amino acid sequence. A “modified” amino acid sequence, as referred to herein thus comprises an amino acid sequence in which one or more amino acids have been mutated, and/or in which any number of amino acids have been inserted, and/or in which any number of amino acids have been deleted.

The term “endogenous” as used herein, refers to substances and/or processes that originate from within a system including but not limited to an organism, tissue, or cell. For example, in the context of this disclosure, “endogenous” refers to a nucleic acid molecule or polypeptide that is normally expressed in a cell or tissue.

Conversely, the term “exogenous” as used herein refers to substances and/or processes that originate from outside of a system including but not limited to an organism, tissue, or cell. Particularly, “exogenous” in the context of the present disclosure is meant a nucleic acid molecule or polypeptide that is not naturally present in a cell. The term “exogenous” would therefore encompass any foreign or heterologous recombinant nucleic acid molecule or polypeptide expressed in a cell, including an exogenous nucleic acid having a different sequence relative to its native endogenous counterpart. As is well known in the art, these exogenous sequences may be introduced by genetic engineering into the cell itself or a progenitor thereof, and may optionally be linked to alternative control sequences, such as a non-native promoter or secretory sequence.

The term “overexpression” as used herein refers to expression at a level exceeding the endogenous expression level of the subject nucleic acid or polypeptide in the cell or tissue. In exemplary embodiments, a receptor of interest (e.g. NKG2D) can be overexpressed in a host cell, where the level of expression of the receptor is greater than the naturally-occurring expression level of same receptor. Methods for overexpressing a nucleic acid or polypeptide of interest are not particularly limited and are discussed in more detail herein e.g., a polypeptide (e.g. NKG2D) can be overexpressed by the transfer of the corresponding nucleic acid using the same or a different expression vector than that encoding the CAD polypeptide. The expression vector is not particularly limited as long as the vector can be used in genetic engineering. For example, a plasmid vector, a virus vector, a cosmid vector, a bacterial artificial chromosome (BAC), a yeast artificial chromosome (YAC), and any of other non-plasmid vectors can be used.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the polynucleotides and cells of the disclosure in prevention of the occurrence of tumor in the first place.

As used herein, the term “autologous” is meant to refer to any material derived from an individual which is later to be re-introduced into the same individual.

As used herein, the term “allogeneic” refers to material derived from an animal which is later introduced into a different animal of the same species.

As used herein, the term “syngeneic” refers to material that is genetically similar or identical and hence immunologically compatible, such that transplantation does not provoke an immune response.

As used herein, the term “agent” refers to any protein, nucleic acid molecule (including chemically modified nucleic acids), compound, antibody, small molecule, organic compound, inorganic compound, other molecule of interest, or cell (e.g., cell engineered to express a chimeric adaptor polypeptide). Agent can include a therapeutic agent, a diagnostic agent or a pharmaceutical agent. A therapeutic or pharmaceutical agent is one that alone or together with an additional agent induces the desired response (such as inducing a therapeutic or prophylactic effect when administered to a subject, including treating a subject suffering cancer, viral infection (e.g., cytomegalovirus (CMV), influenza, hepatitis B, Epstein-Barr, adenovirus, and the like), bacterial infection (e.g., E. coli, M. tuberculosis, etc.) rheumatoid arthritis (RA), or other disease/condition. Discussed herein, an agent may be referred to as a modulatory agent.

The term “diagnosis”, or “diagnosing” as used herein refers to the process of identifying a disease, such as cancer, by its signs, symptoms, and/or results of various tests. A conclusion reached through such a process is a diagnosis. Forms of testing commonly performed include blood tests, medical imaging, urinalysis, biopsy, and the like.

The term “therapeutically effective amount”, or simply “effective amount” refers to the amount of an agent or composition (e.g., composition comprising an agent) that will elicit a biological or medical response of a tissue, system, or subject that is being sought by the researcher, veterinarian, medical doctor or other clinician. The term “therapeutically effective amount” includes that amount of an agent, or a composition comprising an agent, that, when administered, is sufficient to prevent development of, or alleviate to some extent, one or more of the signs or symptoms of the disorder or disease (e.g., hematological or solid tumor) being treated. The therapeutically effective amount will vary depending on the composition, the disease and its severity and the age, weight, etc., of the subject to be treated.

To “treat” a disease as the term is used herein, means to decrease or reduce the frequency or severity of at least one sign or symptom of a disease or disorder experienced by a subject.

The term “decrease” as used herein means to reduce the quality, amount, or strength of something. In one example, a therapy (e.g., administration of a therapeutic agent of the present disclosure) decreases one or more signs or symptoms associated with a disease or condition, for example as compared to the response in the absence of the therapy. For example, administration of a therapeutic agent may in examples provide an anti-tumor effect that decreases one or more signs or symptoms associated with cancer.

As used herein, the term “administration” means to provide or give a subject one or more agents, such as an agent that treats one or more signs or symptoms associated with a condition/disorder or disease including but not limited to cancer, viral infection, bacterial infection, etc., by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained. The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more modulatory agents. In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

The term “cytokine” as used herein refers to a diverse group of soluble proteins and peptides released from cells which act as humoral regulators at nano- to picomolar concentrations, and which, either under normal or pathological conditions, modulate the functional activities of individual cells and tissues. These proteins also mediate interactions between cells directly and regulate processes taking place in the extracellular environment. Many growth factors and cytokines act as cellular survival factors by preventing programmed cell death. Cytokines include both naturally occurring peptides and variants that retain full or partial biological activity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The terms “patient,” “subject,” “individual,” and the like are used interchangeably herein, and refer to any animal, amenable to the methods described herein. In certain non-limiting embodiments, the patient, subject or individual is a human.

By the term “specifically binds”, as used herein with respect to a cell surface receptor, is meant a receptor which recognizes a specific molecule/ligand, but does not substantially recognize or bind other molecules in a sample. For example, a receptor that specifically binds to a molecule from one species may also bind to that molecule from one or more species. But, such cross-species reactivity does not itself alter the classification as specific. In another example, a receptor that specifically binds to a molecule may also bind to different allelic forms of the molecule. However, such cross reactivity does not itself alter the classification as specific. In some instances, the terms “specific binding” or “specifically binding,” can be used in reference to the interaction of a protein (or a peptide) with a second chemical species, to mean that the interaction is dependent upon the presence of a particular structure (e.g., an antigenic determinant or epitope) on the chemical species; for example, a receptor recognizes and binds to a specific a structure rather than to proteins generally. If receptor is specific for epitope “A”, the presence of a molecule containing epitope A (or free, unlabeled A), in a reaction containing labeled “A” and the receptor, will reduce the amount of labeled A bound to the receptor.

In some embodiments, specific binding can be characterized by an equilibrium dissociation constant of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like.

The term “cancer” as used herein refers to a physiological condition in mammals in which a population of cells are characterized by unregulated cell growth. Neoplasia, malignancy, cancer, and tumor may be used interchangeably and refer to abnormal growth of a tissue or cells that results from excessive cell division. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Symptoms of cancer may include but are not limited to persistent cough or blood-tinged saliva, a change in bowel habits, blood in the stool, unexplained anemia (low blood count), breast lump or breast discharge, lumps in testicles, a change in urination, blood in urine, hoarseness, persistent lumps or swollen glands, obvious change of a wart or mole, indigestion, difficulty swallowing, unusual vaginal bleeding or discharge, unexpected weight loss, night sweats, or fever, continued itching in the anal or genital area, nonhealing sores, headaches, back pain, pelvic pain, and bloating, among others.

Hematologic cancers are cancers originating in the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are tumors that comprise a tumor mass of at least about 10 or at least about 100 tumor cells. The solid tumor can be a soft tissue tumor, a primary solid tumor, or a metastatic lesion.

Examples of solid tumors include, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), pancreas, prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. In another embodiment, the cancer is a glioma. Metastatic lesions of the aforementioned cancers can also be treated or prevented using the methods and compositions of the disclosure.

“Expression cassette” refers to a nucleic acid comprising expression control sequences operatively linked to a nucleic acid encoding a transcript or polypeptide to be expressed. An expression cassette comprises sufficient cis-acting elements for expression; other elements for expression can be supplied by the host cell or in an in vitro expression system. Expression cassettes can be a component of a vector such as a cosmid, a plasmid (e.g., naked or contained in a liposome), or a virus (e.g., lentivirus, retrovirus, adenovirus, and adeno-associated virus). An expression cassette can be in a host cell, such as an immune cell (e.g., γδ T cell).

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “substantial identity” or “substantially identical,” when referring to a nucleic acid or fragment thereof, indicates that, when optimally aligned with another nucleic acid (or the complementary strand of the other nucleic acid), there is nucleotide sequence identity in %, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% sequence identity. In some aspects, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-45, herein incorporated by reference. A “moderately conservative” replacement is any change having a nonnegative value in the PAM250 log-likelihood matrix.

Sequence identity and/or similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Sequences also can be compared using the Smith-Waterman homology search algorithm using an affine gap search with a gap open penalty of 12 and a gap extension penalty of 2, BLOSUM matrix of 62. Another preferred algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

### I. Introduction

DNAX-activating protein of 10 kDa (DAP10) is the adaptor molecule that associates with the cell surface, cytotoxic receptor natural killer group 2 member D (NKG2D). The NKG2D receptor is a type II transmembrane-anchored C-type lectin-like protein, which belongs to the CD94/NKG2 family of C-type lectin-like receptors (Houchins et al., (1991) J. Exp. Med. 173: 1017-1020). NKG2D is capable to bind numerous and highly diversified MHC class I-like self-molecules. These ligands are often poorly expressed on normal cells but can be induced on damaged, transformed, or infected cells (Zingoni, A et al. (2018) Front. Immunol. 9(476): 1-12). Said ligands belong to the H60(a-c), RAE (a-c), and MULTI families in mice, and to the MIC (MICA and MICB) and ULBP (ULBP1-ULBP6) families in humans, where their repertoire is more complex that in other species. In fact, MIC molecules are encoded by the most highly polymorphic human genes after the classical HLA molecules (Eagle, R A and Trowsdale, J. (Nat. Rev. Immunol. (2007) 7(9): 737-44).

NKG2D is an activating immune receptor which regulates both innate and adoptive immune responses. NKG2D is abundantly present on all NK cells, CD8 T cells, subsets of 76 T cells and some autoreactive CD4 T cells. NKG2D acts with other costimulatory molecules such as DAP10, to modify the strength and duration of antigen-specific responses mediated by the T cell receptor and to influence the pattern of anti-tumor reactivity by T lymphocytes (see e.g., Maccalli C, et al. (2003) Eur. J. Immunol. 33(7):2033-43).

NKG2D lacks a signaling motif in its cytoplasmic domain. Thus, after ligand binding, NKG2D signal transduction and cellular activation relies upon association of NKG2D with the DAP10 adaptor molecule which promotes and stabilizes NKG2D surface membrane expression (Wu, J., et al., (1999) Science 285: 730-732). However, it is known, for example, that TGF-β is capable of mediating down-regulation of NKG2D (and NKG2DL) surface expression (Lazarova M and Steinle. (2019) Front. Immunol 10(2689): 1-11), and that TGF-β can substantially decrease DAP10 expression both at mRNA and protein levels (Park, Y P et al. (2011) Blood. 118: 3019-27; Lee, J C et al. (2011) Tumori 97:350-7). Accordingly, it is herein recognized that methodologies capable of modulating NKG2D and/or DAP10 expression and/or associated signaling pathways are of therapeutic interest.

The human NKG2D receptor assembles with the DAP10 signaling dimer, with one NKG2D homodimer paired with a DAP10 dimer by formation of two salt bridges between conserved transmembrane (TM) arginine residues (Garrity, D. et al. (2005) PNAS USA 102(21): 7641-7646). The DAP10 dimer carries a pair of aspartic acid residues close to the center of the transmembrane (TM) domains, and these residues interact with the conserved arginine in the TM sequence of NKG2D for assembly with the DAP10 dimer. Thus, the NKG2D homodimer associates with the DAP10 adaptor molecule in its transmembrane domain to form a hexameric structure which can initiate signaling cascades (see e.g., Garrity et al (2005) supra).

The DAP10 dimer is a disulfide-linked homodimer. The amino acid sequence of the wild-type human DAP10 polypeptide is shown below as SEQ ID NO: 1: MIHLGHILFLLLLPVAAAQTTPGERSSLPAFYPGTSGSCSGCGSLSLPLLAGLVAADAVA SLLIVGAVFLCARPRRSPAQEDGKVYINMPGRG (SEQ ID NO: 1). See also Table 3, which lists a number of sequences relevant to the present disclosure.

The DAP10 cytoplasmic domain comprises a tyrosine-based motif (YINM), comprising residues 86-89 of SEQ ID NO: 1. Upon tyrosine (Y86) phosphorylation, DAP10 is capable to bind either the p85 subunit of phosphatidylinositol 3-kinase (PI3K, through YXXM) or the adaptor Grb2 (through YXNX). Because these two binding sites overlap, a single DAP10 chain will bind either p85 or Grb2, but not both (Lanier L L. (2008) Nat. Immunol. 9(5): 495-502). This YINM motif is similar to the motif in CD28, which provides for costimulatory signaling in conjunction with the immunoreceptor tyrosine-based activation motif (ITAM)-based TCR/CD3 complex in T cells.

Both recruitment of the p85 subunit of PI3K or the adaptor protein Grb2 can activate Vav1 and PLC-y2 and thus are essential for the activation of Ca²⁺ mobilization and cytotoxicity toward cells (Upshaw J L et al. (2006) Nat. Immunol. 7(5): 524-32).

DAP10 further comprises a ubiquitinylation site that encompasses the lysine at amino acid 84 of the DAP10 protein sequence (SEQ ID NO: 1). Ligand stimulation of NKG2D on NK cells results in the ubiquitylation of DAP10, which is required for the endocytosis and degradation of the NKG2D-DAP10 complex (see e.g., Molfetta, R., et al. (2014) Eur. J. Immunol. 44, 2761-2770). Furthermore, it has been shown that ubiquitin-dependent receptor endocytosis is required for the activation of extracellular signal-regulated kinase (ERK) and NK cell functions, such as the secretion of cytotoxic granules and the inflammatory cytokine interferon-7. Hence, NKG2D-DAP10 endocytosis represents a means to decrease cell surface receptor abundance, as well as to control signaling in cytotoxic lymphocytes.

In addition to NKG2D, DAP10 is known to associate with a number of other receptors. For example, Ly49H and Ly49D were co-immunoprecipitated with DAP10 from mouse NK cells, and have been shown to associate with DAP10 when co-transfected into 293T cells (Coudert J D et al. (2008) Blood 111: 3571-3578). By co-transfection studies, DAP10 has also been shown to associate with human Sirp-b1 in transfected rat RBL-2H3 cells (Anfossi N et al. (2003) Eur. J. Immunol. 33: 3514-3522). Similarly, human and mouse Siglec-15 (Angata T et al. (2007) Glycobiology 17: 838-846) and Cd3001b (Yamanishi Y et al. (2008) Blood 111: 688-698) have been shown by co-transfection and co-immunoprecipitation to pair with DAP10. As mentioned, certain DAP10-associated receptors (i.e., NKG2D) appear to recognize host-encoded molecules, including carbohydrate and protein ligands, however other DAP10-associated receptors can directly recognize microbial ligands. One example includes the recognition of the mouse CMV-encoded glycoprotein m157 by Ly49H (Lanier L L (2008) Nat. Rev. Immunol. 8(4): 259-68; Smith H R et al. (2002) Proc. Natl. Acad. Sci. USA 99(13): 8826-31). m157 is a GPI-anchored glycoprotein with homology to MHC class I, which is displayed on the surface of mouse CMV-infected cells, resulting in activation of Ly49H+ NK cell-mediated cytotoxicity and cytokine production.

The above-mentioned receptors are meant to be illustrative of the ability of DAP10 to pair with, and hence modulate signaling through, numerous receptors in addition to NKG2D, and is not mentioned to be exhaustive. It is to be understood that with regard to the present disclosure, the nucleic acids, polypeptides encoded thereby, cells, compositions, and methods apply to any and all receptors that DAP10 is capable of partnering with. For example, embodiments herein encompass chimeric receptors comprising a DAP10-interacting domain, thereby imparting the ability of a CAD polypeptide of the present disclosure to modulate signaling through one or more chimeric receptors expressed in a same cell as the CAD polypeptide, as herein disclosed.

### II. Compositions and Methods of the Invention

Unless defined otherwise, technical and scientific terms used herein have the same meaning as commonly understood by a person of ordinary skill in the art. In particular, this disclosure utilizes routine techniques in the field of recombinant genetics, immunology, and biochemistry. Basic texts disclosing the general terms in molecular biology and genetics include e.g., Lackie, Dictionary of Cell and Molecular Biology, Elsevier (5th ed. 2013). Basic texts disclosing methods in recombinant genetics and molecular biology include e.g., Sambrook et al, Molecular Cloning—A Laboratory Manual, Cold Spring Harbor Press 4th Edition (Cold Spring Harbor, N.Y. 2012) and Current Protocols in Molecular Biology Volumes 1-3, John Wiley & Sons, Inc. (1994-1998) and Supplements 1-115 (1987-2016). Basic texts disclosing the general methods and terms in biochemistry include e.g., Lehninger Principles of Biochemistry sixth edition, David L. Nelson and Michael M. Cox eds. W. H. Freeman (2012). Basic texts disclosing the general methods and terms immunology include Janeway's Immunobiology (Ninth Edition) by Kenneth M. Murphy and Casey Weaver (2017) Garland Science; Fundamental Immunology (Seventh Edition) by William E. Paul (2013) Lippincott, Williams and Wilkins.

### A. Chimeric Adaptor Constructs

Aspects of the present disclosure include constructs comprising nucleic acids encoding chimeric adaptor polypeptides. In embodiments, the nucleic acids encode chimeric adaptor polypeptides comprising i) a human DAP10 amino acid sequence and ii) one or more costimulatory domains (e.g., 4-1BB, OX40, ICOS, CD28), wherein the CAD polypeptide specifically lacks an ectodomain. In embodiments, the CAD polypeptides can additionally or alternatively comprise one or more intracellular signaling domains (e.g., CD3ζ) as herein described. In embodiments, the CAD polypeptides can also comprise one or more mutations, for example one or more mutations in DAP10 and/or one or more modifications (e.g., one or more mutations, additions, or deletions) in the costimulatory domain(s) and/or the intracellular signaling domain(s).

In embodiments, the CAD polypeptides function to modulate and/or mute signaling through one or more receptors with which they associate. In embodiments, the CAD polypeptides are designed with modulated/muted attributes (e.g., by way of the one or more mutations) and/or added signaling attributes (e.g., by way of the C-terminal fusion) and expressed in a host cell promote a favorable balance of signaling pathways upon receptor-target engagement (e.g., NKG2D engagement of an extracellular target ligand), which may serve to address the problem of low or lost expression of the target (i.e., antigen escape) for the primary TCR. The term “favorable balance” refers broadly to the introduction of signaling cascades that are complementary to, alternative to, or that act to modulate primary DAP10 signal transduction cascades in a desired manner. Accordingly, the CAD polypeptides disclosed herein provide for altered (e.g., improved) functional properties including but not limited to altered (e.g., enhanced) cytolytic, proliferative, survival and/or costimulatory properties that are elicited upon engagement with the ligands of receptors that partner with DAP10 (e.g., widely expressed ligands of NKG2D). The precise composition of the CAD polypeptides can be designed based on a given disease indication and in some examples on pairing with the specificity and signaling components of other receptor(s) (e.g., TCR receptor(s) and/or chimeric receptors as herein disclosed) that are present on the same cells. As one illustrative example, immunosuppressive signals within a tumor microenvironment (TME) may inhibit anti-tumor T cell responses through inhibitory receptors on T cells, and it is within the scope of this disclosure that via the use of a CAD polypeptide such an inhibitory output may be switched to an immunostimulatory one. Other relevant examples may be found in, e.g., Guo, J., et al., (2021) Journal for Immuno Therapy of Cancer 9:e002628.

In embodiments, the CAD polypeptides may function at least in part to stabilize cell surface receptor(s) (e.g., NKG2D and/or chimeric receptors as herein disclosed) with which they associate. To “stabilize” cell surface receptor(s) as herein disclosed means to decrease a rate at which said cell surface receptors are endocytosed or otherwise removed from the surface of the cell, as compared to the rate at which said cell surface receptors are otherwise removed under similar circumstances in absence of a CAD polypeptide as herein described. Encompassed within the scope of receptor stabilization as herein described includes positive feedback mechanisms through which CAD polypeptide signaling results in increased cell surface expression of the receptor(s) with which endogenous DAP10 and the CAD polypeptides associate (Wu, J., et al., (2000) Journal of Exp Med 192(7): 1059-1068).

The disclosure also provides stably expressed CAD polypeptides. In embodiments, a CAD polypeptide is expressed at a level substantially similar to the level at which endogenous DAP10 is expressed in a host cell of interest. In embodiments, a CAD polypeptide is expressed at a level higher than the level at which endogenous DAP10 is expressed in the host cell of interest. For example, a CAD polypeptide may be expressed at a level 10% higher, or between 10-20% higher, or between 20-30% higher, or between 30-40% higher, or between 40-50% higher, or between 50-60% higher, or between 60-70% higher, or between 70-80% higher, or between 80-90% higher or between 90-100% higher, or even higher, such as 2-fold higher, or 3-fold higher, or 4-fold higher, or 5-fold higher, of 6-fold higher, or 7-fold higher, or 8-fold higher, or 9-fold higher, or 10-fold higher, or 20-fold higher, or 30-fold higher, or 40-fold higher, or 50-fold higher or 100-fold higher, than a corresponding level at which endogenous DAP10 is expressed. Thus, it may be understood that in embodiments, the CAD polypeptides of the subject invention may compete with endogenous DAP10 binding to a receptor (e.g., NKG2D), depending on the particular host cell type.

Aspects of the disclosure include nucleic acids encoding CAD polypeptides, and constructs/vectors containing such nucleic acids. Therefore, described herein are nucleic acids encoding CAD polypeptides that incorporate select mutations and signaling domains that modulate and/or add signaling attributes that impart desired properties including but not limited to e.g., sustained survival, proliferation and/or killing on the host cell expressing said CAD polypeptides. In some embodiments, the nucleic acid encodes a CAD polypeptide comprising a signal peptide at its N-terminus. A signal peptide is a stretch of amino acid sequence in a protein (e.g., at the N-terminus of the protein) for translocating the protein to a specific intracellular location (e.g., the endoplasmic reticulum for secretion). In some embodiments, the nucleic acid encodes a CAD that comprises a mature Dap10 (e.g., SEQ ID NO: 78) or a fragment thereof (e.g., SEQ ID NO: 79, 80, or 81), and the signal peptide SEQ ID NO: 93 at the N-terminus of the mature Dap10 or a fragment thereof. In embodiments, the nucleic acid encodes a CAD that comprises a mature Dap10 (e.g., SEQ ID NO: 78) or a fragment thereof (e.g., SEQ ID NO: 79, 80, or 81), and the signal peptide SEQ ID NO: 45 at the N-terminus of the mature Dap10 or a fragment thereof. In some embodiments, the nucleic acid encodes a CAD that comprises a mature Dap10 (e.g., SEQ ID NO: 78) or a fragment thereof (e.g., SEQ ID NO: 79, 80, or 81), and the signal peptide comprising both SEQ ID NO: 45 and SEQ ID NO: 93 (e.g., SEQ ID NO: 45 at the N-terminus of SEQ ID NO: 95).

### 1. Mutations

In embodiments, a CAD polypeptide carries one or both of an amino acid modification at position K84 and/or position Y86 of SEQ ID NO: 1. In embodiments, K84 is modified to comprise another positively charged amino acid, for example K84R, or K84H, although it is within the scope of this disclosure that the modification at K84 can comprise other amino acid substitutions. In one embodiment, the modification is a K84R modification. In embodiments, a CAD polypeptide with a K84R modification comprises SEQ ID NO: 18. In embodiments, Y86 is modified to another aromatic amino acid, for example Y86F or Y86W, although it is within the scope of this disclosure that the modification at Y86 can comprise other amino acid substitutions. In one embodiment, the modification is a Y86F modification. In embodiments, a CAD polypeptide with a Y86F modification comprises SEQ ID NO: 19. In embodiments, a CAD polypeptide with both a K84R modification and a Y86F modification comprises SEQ ID NO: 20.

In embodiments, the modification at position 86 of SEQ ID NO: 1 reduces or eliminates p85/PI3K binding to the CAD polypeptide, and in turn reduces or eliminates PI3K/AKT/PKCO signaling. Hence, the modification at position 86 may serve to functionally reduce or eliminate one or more of costimulation, calcium-flux, and/or degranulation. In a preferred embodiment, the modification is Y86F.

In additional or alternative embodiments, the modification at position 86 of SEQ ID NO: 1 reduces or eliminates Grb2 binding to the CAD polypeptide, and in turn reduces or eliminates Vav1/SLP-76/PLCγ signaling. Hence, the modification at position 86 may serve to reduce or eliminate one or more of calcium-influx and/or degranulation. In a preferred embodiment, the modification is Y86F.

In additional or alternative embodiments, the modification at position 84 of SEQ ID NO: 1 reduces or completely disrupts ubiquitinylation of the CAD polypeptide, which in turn reduces or completely prevents the internalization of the chimeric DAP10 adaptor polypeptide-endogenous receptor complex at the cellular membrane of the particular host cell (see e.g., Quatrini, L., et al., (2015) Sci Signal 8(400):ra108). In this way, via reliance on the CAD polypeptide harboring at least the K84 modification (e.g., K84R), a receptor that associates with DAP10 (e.g., NKG2Dand/or a chimeric receptor as herein disclosed) can be stabilized at the cell surface. Furthermore, a modification at K84 (e.g., K84R) may reduce or eliminate signaling (e.g., ERK1/2) that otherwise occurs en route to lysosomal degradation. Reduction or elimination of signaling (e.g., ERK1/2) may serve to reduce or eliminate one or more of exhaustion, activation-induced cell death and/or induction of cell cycle arrest upon hyperactivation, one or more of which may otherwise occur in absence of a CAD polypeptide harboring the K84 modification (e.g., K84R).

In additional or alternative embodiments, a CAD polypeptide may include a modification at position 57 of SEQ ID NO: 1, for example a D57A modification, although amino acid modifications other than alanine are within the scope of this disclosure. In embodiments, a chimeric DAP10 adaptor polypeptide with a D57A modification comprises SEQ ID NO: 37. A modification at D57 may in embodiments serve to modify (e.g., reduce or abolish) stable interaction with KLRK1 (Wu, J., et al., (1999) Science 285(5428): 730-2).

In additional or alternative embodiments, a CAD polypeptide may include a modification at position 88 of SEQ ID NO: 1, for example a N88Q modification, although amino acid modification other than glutamine are within the scope of this disclosure. In embodiments, a CAD polypeptide with a N88Q modification comprises SEQ ID NO: 38. A modification at N88 may in embodiments serve to modify (e.g., reduce) cell killing activity and/or interaction with GRB2, while having minimal to no effect on interaction with PIK3R1 (Upshaw, J L., (2006) Nat Immunol 7:524-532).

In additional or alternative embodiments, a CAD polypeptide may include a modification at position 89 of SEQ ID NO: 1, for example a M89Q modification, although amino acid modifications other than glutamine are within the scope of this disclosure. In embodiments, a CAD polypeptide with a M89Q modification comprises SEQ ID NO: 39. A modification at M89Q may in embodiments serve to modify (e.g., reduce) cell killing activity and/or interaction with PIK3R1, while having minimal to no effect on interaction with GRB2 (Upshaw, J L., (2006) Nat Immunol 7:524-532).

It may be understood that the present disclosure encompasses chimeric adaptor polypeptides having any one or more or each of the above-mentioned modifications.

### 2. Costimulatory and Signaling Domains

In embodiments, the endodomain of the chimeric adaptor polypeptides of the present disclosure comprise one or more costimulatory domains, wherein the costimulatory domain comprises functional costimulatory signaling domain derived from e.g., a MHC class I molecule, TNF receptor proteins, immunoglobulin-like proteins, cytokine receptors, integrins, signaling lymphocytic activation molecules (SLAM proteins), activating NK cell receptors, BTLA, a Toll ligand receptor, and the like. For example, it is within the scope of this disclosure that the endodomain of a disclosed CAD polypeptide can include 2, 3, 4 or more costimulatory domains. It is also within the scope of this disclosure that when more than one costimulatory domain is included, the costimulatory domains may be the same, or they may be different. In embodiments, the costimulatory domains are derived from one or more of TLR1, TLR2, TLR3, TLR4, TLR5, TLR6, TLR7, TLR8, TLR9, TLR10, CARD 11, B7-H3, CEACAM1, CRTAM, CD2, CD3C, CD4, CD7, CD8α, CD8β, CD11a, CD11b, CD11c, CD11d, IL2Rβ, IL2γ, IL7Rα, IL4R, IL7R, IL15R, IL21R, CD18, CD19, CD19aCD27, CD28, CD29, CD30, CD40, CDS, CD49a, CD49D, CD49f, CD54 (ICAM), CD69, CD70, CD80, CD83, CD84, CD86, CD96 (Tactile), CD100 (SEMA4D), CD103, CD134 (OX40), CD137 (4-1BB), CD152 (CTLA-4), CD160 (BY55), CD162 (SELPLG), CD244 (2B4), CD270 (HVEM), CD226 (DNAM1), CD229 (Ly9), CD278 (ICOS), ICAM-1, LFA-1 (CD11a/CD18), FcR, FeyRI, FeyRII, FeyRIII, LAT, NKG2C, SLP76, TRIM, ZAP70, GITR, BAFFR, LTBR, LAT, GADS, LIGHT, HVEM (LIGHTR), KIRDS2, ITGA4, ITGA6, ITGAD, ITGAE, ITGAL, ITGAM, ITGAX, ITGB1, ITGB2, ITGB7, NKG2C, NKG2D, IA4, VLA-1, VLA-6, SLAM (SLAMF1, CD150, IPO-3), SLAMF4, SLAMF6 (NTB-A, Ly108), SLAMF7, SLAMF8 (BLAME), SLP-76, PAG/Cbp, NKp80 (KLRF1), NKp44, NKp30, NKp46, BTLA, JAML, CD150, PSGL1, TSLP, TNFR2, and TRANCE/RANKL, or a portion thereof, and combinations thereof.

In some embodiments, the CAD construct encodes at least one 4-1BB costimulatory domain, and optionally a second costimulatory domain selected from 4-1BB, 2B4, ICOS, CD28, OX40, and CD27 costimulatory domains, or any of the above-mentioned costimulatory domains. In some embodiments, the construct encodes at least two 4-1BB costimulatory domains, or at least two 4-1BB costimulatory domains in combination with one, two, three, or four, or more, costimulatory domains selected from 4-1BB, ICOS, CD28, OX40, and CD27, or any of the above-mentioned costimulatory domains. In some embodiments, the 4-1BB costimulatory domain comprises SEQ ID NO: 2 (KRGRKKLLYIFKQPFMRPVQTTQEEDGCSCRFPEEEEGGCEL). In some embodiments, the 4-1BB costimulatory domain comprises an amino acid sequence having at least one, at least two, or at least three or more modifications of an amino acid sequence of SEQ ID NO: 2. In embodiments, the 4-1BB costimulatory domain is substantially similar to the 4-1BB costimulatory domain comprising SEQ ID NO: 2.

In some embodiments, the CAD construct encodes at least one CD27 costimulatory domain, and optionally at least one second costimulatory domain selected from 4-1BB, ICOS, CD28, OX40, 2B4, and CD27 costimulatory domains, or any of the above-mentioned costimulatory domains. In some embodiments, the construct encodes at least one CD27 costimulatory domain, and a 4-IBB costimulatory domain. In some embodiments, the construct encodes two CD27 costimulatory domains, and at least one second costimulatory domain selected from a 4-1BB, ICOS, CD28, and CD27. In some embodiments, the CD27 costimulation domain comprises SEQ ID NO: 5 (QRRKYRSNKGESPVEPAEPCHYSCPREEEGSTIPIQEDYRKPEPACSP). In some embodiments, the CD27 costimulatory domain comprises an amino acid sequence having at least one, at least two, at least three or more modifications of an amino acid sequence of SEQ ID NO: 5. In embodiments, the CD27 costimulatory domain is substantially similar to the CD27 costimulatory domain comprising SEQ ID NO: 5.

In some embodiments, the CAD construct encodes at least one CD28 costimulatory domain, and optionally a second costimulatory domain selected from 4-1BB, 2B4, ICOS, CD28, OX40, and CD27 costimulatory domains, or any of the above-mentioned costimulatory domains. In some embodiments, the CAD construct encodes at least two CD28 costimulatory domains, or at least two CD28 costimulatory domains in combination with one, two, three, or four, or more, costimulatory domains selected from a 4-1BB, ICOS, CD28, OX40, and CD27, or any of the above-mentioned costimulatory domains. In some embodiments, the CD28 costimulatory domain comprises SEQ ID NO: 40 FWVLVVVGGVLACYSLLVTVAFIIFWVRSKRSRLLHSDYMNMTPRRPGPTRKHYQPYA PPRDFAAYRS). In embodiments, the CD28 costimulatory domain comprises SEQ ID NO: 41 (FWVRSKRSRLLHSDYMNMTPRRPGPTRKHYQPYAPPRDFAAYRS). Included in SEQ ID NO: 40 and SEQ ID NO: 41 are three subdomains YMNM, PRRP, and PYAP, that are capable to regulate signaling pathways. In embodiments, a disclosed CAD polypeptide comprises mutation or deletion of one or more of said subdomains (see e.g., WO2019010383). In some embodiments, the CD28 costimulatory domain comprises an amino acid sequence having at least one, at least two, at least three or more modifications of an amino acid sequence of SEQ ID NO: 40, or an amino acid sequence of SEQ ID NO: 41. In some embodiments, the CD28 costimulatory domain is substantially similar to the CD28 costimulatory domain comprising SEQ ID NO: 40. In some embodiments, the CD28 costimulatory domain is substantially similar to the CD28 costimulatory domain comprising SEQ ID NO: 41.

In some embodiments, the CAD construct encodes at least one ICOS costimulatory domain, and optionally a second costimulatory domain selected from 4-1BB, 2B4, ICOS, CD28, OX40, and CD27 costimulatory domains, or any of the above-mentioned costimulatory domains. In some embodiments, the CAD construct encodes at least two ICOS costimulatory domains, or at least two ICOS costimulatory domains in combination with one, two, three, or four, or more, costimulatory domains selected from 4-1BB, ICOS, CD28, OX40, and CD27, or any of the above-mentioned costimulatory domains. In some embodiments, the ICOS costimulatory domain comprises SEQ ID NO: 42. In some embodiments, the ICOS costimulatory domain comprises an amino acid sequence having at least one, at least two, at least three or more modifications of an amino acid sequence of SEQ ID NO: 42 (see e.g., US20170209492). In some embodiments, the ICOS costimulatory domain is substantially similar to the ICOS costimulatory domain comprising SEQ ID NO: 42.

In some embodiments, the CAD construct encodes at least one OX40 costimulatory domain, and optionally a second costimulatory domain selected from 4-1BB, 2B4, ICOS, CD28, OX40, and CD27 costimulatory domains, or any of the above-mentioned costimulatory domains. In some embodiments, the CAD construct encodes at least two OX40 costimulatory domains, or at least two OX40 costimulatory domains in combination with one, two, three, or four, or more, costimulatory domains selected from 4-IBB, ICOS, CD28, OX40, and CD27, or any of the above-mentioned costimulatory domains. In some embodiments, the OX40 costimulatory domain comprises SEQ ID NO: 43 (RRDQRI.PPDAIKPPGGCSFR.TPIQEEQADAHSTLAKI). In some embodiments, the OX40 costimulatory domain comprises an amino acid sequence having at least one, at least two, at least three or more modifications of an amino acid sequence of SEQ ID NO: 43. In some embodiments, the OX40 costimulatory domain is substantially similar to the OX40 costimulatory domain comprising SEQ ID NO: 43.

In embodiments, one or more intracellular signaling domains are included in the chimeric adaptor polypeptides of the subject invention. In embodiments, the one or more intracellular signaling domains are additional to one or more costimulatory domains. In embodiments, the one or more intracellular signaling domains are included to increase proliferation, persistence, and/or cytotoxic activity of the host cell (e.g., NK cell, NKT cell, γδ cell, etc.) harboring the CAD polypeptide as herein disclosed. For example, in some embodiments, the intracellular signaling domain(s) comprise CD3ζ, repeat (e.g., 2-5) DAP10 YINM motifs, signaling domains derived from LFA-1, DAP12, FcRγ, FcRβ, CD3γ, CD3δ, CD3ε, CD79a, CD79b, CD5, CD22, FcεRI, CD66d, and the like. It is within the scope of this disclosure that the endodomain of a disclosed chimeric adaptor polypeptide can include a plurality (e.g., 2, 3, 4, or more) of intracellular signaling domains. In a case where more than one intracellular signaling domain is included, the intracellular signaling domains may be the same, or they may be different.

In some embodiments, an intracellular signaling domain is or comprises a CD3ζ signaling domain. In some embodiments, a CD3ζ signaling domain is or comprises RVKFSRSADAPAYQQGQNQLYNELNLGRREEYDVLDKRRGRDPEMGGKPQRRKNPQE GLYNELQKDKMAEAY SEIGMKGERRRGKGHDGLYQGL STATKDT YDALHMQALPPR (SEQ ID NO: 3) or RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 4) or RVKFSRSADAPAYQQGQNQLYNELNLGRREEYDVLDKRRGRDPEMGGKPQRRKNPQE GLFNELQKDKMAEAF SEIGMKGERRRGKGHDGLFQGLSTATKDTFDALHMQALPPR (SEQ ID NO: 82), also referred to herein as “1XX” (see e.g., US 2020/0317777, the contents of which is incorporated by reference herein in its entirety). Without being bound by theory, inclusion of the 1XX signaling domain may, in some embodiments, lead to improved activation and/or survival of modified immune cells of the present disclosure by limiting overactivation.

In some embodiments the CAD construct encodes one or more costimulatory domain(s) (e.g., 4-1BB costimulation domain), and one or more intracellular signaling domain(s) (e.g., CD3ζ signaling domain). In some embodiments, the CAD construct encodes at least one 4-1BB costimulation domain, at least one CD28 domain, and at least one CD3ζ signaling domain. In other embodiments, the CAD construct encodes one or more first costimulation domains (e.g., 4-1BB, CD28, OX40, ICOS) and one or more second costimulation domains (e.g., 4-1BB, CD28, OX40, ICOS), and one or more intracellular signaling domains (e.g., CD3ζ). In embodiments, the CD3ζ signaling domain is downstream (C-terminal) to the costimulation domain(s) (e.g., 4-1BB). In some embodiments, the CD3ζ signaling domain is upstream (N-terminal) to the costimulation domain(s) (e.g., 4-1BB).

### 3. Cytokine Co-Expression

In additional embodiments, a CAD construct of the subject invention can also encode for one or more multicistronic linker region(s) configured to facilitate translation of the CAD polypeptide and one or more soluble common gamma chain cytokines as separate polypeptides. In embodiments, nucleic acids encoding the cytokine and associated linker region can be positioned at the 3′ end of the isolated nucleic acid, or at the 5′ end of the isolated nucleic acid, or in some examples at both the 5′ end and the 3′ end of the isolated nucleic acid. The one or more soluble common gamma chain cytokines can include but are not limited to IL-2, IL-4, IL-7, IL-9, IL-15, IL-21, IL-23. In embodiments, the linker region(s) can encode a self-cleavage and/or a cleavage polypeptide sequence. In some examples, the self-cleavage sequence is a 2A self-cleaving sequence (e.g., T2A, P2A, E2A, F2A) which can induce ribosomal skipping during translation of the chimeric DAP10 adaptor polypeptide. In embodiments, the cleavage sequence is a furin sequence. In some examples, the cleavage sequence (e.g., furin cleavage sequence) is amino terminal to a self-cleavage sequence. In some embodiments, the multicistronic linker region encodes an internal ribosome entry site. In some embodiments, the multicistronic linker region comprises a sequence of any one of SEQ ID NOs: 9-15, or SEQ ID NO: 44. In embodiments, addition of an optional linker “GSG” or “SGSG” and the like can improve cleavage efficiency. In this way, the included one or more gamma chain cytokines may be released from the chimeric DAP10 adaptor polypeptide, and secreted by the host cell.

For example, in some embodiments, the cleavage sequence is the P2A cleavage sequence of SGSGATNFSLLKQAGDVEENPGP (SEQ ID NO: 9). In some embodiments, the P2A cleavage sequence is the P2A cleavage sequence of GSGATNFSLLKQAGDVEENPGP (SEQ ID NO: 44). In some embodiments, the cleavage sequence is a furin cleavage sequence ofRAKR (SEQ ID NO: 10). In some embodiments, the cleavage sequence is a P2A+furin cleavage (FP2A) sequence of RAKRSGSGATNFSLLKQAG DVEENPGP (SEQ ID NO: 11).

In some embodiments, the cleavage sequence is or comprises a P2A cleavage sequence of ATNFSLLKQAGDVEENPGP (SEQ ID NO: 12). In some embodiments, the cleavage sequence is or comprises an F2A cleavage sequence of VKQTLNNFDLLKLAGDVESNPGP (SEQ ID NO: 13). In some embodiments, the cleavage sequence is or comprises an E2A cleavage sequence of QCTNYALLKLAGDVESNPGP (SEQ ID NO: 14). In some embodiments, the cleavage sequence is or comprises a T2A cleavage sequence of EGRSLLTCGDVEENPGP (SEQ ID NO: 15). In certain aspects, multiple self-cleavage sequences can be encoded carboxy-terminal to a signaling and/or costimulatory domain and amino-terminal to an encoded secreted cytokine (e.g., common gamma chain cytokine such as IL-15), preferably wherein the multiple self-cleavage sequences are independently selected from the group consisting of a P2A cleavage sequence, a T2A cleavage sequence, an E2A cleavage sequence, and an F2A cleavage sequence. In certain aspects, one or more self-cleavage sequences and one or more sequences cleaved by an endogenous protease are encoded in a construct described herein. In certain embodiments, an endogenous protease recognition site is encoded amino terminal to a self-cleavage sequence.

In some embodiments, the multi-cistronic linker region encodes an internal ribosome entry site. An exemplary internal ribosome entry site is encoded by

(SEQ ID NO: 16)
CTAACGTTACTGGCCGAAGCCGCTTGGAATAAGGCCGGTGTGCGTTTGT
CTATATGTTATTTTCCACCATATTGCCGTCTTTTGGCAATGTGAGGGCC
CGGAAACCTGGCCCTGTCTTCTTGACGAGCATTCCTAGGGGTCTTTCCC
CTCTCGCCAAAGGAATGCAAGGTCTGTTGAATGTCGTGAAGGAAGCAGT
TCCTCTGGAAGCTTCTTGAAGACAAACAACGTCTGTAGCGACCCTTTGC
AGGCAGCGGAACCCCCCACCTGGCGACAGGTGCCTCTGCGGCCAAAAGC
CACGTGTATAAGATACACCTGCAAAGGCGGCACAACCCCAGTGCCACGT
TGTGAGTTGGATAGTTGTGGAAAGAGTCAAATGGCTCTCCTCAAGCGTA
TTCAACAAGGGGCTGAAGGATGCCCAGAAGGTACCCCATTGTATGGGAT
CTGATCTGGGGCCTCGGTGCACATGCTTTACATGTGTTTAGTCGAGGTT
AAAAAAACGTCTAGGCCCCCCGAACCACGGGGACGTGGTTTTCCTTTGA
AAAACACGATGATA.

Another exemplary internal ribosome entry site is encoded by

(SEQ ID NO: 17)
AGCAGGTTTCCCCAACTGACACAAAACGTGCAACTTGAAACTCCGCCTG
GTCTTTCCAGGTCTAGAGGGGTAACACTTTGTACTGCGTTTGGCTCCAC
GCTCGATCCACTGGCGAGTGTTAGTAACAGCACTGTTGCTTCGTAGCGG
AGCATGACGGCCGTGGGAACTCCTCCTTGGTAACAAGGACCCACGGGGC
CAAAAGCCACGCCCACACGGGCCCGTCATGTGTGCAACCCCAGCACGGC
GACTTTACTGCGAAACCCACTTTAAAGTGACATTGAAACTGGTACCCAC
ACACTGGTGACAGGCTAAGGATGCCCTTCAGGTACCCCGAGGTAACACG
CGACACTCGGGATCTGAGAAGGGGACTGGGGCTTCTATAAAAGCGCTCG
GTTTAAAAAGCTTCTATGCCTGAATAGGTGACCGGAGGTCGGCACCTTT
CCTTTGCAATTACTGACCAC.

Further suitable internal ribosome entry sites include, but are not limited to, those disclosed e.g., in Nucleic Acids Res. 2010 January; 38(Database issue):D131-6. doi: 10.1093/nar/gkp981. Epub 2009 Nov. 16, those described at iresite.org, those described in WO 2018/215787, the sequence described in GenBank accession No. KP019382.1, and the IRES element disclosed in GenBank accession No. LT727339.1. Additional multi-cistronic linker regions, including cleavage self-cleavage, and IRES elements, are disclosed in US 2018/0360992 and U.S. Pat. No. 8,865,467.

In some embodiments, the construct encodes a secretion signal, e.g., (MALPVTALLLPLALLLHAARP (SEQ ID NO: 6)) operably linked to facilitate secretion of a C-terminal polypeptide, such as a cytokine. In some embodiments, the secretion signal is a secretion signal of MRISKPHLRSISIQCYLCLLLNSHFLTEAGIHVFILGCF SAGLPKTEA (SEQ ID NO: 7). In some embodiments, the construct encodes a secretion signal, e.g., SEQ ID NO: 7 operably linked to facilitate secretion of a common gamma chain cytokine such as IL-15 or an active fragment thereof, e.g., NWVNVISDLKKIEDLIQSMHIDATLYTESDVHPSCKVTAMKCELLELQVISLESGDASIH DTVENLIILANNSLSSNGNVTESGCKECEELEEKNIKEFLQSFVHIVQMFINTS (SEQ ID NO: 8); Other IL-15 sequences, including codon optimized nucleic acid sequences encoding sIL15, are disclosed in WO 2007/037780. Exemplary common gamma chain cytokines include IL-4, IL-7, IL-9, IL-15, IL-21, IL-23. In some embodiments, the common gamma chain cytokine is selected from IL-2, IL-7, and IL-15. In some embodiments, the common gamma chain cytokine is IL-15. IL-15 sequences, including codon optimized nucleic acid sequences encoding sIL15, are disclosed herein and in WO 2007/037780.

Thus, the CAD constructs of the present disclosure encode a CAD polypeptide including at least one costimulatory domain and, optionally, one or more intracellular signaling domains. In embodiments, the CAD constructs can encode for one or more common gamma chain cytokines that are released from the CAD polypeptide (e.g., during translation). As discussed above in some embodiments, the CAD constructs of the present disclosure may further comprise a mutated DAP10, for example DAP10 mutated at K84 and/or Y86, among others, of SEQ ID NO: 1.

In embodiments, the one or more costimulatory domains may be 5′ to the one or more signaling domains. In embodiments, the one or more costimulatory domains may be 3′ to the one or more signaling domains. In some embodiments, the one or more costimulatory domains may be 5′ to the one or more signaling domains, and additionally one or more costimulatory domains may be 3′ to the one or more signaling domains. In some embodiments, one or more signaling domains may be 5′ to one or more costimulatory domains, and additionally one or more signaling domains may be 3′ to one or more costimulatory domains. In some embodiments, the C-terminal fusion may include alternating one or more costimulatory domains and one or more signaling domains.

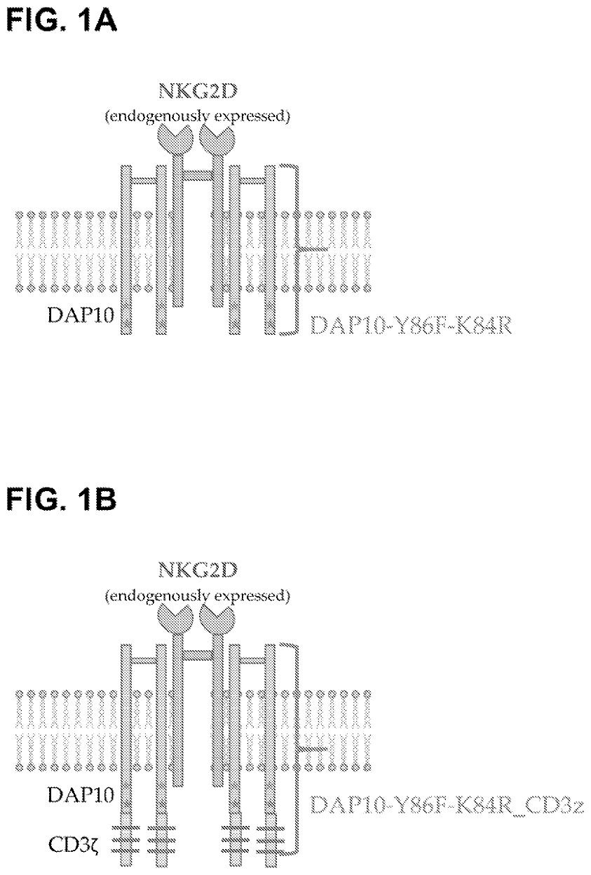

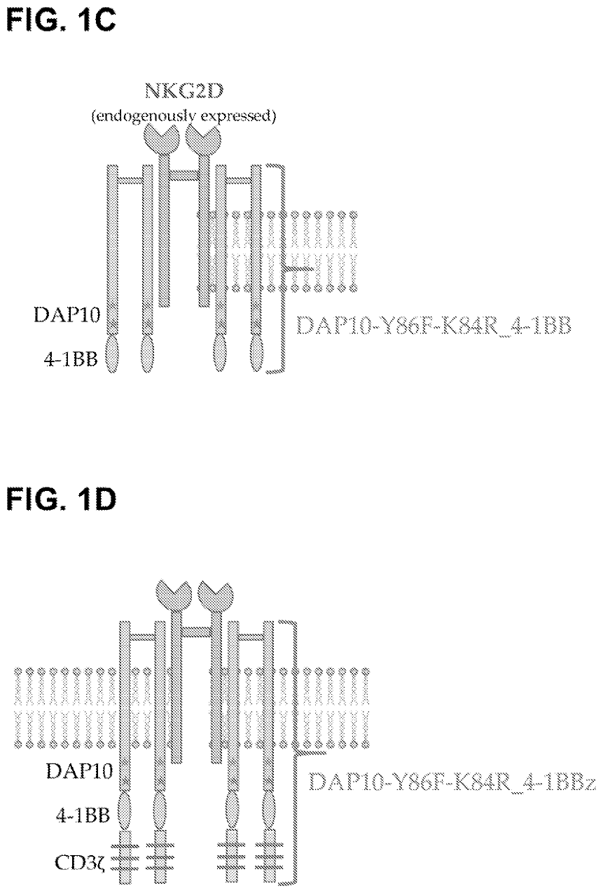

For reference, FIGS. 1A-1D depict exemplary illustrations of various chimeric adaptor polypeptides and their association with a receptor (e.g., illustrated as NKG2D). The renderings are for illustrative purposes only, and are not representative of all of the various permutations of chimeric adaptor polypeptides disclosed herein. FIG. 1A illustratively depicts a chimeric adaptor polypeptide that includes both K84R and Y86F modifications. FIG. 1B illustratively depicts a chimeric adaptor polypeptide that includes both K84R and Y86F modifications, in addition to a C-terminal fusion comprising CD3ζ. FIG. 1C illustratively depicts a chimeric adaptor polypeptide that includes both K84R and Y86F modifications, in addition to a C-terminal fusion comprising 4-1BB. FIG. 1D illustratively depicts a chimeric adaptor polypeptide that includes both K84R and Y86F modifications, in addition to a C-terminal fusion comprising both 4-1BB and CD3ζ.

### 4. Marker Co-Expression

In additional embodiments, a CAD construct of the subject invention can encode for one or more labels or markers, for example to facilitate an ability to monitor CAD expression level, serve as an internal control, and the like. In some embodiments, a CAD construct can encode for a fluorescent protein, examples of which include but are not limited to green fluorescent protein (GFP), red fluorescent protein (RFP), enhanced GFP (EGFP), enhanced cyan fluorescent protein (ECFP), enhanced yellow fluorescent protein (EYFP), and the like. Other examples can include but are not limited to chloramphenicol acetyltransferase, beta-galactosidase, beta-glucuronidase, beta-lactamase, luciferase, and the like.

In other embodiments, the CAD construct can encode for a protein that is expressed on a cell surface to facilitate detection and/or isolation of cells expressing said protein, e.g., via fluorescent activated cell sorting (FACS); or for enrichment through positive selection using an antibody specific to the encoded protein, e.g., use of an antibody to purify or enrich the cells product on a column or apparatus; or for in vivo binding of an antibody to the protein to enhance or eliminate activity, e.g., to facilitate removal of cells expressing the protein in patients as a safety consideration. Exemplary proteins useful for these purposes include, e.g., CD19, CD20 (Rituxumab recognition domain), LNGFR (amino acid sequence as set forth as SEQ ID NO: 89, encoded by SEQ ID NO: 90), a truncated form of the human epidermal growth factor receptor (EGFRt) (amino acid sequence as set forth as SEQ ID NO: 91, encoded by SEQ ID NO: 92), and the like. By way of example, in embodiments a marker protein can be targeted by a clinical stage antibody, where such treatment of a patient with said antibody results in elimination of cells containing a CAD construct and a chimeric receptor as disclosed herein. See e.g., Philip B, et al., (2014) Blood, 124(8): 1277-1287; Wang X, et al., (2011) Blood, 118(5): 1255-1263; Smith J, et al., (2015) Meeting Abstract, ASCO Annual Meeting I; 3069; Gouble A, et al., (2014) Blood, 124(21): 4689.

In embodiments, a linker region, examples of which are described herein, can be used to facilitate translation of the CAD polypeptide and the desired marker. For example, a furin and P2A linker gene may be included in an isolated nucleic acid construct of the present disclosure to facilitate CAD expression and a desired marker. Discussed below with regard to Example 1, a furin and P2A linker gene can be used to express a desired CAD polypeptide along with truncated CD19 that serves as the marker. Such an example is meant to be illustrative, and non-limiting.

### B. Chimeric Receptor Constructs

A chimeric receptor as herein disclosed comprises a polypeptide comprising an extracellular targeting domain that specifically binds to target antigens on a target cell and at least one additional domain not normally found together with the extracellular targeting domain. For example, a chimeric receptor can comprise an extracellular targeting domain, and at least one DAP10-interacting domain, where the extracellular targeting domain is heterologous to the DAP10-interacting domain. The chimeric receptor can optionally further comprise one or more additional domains (e.g., one or more costimulatory domains and/or one or more intracellular signaling domains). In another example, a chimeric receptor can comprise an extracellular targeting domain and a DAP10-interacting domain normally found together (e.g., NKG2D), but where the chimeric receptor further comprises at least one other domain (e.g., one or more costimulatory domains and/or one or more intracellular signaling domains) not normally found together with the extracellular targeting domain and the DAP10-interacting domain. In yet another example, a chimeric receptor can comprise at least two domains not normally found together, and where the chimeric receptor does not include a DAP10-interacting domain. As a representative example, such a chimeric receptor may comprise an extracellular targeting domain, a transmembrane domain (e.g., CD8a TM domain), and a cytoplasmic domain including one or more costimulatory domains and/or one or more intracellular signaling domains. With regard to costimulatory domains and/or intracellular signaling domains, it is to be understood that the costimulatory domains and/or intracellular signaling domains that can be included in chimeric receptors as herein disclosed comprise any one or more of those domains discussed above with regard to the CAD polypeptides. In embodiments, the chimeric receptor may comprise an extracellular targeting domain, a transmembrane domain (e.g., CD8a TM domain), and a cytoplasmic domain including one or more costimulatory domains but not any intracellular signaling domains. For example, such chimeric receptor may be the non-signaling CAR described in Ding et al., Cancer Res (2023) 83 (7 Supplement): 1777.

Furthermore, it is to be understood that a chimeric receptor construct of the subject invention can also encode for one or more multicistronic linker region(s) configured to facilitate translation of the chimeric receptor and one or more soluble common gamma chain cytokines as separate polypeptides, similar to that discussed above with regard to CAD polypeptides.

As mentioned above, DAP10 associates with NKG2D via the formation of two salt bridges between two arginine residues in the NKG2D transmembrane domain, and two aspartic acid residues in the DAP10 transmembrane domain (Garrity, D. et al. (2005) PNAS USA 102(21): 7641-7646). Accordingly, in embodiments, a DAP10-interacting domain comprises an amino acid sequence set forth in SEQ ID NO: 75, said sequence corresponding to the TM domain of human NKG2D. In embodiments, the DAP10-interacting domain comprises an amino acid sequence comprising at least 80%, 85%, 90%, 95%, 97%, or 99% sequence identity to SEQ ID NO: 75. In embodiments, the DAP10-interacting domain comprises an amino acid sequence comprising a portion of the amino acid sequence set forth in SEQ ID NO: 75.

In embodiments where the chimeric receptor comprises a DAP10-interacting domain coupled to an extracellular targeting domain normally found together with the DAP10-interacting domain, in an embodiment the chimeric receptor can comprise an amino acid sequence set forth in SEQ ID NO: 76, or at least a portion of the amino acid sequence set forth in SEQ ID NO: 74, and additionally one or more costimulatory domains (e.g., 4-1BB, CD28, OX40, ICOS) and/or one or more intracellular signaling domains (e.g., CD3ζ). In some embodiments, the chimeric receptor can comprise at least the extracellular targeting domain and DAP10-interacting domain of other receptors that normally include an extracellular targeting domain and a DAP10-interacting domain, for example Ly49H, Ly49D, Sirp-b1, Siglec-15, Cd3001b, and the like. In some embodiments where the chimeric receptor comprises a DAP10-interacting domain coupled to an extracellular targeting domain normally found together with the DAP10-interacting domain, the chimeric receptor can comprise an amino acid sequence comprising at least 20%, 30%, 40%, 50%, 60%, 70%, 80%, 85%, 90%, 95%, 97%, or 99% sequence identity to SEQ ID NO: 74, or an amino acid sequence comprising at least 80%, 85%, 90%, 95%, 97%, or 99% sequence identity to SEQ ID NO: 76. In exemplary embodiments, the chimeric receptor additionally comprises at least one intracellular signaling domain and/or at least one intracellular costimulatory domain.

In embodiments where the chimeric receptor comprises an extracellular targeting domain and at least one DAP10-interacting domain, where the extracellular targeting domain is heterologous to the DAP10-interacting domain, the DAP10-interacting domain can comprise an amino acid sequence corresponding to at least a portion of a TM domain of a receptor that normally includes an extracellular targeting domain and a DAP10-interacting domain. As a representative example, at least a portion of the DAP10 interacting domain of NKG2D (SEQ ID NO: 75) can be coupled to a heterologous extracellular targeting domain, where the resultant chimeric receptor optionally further comprises one or more costimulatory domains and/or one or more intracellular signaling domains. Other DAP10 interacting domains from other receptors (e.g., Ly49H, Ly49D, Sirp-b1, Siglec-15, Cd3001b) can alternatively be used.

Also encompassed by the present disclosure are chimeric receptors that lack a DAP10-interacting domain. Such chimeric receptors can be designed to comprise a TM domain that is fused to the extracellular ligand-binding domain of the chimeric receptor, but where said TM domain does not interact with DAP10 (i.e., does not comprise a DAP10-interacting domain). In one embodiment, the transmembrane domain that naturally is associated with one of the domains in the chimeric receptor is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) 4-1BB/CD137, activating NK cell receptors, an Immunoglobulin protein. B7-H3, BAFF R, BLAME (SLAMF8), BTLA, CD28, CD3 epsilon, CD45, CD4, CD5, CD8, CD9, CD16, CD22, CD33, CD37, CD64, CD80, CD86, CD134, CD137, or CD154, CD 100 (SFMA4D), CD103, CDI160 (BY55), CD18, CD 19, CD19a, D2, CD247, CD2 CD276 (B71H3), CD28, CD29, CD3 delta, CD3 epsilon, CD3 gamma, CD3 zeta, CD30, CD4, CD40, CD49a, CD49D, CD49f CD69, CD7, CD84, CD8, CD8alpha, CIDSbeta, CD96 (Tactile), CD11a, CD11b, CD11c, CD11d, CDS, CFACAM 1, CRT AM, cytokine receptor, DAP10, DNAM1 (CD226), Fc gamma receptor, GADS, GITR, HVEM (LIGHTR), 1A4, ICAM-1, 1g alpha (CD79a), IL-2R beta, IL-2R gamma, IL-7K alpha, inducible T cell costimulator (ICOS), integrins, ITGA4, ITGA6, ITGAD, ITGAE, ITGAL, ITGAM, ITGAX, 1TGB2, 1TGB7, ITGB1, KIRDS2, LAT, LFA-1, a ligand that specifically binds with CD83, LIGHT, LTBR, Ly9 (CD229), lymphocyte function-associated antigen-1 (LFA-1; CD11a/CD8), MHC class I molecule, NKG2C, NKG2D, NKp30, NKp44, NKp16, NKp80 (KLRF1), OX-40, PAG/Cbp, programmed death-I (PD-1), PSGL1, SELPLG (CD 162), Signaling Lymphocytic Activation Molecules (SLAM proteins), SLAM (SLAMF1: CD150; IPO-3), SLAMF4 (CD244, 2B4), SLAMF6 (NTB-A; Lyl08), SLAMF7, SLP-76, TNF receptor proteins, TNFR2, TNFSF14, a Toll ligand receptor, TRANCE/RANKL, VLA1, or VLA-6, or a fragment, truncation, or a combination thereof. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In embodiments, the transmembrane domain in a chimeric receptor lacking a DAP10-interacting domain is the CD8 transmembrane domain. In one embodiment, the CD8 transmembrane domain comprises the nucleic acid sequence of SEQ ID NO: 16 of U.S. Pat. No. 9,102,760. In one embodiment, the CD8 transmembrane domain comprises the nucleic acid sequence that encodes the amino acid sequence of SEQ ID NO: 22 of U.S. Pat. No. 9,102,760. In another embodiment, the CD8 transmembrane domain comprises the amino acid sequence of SEQ ID NO: 22 of U.S. Pat. No. 9,102,760.

In embodiments, the chimeric receptor may comprise a linker between various domains, added for appropriate spacing and conformation of the molecule. For example, in one embodiment, there may be a linker between the binding domain VH or VL which may be between 1 and 20 amino acids long. In other embodiments, the linker between any of the domains of the chimeric antigen receptor may be between 1 and 15 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long. Ranges including the numbers described herein are also included herein, e.g., a linker 10-30 amino acids long.

In certain embodiments, linkers suitable for use in the chimeric receptors described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine (GS)n polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the chimeric receptors described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a chimeric receptor can include linkers that are all or partially flexible, such that the linker can include a flexible linker as well as one or more portions that confer less flexible structure to provide for a desired chimeric receptor structure. Specific linkers include (G4S). linkers, wherein n=1-3, and GSTSGSGKPGSGEGSTKG (SEQ ID NO: 77). In some embodiments, the linker comprises 3-20 amino acids and an amino acid sequence at least 80% 81%, 82%, 83%, 84%, 85%, 86%, 87%, 88%, 89%, 90%, 91%, 92%, 93%, 94%, 95%, 96%, 97%, 98%, 99% or 100% identical to GSTSGSGKPGSGEGSTKG (SEQ ID NO: 77). A linker can be present between the LCVR and HCVR regions of a scFv fragment, between a variable region (such as an HCVR) and a hinge region (such as a CD8a hinge), or both. For example, the present disclosure provides a chimeric receptor comprising a (G4S)3 linker between an LCVR and an HCVR, and a (G4S)1 linker between an HCVR and a CD8α hinge. While described here in the context of chimeric receptors, use of such linkers in the context of the CAD polypeptides described herein is also within the scope of this disclosure.

The extracellular targeting domain of chimeric receptors of the present disclosure may in some examples be followed by a “spacer,” or, “hinge,” which refers to the region that moves the extracellular targeting domain away from the effector cell surface to enable proper cell/cell contact, target (i.e., ligand, antigen) binding and, in examples, activation (Patel et al., Gene Therapy, 1999; 6: 412-419). The hinge region in chimeric receptors as described herein is generally between the transmembrane (TM) and the targeting domain. In embodiments herein, at least a portion of the TM domain of a chimeric receptor comprises a DAP10-interacting domain. In certain embodiments, a hinge region is an immunoglobulin hinge region and may be a wild type immunoglobulin hinge region or an altered wild type immunoglobulin hinge region. Other exemplary hinge regions used in the chimeric receptors described herein include the hinge region derived from the extracellular regions of type 1 membrane proteins such as CD8alpha, CD8beta, CD4, CD28, CD28T, 4-1BB, and CD7, which may be wild-type hinge regions from these molecules or may be altered. In some embodiments, the spacer domains may include a chemically induced dimerizer to control expression upon addition of a small molecule. In some embodiments, a spacer is not used.

The extracellular targeting domain of chimeric receptors as herein disclosed can comprise a targeting domain derived from various receptors including, without limitation, pattern recognition receptors (PRRs), Toll-like receptors (TLRs), killer activated and killer inhibitor receptors (KARs and KIRs, respectively), complement receptors, Fc receptors, B cell receptors, and T cell receptors. In some embodiments, the extracellular targeting domain can comprise polypeptides derived from, for example, monoclonal antibodies, referred to herein as antigen-binding fragments. Non-limiting examples of antigen-binding fragments derived from antibodies include Fab fragments, F(ab′)2 fragments, Fd fragments, Fv fragments, scFv molecules, dAb fragments, and minimal recognition units consisting of the amino acid residues that mimic the hypervariable region of an antibody (e.g., an isolated complementarity determining region (CDR) such as a CDR3 peptide, or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

In some embodiments, the extracellular targeting domain specifically binds to a target antigen. In some embodiments, the target antigen is a tumor antigen. In some embodiments, the antigen is selected from a tumor-associated surface antigen, CD20, TyrD, B7H6, CD3, CD19; CD123; CD22; CD30; CD70, CD171; CD6, CS-1 (also referred to as CD2 subset 1, Claudin 18.2, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44, CD44v6; CD44v7/8; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); IL-11 Ralpha; Mesothelin; Interleukin 11 receptor alpha (IL-URa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); ErbB3, Erb B4, Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor); IGF-II receptor; carbonic anhydrase IX (CAIX); Proteasome (Prosome, Macropain) Subunit, Beta Type, 9 (LMP2); glycoprotein 100 (gplOO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); melanoma-associated antigen; o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD 179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); MAGE-A4; MAGE-A9; ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); 5T4, 81H9, ALCAM, B7-1 (CD80), B37-2 (CD86), B7-H4, B7-H6, B-human chorionic gonadotropin, CA-9, CA-125, CD133, CD138, CD23, CD²⁵, CD34, CD4, CD-40, CD56, CD8, c-Net, CS-PG4, CMV-specific antigen, cytotoxic T-lymphocyte-associated antigen 4 (CTLA-4), DLL3, disialoganglioside GD2, GD3, ductal-epithelial mucine, EBV-specific antigen, EGP-2, EGP-40, endoglin, epithelial tumor antigen, fetal acetylcholine receptor, FBP, folate binding protein, folate receptor-alpha, glioma-associated antigen, glycosphingolipids, gp36, G250/CAIX, HBV-specific antigen, HCV-specific antigen, HER1-HER2, HER2-HER3 in combination, HERV-K, -HIV-1 envelope glycoprotein gp41, H1PV-specific antigen, KDR, kappa chain, insulin growth factor (IGF1)-1, ligands belonging to the MIC (MICA and MICB) and ULBP (ULBP1-ULBP6) families in humans, L AGA-la, Lewis Y, lambda chain, lectin-reactive AF P, L1-cell adhesion molecule, MAGE, major histocompatibility complex (NUIC) molecule, major histocompatibility complex (MH1C) molecule presenting a tumor-specific peptide epitope, M-CSF, MN-CA IX, MUC-1, MUC-16, NKG2D, NKG2D ligands, neutrophil elastase, Nkp30, oncofetal antigen (hMT4)p53, prostate specific antigen (PSA_, PSC1, prostate-specific antigen protein, STEAP1, STEAP2, surface adhesion molecule, surviving and telomerase, TAG-72, the extra domain A (EDA) and extra domain B (EDB) of fibronectin and the Al domain of tenascin-C (TnC Al), thyroglobulin, Tem8, tumor stromal anuigens, VEIGFF-A, and immunoglobulin lambda-like polypeptide 1 (IGLL1).

In some additional or alternative embodiments, the target antigen is a viral antigen, such as the Epstein Barr virus antigens EBVA and the human papillomavirus (HPV) antigens E6 and E7, viral antigens present on cytomegalovirus (CMV), human immunodeficiency virus (HIV), influenza virus, coronavirus, and the like.

Other additional or alternative examples of antigens that can be targeted by chimeric antigens of the present disclosure include, without limitation, those described in WO 2016/172583, WO 2017/062820, WO 2016/149254, WO 2017/149515, WO 2014/039523, WO 2013/123061, WO 2016/166544, WO 2017/222593, and WO 2018/165228.

Targeting domains used to specifically bind antigens such as those discussed above are known, and it is to be understood that such targeting domains (e.g., monoclonal antibodies or fragments thereof, or at least one or more CDRs thereof) can readily be incorporated into chimeric receptors of the present disclosure. For example, targeting domains for use as herein disclosed can comprise or be derived from any of the targeting domains exemplified for use in chimeric receptors as described by Sadelain et al. (Cancer Discov. 3(4):388-398 (2013), see e.g., Table 1 and references cited therein) (i.e., anti-α folate receptor, anti-CAIX, anti-CD 19, anti-CD20, anti-CD22, arti-CD23, anti-CD24, arti-CD30, anti-CD33, anti-CD38, anti-CD44v7/8, antie-CEA, anti-EGFRvAIII, anti-EGP-2, anti-EGP-40, anti-EphA2, anti-erb-B2,3,4, anti-1FBP, anti-fetal acetylcholine e receptor, anti-Gm2, anti-G., anti-Her-2, anti-HMW-MAA, anti-IL-11Rα, anti-IL-13-α2, anti-KDR, anti-κ light chain, anti-Lewis Y, anti-L1-cell adhesion molecule, anti-MAGE-A1, anti-mesothelin, anti-MUC1, anti-MUC16, anti-NKG2D ligands, anti-NY-ESO-1 (157-165), anti-oncofetal antigen, ani-PSCA, anti-PSMA, anti-ROR1, anti-IgE, anti-TAG-72, anti-VEGF-R2). In some embodiments, known targeting domains can comprise or be derived from, without 1 imitation those described in U.S. patent application Ser. No. 08/940,544, and/or cited by reference therein (e.g, anti-CD28). In some embodiments, known targeting domains can comprise or be derived from, without limitation, those described in WO 2016199141, and/or cited by reference therein (e.g., anti-TyrD, anti-WT1, anti-MAGE-A4, anti-MAGE-A9, anti-PAP). In some embodiments, known targeting domains can comprise or be derived from, without limitation, those described in WO 2020/072536, and/or cited by reference therein (e.g., anti-BCMA. anti-CD20). In some embodiments, known targeting domains can comprise or be derived from, without limitation, those described in WO 2020/072546, WO2014180306, and/or U.S. Provisional Patent Application No. 63/235,093, and/or cited by reference therein (e.g., anti-GPC3, anti-TyrD). In some embodiments, known targeting domains can comprise or be derived from, without limitation, those described in WO 2015123642, and/or cited by reference therein (e.g., anti-CD19, anti-CD4, anti-CD44v6, anti-CD45, anti-CD28, anti-CD3, anti-CD3e, anti-CD123, anti-CD138, anti-CD52, anti-CD56, anti-CD74, anti-CD30, anti-Gp75, anti-CD38, anti-CD33, anti-CD20, anti-Her-3, anti-ROR1, anti-c-Met, anti-c-Myc, anti-EGFR, anti-Dectin, anti-Ebola virus, anti-fungal antigens, anti-HERVK, anti-NY-ESO-1, anti-VEGF-R2, anti-TGF-bR2, anti-IgG4, anti-biotin, anti-CSI protein, anti-mesothelin, anti-phosphatidylserine). In some embodiments, known targeting domains can comprise or be derived from, without limitation, those described in WO 2017/222593, and/or cited by reference therein (e.g., anti-CD19, anti-CD20, anti-CD22, anti-CD123, anti-CD33, anti-BAFF-R, anti-CD269, anti-CS-1, anti-CD45). In some embodiments, known targeting domains can comprise or be derived from, without limitation, those described in WO 2017/149515, and/or cited by reference therein (e.g., anti-mesothelin, anti-EGFRvIII, anti-claudin6, anti-GD2, anti-Tn, anti-sTn, anti-Tn-O-glycopeptide antigen, sTn-O-glycopeptide, anti-PSMA, anti-CD97, anti-CD44v6, anti-CEA, anti-EPCAM, anti-KIT, anti-CD171, anti-PSCA, anti-MAD-CT-2, anti-Folate receptor alpha, anti-ERBB2, anti-MUC1, anti-EGFR, anti-NCAM, anti-CAIX, anti-Fos-related antigen I, anti-SSEA-4, anti-PDGFR-beta, anti-ALK, anti-polysialic acid, anti-PLAC1, anti-GloboH, anti-NY-BR-1, anti-sperm protein 17, anti-TRP-2, anti-CYP1B1, anti-RAGE-1, anti-human telomerase transcriptase, anti-carboxyl esterase, anti-mut hsp70-2, anti-MAD-CT-2, anti-CD123, anti-CD34, anti-Flt3, anti-CD33, anti-CLL-1, anti-CD19, anti-BCMA, anti-ROR1, anti-CD22, anti-CD20), or WO 2016/044605 (e.g., anti-mesothelin). In some embodiments, known targeting domains can comprise or be derived from, without limitation, those described in WO 2017/025038, and/or cited by reference therein (e.g., anti-CD19, anti-CD20, anti-BCMA, anti-CD38). In some embodiments, known targeting domains can comprise or be derived from, without limitation, those described in WO 2019215500, and/or cited by reference therein (e.g., anti-CD19, anti-CD33, anti-CD70, anti-BCMA). In some embodiments, known targeting domains can comprise or be derived from, without limitation, those described in WO 2016/016343, and/or cited by reference therein (e.g., anti-ROR1). In some embodiments, known targeting domains can comprise or be derived from, without limitation, those described in WO 2019/028051, and/or cited by reference therein (e.g., anti-CD19, anti-CD20, anti-CD33). In some embodiments, known targeting domains can comprise or be derive from, without limitation, those described in WO2012058460, or WO2016093878 (e.g., anti-CD70). In some embodiments, known targeting domains can comprise or be derived from, without limitation, those described in WO2013169691A1 (e.g., anti-B7-H6). In some embodiments, known targeting domains can comprise or be derived from, without limitation, those described in WO2016160620, WO2014100439, WO2013025779 and/or cited by reference therein (e.g., anti-B7-14). In some embodiments, known targeting domains can comprise or be derived from, without limitation, those described in WO2019219089, WO2020200196, WO2020043044, WO2020147321 and/or cited by reference therein (e.g., anti-Claudin 18.2). In some embodiments, known targeting domains can comprise or be derived from, without limitation, those described in WO 2016/090337, WO 2017/096120, WO 2014/210064, U.S. Pat. No. 7,999,077, WO 2016/205520, U.S. Pat. No. 7,105,149, WO 2006/076691, WO 2010/114940, WO 2010/120561, and/or cited by reference therein (e.g., anti-FCRL5/FCRH5/CD307). In some embodiments, known targeting domains can comprise or be derived from, without limitation, those described in U.S. Provisional Patent Application No. 63/411,988 and/or cited by reference therein (e.g., anti-B7H6). In some embodiments, known targeting domains can comprise or be derived from, without limitation, those described in U.S. Provisional Patent Application No. 63/393,787 and/or cited by reference therein (e.g., anti-CD70). In some embodiments, known targeting domains can comprise or be derived from, without limitation, those described in U.S. Provisional Patent Application No. 63/397,296 and/or cited by reference therein (e.g., anti-PSMA). In some embodiments, known targeting domains can comprise or be derived from, without limitation, those described in Tran E. et al, Immune targeting of fibroblast activation protein triggers recognition of multipotent bone marrow stromal cells and cachexia, J Exp Med., 2013 Jun. 3; 210(6)1125-35 (e.g., anti-FAP).

In embodiments, a chimeric receptor that does not comprise a DAP10-interacting domain comprises an amino acid sequence set forth in SEQ ID NO: 8, or SEQ ID NO: 9, or SEQ ID NO: 10, or SEQ ID NO: 11, or SEQ ID NO: 12, or SEQ ID NO: 46, or SEQ ID NO: 48 of WO 2020/072536, wherein said chimeric receptor comprises an anti-CD20 extracellular targeting domain. In each of SEQ ID NOs: 8, 9, 10, 11, 12, 46, and 48 of WO 2020/072536, the chimeric receptor comprises a CD8α TM domain. Accordingly, in embodiments, the CD8a TM domain can be replaced with a DAP10-interacting domain, for example the DAP10-interacting domain comprising the amino acid sequence set forth in SEQ ID NO: 75 herein to yield an anti-CD20 chimeric receptor that associates with a CAD polypeptide of the present disclosure, Similar logic applies equally to other chimeric receptors disclosed in WO 2020/072536.

In embodiments, a chimeric receptor that does not comprise a DAP10-interacting domain comprises an amino acid sequence set forth in SEQ ID NO: 20, or SEQ ID NO: 22 of WO 2020/072546, wherein said chimeric receptor comprises an anti-GPC3 extracellular targeting domain. In each of SEQ ID NOs: 20 and 22 of WO 2020/072546, the chimeric receptor comprises a CD8α TM domain. Accordingly, in embodiments, the CD8a TM domain can be replaced with a DAP10-interacting domain, for example the DAP10-interacting domain comprising the amino acid sequence set forth in SEQ ID NO: 75 herein, to yield an anti-GPC3 chimeric receptor that associates with a CAD polypeptide of the present disclosure. Similar logic applies equally to other chimeric receptors disclosed in WO 2020/072546.

Thus, it is to be understood that in embodiments, chimeric receptors known in the art can be used in the methodology of the present disclosure with or without modification. For example, and without limitation, any of the chimeric receptors listed in Table I of Sadelain et al. (Cancer Discov. 3(4):388-398 (2013) can be used without modification, or they can be modified in a manner to incorporate a DAP10-interacting domain, such that the chimeric receptor associates with DAP10, and by extrapolation, associates with CAD polypeptides of the present disclosure. Of course, other modifications are within the scope of this disclosure, including but not limited to incorporation of one or more additional or alternative intracellular domains (e.g., costimulatory and/or intracellular signaling domains). Similar logic applies equally to other chimeric receptors known in the art, for example and without limitation, those disclosed in WO 20 16/000337.

### C. Expression of Chimeric Constructs

As used herein, an isolated nucleic acid is intended to mean a DNA molecule which can be transformed or introduced into a host cell (e.g., a T cell, NK cell, NKT cell, etc.) and be transcribed and translated to produce a product (e.g., a chimeric adaptor polypeptide or chimeric receptor as herein described). In the isolated nucleic acids of the present invention, a promoter is operably linked to the nucleic acid sequence encoding the chimeric adaptor polypeptide or chimeric receptor of the present invention, i.e., they are positioned so as to promote transcription of the messenger RNA from the DNA encoding the chimeric adaptor polypeptide or chimeric receptor. The term “operatively linked” refers to a juxtaposition wherein the components so described are in a relationship permitting them to function in their intended manner.

The promoter can be of genomic origin or synthetically generated. A variety of promoters for use in host cells relevant to the present disclosure are well-known in the art (e.g., the CD4 promoter disclosed by Marodon, et al. (2003) Blood 101(9):3416-23). The promoter can be constitutive or inducible, where induction is associated with the specific cell type or a specific level of maturation. Alternatively, a number of well-known viral promoters may also be suitable. Promoters of interest include the 3-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter. In embodiments, expression of a chimeric adaptor polypeptide is under the control of an inducible promoter, for example a promoter that is inducible by a molecule present in a tumor microenvironment (e.g., TGFβ).

The sequence of the open reading frame encoding the various segments of the chimeric adaptor polypeptides and chimeric receptors of the present disclosure can be obtained from a genomic DNA source, a cDNA source, or can be synthesized (e.g., via PCR), or combinations thereof.

In embodiments, for expression of a chimeric adaptor polypeptide of the present invention, the naturally occurring or endogenous transcriptional initiation region of the nucleic acid sequence encoding N-terminal component of DAP10 can be used to generate the chimeric adaptor polypeptide in the host cell. Alternatively, an exogenous transcriptional initiation region can be used which allows for constitutive or inducible expression, wherein expression can optionally be controlled depending upon the host cell, the level of expression desired, the nature of the host cell, and the like.

A termination region encoding a C-terminal component of the chimeric adaptor polypeptide or chimeric receptor can be included. Generally speaking, the source of the termination region is not considered to be critical to the expression of a recombinant protein and a wide variety of termination regions can be employed without adversely affecting expression.

The isolated nucleic acid, which encodes the chimeric adaptor polypeptide or a chimeric receptor according to this invention can be prepared in conventional ways. Sequences (natural or synthetic) are isolated and manipulated, as appropriate, so as to allow for the proper joining of the various components. Thus, various nucleic acid sequences encoding for the various segments of the chimeric adaptor polypeptide can be isolated, e.g., by employing the polymerase chain reaction (PCR), using appropriate primers. If necessary, specific primers can be designed which result in deletion of undesired portions of a nucleic acid sequence being used as a template. Additionally or alternatively, restriction digests of cloned genes can be used to generate the isolated nucleic acid constructs of the present disclosure. In either case, the sequences can be selected to provide for restriction sites which are blunt-ended, or have complementary overlaps for facilitating incorporation into various vectors. In examples, modification of a nucleic acid sequence (e.g., to introduce one or more point mutations, insertions or deletions), is performed. In one embodiment with regard to chimeric adaptor polypeptides of the present disclosure, the modification can, for example, comprise an amino acid change at position Y86 and/or K84 of SEQ ID NO: 1. Methods for introducing modifications into nucleic acid sequences are known in the art and can include the use of various kits available for purchase (e.g., QuickChange Site Directed Mutagenesis Kit, Agilent, Santa Clara, CA).

The various manipulations for preparing the isolated nucleic acid encoding a chimeric adaptor polypeptide or a chimeric receptor of the present disclosure can be carried out in vitro. In particular embodiments a sequence encoding a chimeric adaptor polypeptide is introduced into vectors for cloning and expression in an appropriate host cell using standard transformation or transfection methods. Thus, after each manipulation, the resulting construct from joining of the DNA sequences is cloned, the vector isolated, and the sequence screened to insure that the sequence encodes the desired chimeric adaptor polypeptide. The sequence can be screened by restriction analysis, sequencing, or the like.

It is contemplated that the isolated nucleic acid can be introduced into host cells as naked DNA or in a suitable vector. Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994). Alternatively, the polynucleotides and vectors of the disclosure can be reconstituted into liposomes for delivery to target cells.

Methods of stably transfecting host cells by electroporation using naked DNA are known in the art (see for example U.S. Pat. No. 6,410,319 disclosing T cell transfection). Naked DNA generally refers to the DNA encoding a chimeric adaptor polypeptide or chimeric receptor of the present invention contained in a plasmid expression vector in proper orientation for expression. Advantageously, the use of naked DNA reduces the time required to produce host cells expressing a chimeric polypeptide(s) of the present invention.

Alternatively, a viral vector (e.g., a retroviral vector, adenoviral vector, adeno-associated viral vector, or lentiviral vector) can be used to introduce the isolated nucleic acid encoding a chimeric polypeptide of the present invention into host cells. Suitable vectors for use in accordance with the method of the present invention are non-replicating in the T cells. A large number of vectors are known which are based on viruses, where the copy number of the virus maintained in the cell is low enough to maintain the viability of the cell. Illustrative vectors include the pFB-neo vectors (STRATAGENE®) as well as vectors based on HIV, SV40, EBV, HSV or BPV.

Thus, it may be understood that in some embodiments, the isolated nucleic acid is a circular nucleic acid. In some embodiments, the isolated nucleic acid is a vector, such as a plasmid vector, an adenoviral vector, an adeno-associated viral vector, a viral vector, a retroviral vector, (e.g, a gamma retroviral vector), or a lentiviral vector. In some embodiments, the isolated nucleic acid, or an, e.g., contiguous, portion thereof containing a DAP10 sequence (e.g., at least a portion of SEQ ID NO:1, modified or not), and one or more signaling and/or costimulatory domains is integrated into the genome of a host cell, such as a host γδ T cell. In an exemplary embodiment, the isolated nucleic acid is retroviral vector.

### D. Host Cells

Chimeric polypeptides of the present disclosure including CAD polypeptides and chimeric receptors may be expressed via their corresponding chimeric nucleic acid constructs in a wide variety of host cells. In embodiments, the host cells are mammalian cells. In embodiments, the CAD polypeptides are expressed in a host cell type that exhibits endogenous expression of a receptor that associates with DAP10. For example, the CAD polypeptide may be expressed in a host cell that expresses NKG2D. In embodiments, the CAD polypeptides are expressed in a host cell type that exhibits some level of endogenous expression of a receptor that associates with DAP10 (e.g., NKG2D, Ly49H, Ly49D, Sirp-b1, Siglec-15, Cd300lb, etc.). In embodiments, the host cell type can also be engineered to express the same receptor (e.g., NKG2D), for example to increase expression level of the receptor over the naturally-occurring endogenous expression level. In embodiments, the CAD polypeptides are expressed in a cell type that does not exhibit endogenous expression of a receptor that associates with DAP10, in which case the host cell is engineered to express such a receptor (e.g., expression of NKG2D in a cell type that does not otherwise express NKG2D, or expression of a chimeric receptor comprising a DAP10-interacting domain). In embodiments, the CAD polypeptides are expressed in a cell type that exhibits endogenous expression of a receptor that associates with DAP10 (e.g., NKG2D), where the cell is further engineered to express a chimeric receptor that lacks a DAP10-interacting domain. In embodiments, the CAD polypeptides are expressed in a cell type that exhibits endogenous expression of a receptor that associates with DAP10 (e.g., NKG2D), where the cell is further engineered to express at least one chimeric receptor that includes a DAP10-interacting domain, said chimeric receptor comprising at least an extracellular targeting domain and a DAP10-interacting domain, optionally further comprising one or more costimulatory domains and/or one or more intracellular domains. In embodiments, the CAD polypeptides are expressed in a cell type that lacks endogenous expression of a receptor that associates with DAP10, and is engineered to express a non-chimeric receptor that associates with DAP10, and is further engineered to express a chimeric receptor, where in some examples the chimeric receptor includes a DAP10-interacting domain, but in other examples lacks such a DAP10-interacting domain, and where said chimeric receptor includes one more costimulatory domains and/or one or more intracellular ligand binding domains.

In embodiments, expression of a CAD polypeptide of the present disclosure in a host cell results in the CAD polypeptide competing with endogenous cellular DAP10 (e.g., WT DAP10). Via the competing, intracellular signaling through a receptor (e.g., endogenous, exogenous, or chimeric) that associates with DAP10 can be redirected through the chimeric DAP10 adaptor polypeptide. To be effective for a desired outcome (e.g., tumor cell killing), the signaling need not be redirected 100% through the CAD polypeptide, although such a percentage is within the scope of this disclosure. Signaling through the CAD polypeptide may comprise anywhere from 20% to 100% of signaling through the DAP10, e.g., between 90-100%, between 80-100%, between 70-100%, between 60-100%, between 50-100%, etc. As a representative example for illustrative purposes, a host cell in which 80% of signaling via a receptor that associates with DAP10 is re-routed through a CAD polypeptide means that just 20% of such signaling remains through the endogenous DAP10, while 80% of signaling is through the CAD polypeptide.

Host cells, as described herein, can be stored, e.g., cryopreserved, for use in adoptive cell transfer. In embodiments, the host cells are stored prior to engineering the cells to express a CAD polypeptide and a chimeric receptor. In embodiments, the cells are engineered to express a chimeric DAP10 adaptor polypeptide and then the cells are stored.

Preferred host cells for use with the CAD polypeptides and chimeric receptors of the present disclosure comprise immune cells. Such cells may be obtained from the subject to be treated (i.e. are autologous) or, alternatively, immune cell lines or donor immune cells (allogeneic, syngeneic) can be used. Immune cells can be obtained from a number of sources, including from peripheral blood mononuclear cells, bone marrow, lymph node tissue, cord blood, thymus tissue, tissue from a site of infection, ascites, pleural effusion, spleen tissue, and tumors. Immune cells can be obtained from blood collected from a subject using any number of techniques known to the skilled artisan, such as Ficoll™ separation. For example, cells from the circulating blood of an individual may be obtained by apheresis. In some embodiments, immune cells are isolated from peripheral blood lymphocytes by lysing the red blood cells and depleting the monocytes, for example, by centrifugation through a PERCOLL™ gradient or by counterflow centrifugal elutriation. A specific subpopulation of immune cells can be further isolated by positive or negative selection techniques. For example, immune cells can be isolated using a combination of antibodies directed to surface markers unique to the positively selected cells, e.g., by incubation with antibody-conjugated beads for a time period sufficient for positive selection of the desired immune cells. Alternatively, enrichment of immune cell populations can be accomplished by negative selection using a combination of antibodies directed to surface markers unique to the negatively selected cells. Other specific manners of isolation and/or enrichment are disclosed herein.

In some embodiments, the immune cells comprise any leukocyte involved in defending the body against infectious disease and foreign materials. For example, the immune cells can comprise lymphocytes, monocytes, macrophages, dendritic cells, mast cells, neutrophils, basophils, eosinophils, or any combinations thereof. For example, immune cells relevant to the present disclosure can include but are not limited to up T cells, γδ T cells, NK cells, NKT cells, γδ NKT cells, B cells, innate lymphoid cells (ILCs), cytokine induced killer (CIK) cells, cytotoxic T lymphocytes (CTLs), lymphokine activated killer (LAK) cells, regulatory T cells, and the like. In embodiments, preferred immune cells comprise up T cells, γδ T cells, NK cells, NKT cells, γδ NKT cells, and/or, in some examples, macrophages. In embodiments, preferred immune cells comprise γδ T cells. In embodiments, the immune cells relevant to the present disclosure comprise allogeneic cells, autologous cells, or syngeneic cells.

Aspects of the disclosure include immune cells having in vitro or in vivo cytotoxic activity against a hematological or solid tumor cell that exhibits cell surface expression of a tumor associated antigen (TAA), virally infected cells displaying a virally-derived antigen, bacterial cells, etc. In embodiments, the cytotoxic activity is innate activity. In embodiments, the immune cells that functionally express both a CAD polypeptide and at least one chimeric receptor exhibit cell killing activity greater than the level of in vitro and/or in vivo cell killing activity in a control immune cell that does not comprise the CAD polypeptide and/or the at least one chimeric receptor of the present disclosure.

In embodiments, cytotoxicity is significantly (>about 25%) enhanced or improved by the presence of both a CAD polypeptide and at least one chimeric receptor, as compared to cytotoxicity in absence of the CAD polypeptide and/or the at least one chimeric receptor. In some cases, the cytotoxicity is at least in part, significantly (>about 25%), or entirely, due to the presence of both the CAD polypeptide and the at least one chimeric receptor.

In embodiments, engineered immune cells relevant to the present disclosure can exhibit robust and/or persistent cell killing activity (e.g., tumor cell, virally-infected cell) through direct and/or indirect mechanisms. In some cases, the cell killing activity persists for at least about 6 days to 120 days, or for at least about 6 days to 180 days, from first contact with a target cell. In some cases, the cell killing activity of an immune cell disclosed herein, engineered to express a CAD polypeptide and at least one chimeric receptor, or a progeny thereof, persists for at least about 6 days to 120 days, or for at least about 6 days to 180 days, from first contact with a target cell, or from administration of the engineered immune cells disclosed herein. This persistent cell killing activity may be exhibited in vitro, in vivo, or both in vitro and in vivo.

Aspects of the disclosure, in embodiments, include immune cells that proliferate in response to contact with cells that exhibit cell surface expression of a ligand that is recognized by a receptor that associates with DAP10, and specifically, associates with CAD polypeptides of the present disclosure. One example of such a receptor is NKG2D, although the present disclosure is not limited to CAD polypeptides interacting with NKG2D, and can include other receptor(s) that also associate with DAP10, for example a chimeric receptor engineered to include a DAP10-interacting domain. In embodiments, the proliferation is at least in part, significantly (>about 20% or >about 25%, or >about 50%, or >about 80%), or entirely (e.g., 100%), due to the presence of a CAD polypeptide construct that associates with a receptor (e.g., endogenous or exogenous NKG2D, chimeric receptor engineered to include a DAP10-interacting domain) expressed on the host cell. In some cases, the immune cells exhibit a greater level of in vitro and/or in vivo proliferation as compared to a control immune cell (e.g., same type of immune cell) that does not comprise both a CAD polypeptide and at least one chimeric receptor. The greater level of proliferation may comprise a 20-50% increase, a 50-80% increase, an 80-100% increase, or even a 2-fold increase, 3-fold increase, 4-fold increase, 5-fold increase, 5-10-fold increase, 10-20-fold increase, or even greater than a 20-fold increase, such as e.g., a 50-100-fold increase or more as compared to a control immune cell that lacks a CAD polypeptide and/or at least one chimeric receptor as herein described.

In some embodiments, the immune cells engineered to comprise both a CAD polypeptide and at least one chimeric receptor described herein express and secrete, or persistently express and secrete, one or more pro-inflammatory cytokines, for example after contact with a cell that expresses a ligand that is recognized by a cell surface receptor on the immune cell that associates with DAP10. In embodiments, the expression and secretion associated with an immune cell engineered to express both a CAD polypeptide and at least one chimeric receptor is at least in part, significantly (>about 20%, or >about 25%, or >about 50%, or >about 80%), or entirely due to one or both of the CAD polypeptide and/or the at least one chimeric receptor. In embodiments, the expression and/or secretion is greater by at least 10%, 20%, 30%, 40%, 50%, 60%, 70%, 80%, 90%, 100%, or even greater than 100%, for example 2-fold higher, 3-fold higher, 4-fold higher, 5-fold higher, 5-10-fold higher, 10-20-fold higher, or even greater than 20-fold higher, such as e.g., 50-100-fold higher or more as compared to expression and/or secretion otherwise observed in control immune cells (e.g., same type of immune cells) lacking expression of the CAD polypeptide and/or the at least one chimeric receptor.

In embodiments, engineered immune cells of the present disclosure may function to alter a cellular microenvironment (e.g., TME) in favor of, for example, an anti-tumor response. For example, solid tumors can recruit inhibitory cells such as myeloid-derived suppressor cells (MDSCs), which can strengthen a suppressive TME. Frequency of circulating or intratumoral MDSCs correlates with cancer stage, disease progression, and resistance to standard chemotherapy and radiotherapy. Certain ligands (e.g., NKG2D ligands) are expressed at high levels on several solid tumors and on tumor-infiltrating MDSCs, hence engineered immune cells of the present disclosure may in embodiments be used to alter a TME in favor of an anti-tumor response by reducing or eliminating suppressive molecules of the TME, such as TGF-β. In embodiments, engineered immune cells of the present disclosure are cytotoxic against MDSCs, but spare (i.e., are non-toxic to) NKG2D ligand-expressing normal tissues (see e.g., Parihar, R., et al., (2019) Cancer Immunol Res 7(3): 363-375). In some embodiments, cell killing activity pertaining to killing of e.g., MDSCs, by engineered host cells of the present disclosure decreases a suppressive effect of a TME. For example, the suppressive effect may be decreased by about 20%, about 30%, about 40%, about 50%, about 60%, about 70%, about 80%, about 90%, about 95%, or more, as compared to a suppressive effect of a TME in absence of host cells engineered to express a CAD polypeptide and a chimeric receptor as herein described.

In embodiments, engineered immune cells of the present disclosure may function to decrease growth and/or proliferation of a target cell. For example, an immune cell engineered to express both a CAD polypeptide and at least one chimeric receptor as herein described may decrease growth and/or proliferation of a target cell by about 20%, about 30%, about 40%, about 50%, about 60%, about 70%, about 80%, about 90%, or even about 100%, or any percentage there between, as compared to growth and/or proliferation of the target cell in absence of the engineered immune cell, or in the presence of an engineered immune cell expressing a CAD polypeptide or a chimeric receptor, but not both. In embodiments, the target cell expresses at least one cell-surface antigen that is recognized by at least one receptor (e.g., NKG2D, chimeric receptor) on the cell surface of the engineered immune cell, where said receptor associates, at least in part, with the CAD polypeptide.

In embodiments, engineered immune cells of the present disclosure may function to exert cytotoxicity against a target cell. For example, an immune cell engineered to express both a CAD polypeptide and at least one chimeric receptor as herein described may result in increased death of a population of one or more target cells, as compared to an amount of cell death in the absence of the engineered immune cell, or in the presence of an engineered immune cell expressing a CAD polypeptide or a chimeric receptor, but not both. The amount of cell death may be about 20%, about 30%, about 40%, about 50%, about 60%, about 70%, about 80%, about 90%, or even about 100%, or any percentage there between, as compared to the amount of cell death in absence of the engineered immune cell, or in the presence of an engineered immune cell expressing a CAD polypeptide or a chimeric receptor, but not both.

Expression of CAD polypeptides comprising heterologous signaling domains that influence cell survival and proliferation (e.g., 4-1BB, OX40, CD28, ICOS, IL2R, CD27, etc.) enables logic gating strategies (e.g., “AND” gating) that can increase stringency and/or potency of target attack when paired with chimeric receptors on the same host cell (e.g., T cell or NK cell) that engage targets that have low level expression in normal tissue. Accordingly, host cells with both a chimeric receptor that recognizes a primary target (e.g., primary cancer target, viral antigen, autoimmune antigen, etc.) and an appropriate CAD polypeptide may promote sustained survival, proliferation and killing of target cells that express both the primary target(s) and ligands of a receptor (e.g., NKG2D) that associates with DAP10. In such examples, the chimeric receptor can, in embodiments, include a DAP10 interacting domain. Examples of logic gating strategies applicable to the present disclosure can be found, e.g., in WO2019118518, WO2020154635, WO2020223445, WO2021035093, WO2019222642A1, WO2018236825A1, WO2019164979, and Chang, Z L, and Chen Y Y (2017) Trends Mol Med 23(5): 430-450).

Accordingly, in embodiments a host cell can comprise a CAD polypeptide comprising a DAP10 domain, at least one of a costimulatory domain or an intracellular domain, and specifically lacking an ectodomain, wherein the host cell further expresses at least one chimeric receptor comprising at least an extracellular targeting domain, an intracellular signaling domain, and/or a costimulatory domain, wherein when the chimeric receptor comprises an intracellular signaling domain, the CAD polypeptide comprises a costimulatory domain, and vice versa. In some embodiments, the chimeric receptor lacks a DAP10-interacting domain, wherein the CAD polypeptide interacts with a receptor harboring a DAP10-interacting domain (e.g., NKG2D). In other embodiments, the chimeric receptor includes a DAP-10 interacting domain. As an example, a CAD polypeptide can include at least one 4-1BB costimulatory domain but lack an intracellular signaling domain, where the chimeric receptor includes at least one intracellular signaling domain (e.g., CD3ζ), and vice versa. As another example, a CAD polypeptide can include at least one CD28 costimulatory domain, or at least one OX40 costimulatory domain, or at least one ICOS costimulatory domain, but lack an intracellular signaling domain, where the chimeric receptor includes at least one intracellular signaling domain (e.g., CD3ζ), and vice versa. In some embodiments, a CAD polypeptide can include at least two different costimulatory domains, for example a CAD polypeptide can include at least one 4-1BB and at least one CD28 (or OX40, or ICOS, etc.) costimulatory domain but lack an intracellular signaling domain, where the chimeric receptor includes at least one intracellular signaling domain (e.g., CD3ζ), optionally where the chimeric receptor includes at least two different intracellular signaling domains, and vice versa.

Hence, in embodiments methods of making a cell comprising both a CAD polypeptide and at least one chimeric receptor are provided comprising introducing into the cell an isolated nucleic acid that encodes the CAD polypeptide and at least one isolated nucleic acid that encodes the at least one chimeric receptor, such that the cell expresses both the CAD polypeptide and the at least one chimeric receptor. In embodiments, the cell is an immune cell as herein described, for example an NK cell, an NKT cell, a 76 T cell, an ca T cell, or an γδ NKT cell. In some embodiments, the method of making the cell further comprises introducing into the cell another isolated nucleic acid that encodes for a receptor capable of associating with the CAD polypeptide.

### E. Methods of Use

In one aspect, the present disclosure provides a method of modulating a signal transduced through at least one receptor of a host cell, the host cell comprising an immune cell engineered to express both a CAD polypeptide and at least one chimeric receptor as described herein, and preferably wherein the immune cell is cytotoxic, more preferably wherein the immune cell is a γδ T cell. In embodiments, the receptor is endogenous to the immune cell, and is endogenously expressed therein. In additional or alternative embodiments, the receptor is expressed by way of introducing into the immune cell an isolated nucleic acid encoding the receptor. In additional or alternative embodiments, the receptor is a chimeric receptor as herein described, and optionally includes a DAP10-interacting domain.

In embodiments, the method of modulating the signal transduced through the at least one receptor results in stimulation of the immune cell and/or activation of the immune cell.

In embodiments, the method of modulating the signal transduced through the at least one receptor results in an increased level of proliferation of the immune cell as compared to a level of proliferation of a control immune cell lacking the CAD polypeptide.

In embodiments, the method of modulating the signal transduced through the receptor results in increased expression and secretion of one or more cytokines, as compared to a level of expression and secretion of said one or more cytokines in a control immune cell lacking the CAD polypeptide and/or the chimeric receptor.

In embodiments, modulating the signal transduced through the receptor comprises routing at least a portion of the signal through the CAD polypeptide, as opposed to endogenous DAP10. In embodiments, the portion of the signal routed through the CAD polypeptide is 80% or higher, for example 90-95% or higher, for example 99% or 100%.

### F. Methods of Treatment

Pharmaceutical compositions comprising engineered host cells that express both a CAD polypeptide and at least one chimeric receptor, and/or admixtures thereof, as described herein may be administered for prophylactic and/or therapeutic treatments. In preferred embodiments, a pharmaceutical composition comprises 76 T cells engineered to express both a CAD polypeptide and at least one chimeric receptor. An admixture may comprise different types of host cells engineered to express a same or a different CAD polypeptide and a same or different chimeric receptor as herein described. For example, and without limitation, an admixture may comprise a population of NK cells expressing a first CAD polypeptide and a first chimeric receptor, and a population of γδ cells engineered to express a second CAD polypeptide and a second chimeric receptor, where the first CAD polypeptide is the different from the second CAD polypeptide and where the first chimeric receptor is the same or different from the second chimeric receptor. As another example and without limitation, an admixture may comprise a population of NK cells and a population of γδ cells each engineered to express a same CAD polypeptide, and the same or different chimeric receptors. As yet another example, an admixture may comprise a population of engineered host cells, and may additionally comprise a non-engineered cell population. For example, and without limitation, an admixture may comprise a population of γδ cells or NK cells engineered to express both a CAD polypeptide and at least one chimeric receptor as herein described, and another non-engineered population of cells e.g., NK cells, NKT cells, γδ cells, αβ cells, etc. In therapeutic applications, the compositions can be administered to a subject already suffering from a disease or condition in an amount sufficient to decrease at least one sign or symptom associated with the disease or condition. In some embodiments, the amount is sufficient to cure the disease or condition.

An engineered host cell population and/or admixtures thereof can also be administered to lessen a likelihood of developing, contracting, or worsening a condition. Effective amount of a population of engineered host cells, non-engineered host cell, and/or admixtures thereof, for therapeutic use can vary based on the severity and course of the disease or condition, previous therapy, the subject's health status, weight, and/or response to various drugs, and/or the judgement of a treating physician.

In embodiments, the one or multiple engineered host cell populations, non-engineered cells and/or admixtures thereof, of the present disclosure can be used to treat a subject in need of treatment for a condition. Examples of such conditions include but are not limited to cancer, infectious disease, and autoimmune disorder. Subjects can be humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants.

A method of treating a condition (e.g., ailment) in a subject may comprise administering to the subject a therapeutically-effective amount of one or more engineered host cell populations (e.g., to express both a CAD polypeptide and at least one chimeric receptor), non-engineered cells and/or admixtures thereof. The one or multiple engineered host cell populations, non-engineered cells, and/or admixtures thereof can be administered at various regimens (e.g., timing, concentration, dosage, spacing between treatment, and/or formulation). A subject can also be preconditioned with, for example, chemotherapy, radiation, or a combination of both, prior to receiving the therapeutically-effective amount of one or multiple engineered host cell populations, non-engineered cells, and/or admixtures thereof. As part of a treatment, one or multiple engineered host cell populations, non-engineered cells, and/or admixtures thereof may be administered to a subject at a first regimen and the subject may be monitored to determine whether the treatment at the first regimen meets a given level of therapeutic efficacy. In some cases, the one or multiple engineered host cell populations, non-engineered cells, and/or admixtures thereof may be administered to the subject at a second regimen, based on information gleaned from providing the subject with the first regimen.

In embodiments, a pharmaceutical composition comprising at least one host cell engineered to express both a CAD polypeptide and at least one chimeric receptor may be administered in a first regimen. The subject may be monitored, for example by a healthcare provider (e.g., treating physician or nurse). In some examples, the subject is monitored to determine or gauge an efficacy of the engineered host cell in treating the condition of the subject. In some situations, the subject may also be monitored to determine the in vivo expansion of an engineered host cell population in the subject. Another pharmaceutical composition comprising at least one host cell engineered to express a CAD polypeptide and at least one chimeric receptor may be administered to the subject in a second regimen. The pharmaceutical composition administered in the second regimen may comprise a same type of host cell expressing a same CAD polypeptide and at least one chimeric receptor as that administered to the subject in the first regimen. However, it is within the scope of this disclosure that the pharmaceutical composition administered in the second regimen may comprise a different type of host cell, optionally expressing a different CAD polypeptide (e.g., a CAD polypeptide with different mutations and/or costimulatory or signaling domains) and/or different chimeric receptor(s). In some examples, the second regimen is not performed, for example if the first regimen is found to be effective (e.g., a single round of administration may be sufficient to treat the condition). In some embodiments, a population of engineered host cells can be administered to various subjects (e.g., where the host cell has universal donor characteristics).

A therapeutically-effective amount of one or multiple engineered host cell populations (e.g., expressing both a CAD polypeptide and at least one chimeric receptor), non-engineered cells and/or admixtures thereof may be used to treat various conditions. In some cases, a therapeutically-effective amount of one or multiple engineered host cell populations (e.g., expressing both a CAD polypeptide and at least one chimeric receptor), non-engineered cells and/or admixtures thereof may be used to treat cancer, including solid tumors and hematologic malignancies. In some cases, a therapeutically-effective amount of one or multiple engineered host cell populations (e.g., expressing both a CAD polypeptide and at least one chimeric receptor), non-engineered cells and/or admixtures thereof may be used to treat an infectious disease caused, for example, by a pathogenic bacterium or by a virus.

Treatment with one or multiple engineered host cell populations (e.g., expressing both a CAD polypeptide and at least one chimeric receptor), non-engineered cells and/or admixtures thereof, of the disclosure may be provided to the subject before, during, and after the clinical onset of the condition. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition comprising one or multiple engineered host cell populations (e.g. expressing both a CAD polypeptide and at least one chimeric receptor), non-engineered cells and/or admixtures thereof, of the disclosure.

In some cases, administration of the one or multiple engineered host cell populations (e.g., expressing both a CAD polypeptide and at least one chimeric receptor), non-engineered cells and/or admixtures thereof, of the disclosure, modulates the activity of endogenous lymphocytes in a subject's body. In some cases, administration of the one or multiple engineered host cell populations (e.g., expressing both a CAD polypeptide and at least one chimeric receptor), non-engineered cells and/or admixtures thereof, of the disclosure, results in activation of cytotoxicity of another immune cell. In some cases, the other immune cell is a CD8+ T-cell. In some cases, the other immune cell is a Natural Killer T-cell. Other examples of other immune cells are encompassed by the present disclosure. In some cases, administration of the one or multiple engineered host cell populations (e.g., expressing a both a CAD polypeptide and at least one chimeric receptor), non-engineered cells and/or admixtures thereof, of the disclosure, suppresses a regulatory T-cell. In some cases, the regulatory T-cell is a Fox3+ Treg cell. In some cases, the regulatory T-cell is a Fox3− Treg cell. Non-limiting examples of cells whose activity can be modulated by administration of the one or multiple engineered host cell populations (e.g., expressing both a CAD polypeptide and at least one chimeric receptor), non-engineered cells and/or admixtures thereof, of the disclosure, include hematopioietic stem cells; B cells; CD4+ cells; CD8+ cells; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells.

One or multiple engineered host cell populations, non-engineered cells, and/or admixtures thereof, having cytotoxic activity against, for example and without limitation, a hematological or solid tumor cell, or a virally-infected cell, or a bacterial cell, can be administered to a subject in any order or simultaneously. If simultaneously, the engineered host cell(s), and/or admixtures thereof, of the disclosure can be provided in a single, unified form, such as an intravenous injection, or in multiple forms, for example, as multiple intravenous infusions, s.c, injections or pills. The one or multiple engineered host cell populations, non-engineered cells, and/or admixtures thereof of the disclosure can be packed together or separately, in a single package or in a plurality of packages. One or all of the one or multiple engineered host cell populations, non-engineered cells, and/or admixtures thereof of the disclosure can be given in multiple doses. If not simultaneous, the timing between the multiple doses may vary to as much as about a week, a month, two months, three months, four months, five months, six months, or about a year. In some cases, an engineered host cell of the disclosure can proliferate within a subject's body, in vivo, after administration to a subject. The one or multiple engineered host cell populations, non-engineered cells, and/or admixtures thereof of the present disclosure can be frozen to provide cells for multiple treatments with the same cell preparation. The one or multiple engineered host cell populations, non-engineered cells, and/or admixtures thereof, and pharmaceutical compositions comprising the same, can be packaged as a kit. A kit may include instructions (e.g., written instructions) on the use of the one or multiple engineered host cell populations, non-engineered cells, and/or admixtures thereof, and compositions comprising the same.

In some cases, a method of treating a subject in need thereof comprises administering to the subject a therapeutically-effective amount of one or multiple engineered host cell populations, non-engineered cells, and/or admixtures thereof, of the disclosure, wherein the administration treats a particular condition (e.g., cancer, viral or bacterial infection, autoinflammatory disease). In some embodiments the therapeutically-effective amount of the one or multiple engineered host cell populations, non-engineered cells, and/or admixtures thereof, is administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year. In some embodiments the therapeutically-effective amount of the one or multiple engineered host cell populations, non-engineered cells, and/or admixtures thereof, is administered for at least one week. In some embodiments the therapeutically-effective amount of the one or multiple engineered host cell populations, non-engineered cells, and/or admixtures thereof, of the disclosure, is administered for at least two weeks.

The one or multiple engineered host cell populations, non-engineered cells, and/or admixtures thereof, described herein can be administered before, during, or after the occurrence of a disease or condition, and the timing of administering a pharmaceutical composition containing the engineered host cell population can vary. For example, the one or multiple engineered host cell populations, non-engineered cells, and/or admixtures thereof can be used as a prophylactic and can be administered continuously to subjects with a propensity to conditions or diseases in order to lessen a likelihood of the occurrence of the disease or condition. The initial administration can be via any route practical, such as by any route described herein using any formulation described herein. In some examples, the administration of the one or multiple engineered host cell populations, non-engineered cells, and/or admixtures thereof of the disclosure is an intravenous administration. One or multiple dosages of the one or multiple engineered host cell populations, non-engineered cells, and/or admixtures thereof can be administered as soon as is practicable after the onset of a particular condition (e.g., hematological or solid cancer, viral infection, bacterial infection, autoimmune disorder, etc.) and for a length of time necessary for the treatment of the disease/condition, such as, for example, from about 24 hours to about 48 hours, from about 48 hours to about 1 week, from about 1 week to about 2 weeks, from about 2 weeks to about 1 month, from about 1 month to about 3 months. In some embodiments, one or multiple dosages of the one or multiple engineered host cell populations, non-engineered cells, and/or admixtures thereof can be administered years after onset of the disease/condition (e.g., cancer) and before or after other treatments.

In some embodiments, the one or multiple engineered host cell populations, non-engineered cells, and/or admixtures thereof, of the disclosure, is administered simultaneously or sequentially with one or more methods to elevate common gamma chain cytokine(s). As used herein, “one or more methods to elevate common gamma chain cytokine(s): refers to a method, or combination of methods, that alters the physiological state of a subject, such that at least one common gamma chain cytokine level is elevated in the subject. In some embodiments, the method elevates the level of one or more common gamma chain cytokine(s) selected from the group consisting of IL-2, IL-4, IL-7, IL-15, and IL-21 in the subject. In some embodiments, the method comprises lymphodepletion. In some embodiments, the method comprises administering one or more common gamma chain cytokine(s) to the subject. In some cases, IL-2, IL-4, IL-7, IL-15, and/or IL-21 are administered. In some embodiments, the method comprises secreting common gamma chain cytokine(s) from an administered engineered host cell. In some cases, IL-2, IL-4, IL-7, IL-15, and/or IL-21 are secreted.

In some embodiments, the administering one or more methods to elevate common gamma chain cytokine(s) comprises lymphodepletion before introducing the one or multiple engineered host cell populations, non-engineered cells, and/or admixtures thereof, of the disclosure. In some embodiments, the administering one or more methods to elevate common gamma chain cytokine(s) comprises administering simultaneously with introducing the one or multiple engineered host cell populations, non-engineered cells, and/or admixtures thereof, or sequentially an amount of common gamma chain cytokine(s) effective to increase proliferation, cytotoxic activity, persistence, or the combination thereof of the introduced one or multiple engineered host cell populations, non-engineered cells, and/or admixtures thereof. The amount of administered common gamma chain cytokine(s) can be an amount effective to increase proliferation, cytotoxic activity, persistence, or the combination thereof of the one or multiple engineered host cell populations, non-engineered cells, and/or admixtures thereof. Exemplary amounts of IL-15 include, without limitation between 0.01 10 μg/kg/dose every 24 hours for IL-15. Exemplary amounts of IL-2 include, without limitation, between about 3×10⁶ and about 22×10⁶ units every 8-48 hours. For example, the dosing regimen for IL2 in RCC is 600,000 International Units/kg (0.037 mg/kg) IV 48 hr infused over 15 minutes for a maximum 14 doses.

In some embodiments, the administering one or more methods to elevate common gamma chain cytokine(s) comprises lymphodepletion before administering the one or multiple engineered host cell populations, non-engineered cells, and/or admixtures thereof and administering simultaneously with introducing the one or multiple engineered host cell populations, non-engineered cells, and/or admixtures thereof, or sequentially, an amount of common gamma chain cytokine(s) effective to increase proliferation, cytotoxic activity, persistence, or the combination thereof of the introduced one or multiple engineered host cell populations, non-engineered cells, and/or admixtures thereof.

In some embodiments, elevating common gamma chain cytokine(s) is accomplished, at least in part, via the engineered host cell(s), where the common gamma chain cytokine(s) are expressed from a CAD construct and/or a chimeric receptor construct as disclosed herein. In such an example, it is within the scope of this disclosure that one or more additional gamma chain cytokine(s) are additionally administered in a manner to elevate said additional gamma chain cytokine(s).

### G. Dosages

One or multiple engineered host cell populations, non-engineered cells, and/or admixtures thereof, of the present disclosure, may be formulated in unit dosage forms suitable for single administration of precise dosages. In some cases, the unit dosage forms comprise additional lymphocytes. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose re-closable containers can be used, for example, in combination with a preservative or without a preservative. In some examples, the pharmaceutical composition does not comprise a preservative. Formulations for parenteral injection can be presented in unit dosage form, for example, in ampoules, or in multi-dose containers with a preservative.

One or multiple engineered host cell populations, non-engineered cells, and/or admixtures thereof, of the present disclosure, may be present in a composition in an amount of at least 5 cells, at least 10 cells, at least 20 cells, at least 30 cells, at least 40 cells, at least 50 cells, at least 60 cells, at least 70 cells, at least 80 cells, at least 90 cells, at least 100 cells, at least 200 cells, at least 300 cells, at least 400 cells, at least 500 cells, at least 600 cells, at least 700 cells, at least 800 cells, at least 900 cells, at least 1×10³ cells, at least 2×10³ cells, at least 3×10³ cells, at least 4×10³ cells, at least 5×10³ cells, at least 6×10³ cells, at least 7×10³ cells, at least 8×10³ cells, at least 9×10³ cells, at least 1×10⁴ cells, at least 2×10⁴ cells, at least 3×10⁴ cells, at least 4×10⁴ cells, at least 5×10⁴ cells, at least 6×10⁴ cells, at least 7×10⁴ cells, at least 8×10⁴ cells, at least 9×10⁴ cells, at least 1×10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells, at least 2×10⁷ cells, at least 3×10⁷ cells, at least 4×10⁷ cells, at least 5×10⁷ cells, at least 6×10⁷ cells, at least 7×10⁷ cells, at least 8×10⁷ cells, at least 9×10⁷ cells, at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, or more.

The therapeutically effective dose of one or multiple engineered host cell populations, non-engineered cells, and/or admixtures thereof, of the invention can be from about 1 cell to about 10 cells, from about 1 cell to about 100 cells, from about 1 cell to about 10 cells, from about 1 cell to about 20 cells, from about 1 cell to about 30 cells, from about 1 cell to about 40 cells, from about 1 cell to about 50 cells, from about 1 cell to about 60 cells, from about 1 cell about 70 cells, from about 1 cell to about 80 cells, from about 1 cell to about 90 cells, from about 1 cell to about 100 cells, from about 1 cell to about 1×10³ cells, from about 1 cell to about 2×10³ cells, from about 1 cell to about 3×10³ cells, from about 1 cell to about 4×10³ cells, from about 1 cell to about 5×10³ cells, from about 1 cell to about 6×10³ cells, from about 1 cell to about 7×10³ cells, from about 1 cell to about 8×10³ cells, from about 1 cell to about 9×10³ cells, from about 1 cell to about 1×10⁴ cells, from about 1 cell to about 2×10⁴ cells, from about 1 cell to about 3×10⁴ cells, from about 1 cell to about 4×10⁴ cells, from about 1 cell to about 5×10⁴ cells, from about 1 cell to about 6×10⁴ cells, from about 1 cell to about 7×10⁴ cells, from about 1 cell to about 8×10⁴ cells, from about 1 cell to about 9×10⁴ cells, from about 1 cell to about 1×10⁵ cells, from about 1 cell to about 2×10⁵ cells, from about 1 cell to about 3×10⁵ cells, from about 1 cell to about 4×10⁵ cells, from about 1 cell to about 5×10⁵ cells, from about 1 cell to about 6×10⁵ cells, from about 1 cell to about 7×10⁵ cells, from about 1 cell to about 8×10⁵ cells, from about 1 cell to about 9×10⁵ cells, from about 1 cell to about 1×10⁶ cells, from about 1 cell to about 2×10⁶ cells, from about 1 cell to about 3×10⁶ cells, from about 1 cell to about 4×10⁶ cells, from about 1 cell to about 5×10⁶ cells, from about 1 cell to about 6×10⁶ cells, from about 1 cell to about 7×10⁶ cells, from about 1 cell to about 8×10⁶ cells, from about 1 cell to about 9×10⁶ cells, from about 1 cell to about 1×10⁷ cells, from about 1 cell to about 2×10⁷ cells, from about 1 cell to about 3×10⁷ cells, from about 1 cell to about 4×10⁷ cells, from about 1 cell to about 5×10⁷ cells, from about 1 cell to about 6×10⁷ cells, from about 1 cell to about 7×10⁷ cells, from about 1 cell to about 8×10⁷ cells, from about 1 cell to about 9×10⁷ cells, from about 1 cell to about 1×10⁸ cells, from about 1 cell to about 2×10⁸ cells, from about 1 cell to about 3×10⁸ cells, from about 1 cell to about 4×10⁸ cells, from about 1 cell to about 5×10⁸ cells, from about 1 cell to about 6×10⁸ cells, from about 1 cell to about 7×10⁸ cells, from about 1 cell to about 8×10⁸ cells, from about 1 cell to about 9×10⁸ cells, or from about 1 cell to about 1×10⁹ cells.

In some cases, the therapeutically effective dose of therapeutically effective dose of one or multiple engineered host cell populations, non-engineered cells, and/or admixtures thereof, of the invention can be from about 1×10³ cells to about 2×10³ cells, from about 1×10³ cells to about 3×10³ cells, from about 1×10³ cells to about 4×10³ cells, from about 1×10³ cells to about 5×10³ cells, from about 1×10³ cells to about 6×10³ cells, from about 1×10³ cells to about 7×10³ cells, from about 1×10³ cells to about 8×10³ cells, from about 1×10 cells to about 9×10³ cells, from about 1×10 cells to about 1×10⁴ cells, from about 1×10³ cells to about 2×10⁴ cells, from about 1×10³ cells to about 3×10⁴ cells, from about 1×10³ cells to about 4×10⁴ cells, from about 1×10³ cells to about 5×10⁴ cells, from about 1×10³ cells to about 6×10⁴ cells, from about 1×10³ cells to about 7×10⁴ cells, from about 1×10³ cells to about 8×10⁴ cells, from about 1×10³ cells to about 9×10⁴ cells, from about 1×10³ cells to about 1×10⁵ cells, from about 1×10³ cells to about 2×10⁵ cells, from about 1×10³ cells to about 3×10⁵ cells, from about 1×10³ cells to about 4×10⁵ cells, from about 1×10³ cells to about 5×10⁵ cells, from about 1×10³ cells to about 6×10⁵ cells, from about 1×10³ cells to about 7×10⁵ cells, from about 1×10³ cells to about 8×10⁵ cells, from about 1×10³ cells to about 9×10⁵ cells, from about 1×10³ cells to about 1×10⁶ cells, from about 1×10³ cells to about 2×10⁶ cells, from about 1×10³ cells to about 3×10⁶ cells, from about 1×10³ cells to about 4×10⁶ cells, from about 1×10³ cells to about 5×10⁶ cells, from about 1×10³ cells to about 6×10⁶ cells, from about 1×10³ cells to about 7×10⁶ cells, from about 1×10³ cells to about 8×10⁶ cells, from about 1×10³ cells to about 9×10⁶ cells, from about 1×10³ cells to about 1×10⁷ cells, from about 1×10³ cells to about 2×10⁷ cells, from about 1×10³ cells to about 3×10⁷ cells, from about 1×10³ cells to about 4×10⁷ cells, from about 1×10³ cells to about 5×10⁷ cells, from about 1×10³ cells to about 6×10⁷ cells, from about 1×10³ cells to about 7×10⁷ cells, from about 1×10³ cells to about 8×10⁷ cells, from about 1×10³ cells to about 9×10⁷ cells, from about 1×10³ cells to about 1×10⁸ cells, from about 1×10³ cells to about 2×10⁸ cells, from about 1×10³ cells to about 3×10³ cells, from about 1×10³ cells to about 4×10⁸ cells, from about 1×10³ cells to about 5×10⁸ cells, from about 1×10 cells to about 6×10⁸ cells, from about 1×10 cells to about 7×10⁸ cells, from about 1×10³ cells to about 8×10⁸ cells, from about 1×10³ cells to about 9×10⁸ cells, or from about 1×10³ cells to about 1×10⁹ cells.

In some cases, the therapeutically effective dose of therapeutically effective dose of one or multiple engineered host cell populations, non-engineered cells, and/or admixtures thereof, of the invention can be from about 1×10⁶ cells to about 2×10⁶ cells, from about 1×10⁶ cells to about 3×10⁶ cells, from about 1×10⁶ cells to about 4×10⁶ cells, from about 1×10⁶ cells to about 5×10⁶ cells, from about 1×10⁶ cells to about 6×10⁶ cells, from about 1×10⁶ cells to about 7×10⁶ cells, from about 1×10⁶ cells to about 8×10⁶ cells, from about 1×10⁶ cells to about 9×10⁶ cells, from about 1×10⁶ cells to about 1×10⁷ cells, from about 1×10⁶ cells to about 2×10⁷ cells, from about 1×10⁶ cells to about 3×10⁷ cells, from about 1×10⁶ cells to about 4×10⁷ cells, from about 1×10⁶ cells to about 5×10⁷ cells, from about 1×10⁶ cells to about 6×10⁷ cells, from about 1×10⁶ cells to about 7×10⁷ cells, from about 1×10⁶ cells to about 8×10⁷ cells, from about 1×10⁶ cells to about 9×10⁷ cells, from about 1×10⁶ cells to about 1×10⁸ cells, from about 1×10⁶ cells to about 2×10⁸ cells, from about 1×10⁶ cells to about 3×10⁸ cells, from about 1×10⁶ cells to about 4×10⁸ cells, from about 1×10⁶ cells to about 5×10⁸ cells, from about 1×10⁶ cells to about 6×10⁸ cells, from about 1×10⁶ cells to about 7×10⁸ cells, from about 1×10⁶ cells to about 8×10⁸ cells, from about 1×10⁶ cells to about 9×10⁸ cells, from about 1×10⁶ cells to about 1×10⁹ cells, from about 1×10⁶ cells to about 2×10⁹ cells, from about 1×10⁶ cells to about 3×10⁹ cells, from about 1×10⁶ cells to about 4×10⁹ cells, from about 1×10⁶ cells to about 5×10⁹ cells, from about 1×10⁶ cells to about 6×10⁹ cells, from about 1×10⁶ cells to about 7×10⁹ cells, from about 1×10⁶ cells to about 8×10⁹ cells, from about 1×10⁶ cells to about 9×10⁹ cells, from about 1×10⁷ cells to about 1×10⁹ cells, from about 1×10⁷ cells to about 2×10⁹ cells, from about 1×10⁷ cells to about 3×10⁹ cells, from about 1×10⁷ cells to about 4×10⁹ cells, from about 1×10⁷ cells to about 5×10⁹ cells, from about 1×10⁷ cells to about 6×10⁹ cells, from about 1×10⁷ cells to about 7×10⁹ cells, from about 1×10⁷ cells to about 8×10⁹ cells, from about 1×10⁷ cells to about 9×10⁹ cells, from about 1×10⁸ cells to about 1×10⁹ cells, from about 1×10⁸ cells to about 2×10⁹ cells, from about 1×10⁸ cells to about 3×10⁹ cells, from about 1×10⁸ cells to about 4×10⁹ cells, from about 1×10⁸ cells to about 5×10⁹ cells, from about 1×10⁸ cells to about 6×10⁹ cells, from about 1×10⁸ cells to about 7×10⁹ cells, from about 1×10⁸ cells to about 8×10⁹ cells, from about 1×10⁸ cells to about 9×10⁹ cells, or from about 1×10⁸ cells to about 1×10¹⁰ cells.

### H. Preservation

In some embodiments, the one or multiple engineered host cell populations, non-engineered cells, and/or admixtures thereof, of the invention may be formulated in freezing media and placed in cryogenic storage units such as liquid nitrogen freezers (−195 C) or ultra-low temperature freezers (−65 C, −80 C or −120 C) for long-term storage of at least about 1 month, 2 months, 3 months, 4 months, 5 months, 6 months, 1 year, 2 years, 3 years, or at least 5 years. The freeze media can contain dimethyl sulfoxide (DMSO), and/or sodium chloride (NaCl), and/or dextrose, and/or dextran sulfate and/or hydroyethyl starch (HES) with physiological pH buffering agents to maintain pH between about 6.0 to about 6.5, about 6.5 to about 7.0, about 7.0 to about 7.5, about 7.5 to about 8.0 or about 6.5 to about 7.5. In embodiments, the cryopreserved cells can be thawed and further processed, for example by stimulation with antibodies, proteins, peptides, and/or cytokines as mentioned herein. The cryopreserved cells can be thawed and genetically modified with viral vectors (including retroviral and lentiviral vectors) or non-viral means (including RNA, DNA, and proteins) as described herein. Alternatively, host cells as described herein can be, e.g., optionally expanded by the methods described herein, genetically modified, and then cryopreserved.

Thus, genetically engineered and/or non-engineered cells as disclosed herein can be cryopreserved to generate cell banks in quantities of at least about 1, 5, 10, 100, 150, 200, 500 vials at about at least 10¹, 10², 10³, 10⁴, 10⁵, 10⁶, 10⁷, 10⁸, 10⁹, or at least about 10¹⁰ cells per mL in freeze media. The cryopreserved cell banks may retain their functionality and can be thawed and, optionally, be activated/stimulated and/or expanded. In some aspects, thawed cells can be stimulated and expanded in suitable closed vessels such as cell culture bags and/or bioreactors to generate quantities of cells as allogeneic cell product. In other examples, the croyperserved cells comprise an autologous cell product. Cryopreserved cells can maintain their biological functions for at least about 6 months, 7 months, 8 months, 9 months, 10 months, 11 months, 12 months, 13 months, 15 months, 18 months, 20 months, 24 months, 30 months, 36 months, 40 months, 50 months, or at least about 60 months under cryogenic storage condition. In some aspects, no preservatives are used in the formulation. In some embodiments, the cryopreserved cells can be thawed and infused into multiple patients as allogeneic off-the-shelf cell product.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Construction of DAP10 Constructs

DAP10 CAD constructs were constructed with 0-4 elements selected from Y86F mutation, K84R mutation, 4-1BB costimulatory domain, CD3ζ signaling domain. For construction, the pSIN vector (Hariharan, M J et al., (1998) Journal of Virology 72(2): 950-958) was used. Briefly, the pSIN vector backbone was fully synthesized by Genewiz® (South Plainfield, NJ) from sequence provided by EUFETS (Germany) a subsidiary of BioNTech (Germany). The base plasmid used for all constructs is referred to as pL077 pRetroSIN-GFP, which has a green fluorescent protein (GFP) cassette that is replaced by a gene-of-interest. The various constructs are depicted in Table 1 below. With regard to the following constructs, FP2A refers to a furin and P2A linker gene, and CD19t refers to a truncated CD19 marker. SEQ ID corresponds to amino acid sequence. Tables 4-19 below annotate the nucleic acid sequences that encode the amino acid sequences for the constructs depicted at Table 1, and select sequences from Table 3. Although Table 1 depicts DAP 10 constructs including CD 19t, other variations are within the scope of this disclosure, for example similar DAP10 constructs incorporating EGFRt, and the like. Examples of such sequences are provided in Table 3.

TABLE 1
DAP10 CAD Constructs

| | | Signal Null? | Ubiquitin | | |
|---|---|---|---|---|---|
| Long Name | Short Name | (Y86F) | mutant (K84R) | 4-1BB | CD3ζ |
| pSIN- | DAP10.0 (wt) | No | No | No | No |
| DAP10.0- | (SEQ ID NO: | | | | |
| FP2A-CD19t | 21) | | | | |
| pSIN- | DAP10.3 | No | No | No | Yes |
| DAP10.3- | (SEQ ID NO: | | | | |
| F2PA-CD19t | 23) | | | | |
| pSIN- | DAP10.4 | No | No | Yes | Yes |
| DAP10.4- | (SEQ ID NO: | | | | |
| FP2A-CD19t | 25) | | | | |
| pSIN- | DAP10.5 | Yes | No | Yes | Yes |
| DAP10.5- | (SEQ ID NO: | | | | |
| FP2A-CD19t | 27) | | | | |
| pSIN- | DAP10.6 | Yes | Yes | Yes | Yes |
| DAP10.6- | (SEQ ID NO: | | | | |
| FP2A-CD19t | 29) | | | | |
| pSIN- | DAP10.13 | No | Yes | No | No |
| DAP10.13- | (SEQ ID NO: | | | | |
| FP2A-CD19t | 31) | | | | |
| pSIN- | DAP10.14 | No | Yes | No | Yes |
| DAP10.14- | (SEQ ID NO: | | | | |
| FP2A-CD19t | 33) | | | | |
| pSIN- | DAP10.15 | No | Yes | Yes | Yes |
| DAP10.15- | (SEQ ID NO: | | | | |
| FP2A-CD19t | 35) | | | | |
| pSIN- | DAP10.16 | Yes | Yes | Yes | Yes |
| DAP10.16- | (SEQ ID NO: | | | | (1XX |
| FP2A-CD19t | 96) | | | | modification) |
| pSIN- | DAP10.17 | Yes | Yes | No | Yes |
| DAP10.17- | (SEQ ID NO: | | | | (1XX |
| FP2A-CD19t | 104) | | | | modification) |

### Example 2. Tumor Control of Various DAP10 CAD Constructs in PLC/PRF/5 Assay

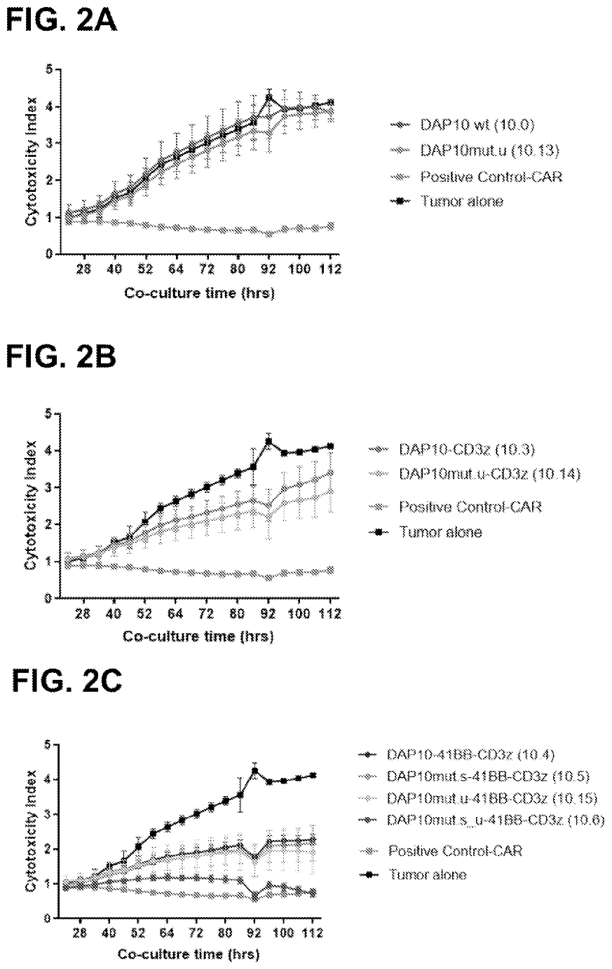

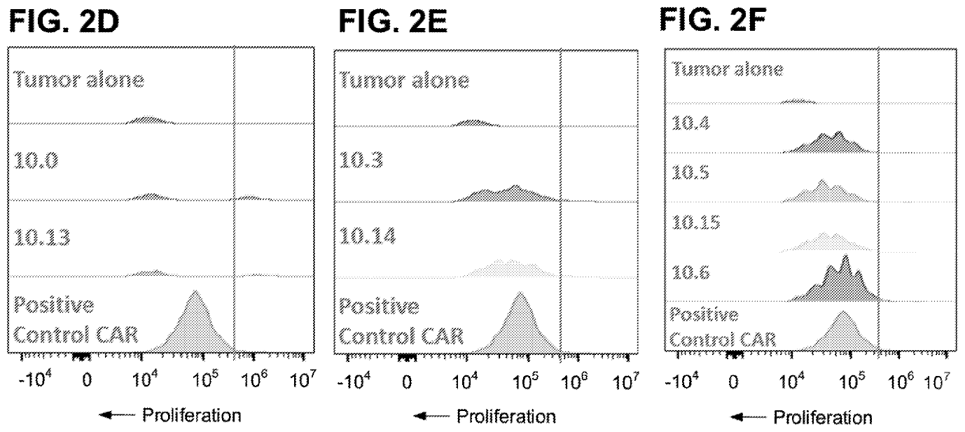

Various DAP10 CAD constructs from Table 1 above were tested in a PLC/PRF/5 cell lysis assay. FIG. 2A illustrates that DAP10.0 (DAP10 wild-type) and DAP10.13 (DAP10-K84R) do not display good tumor control in the PLC/PRF/5 assay. FIG. 2B illustrates some amount of tumor control for the DAP10.3 (DAP10-CD3ζ) and DAP10.14 (DAP10-CD3ζ-K84R) constructs. FIG. 2C illustrates that DAP10 CAD constructs that include both the 4-1BB costimulatory domain and the CD3ζ signaling domain show good tumor control in absence of additional mutation (i.e., DAP10.4), and that addition of just the Y86F mutation (i.e., DAP10.5) or just the K84R mutation (i.e., DAP10.15) does not substantially further improve tumor control over the DAP10.4 construct. However, tumor control was substantially improved in the DAP10 CAD construct DAP10.6 that incorporated each of the CD3ζ signaling domain, the 4-1BB costimulatory domain, and both mutations (i.e., Y86F and K84R) (FIG. 2C). At each of FIGS. 2A-2C, also depicted is tumor control tested with a positive control CAR, as well as a plot showing cytotoxicity index of the tumor cells alone.

Vδ1 γδ cell proliferation was also examined for the test conditions corresponding to FIGS. 2A-2C, and the results are shown at FIGS. 2D-2F, respectively. Good proliferation was observed from most DAP10 CAD constructs, as shown.

### Example 3. Tumor Control of Various DAP10 CAD Constructs in HepG2 Assay

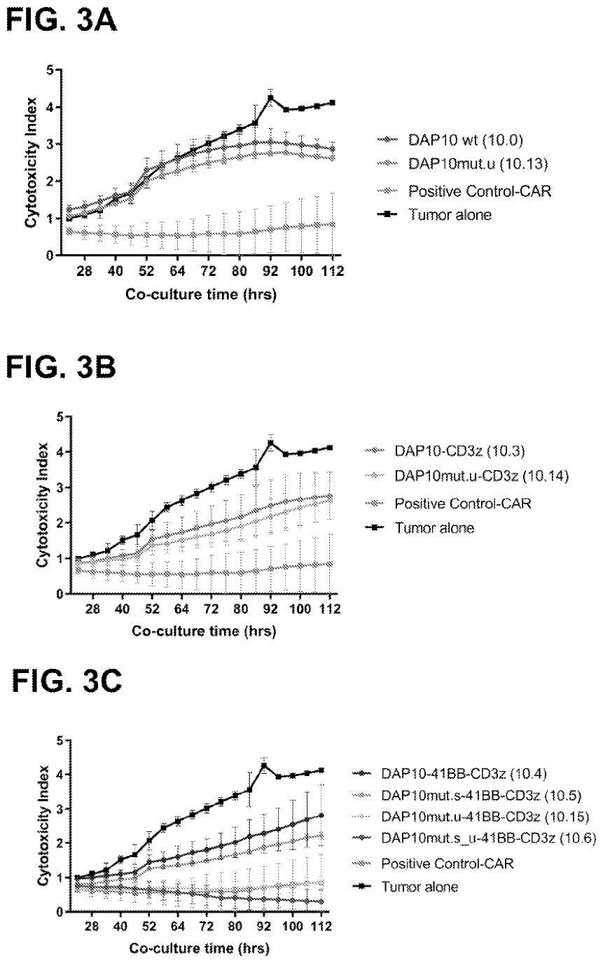

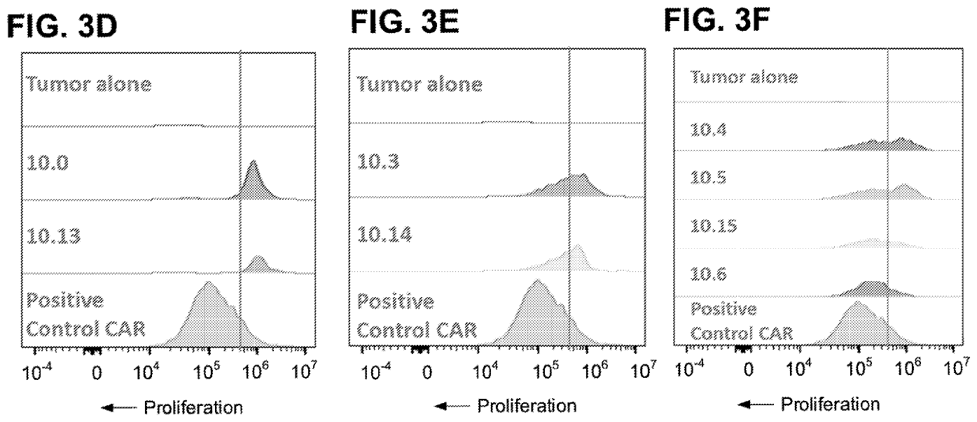

Various DAP10 CAD constructs from Table 1 above were tested in a HepG2 assay. FIG. 3A illustrates data obtained using the DAP10.0 (DAP10 wild-type) and DAP10.13 (DAP10-K84R) constructs, FIG. 3B illustrates data obtained using the DAP10.3 (DAP10-CD3ζ) and DAP10.14 (DAP10-CD3ζ-K84R) constructs, and FIG. 3C illustrates data obtained using the DAP10.4, DAP10.5, DAP10.6, and DAP10.15 constructs. At each of FIGS. 3A-3C, also depicted is tumor control tested with a positive control CAR, as well as a plot showing cytotoxicity index of the tumor cells alone.

Vδ1 γδ cell proliferation was also examined for the test conditions corresponding to FIGS. 3A-3C, and the results are shown at FIGS. 3D-3F, respectively. In the Hep2G assay, good proliferation and cytotoxicity was observed in constructs that included both the CD3ζ and 4-1BB domains, as well as at least the Y86F mutation (i.e., DAP10.15 and DAP10.6), with the DAP10.6 construct (which includes each of CD3ζ, 4-1BB, Y86F, and K84R) showing the best performance in terms of cytotoxicity and Vδ1 γδ cell proliferation.

### Example 4. Survival of Vδ1 Cells Transduced with DAP10 Constructs after Tumor Co-culture

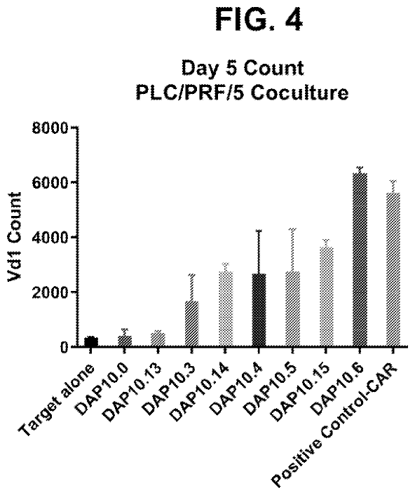

In this Example, survival of Vδ1 cells transduced with various DAP10 constructs of Table 1 was assessed. Specifically, survival was assessed following 5 days of PLC/PRF/5 co-culture. As shown at FIG. 4, cells transduced with DAP10.6 showed the best Vδ1 survival after 5 days of co-culture with PLC/PRF/5.

### Example 5. In Vivo Tumor Control of DAP10 CAD Constructs in a Mouse Model

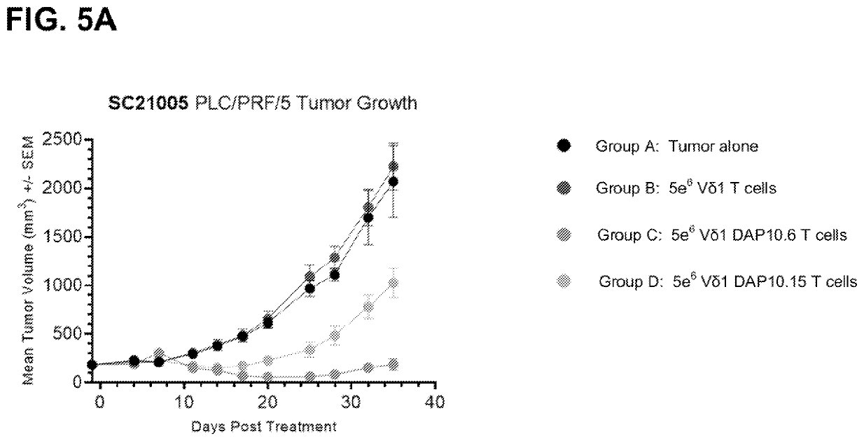

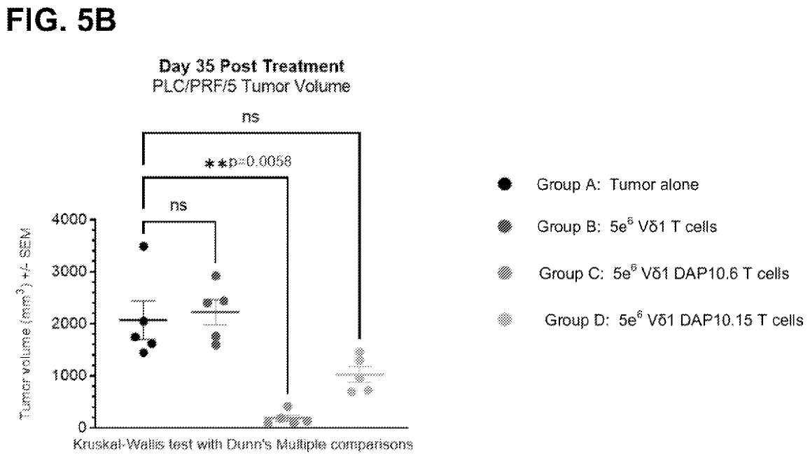

In this Example DAP10 CAD constructs (DAP10.6 and DAP10.15, refer to Table 1) were examined for their effectiveness at controlling PLC/PRF/5 tumor growth in a mouse model. As shown at FIG. 5A, mean tumor volume steadily increased over the course of 35 days in mice harboring tumor alone (group A). Similar results were observed in mice harboring the tumor and injected with 5e⁶ Vδ1 T cells (untransduced, group B). Tumor growth was significantly reduced when mice were injected with Vδ1 T cells transduced with either DAP10.6 (group C) or DAP10.15 (group D), with the most robust efficacy in terms of tumor control observed for the DAP10.6 CAD. FIG. 5B shows the data obtained in FIG. 5A at day 35, as analyzed via Kruskal-Wallis test with Dunn's multiple comparisons and plotted as a function of tumor volume as shown. For each of groups A-D at FIGS. 5A and 5B, N=5, and the number of tumor cells dosed was 4e⁶.

### Example 6. NKG2D Expression in Vδ1 T Cells Transduced with DAP10 CAD

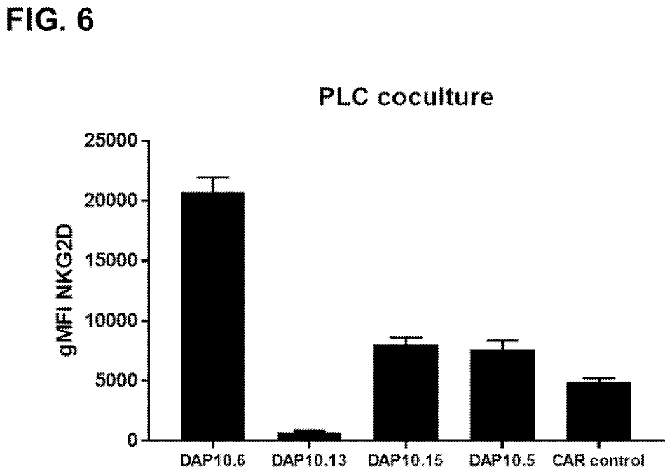

This Example demonstrates increased NKG2D expression levels on cells transduced with a particular DAP10 CAD (e.g., DAP10.6, refer to Table 1). Briefly, Vδ1 T cells were transduced with one of DAP10.6, DAP10.13, DAP10.15, DAP10.5, and a control CAR, and said transduced cells were then co-cultured with PLC cells. Fluorescent labeling of NKG2D in conjunction with FACS analysis was used to assess Vδ1 NKG2D expression level. At FIG. 6, expression levels for the cells containing different DAP10 CADs or CAR control are plotted as geometric mean fluorescence intensity (gMFI), illustrating that the DAP10.6 CAD construct expressed in Vδ1 T cells results in significantly higher NKG2D expression levels as compared to other DAP10 CADs (10.13, 10.15, 10.5) and the CAR control. Notably, the DAP10.6 CAD construct includes each of the K84R mutation, the Y86F mutation, a 4-1BB costimulatory domain and a CD3ζ signaling domain. A smaller but still statistically significant increase in NKG2D expression over the CAR control was observed with DAP10.15 (K84R+4-1BB+CD3ζ) and with DAP10.5 (Y86F+4-1BB+CD3ζ).

### Example 7. DAP10 CAD Expression

This Example demonstrates that DAP10 CAD expression is similar across different lots of Vδ1 cells.

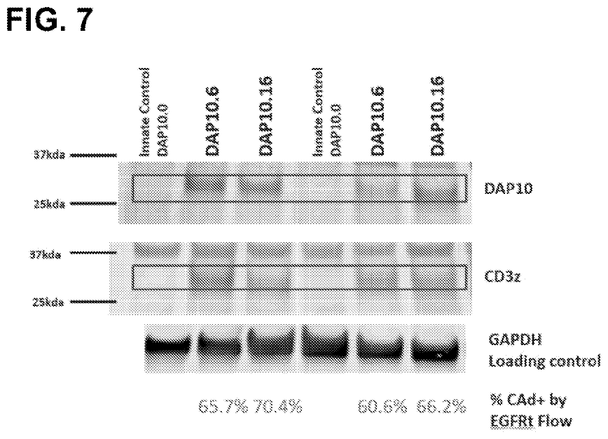

The DAP10.6.3 construct (SEQ ID NO: 77) includes truncated EGFR as a marker, as compared to, e.g., the DAP10.6 construct (SEQ ID NO: 29) that includes CD19. A DAP10 construct referred to herein as DAP10.16 (SEQ ID NO: 96) is the same as the DAP10.6.3 construct, but includes the 1XX mutation in the CD3ζ signaling domain. VS1 cells transfected with DAP10.6 or DAP10.16 constructs were found to exhibit substantially similar expression levels. CAD protein was directly detected using anti-DAP10 and anti-CD3ζ antibodies by western blot analysis (n=2 donors) (FIG. 7).

### Example 8. Cytotoxic Activity of DAP10 CAD is Mediated by NKG2D

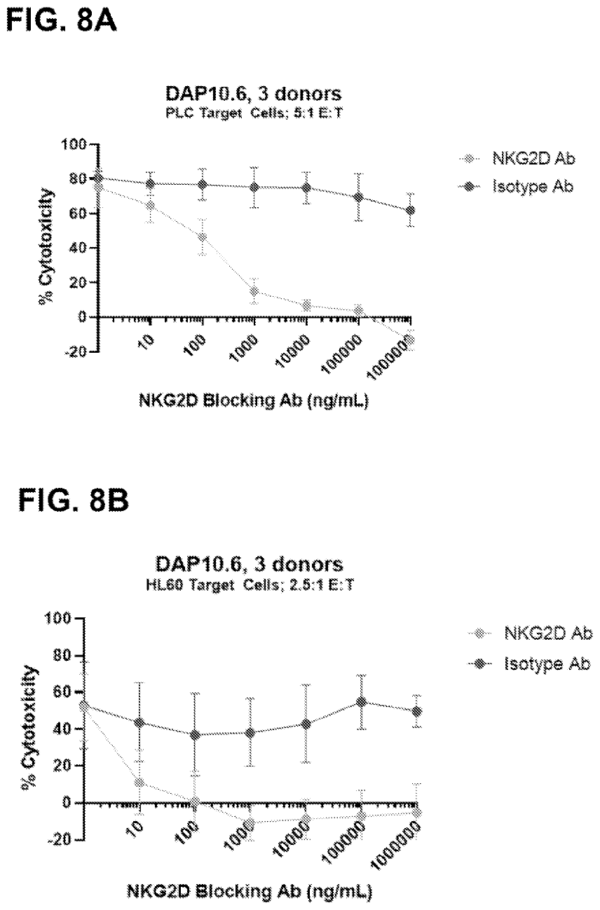

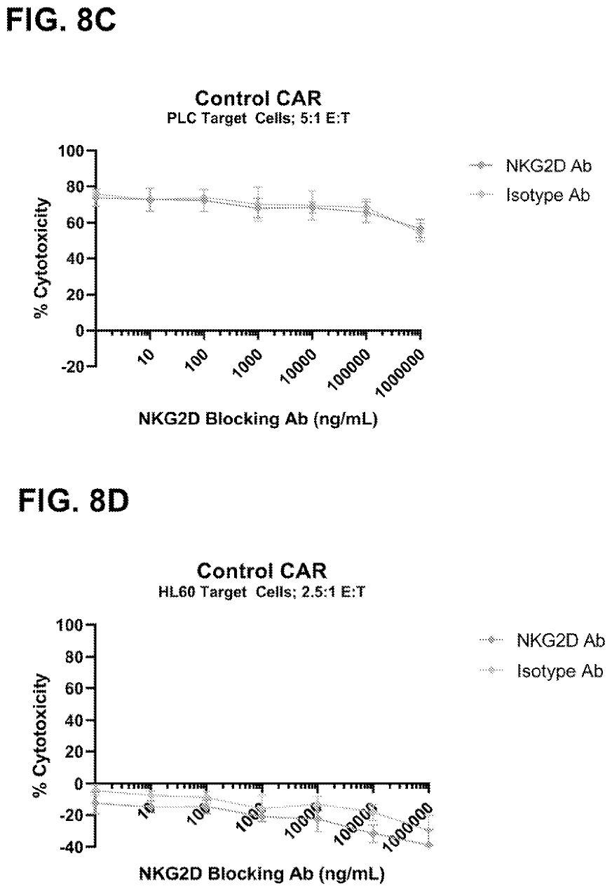

This Example demonstrates that blocking NKG2D eliminates cytotoxic activity. Specifically, blocking NKG2D via the use of an NKG2D blocking antibody eliminated cytotoxic activity of DAP10 CAD expressing Vδ1 cells in three donors across 2 cell lines (FIGS. 8A-8B). Cytotoxic activity of control Vδ1 cells transfected with a CAR was unaffected (FIGS. 8C-8D). For FIGS. 8A, 8C, PLC target cells were used, 5:1 E:T ratio. For FIGS. 8B, 8D, HL60 target cells were used, 2.5:1 E:T ratio. The NKG2D blocking antibody also had no effect on cells expressing the DAP10.0 construct (i.e., lacking costimulatory and intracellular signaling domain) (data not shown).

### Example 9. DAP10 CAD Molecular Activation Signature

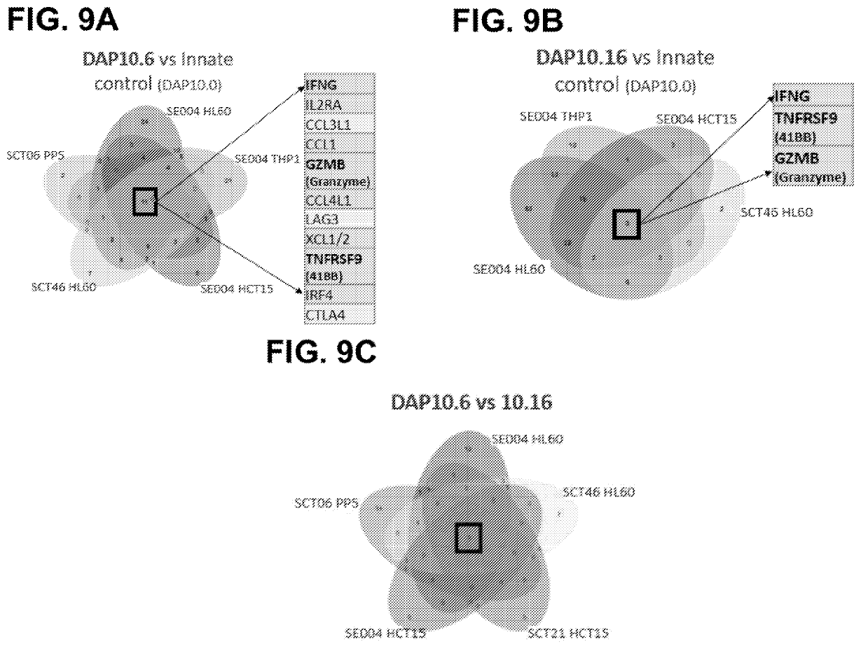

This Example demonstrates consistent DAP10 CAD activation signature across multiple donors and cell lines. For this Example, Nanostring analysis (Nanostring Technologies, Seattle, WA) was conducted post-stimulation to assess molecular activation signature. Cell lines used for stimulation included PLC (HCC), HL60, THP1 (AML), and HCT15 (CRC). Data illustrated consistent activation signature medicated through interferon gamma, 4-1BB, and Granzyme B. FIG. 9A depicts data for DAP10.6 vs innate control (DAP 10.0), and FIG. 9B depicts data for DAP10.16 vs innate control (DAP10.0). No consistently detectable differences between DAP10.6 and DAP10.16 were observed (FIG. 9C).

### Example 10. Broad Anti-Cancer Activity of Vδ1 Cells Transduced with DAP10 CAD

This Example demonstrates that VS1 cells transduced with DAP10 CADs of the present disclosure exhibit anti-cancer activity against various cancer types having a broad range of NKG2D ligand expression levels/patterns.

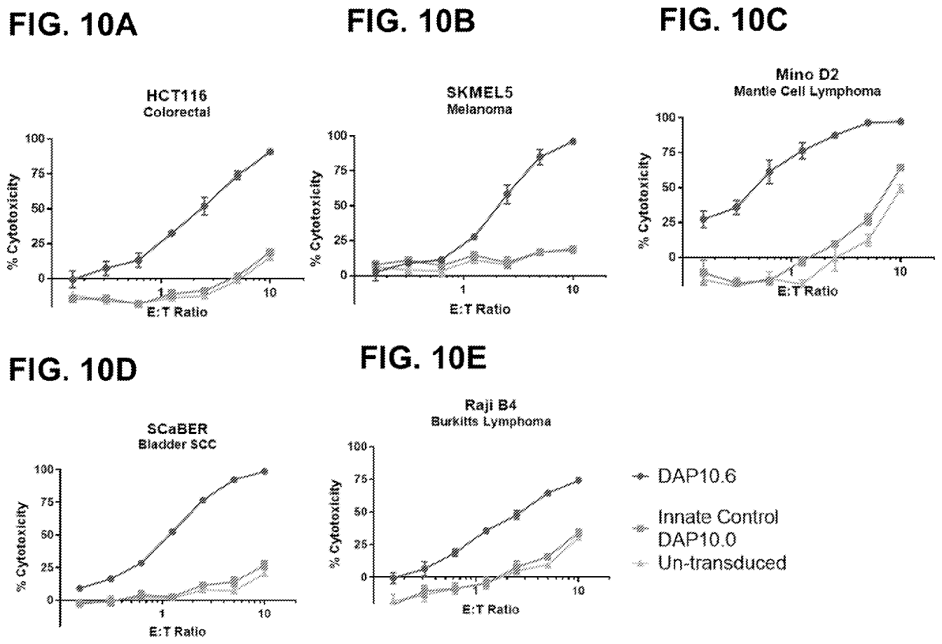

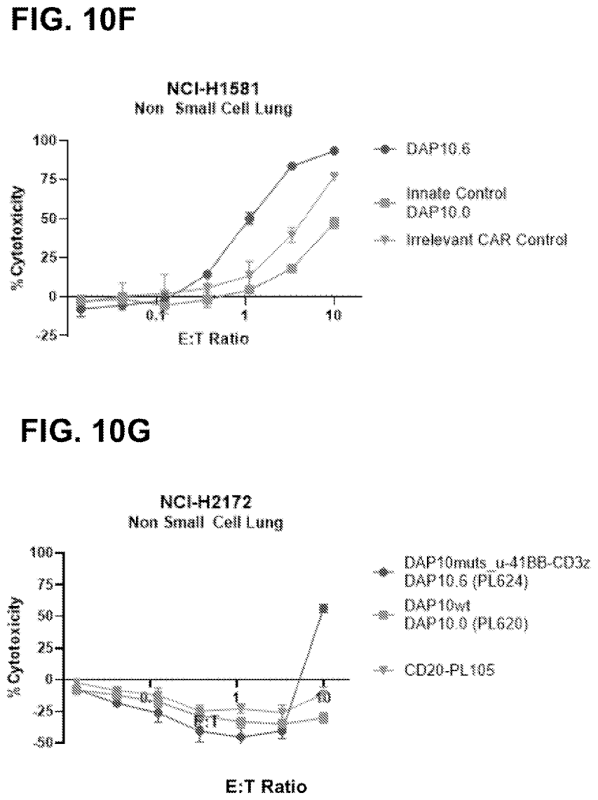

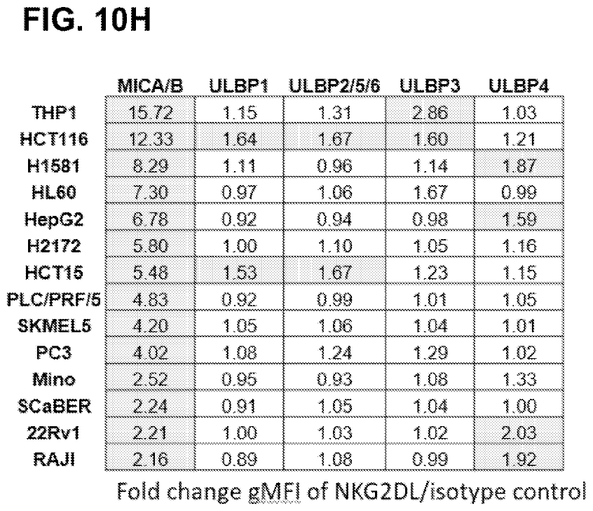

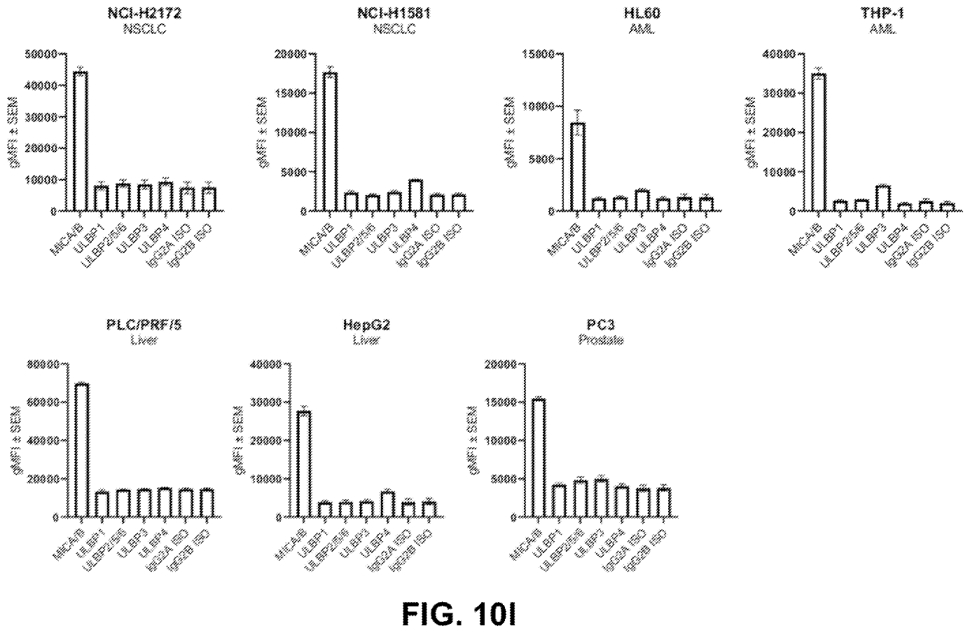

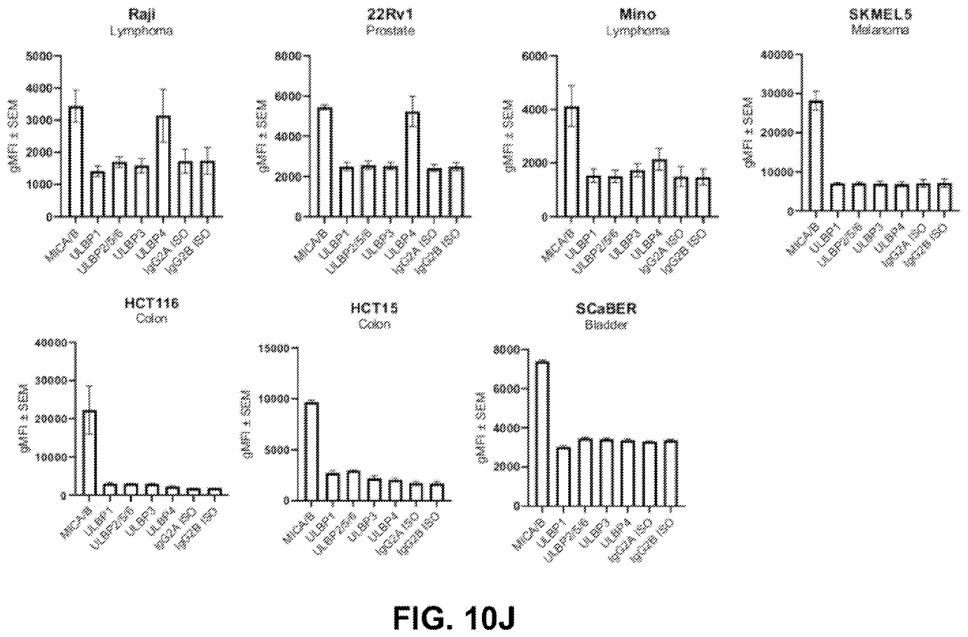

FIGS. 10A-10E are graphs illustrating % cytotoxic activity as a function of E:T ratio for Vδ1 cells transduced with DAP10.6 as compared to Vδ1 cells transduced with DAP10.0, or cells not transduced with a DAP10 CAD, in an 18-hour assay. Target cell lines included HCT116 (FIG. 10A), SKMEL5 (FIG. 10B), Mino D2 (FIG. 10C), ScaBER (FIG. 10D), and Raji B4 (FIG. 10E). FIGS. 10F-10G are graphs showing % cytotoxicity of Vδ1 cells transduced with DAP10.6 as compared to innate control (Vδ1 cells transduced with DAP10.0), or an irrelevant CAR control, in an 18-hour assay. Target cell lines for FIGS. 10F-10G were NCI-H1581 and NCI-H2172, respectively. As shown, cytotoxic potency was significantly increased relative to controls. FIG. 10H illustrates that selected cell lines used for assays represent a broad range of NKG2D ligand expression levels/patterns. To obtain the data at FIG. 10H, different cancer cell lines as indicated, derived from a variety of hematologic and solid tumors, were assessed for NKG2D ligands by flow cytometry. 5 antibodies used for staining detected MICA/MICB, ULBP1, ULBP2/5/6, ULBP3, and ULBP4. Data is presented as fold change mean fluorescence intensity (MFI) of NKG2D ligand over relevant isotype control. Raw data is shown at FIGS. 10I-10J (cancer cell lines were stained in triplicate).

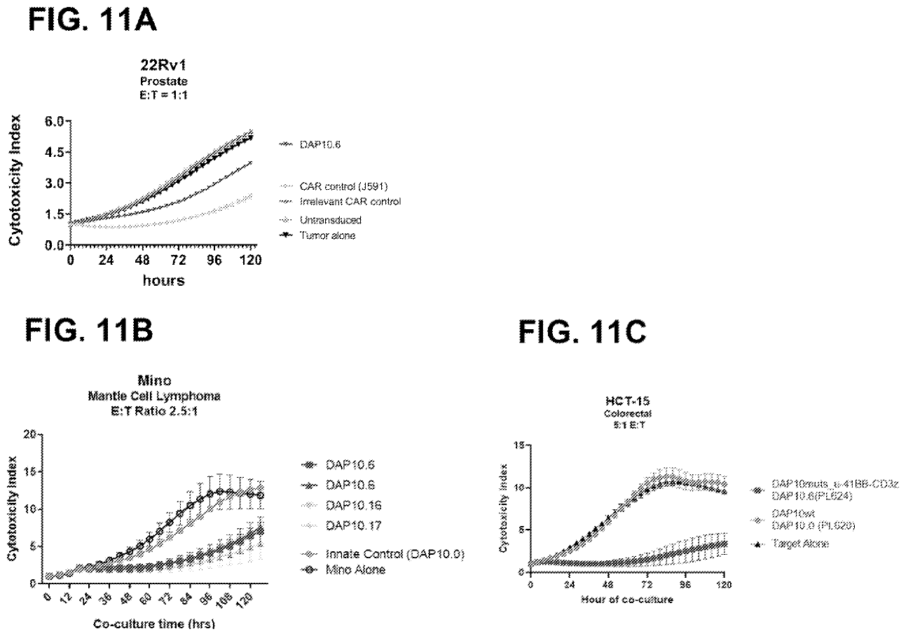

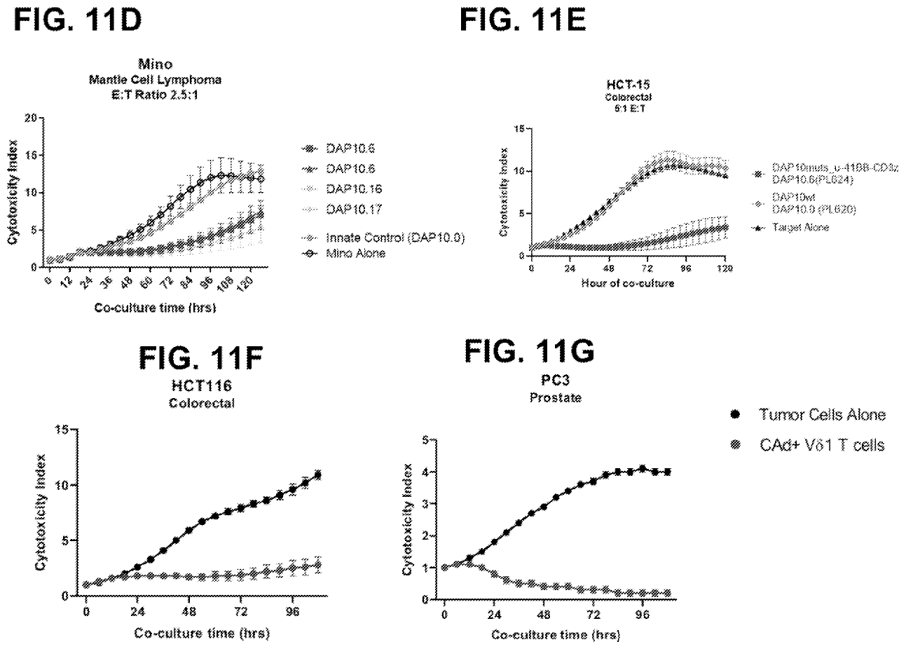

FIGS. 11A-11G are graphs illustrating cytotoxicity index as a function of target and effector co-culture time. Target cells included 22Rv1, Mino, HCT116, and HCT-15. Effectors included VS1 cells transduced with DAP10 CAR, CAR control, or untransduced cells. Enhanced cytotoxicity was observed when Vδ1 cells were transduced with a DAP10 CAD of the present disclosure, as compared to controls. Degree of cytotoxic potential was found to be dependent on cell line and E:T ratio. Cell lines tested represent various levels/patterns of NKG2D ligand expression (see FIG. 10H-10J). Effector cells were co-cultured with NucRed-expressing target cells at submaximal E:T ratios of 5:1 or 1.5:1 depending on the cell line. Cytotoxicity index was calculated by dividing total NucRed object area (mm²/well) of each time point by the value at time=0.

### Example 11. Comparable Cytotoxic Activity Across Multiple Lots of Vδ1 Cells Transduced with DAP10 CADs

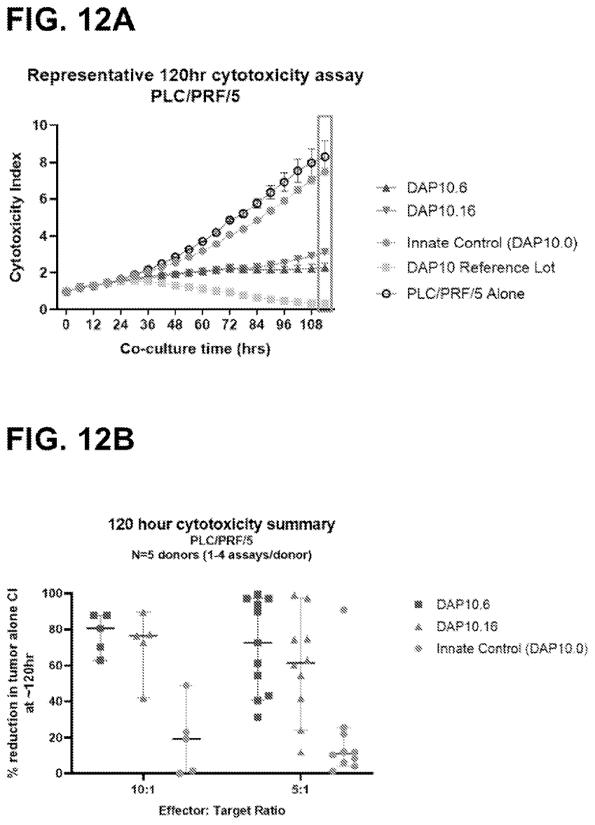

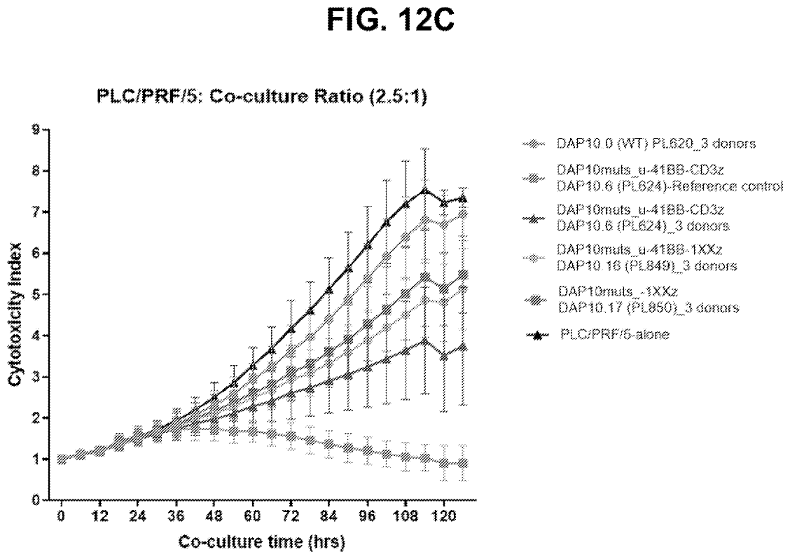

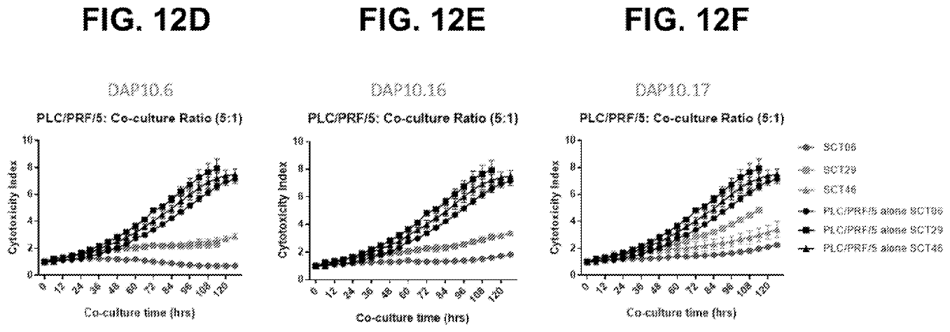

This Example demonstrates that cytotoxic activity of VS1 cells transduced with DAP10 CADs is comparable for different lots of Vδ1 cells and DAP10 CADs. FIG. 12A is a graph showing data for a representative 120 hour cytotoxicity assay. Target cells were PLC/PRF/5. Vδ1 cells used in the assay were transduced with DAP10.6, DAP10.16, DAP10 reference lot (i.e., positive control batch of expanded Vδ1 cells), and DAP10.0 (control). Also shown is data from PLC/PRF/5 cells alone (i.e., no co-culture with Vδ1 cells). FIG. 12B is a graph depicting % reduction in cytotoxicity of tumor alone relative to tumor treated with Vδ1 cells transduced with DAP10 CAD using the final time point of the 120-hour assay.

Cytotoxicity of DAP10.6 vs DAP10.16 vs DAP10.17 constructions incorporated into Vδ1 cells was examined in a 120-hour cytotoxicity assay. Vδ1 cells from three donors were tested with the DAP10 constructs, using PLC/PRF/5 target cells. Each construct shown at FIG. 12C is an aggregate of all three donors. Using a stringent E:T ratio, in this assay the DAP10.6 construct exhibited slightly better average cytotoxicity than DAP10.16 and DAP10.17.

Efficacy of DAP10.6 vs DAP10.16 vs. DAP10.17 showed some donor dependence. Cytotoxicity index was measured in a co-culture experiment with Vδ1 cells from three different donors (SCT06, SCT29, SCT46) transduced with either DAP10.6, DAP10.16, or DAP10.17, or non-transduced cells from the same donors. Target cells in the co-culture experiments were PLC/PRF/5 cells. Co-culture time was 120 hours. As shown in FIGS. 12D-12E, the DAP10.6 and DAP10.16 show very similar profiles. DAP10.17 was found to be the most donor dependent (FIG. 12F). A reference lot of Vδ1 cells transduced with DAP10.6 was used in all assays and served as a reproducible control (data not shown).

### Example 12. Cytokine Profile Corresponding to DAP10 CAD Stimulation

This Example demonstrates that DAP10 CAD stimulation results in a polyfunctional cytokine profile.

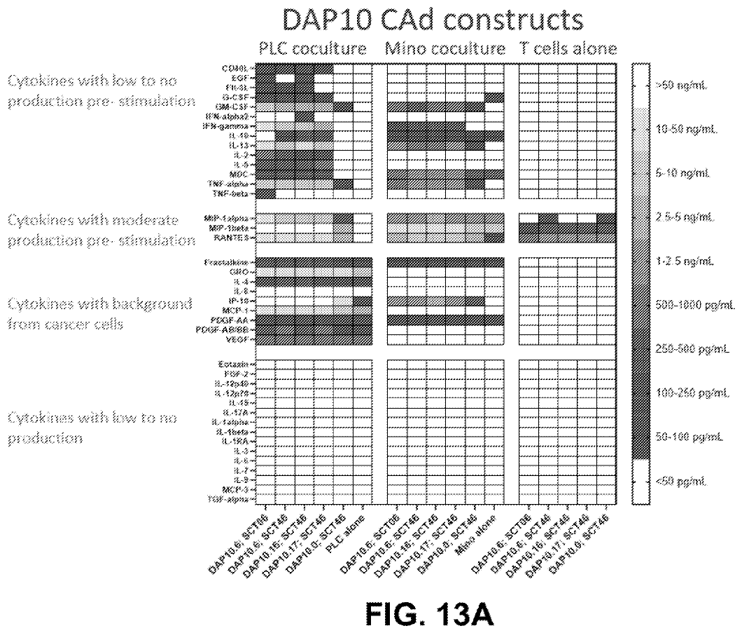

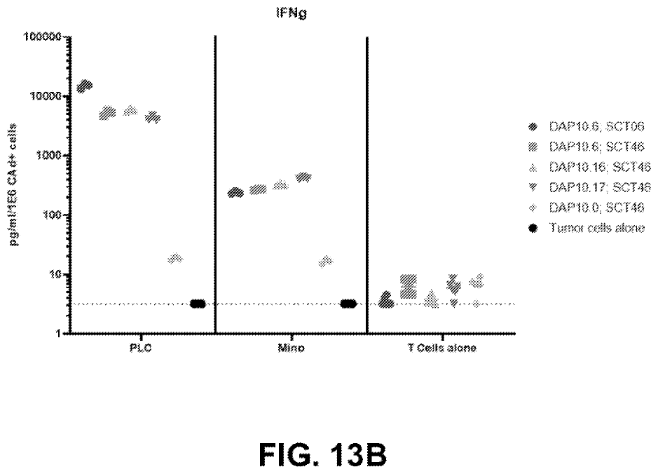

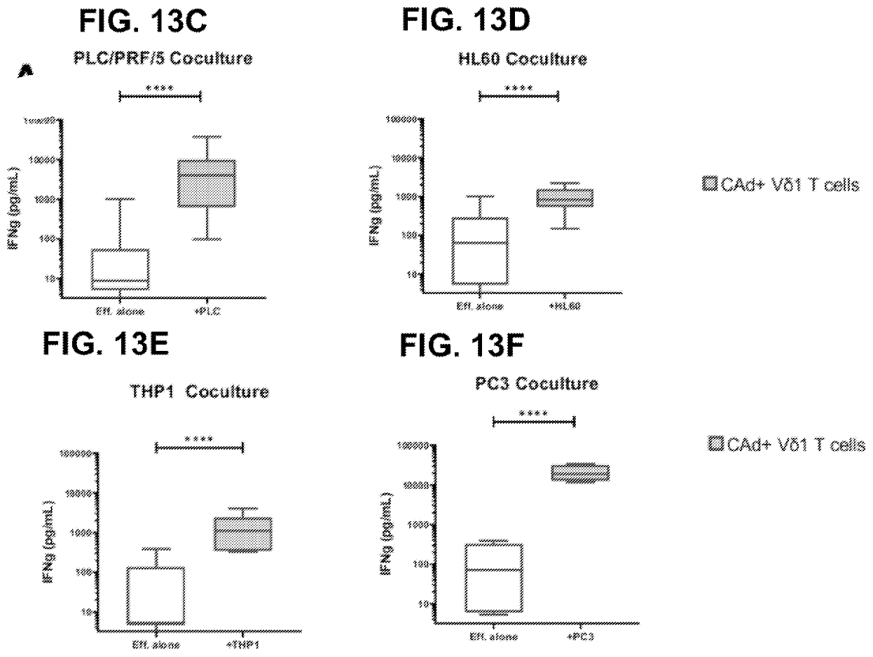

FIG. 13A illustrates cytokine profile as a function of different DAP10 constructs of the present disclosure. As illustrated, degree of cytokine activation was cell line specific. Cell lines tested included PLC, Mino, and T cells alone. Importantly, no detection of potentially problematic cytokines (e.g., IL-6 and IL-17) was observed. FIG. 13B is a graph showing interferon gamma induction (pg/ml/1E⁶ CAD⁺ cells) in PLC, Mino, and T cells alone, for various DAP10 CADs (from various donors, e.g., SCT06, SCT 46). FIGS. 13C-13F are representative experiments illustrating interferon gamma secretion from CAD+Vδ1 cells alone and after ˜18-hour co-culture at submaximal E:T ratios with PLC/PRF/5, HL60, THP1, and PC3 target cells, respectively.

### Example 13. Cytokine Profile of DAP10 CAD Compared to Chimeric Antigen Receptor (CAR)

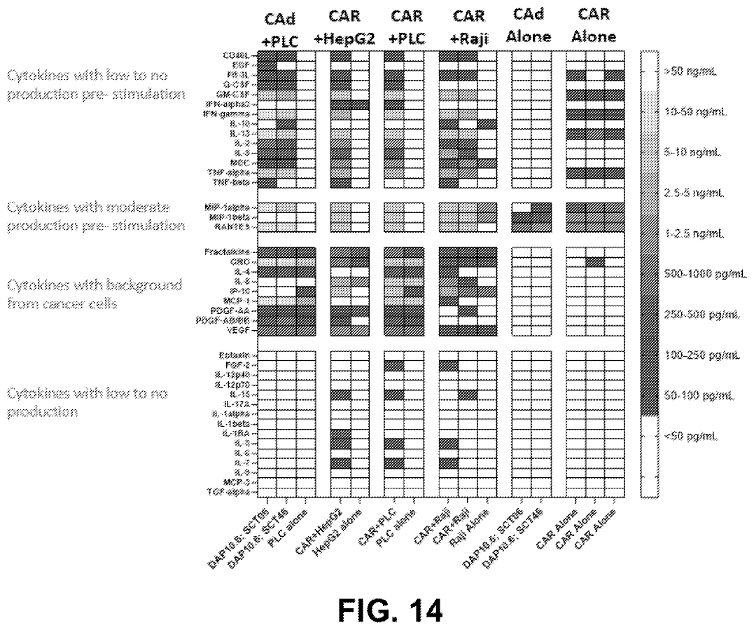

This Example demonstrates a high degree of similarity in cytokine profiles from DAP10 CADs of the present disclosure, as compared to CARs (FIG. 14). Conditions examined included Vδ1 cells transduced with a DAP10 CAD plus PLC cells, or alone (i.e., minus target cell), and Vδ1 cells transduced with a CAR plus target cell (HepG2, PLC, Raji), or alone (i.e., minus target cell). Notably, Vδ1 cells transduced with a DAP10 CAD in absence of target cell show less background cytokine secretion than Vδ1 cells transduced with a CAR in absence of target cell.

### Example 14. DAP10 CAD Stimulation Drives Proliferation Across Multiple Donors

This Example demonstrates that DAP10 CAD stimulation drives Vδ1 cell proliferation in all donors tested. Specifically, for this Example, Vδ1 cell proliferation was assessed following transduction of DAP10 CAD constructs of the present disclosure (DAP10.6, DAP10.16, DAP10.17), by co-culture with PLC/PRF/5 (5:1 E:T ratio). Controls included Vδ1 cells transduced with DAP10 control batch, or DAP10.0). Less/slower proliferation of VS1 cells transduced with DAP10.16 is likely the result of the 1XX CD3ζ signaling domain. In this regard, enhanced regulation of activation/proliferation may be beneficial to long term efficacy/survival of engineered Vδ1 cells, due to reduced overstimulation/exhaustion.

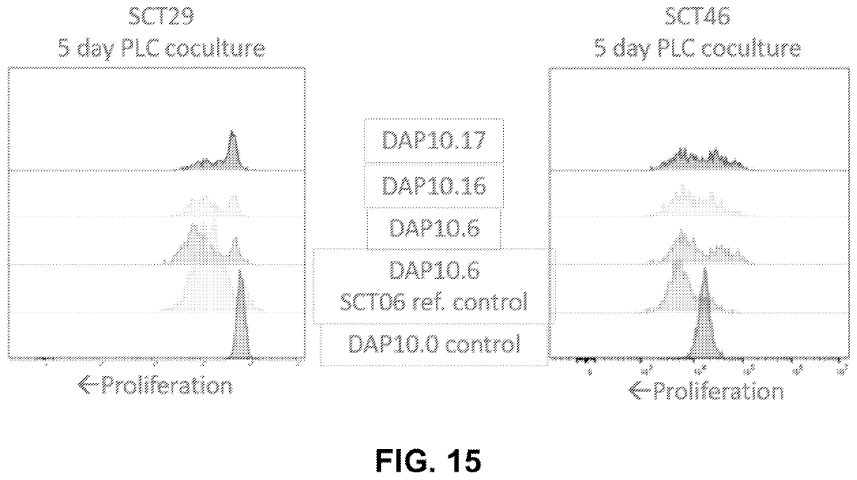

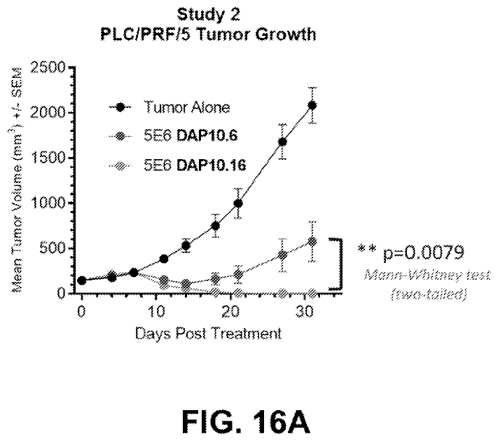

Vδ1 cells obtained from two different donors (SCT29 and SCT46) were used in the co-culture experiment. As shown in FIG. 15, robust proliferation of Vδ1 cells transduced with DAP10.6 and DAP10.16 was observed for both donors, whereas proliferation was somewhat more donor dependent for Vδ1 cells transduced with DAP10.17.

### Example 15. In Vivo Tumor Control by Vδ1 Cells Transduced with a DAP10 CAD Containing a 1XX CD3ζ Intracellular Signaling Domain

This Example demonstrates that incorporation of a 1XX CD3ζ intracellular signaling domain may improve tumor control in vivo.

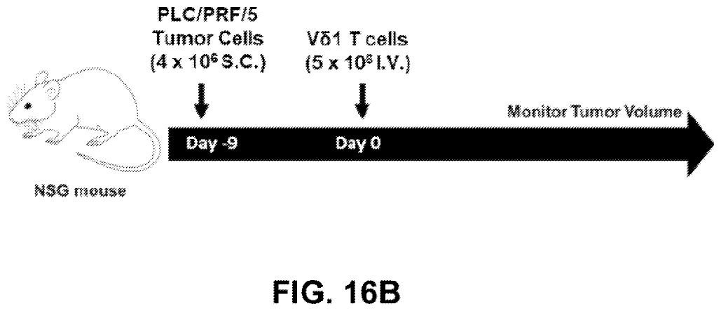

In this Example, DAP10 CAD constructs DAP10.6 and DAP10.16 were examined for effectiveness at controlling PLC/PRF/5 tumor grown in a mouse model. As shown in FIG. 16A, tumor control in mice treated with Vδ1 cells transduced with DAP10.16 CAD (in which the CD3ζ intracellular signaling domain includes the 1XX mutation) is improved upon direct comparison to mice treated with Vδ1 cells transduced with DAP10.6 CAD (p=0.0079, Mann-Whitney test (two-tailed). A schematic of the experimental procedure used for this Example is depicted at FIG. 16B.

### Example 16. Comparison of In Vivo Anti-Tumor Activity of DAP10 CAD Vδ1 Cells with CAR Vδ1 Cells

This Example demonstrates that DAP10 CAD+Vδ1 cells exhibit anti-tumor activity with kinetics similar to CAR Vδ1 cells

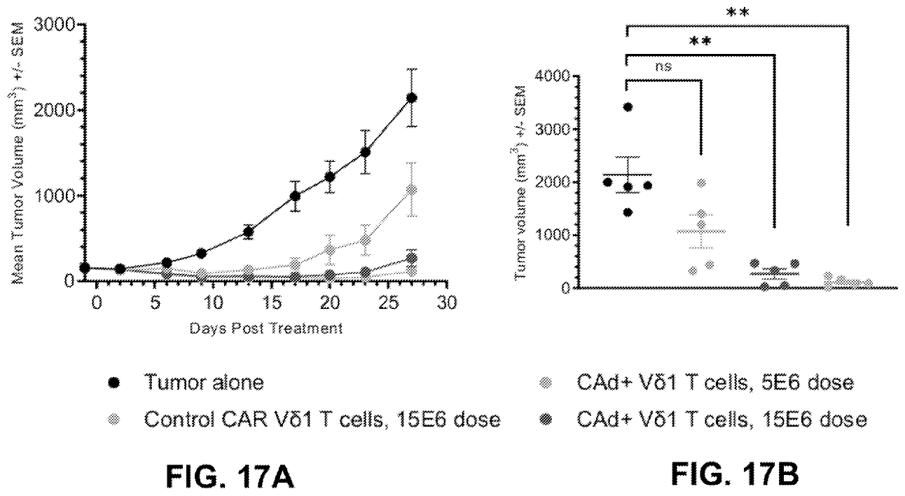

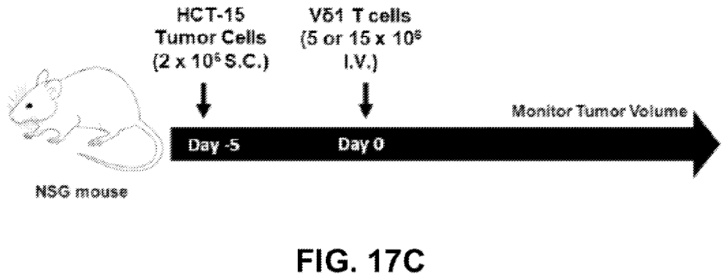

In vivo tumor growth kinetics for DAP10 CAD+Vδ1 cells (5e⁶ cells/dose and 15e⁶ cells/dose as compared to control CAR Vδ1 cells and a tumor alone condition in an HCT-15 mouse xenograft model is shown at FIG. 17A. Tumor volumes as quantified at day 27 are shown at FIG. 17B. A schematic of the experimental procedure used for this Example is depicted at FIG. 17C. Data is shown as mean±SEM for 5 mice/group. A Kruskal-Wallis test with Dunn's multiple comparisons was used to assess final statistical significance amongst complete cohorts for each treatment (ns=not significant).

### Example 17. In Vivo Proliferation, Persistence and Targeting of Engineered Vδ1 Cells

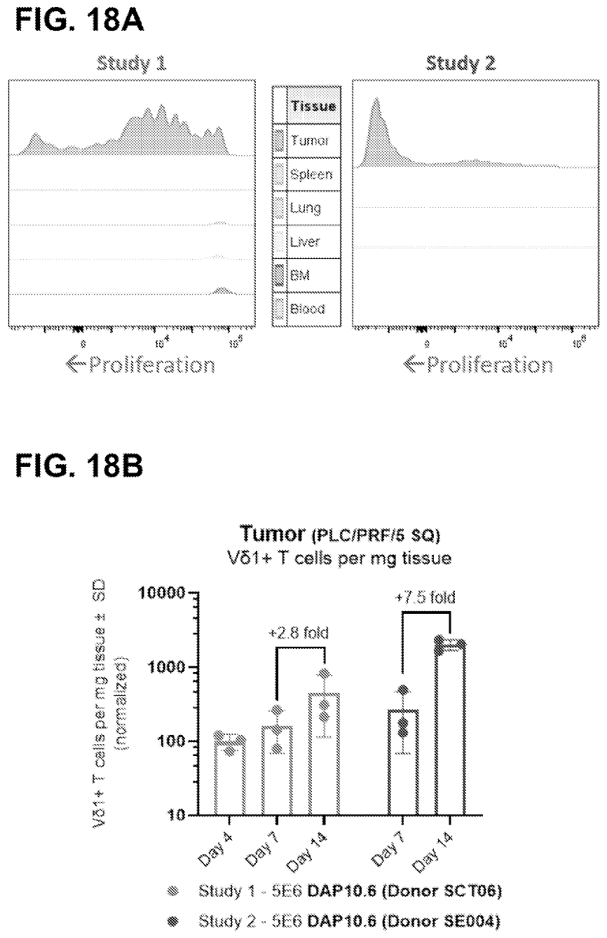

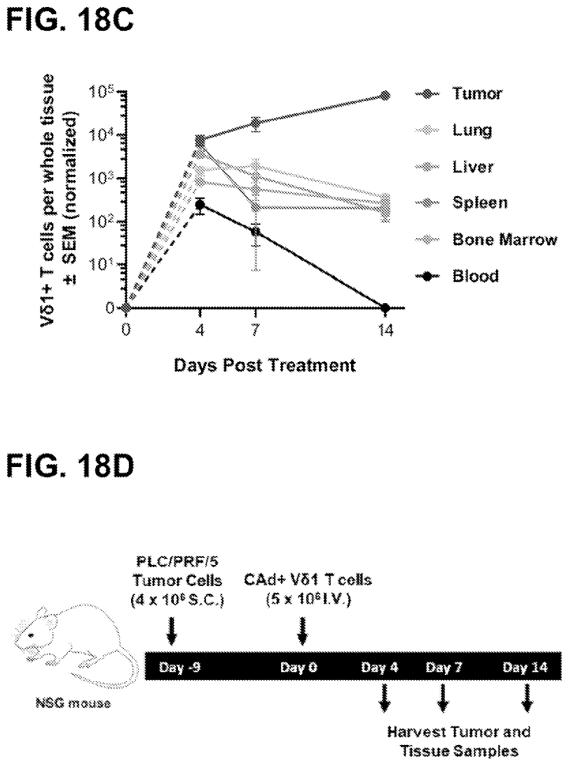

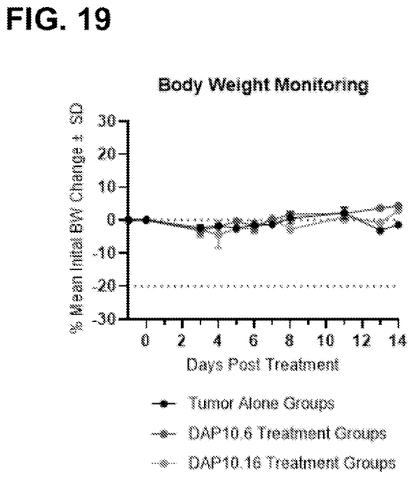

This Example demonstrates that Vδ1 cells transduced with a DAP10 CAD of the present disclosure proliferates in tumors in vivo in a mouse model, but not in other organs. For this Example, two separate studies were conducted. As shown at FIG. 18A, proliferation of Vδ1 cells transduced with DAP10.6 was observed in tumor tissue (subcutaneous PLC/PRF/5 cells), but not in spleen, lung, liver, bone marrow, or blood on day 7 post treatment. Vδ1 cells used in Study 1 were obtained from a different donor than Vδ1 cells used in Study 2. For FIG. 18A, HuCD45+, Vδ1+ population is shown. FIG. 18B is a graph quantifying engineered Vδ1 cells per mg tumor tissue in respective studies on day 4, day 7, and day 14 (Study 1) and on day 7 and day 14 (Study 2). For each study, 5e⁶ engineered Vδ1 cells were used. Notably, an increase in total Vδ1 cells within the tumor was observed throughout each study. FIG. 18C is a graph showing quantification of Vδ1 cells in tumor tissue, or other tissues (lung, liver, spleen, bone marrow, blood) taken 4, 7, or 14 days after treatment as assessed by flow cytometry, and represents cumulative analysis across the two independent studies presented in this Example. FIG. 18D is a schematic showing the experimental procedure corresponding to this Example for reference. No significant changes in body weight or acute clinical signs of off toxicity or xenogeneic graft vs. host disease (GvHD) was observed among treated mice (FIG. 19). Data shown at FIG. 19 is for n=4 independent efficacy studies.

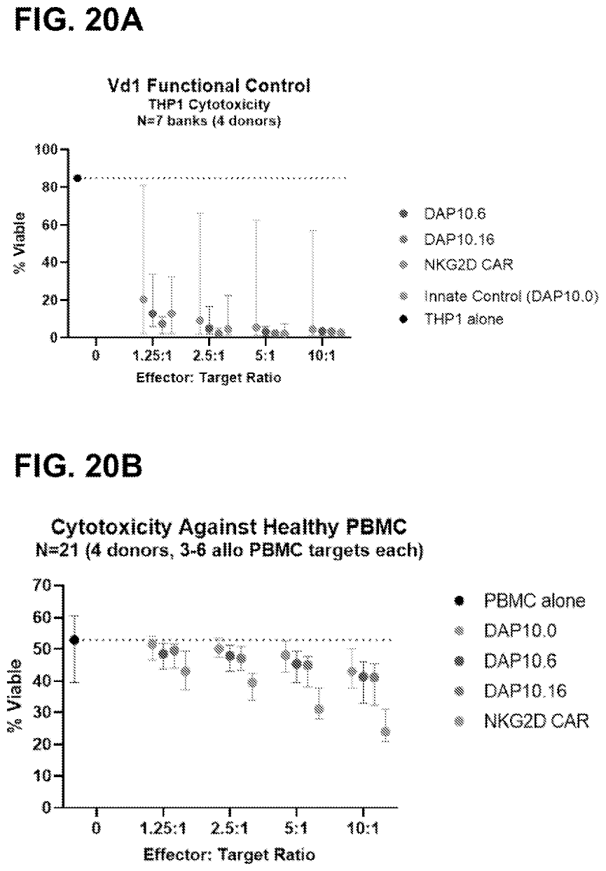

It was found that Vδ1 cells transduced with a DAP10 CAD of the present disclosure efficiently target tumor cells while sparing normal (i.e., non-tumor) cells. Specifically a short-term cytotoxicity assay was adapted to an annexin/DAPI flow based method for analysis of primary cell targets. FIG. 20A is a graph showing that both VS1 cells transduced with a DAP10 CAD of the present disclosure (DAP10.6, DAP10.16), and Vδ1 cells transduced with an NKG2D CAR significantly reduced THP1 cell viability, as compared to THP1 alone and Vδ1 cells transduced with an innate control (DAP10.0). FIG. 20B is a graph showing a substantial lack of targeting of healthy PBMCs by Vδ1 cells transduced with a DAP10 CAD of the present disclosure (DAP10.6, DAP10.16), similar to innate control (VS1 cells transduced with DAP10.0). Notably, Vδ1 cells transduced with a DAP10 CAD consistently demonstrated lower PBMC targeting than an NKG2D CAR reference (FIG. 20B).

### Example 18. Small Scale Donor Screens

This Example demonstrates that expansion of Vδ1 cells transduced with a DAP10 CAD that includes a CD3ζ 1XX modification may be improved over similar constructs in which the CD3ζ intracellular signaling domain does not include the 1XX.

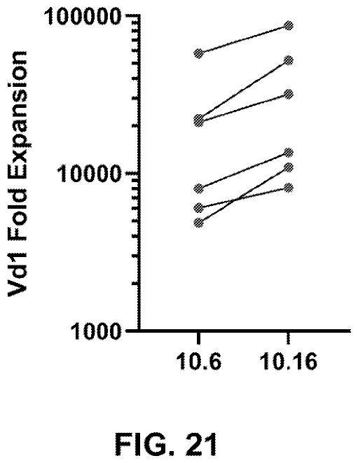

For this Example, Vδ1 cells from 6 donors (SE001, ARC007, HC45, DLS003, SE015, and SCT029) were tested over two experiments in small scale shake flask expansions. Specifically, Vδ1 cells from the different donors were transduced with either DAP10.6 or DAP10.16. 50-135% more Vδ1 cells were measured from DAP10.16 transduced cells as compared to DAP10.6 transduced cells across all donors (FIG. 21). Expansion was measured on day 14, of day 15.

### Example 19. DAP10 CADVδ1 Cell Growth Kinetics

This Example demonstrates that there is a distinct shift in cultures to higher % Vδ1 cells transduced with lead DAP10 CAD constructs (DAP10.6, DAP10.16, DAP10.17) as compared to controls (DAP10.0, CAR control).

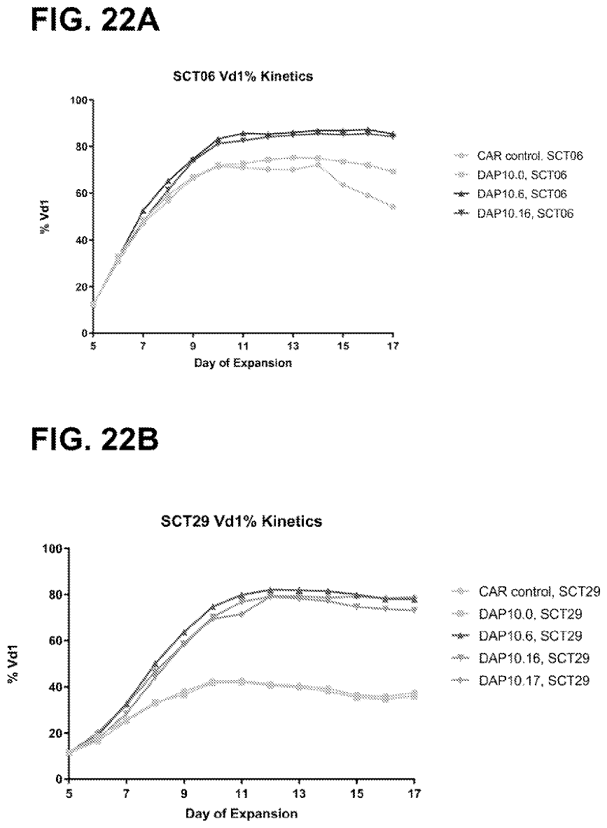

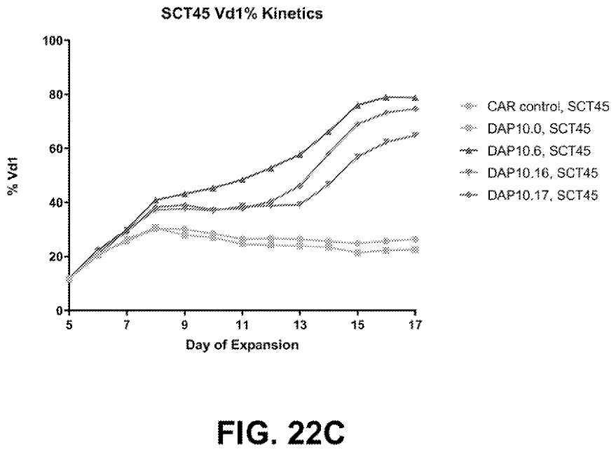

For this Example, Vδ1 cells from three different donors (SCT06, FIG. 22A, SCT29, FIG. 22B, SCT45, FIG. 22C) were transduced with lead DAP10 constructs or controls, and % Vδ1 of total cells was expanded as a function of expansion time. As shown in each of FIGS. 22A-22C, a distinct shift to higher VS1 percentages was observed for the lead DAP10 constructs as compared to controls.

### Example 20. Expansion of Cells Transduced with DAP10 CAD Constructs

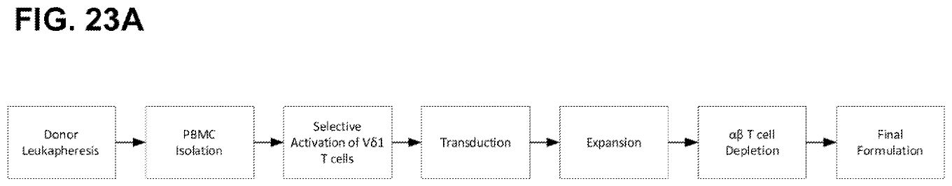

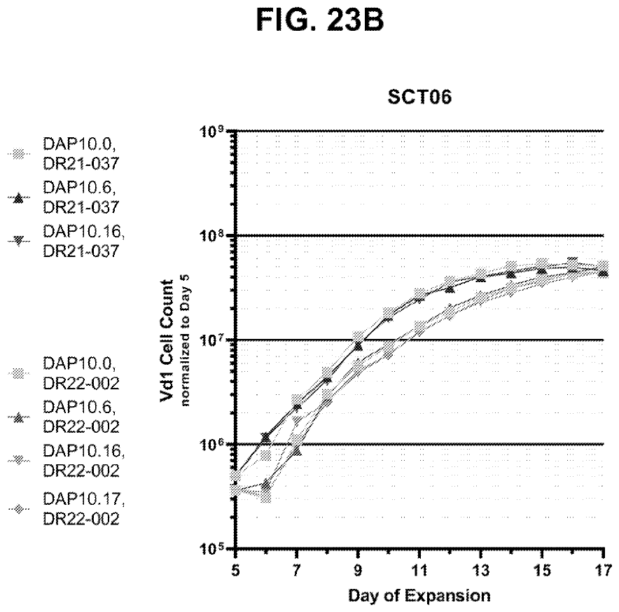

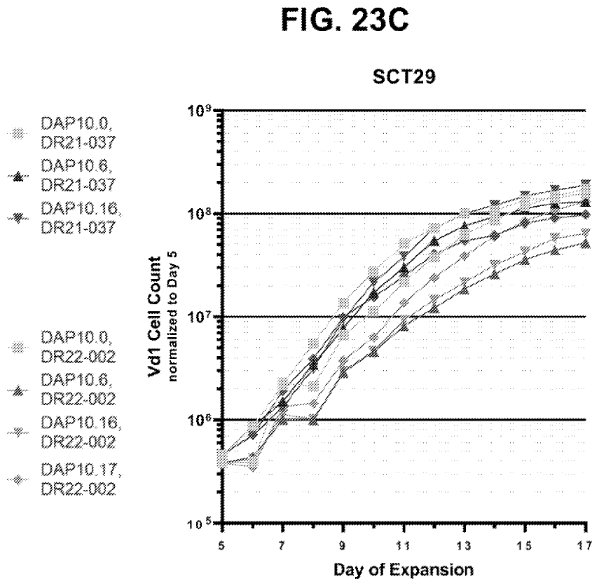

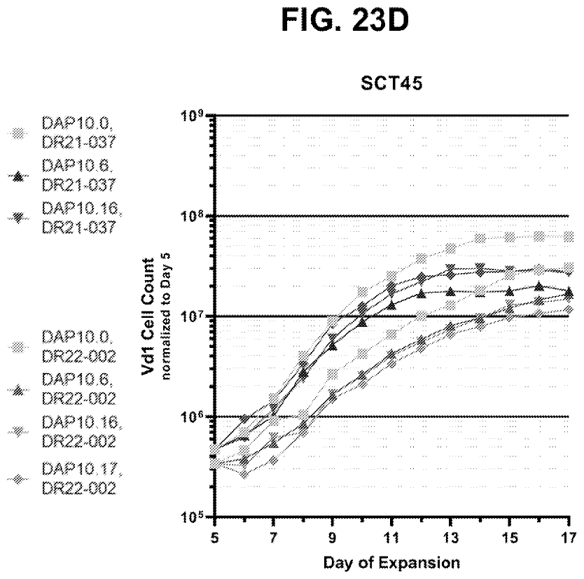

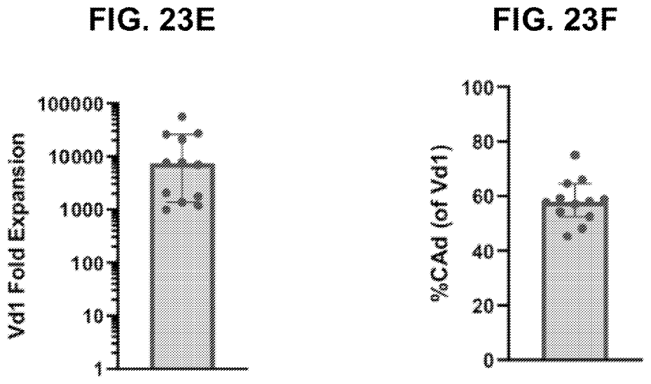

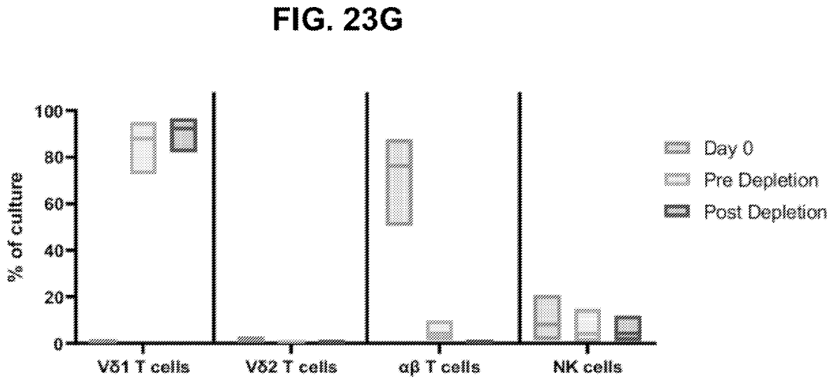

This Example demonstrates V51 cells can be efficiently expanded and transduced with DAP10 CADs of the present disclosure. FIG. 23A depicts a schematic representing a process for generating “off-the-shelf” allogeneic CAD VS1 cells. Data showing independent expansions of lead DAP10 CAD constructs (DAP10.6, DAP10.16, DAP10.17) transduced into Vδ1 cells obtained from three different donors (SCT06, SCT29, SCT45) is depicted at FIGS. 23B, 23C, 23D, respectively. Independent expansions (blue vs red) show similar trends in growth profiles of DAP10 CAD constructs. In two of the three donors, growth was seen to be construct dependent.

A representative experiment illustrates that ex vivo culture of Vδ1 cells results in substantial fold-expansion (FIG. 23E) and robust DAP10 CAD transduction (FIG. 23F) Vδ1 cells. The data of FIGS. 23E-23F was obtained from 12 independent cultures using PBMCs from 7 different donors. Shown at FIG. 23G is a series of graphs showing cellular composition (Vδ1 cells, Vδ2 cells, up cells, and NK cells) over time (day 0, pre-up T cell depletion, and post-αβ T cell depletion) expressed as % of culture.

### Example 21. CAR-CAD Co-Expressing Constructs

Vδ1 cells co-expressing CAR and Dap10 CAD were generated using the constructs in Table 2 below. In brief, the CAR-DAP10 CAD constructs were constructed with 0-4 elements selected from Y86F mutation, K84R mutation, 4-1BB costimulatory domain, CD3ζ signaling domain. For construction, the pSIN was used. Briefly, the pSIN vector backbone was fully synthesized by Genewiz® (South Plainfield, NJ) from sequence provided by EUFETS (Germany) a subsidiary of BioNTech (Germany). The base plasmid used for all constructs is referred to as pL077 pRetroSIN-GFP, which has a green fluorescent protein (GFP) cassette that is replaced by a gene-of-interest. The various constructs are depicted in Table 2 below. With regard to the following constructs, 3H7-5.1 refers to a CAR binding to CD20 (see e.g., Nishimoto et al., Clin Transl Immunology. 2022; 11(2): e1373), and P2A refers to a P2A cleavage sequence.

TABLE 2
CAR-DAP10 CAD Constructs

| | | DAP10 elements | | | |
|---|---|---|---|---|---|
| | Coding | Signal null? | Ubiquitin | | |
| | sequence | (Y86F) | mutant (K84R) | 41BB | CD3z |
| pSIN-3H7-5.1-P2A- | SEQ ID | Yes | No | No | No |
| DAP10.1 | NO: 112 | | | | |
| | (protein: | | | | |
| | SEQ ID | | | | |
| | NO: 113) | | | | |
| pSIN-3H7-5.1-P2A- | SEQ ID | Yes | Yes | No | No |
| DAP10.2 | NO: 114 | | | | |
| | (protein: | | | | |
| | SEQ ID | | | | |
| | NO: 115) | | | | |
| pSIN-3H7-5.1-P2A- | SEQ ID | No | No | No | Yes |
| DAP10.3 | NO: 116 | | | | |
| | (protein: | | | | |
| | SEQ ID | | | | |
| | NO: 117) | | | | |
| pSIN-3H7-5.1-P2A- | SEQ ID | No | No | Yes | Yes |
| DAP10.4 | NO: 118 | | | | |
| | (protein: | | | | |
| | SEQ ID | | | | |
| | NO: 119) | | | | |
| pSIN-3H7-5.1-P2A- | SEQ ID | Yes | No | Yes | Yes |
| DAP10.5 | NO: 120 | | | | |
| | (protein: | | | | |
| | SEQ ID | | | | |
| | NO: 121) | | | | |
| pSIN-3H7-5.1-P2A- | SEQ ID | Yes | Yes | Yes | Yes |
| DAP10.6 | NO: 122 | | | | |
| | (protein: | | | | |
| | SEQ ID | | | | |
| | NO: 123) | | | | |
| pSIN-3H7-5.1-P2A- | SEQ ID | No | No | Yes | No |
| DAP10.7 | NO: 124 | | | | |
| | (protein: | | | | |
| | SEQ ID | | | | |
| | NO: 125) | | | | |
| pSIN-3H7-5.1-P2A- | SEQ ID | Yes | No | Yes | No |
| DAP10.8 | NO: 126 | | | | |
| | (protein: | | | | |
| | SEQ ID | | | | |
| | NO: 127) | | | | |
| pSIN-3H7-5.1-P2A- | SEQ ID | Yes | Yes | Yes | No |
| DAP10.9 | NO: 128 | | | | |
| | (protein: | | | | |
| | SEQ ID | | | | |
| | NO: 129) | | | | |
| pSIN-3H7-5.1-P2A- | SEQ ID | No | No | No | No |
| DAP10.0 | NO: 130 | | | | |
| | (protein: | | | | |
| | SEQ ID | | | | |
| | NO: 131) | | | | |

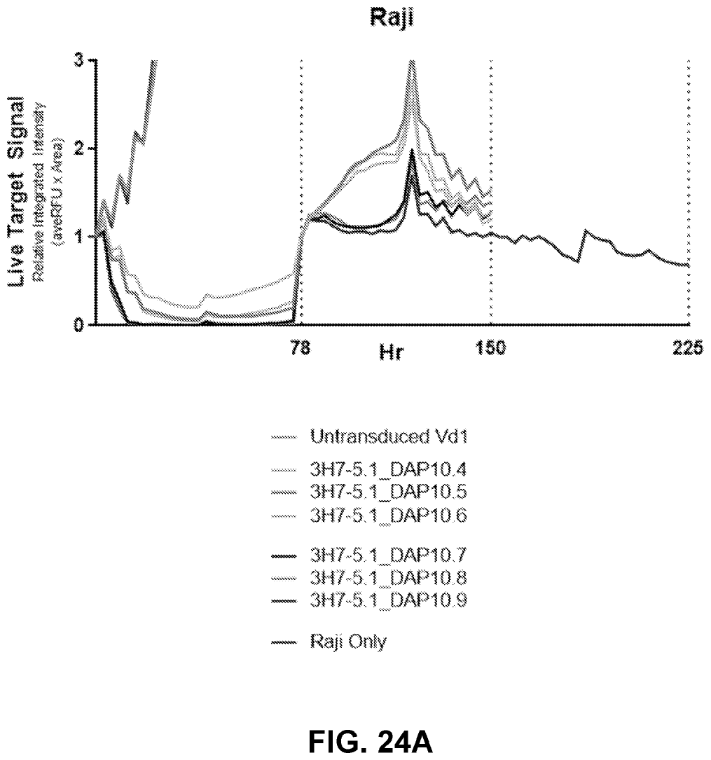

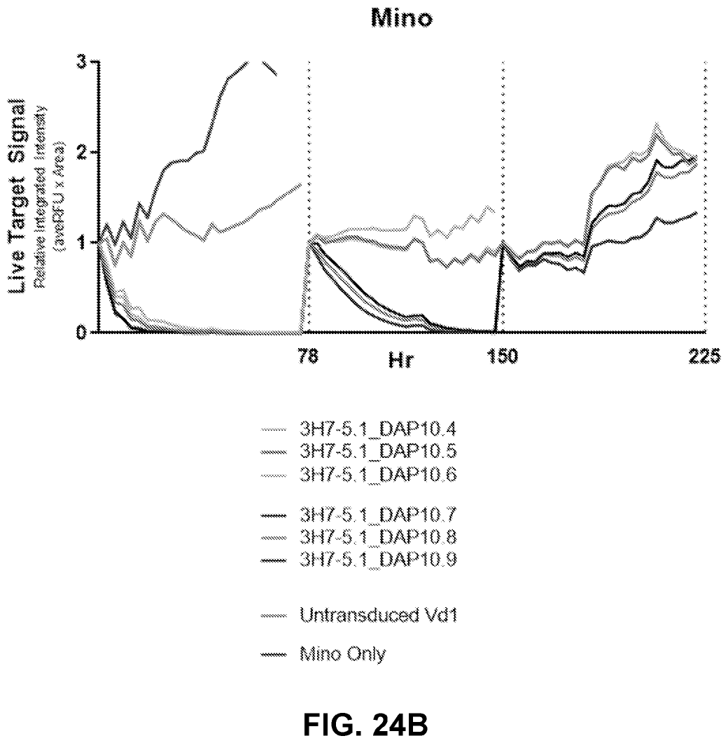

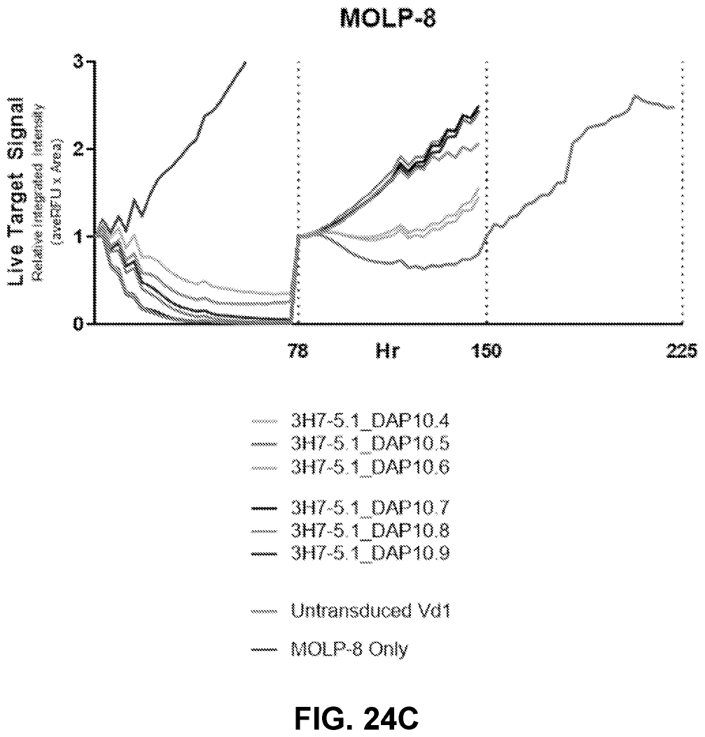

The Vδ1 γδ cells expressing the CAR-Dap10 CAD constructs were tested by Incucyte-based restimulation assay with Raji, Mino, and MOLP-8 cell lines, in which the Raji and Mino cell lines expressed the target of the CAR but the MOLP-8 cell line did not. FIGS. 24A-24C show the results with the Raji, Mino, and MOLP8 cell lines, respectively. V1 cells transduced with CAR-Dap10 CAD constructs showed prolonged cytotoxic activity against targets with and without the CAR antigen as compared to untransduced controls.

Certain modifications and improvements will occur to those skilled in the art upon a reading of the foregoing description. It should be understood that all such modifications and improvements have been deleted herein for the sake of conciseness and readability but are properly within the scope of the following claims.

TABLE 3
Sequence information

| SEQ ID | | |
|---|---|---|
| No. | Name | Sequence |
| 1 | DAP10 | MIHLGHILFLLLLPVAAAQTTPGERSSLPAFYPGTSGSCSGCGSLSL |
| | polypeptide | PLLAGLVAADAVASLLIVGAVFLCARPRRSPAQEDGKVYINMPG |
| | | RG |
| 2 | 4-1BB | KRGRKKLLYIFKQPFMRPVQTTQEEDGCSCRFPEEEEGGCEL |
| | costimulation | |
| | endodomain | |
| | polypeptide | |
| 3 | CD3z signaling | RVKFSRSADAPAYQQGQNQLYNELNLGRREEYDVLDKRRGRDP |
| | domain | EMGGKPQRRKNPQEGLYNELQKDKMAEAYSEIGMKGERRRGK |
| | polypeptide 1 | GHDGLYQGLSTATKDTYDALHMQALPPR |
| 4 | CD3z signaling | RVKFSRSADAPAYQQGQNQLYNELNLGRREEYDVLDKRRGRDP |
| | domain | EM |
| | polypeptide 2 | GGKPRRKNPQEGLYNELQKDKMAEAYSEIGMKGERRRGKGHD |
| | | GLYQGLSTATKDTYDALHMQALPPR |
| 5 | CD27 | QRRKYRSNKGESPVEPAEPCHYSCPREEEGSTIPIQEDYRKPEPAC |
| | costimulatory | SP |
| | domain | |
| | polypeptide | |
| 6 | secretion signal | MALPVTALLLPLALLLHAARP |
| | 1 polypeptide | |
| 7 | secretion signal | MRISKPHLRSISIQCYLCLLLNSHFLTEAGIHVFILGCF |
| | 2 polypeptide | SAGLPKTEA |
| 8 | IL-5 | NWVNVISDLKKIEDLIQSMHIDATLYTESDVHPSCKVTAMKCELL |
| | polypeptide | ELQVISLESGDASIHDTVENLIILANNSLSSNGNVTESGCKECEELE |
| | | EKNIKEFLQSFVHIVQMFINTS |
| 9 | P2A cleavage | SGSGATNFSLLKQAGDVEENPGP |
| | motif 1 | |
| | polypeptide | |
| 10 | furin cleavage | RAKR |
| | site polypeptide | |
| 11 | cleavable linker | RAKRSGSGATNFSLLKQAGDVEENPGP |
| | polypeptide | |
| 12 | P2A cleavage | ATNFSLLKQAGDVEENPGP |
| | motif 2 | |
| | polypeptide | |
| 13 | F2A cleavage | VKQTLNNFDLLKLAGDVESNPGP |
| | motif | |
| | polypeptide | |
| 14 | E2A cleavage | QCTNYALLKLAGDVESNPGP |
| | motif | |
| | polypeptide | |
| 15 | T2A cleavage | EGRSLLTCGDVEENPGP |
| | motif | |
| | polypeptide | |
| 16 | IRES 1 | CTAACGTTACTGGCCGAAGCCGCTTGGAATAAGGCCGGTGTG |
| | nucleotide | CGTTTGTCTATATGTTATTTTCCACCATATTGCCGTCTTTTGGC |
| | | AATGTGAGGGCCCGGAAACCTGGCCCTGTCTTCTTGACGAGCA |
| | | TTCCTAGGGGTCTTTCCCCTCTCGCCAAAGGAATGCAAGGTCT |
| | | GTTGAATGTCGTGAAGGAAGCAGTTCCTCTGGAAGCTTCTTGA |
| | | AGACAAACAACGTCTGTAGCGACCCTTTGCAGGCAGCGGAAC |
| | | CCCCCACCTGGCGACAGGTGCCTCTGCGGCCAAAAGCCACGT |
| | | GTATAAGATACACCTGCAAAGGCGGCACAACCCCAGTGCCAC |
| | | GTTGTGAGTTGGATAGTTGTGGAAAGAGTCAAATGGCTCTCCT |
| | | CAAGCGTATTCAACAAGGGGCTGAAGGATGCCCAGAAGGTAC |
| | | CCCATTGTATGGGATCTGATCTGGGGCCTCGGTGCACATGCTT |
| | | TACATGTGTTTAGTCGAGGTTAAAAAAACGTCTAGGCCCCCCG |
| | | AACCACGGGGACGTGGTTTTCCTTTGAAAAACACGATGATA |
| 17 | IRES 2 | AGCAGGTTTCCCCAACTGACACAAAACGTGCAACTTGAAACT |
| | nucleotide | CCGCCTGGTCTTTCCAGGTCTAGAGGGGTAACACTTTGTACTG |
| | | CGTTTGGCTCCACGCTCGATCCACTGGCGAGTGTTAGTAACAG |
| | | CACTGTTGCTTCGTAGCGGAGCATGACGGCCGTGGGAACTCCT |
| | | CCTTGGTAACAAGGACCCACGGGGCCAAAAGCCACGCCCACA |
| | | CGGGCCCGTCATGTGTGCAACCCCAGCACGGCGACTTTACTGC |
| | | GAAACCCACTTTAAAGTGACATTGAAACTGGTACCCACACACT |
| | | GGTGACAGGCTAAGGATGCCCTTCAGGTACCCCGAGGTAACA |
| | | CGCGACACTCGGGATCTGAGAAGGGGACTGGGGCTTCTATAA |
| | | AAGCGCTCGGTTTAAAAAGCTTCTATGCCTGAATAGGTGACCG |
| | | GAGGTCGGCACCTTTCCTTTGCAATTACTGACCAC |
| 18 | DAP10-K84R | MIHLGHILFLLLLPVAAAQTTPGERSSLPAFYPGTSGSCSGCGSLSL |
| | polypeptide | PLLAGLVAADAVASLLIVGAVFLCARPRRSPAQEDGRVYINMPG |
| | | RG |
| 19 | DAP10-Y86F | MIHLGHILFLLLLPVAAAQTTPGERSSLPAFYPGTSGSCSGCGSLSL |
| | polypeptide | PLLAGLVAADAVASLLIVGAVFLCARPRRSPAQEDGKVFINMPG |
| | | RG |
| 20 | DAP10-K84R- | MIHLGHILFLLLLPVAAAQTTPGERSSLPAFYPGTSGSCSGCGSLSL |
| | Y86F | PLLAGLVAADAVASLLIVGAVFLCARPRRSPAQEDGRVFINMPGR |
| | polypeptide | G |
| 21 | DAP10.0 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGKVYINMPGRGRAKRSGSGATNFS |
| | | LLKQAGDVEENPGPMPPPRLLFFLLFLTPMEVRPEEPLVVKV |
| | | EEGDNAVLQCLKGTSDGPTQQLTWSRESPLKPFLKLSLGLPG |
| | | LGIHMRPLAIWLFIFNVSQQMGGFYLCQPGPPSEKAWQPGW |
| | | TVNVEGSGELFRWNVSDLGGLGCGLKNRSSEGPSSPSGKLM |
| | | SPKLYVWAKDRPEIWEGEPPCLPPRDSLNQSLSQDLTMAPGS |
| | | TLWLSCGVPPDSVSRGPLSWTHVHPKGPKSLLSLELKDDRPA |
| | | RDMWVMETGLLLPRATAQDAGKYYCHRGNLTMSFHLEITA |
| | | RPVLWHWLLRTGGWKVSAVTLAYLIFCLCSLVGILHLQRAL |
| | | VLRRKRKRMTDPTRRF |
| 22 | DAP10.0 | atgtccgtgcctacccaggtgctgggcctgctgctgctgtggctgaccgacgccagatgc |
| | Nucleic Acid | atgatccacctgggccacatcctgtttctgctgctgcttcctgtggccgctgctcagaca |
| | | acacctggcgagagatctagcctgcctgccttctatcctggcaccagcggctcttgttct |
| | | ggctgtggatctctgagcctgcctctgctggctggactggttgctgctgatgctgtggcc |
| | | tctctgctgattgtgggcgccgtgttcctgtgtgcccggcctagaagatctcccgctcaa |
| | | gaggatggcaaggtgtacatcaacatgcccggcagaggacgcgcgaagcgatcaggcagc |
| | | ggggcgacaaatttcagccttctgaaacaagcaggcgacgtggaagaaaaccccggtcca |
| | | atgcctcctcctcggctgctgttcttcctgctgtttctgacccctatggaagtgcggccc |
| | | gaggaacctctggtggtcaaagttgaagagggcgacaacgccgtgctgcagtgtctgaag |
| | | ggcacatctgatggccccacacagcagctgacctggtctagagagagccctctgaagccc |
| | | ttcctgaagctgtctctgggactgcctggactgggcatccatatgaggcctctggccatc |
| | | tggctgttcatcttcaacgtgtcccagcagatgggcggcttctacctgtgtcaacctgga |
| | | cctccaagcgagaaggcttggcagcctggctggaccgtgaatgtggaaggatccggcgag |
| | | ctgttccggtggaatgtgtctgatctcggcggcctcggatgcggcctgaagaatagatct |
| | | agcgagggccctagcagccccagcggaaaactgatgagccccaagctgtacgtgtgggcc |
| | | aaagacagacccgagatttgggagggcgagcctccttgtctgcctcctagagacagcctg |
| | | aaccagagcctgagccaggacctgacaatggcccctggatctacactgtggctgagctgt |
| | | ggcgtgccacctgacagtgtgtctagaggccctctgtcttggacccacgtgcaccctaag |
| | | ggccctaagtctctgctgagcctggaactgaaggacgacaggcccgccagagatatgtgg |
| | | gtcatggaaacaggcctgctgctgcctagagccacagcacaggatgccggcaagtactac |
| | | tgccacagaggcaacctgaccatgagcttccacctggaaatcaccgccagacctgtcctg |
| | | tggcactggctgcttagaaccggcggctggaaagtgtctgccgtgactctggcctacctg |
| | | atcttctgcctgtgtagcctcgtgggcatcctgcatctgcagagagcactggtcctgcgg |
| | | cggaagcggaagagaatgaccgatcctaccagacggttctga |
| 23 | DAP10.3 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGKVYINMPGRGRVKFSRSADAPAY |
| | | QQGQNQLYNELNLGRREEYDVLDKRRGRDPEMGGKPQRRK |
| | | NPQEGLYNELQKDK |
| | | MAEAYSEIGMKGERRRGKGHDGLYQGLSTATKDTYDALHM |
| | | QALPPRRAKRSGSGATNFSLLKQAGDVEENPGPMPPPRLLFF |
| | | LLFLTPMEVRPEEPLVVKVEEGDNAVLQCLKGTSDGPTQQLT |
| | | WSRESPLKPFLKLSLGLPGLGIHMRPLAIWLFIFNVSQQMGGF |
| | | YLCQPGPPSEKAW |
| | | QPGWTVNVEGSGELFRWNVSDLGGLGCGLKNRSSEGPSSPS |
| | | GKLMSPKLYVWAKDRPEIWEGEPPCLPPRDSLNQSLSQDLT |
| | | MAPGSTLWLSCGVPPDSVSRGPLSWTHVHPKGPKSLLSLELK |
| | | DDRPARDMWVMETGLLLPRATAQDAGKYYCHRGNLTMSF |
| | | HLEITARPVLWHWLLRT |
| | | GGWKVSAVTLAYLIFCLCSLVGILHLQRALVLRRKRKRMTD |
| | | PTRRF |
| 24 | DAP10.3 | atgtccgtgcctacccaggtgctgggcctgctgctgctgtggctgaccgacgccagatgcat |
| | Nucleic Acid | gatccacctgggccacatcctgtttctgctgctgcttcctgtggccgctgctcagaca |
| | | acacctggcgagagatctagcctgcctgccttctatcctggcaccagcggctcttgttctgg |
| | | ctgtggatctctgagcctgcctctgctggctggactggttgctgctgatgctgtggcctctc |
| | | tgctgattgtgggcgccgtgttcctgtgtgcccggcctagaagatctcccgctcaa |
| | | gaggatggcaaggtgtacatcaacatgcccggcagaggaagagtgaagttcagcagaagcgc |
| | | cgatgcccctgcctatcagcagggccagaaccagctgtacaacgagctgaacctgggcagac |
| | | gcgaggaatacgacgtgctggacaagcggcggggacgggaccccgagatgggcggc |
| | | aagcctcaaagaaggaagaacccccaggagggcctgtataatgagctgcagaaagataagat |
| | | ggccgaggcctacagcgagatcggcatgaaaggcgagagacggcggggcaagggccacga |
| | | cggcctgtaccagggcctgagcaccgccacaaaggacacctacgacgccctgcacatg |
| | | caggccctgccacctagacgcgcgaagcgatcaggcagcggggcgacaaatttcagccttct |
| | | gaaacaagcaggcgacgtggaagaaaaccccggtccaatgcctcctcctcggctgctgttct |
| | | tcctgctgtttctgacccctatggaagtgcggcccgaggaacctctggtggtcaaa |
| | | gttgaagagggcgacaacgccgtgctgcagtgtctgaagggcacatctgatggccccacaca |
| | | gcagctgacctggtctagagagagccctctgaagcccttcctgaagctgtctctgggactgc |
| | | ctggactgggcatccatatgaggcctctggccatctggctgttcatcttcaacgtg |
| | | tcccagcagatgggggcttctacctgtgtcaacctggacctccaagcgagaaggcttggcag |
| | | cctggctggaccgtgaatgtggaaggatccggcgagctgttccggtggaatgtgtctgatct |
| | | cggcggcctcggatgcggcctgaagaatagatctagcgagggccctagcagccccagcggaa |
| | | aactgatgagccccaagctgtacgtgtgggccaaagacagacccgagatttgggagggcgag |
| | | cctccttgtctgcctcctagagacagcctgaaccagagcctgagccaggacctgacaatggc |
| | | ccctggatctacactgtggctgagctgtggcgtgccacctgacagtgtgtctagaggccctc |
| | | tgtcttggacccacgtgcaccctaagggccctaagtctctgctgagcctggaactgaaggac |
| | | gacaggcccgccagagatatgtgggtcatggaaacaggcctgctgctgcctagagccacagc |
| | | acaggatgccggcaagtactactgccacagaggcaacctgaccatgagcttccacctggaaa |
| | | tcaccgccagacctgtcctgtggcactggctgcttagaaccggcggctggaaagtgtctgcc |
| | | gtgactctggcctacctgatcttctgcctgtgtagcctcgtgggcatcctgcatctgcagag |
| | | agcactggtcctgcggcggaagcggaagagaatgaccgatcctaccagacggttctga |
| 25 | DAP10.4 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGKVYINMPGRGKRGRKKLLYIFKQ |
| | | PFMRPVQTTQEEDGCSCRFPEEEEGGCELRVKFSRSADAPAY |
| | | QQGQNQLYNELN |
| | | LGRREEYDVLDKRRGRDPEMGGKPQRRKNPQEGLYNELQK |
| | | DKMAEAYSEIGMKGERRRGKGHDGLYQGLSTATKDTYDAL |
| | | HMQALPPRRAKRSGSGATNFSLLKQAGDVEENPGPMPPPRL |
| | | LFFLLFLTPMEVRPEEPLVVKVEEGDNAVLQCLKGTSDGPTQ |
| | | QLTWSRESPLKPFLKLS |
| | | LGLPGLGIHMRPLAIWLFIFNVSQQMGGFYLCQPGPPSEKAW |
| | | QPGWTVNVEGSGELFRWNVSDLGGLGCGLKNRSSEGPSSPS |
| | | GKLMSPKLYVWAKDRPEIWEGEPPCLPPRDSLNQSLSQDLT |
| | | MAPGSTLWLSCGVPPDSVSRGPLSWTHVHPKGPKSLLSLELK |
| | | DDRPARDMWVMETG |
| | | LLLPRATAQDAGKYYCHRGNLTMSFHLEITARPVLWHWLLR |
| | | TGGWKVSAVTLAYLIFCLCSLVGILHLQRALVLRRKRKRMT |
| | | DPTRRF |
| 26 | DAP10.4 | atgtccgtgcctacccaggtgctgggcctgctgctgctgtggctgaccgacgccagatgcat |
| | Nucleic Acid | gatccacctgggccacatcctgttcctgctgctgctgcctgtggccgccgctcaaacaaccc |
| | | ctggcgagagatctagcctgccagccttctaccccggtacaagcggatcttgcagc |
| | | ggctgcggcagcctgtctctgcctctgctggccggcctggtggctgctgatgctgtggccag |
| | | cctgctgatcgtgggcgccgtgttcctgtgcgccagacctagaaggtcccctgcccaggagg |
| | | atggaaaggtgtacatcaacatgcctggcagaggcaagagaggcagaaagaagctg |
| | | ctgtacatcttcaagcagcctttcatgagaccagtgcagaccacccaggaggaggacggatg |
| | | tagctgcagattccccgaggaagaagaaggcggctgtgaactgagagtgaagttcagcagaa |
| | | gcgccgacgcccctgcttatcagcagggccagaaccagctgtacaacgagctgaac |
| | | ctgggcagaagggaagagtacgacgtgctggacaagcggcggggacgggaccccgagatg |
| | | ggcggaaaacctcaaagacggaagaacccccaggagggcctttataatgagctgcagaaaga |
| | | taagatggccgaggcctacagcgagatcggcatgaaaggagaaagacggcggggcaaa |
| | | ggccacgacggcctgtaccagggcctgagcaccgccacaaaggacacctacgacgccctgc |
| | | acatgcaggccctgccccccagacgcgcgaagcgatcaggcagcggggcgacaaatttcagc |
| | | cttctgaaacaagcaggcgacgtggaagaaaaccccggtccaatgcctcctcctcgg |
| | | ctgctgttcttcctgctgtttctgacccctatggaagtgcggcccgaggaacctctggtggt |
| | | caaagttgaagagggcgacaacgccgtgctgcagtgtctgaagggcacatctgatggcccca |
| | | cacagcagctgacctggtctagagagagccctctgaagcccttcctgaagctgtct |
| | | ctgggactgcctggactgggcatccatatgaggcctctggccatctggctgttcatcttcaa |
| | | cgtgtcccagcagatgggcggcttctacctgtgtcaacctggacctccaagcgagaaggctt |
| | | ggcagcctggctggaccgtgaatgtggaaggatccggcgagctgttccggtggaat |
| | | gtgtctgatctcggcggcctcggatgcggcctgaagaatagatctagcgagggccctagcag |
| | | ccccagcggaaaactgatgagccccaagctgtacgtgtgggccaaagacagacccgagattt |
| | | gggagggcgagcctccttgtctgcctcctagagacagcctgaaccagagcctgagc |
| | | caggacctgacaatggcccctggatctacactgtggctgagctgtggcgtgccacctgacag |
| | | tgtgtctagaggccctctgtcttggacccacgtgcaccctaagggccctaagtctctgctga |
| | | gcctggaactgaaggacgacaggcccgccagagatatgtgggtcatggaaacaggc |
| | | ctgctgctgcctagagccacagcacaggatgccggcaagtactactgccacagaggcaacct |
| | | gaccatgagcttccacctggaaatcaccgccagacctgtcctgtggcactggctgcttagaa |
| | | ccggcggctggaaagtgtctgccgtgactctggcctacctgatcttctgcctgtgt |
| | | agcctcgtgggcatcctgcatctgcagagagcactggtcctgcggcggaagcggaagagaat |
| | | gaccgatcctaccagacggttctga |
| 27 | DAP10.5 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGKVFINMPGRGKRGRKKLLYIFKQP |
| | | FMRPVQTTQEEDGCSCRFPEEEEGGCELRVKFSRSADAPAYQ |
| | | QGQNQLYNELNLGRREEYDVLDKRRGRDPEMGGKPQRRKN |
| | | PQEGLYNELQKDKMAEAYSEIGMKGERRRGKGHDGLYQGL |
| | | STATKDTYDALHMQALPPRRAKRSGSGATNFSLLKQAGDVE |
| | | ENPGPMPPPRLLFFLLFLTPMEVRPEEPLVVKVEEGDNAVLQ |
| | | CLKGTSDGPTQQLTWSRESPLKPFLKLSLGLPGLGIHMRPLAI |
| | | WLFIFNVSQQMGGFYLCQPGPPSEKAWQPGWTVNVEGSGEL |
| | | FRWNVSDLGGLGCGLKNRSSEGPSSPSGKLMSPKLYVWAKD |
| | | RPEIWEGEPPCLPPRDSLNQSLSQDLTMAPGSTLWLSCGVPP |
| | | DSVSRGPLSWTHVHPKGPKSLLSLELKDDRPARDMWVMET |
| | | GLLLPRATAQDAGKYYCHRGNLTMSFHLEITARPVLWHWLL |
| | | RTGGWKVSAVTLAYLIFCLCSLVGILHLQRALVLRRKRKRM |
| | | TDPTRRF |
| 28 | DAP10.5 | atgtccgtgcctacccaggtgctgggcctgctgctgctgtggctgaccgacgccagatgcat |
| | Nucleic Acid | gatccacctgggacacatcctgttcctgctgctgcttccagtggccgccgctcaaacaaccc |
| | | ctggcgagagaagcagcctgcccgccttctaccccggtacatctggcagctgcagc |
| | | ggctgcggcagcttgtctctgcctctgctggccggactggtggctgctgatgccgtggccag |
| | | cctgctgatcgtgggcgccgtgttcctgtgcgccagacccagaaggtcccctgcccaggagg |
| | | acggcaaggtgttcatcaacatgcctggcagaggcaaaagaggcagaaagaagctg |
| | | ctgtacatcttcaagcagcctttcatgcggcctgtgcagaccacccaggaggaagatggctg |
| | | ctcttgtcgatttccagaagaggaagaaggcggctgtgaactgagagtgaagttcagcagat |
| | | ccgccgacgcccctgcttatcagcagggccagaaccagctgtataatgagctgaac |
| | | ctgggccggcgggaagagtacgacgtgctggacaagcggcggggcagagatcctgagatgg |
| | | gcggaaaacctcaaagaaggaagaacccccaggagggactgtacaacgagctgcagaag |
| | | gataagatggccgaggcctacagcgagatcggcatgaaaggagaaagaagaagaggaaaag |
| | | gccacgacggcctgtaccagggcctgagcaccgccaccaaggacacctacgacgccctgcac |
| | | atgcaggccctgccacctagacgcgcgaagcgatcaggcagcggggcgacaaatttcagcct |
| | | tctgaaacaagcaggcgacgtggaagaaaaccccggtccaatgcctcctcctcggctgctgt |
| | | tcttcctgctgtttctgacccctatggaagtgcggcccgaggaacctctggtggtcaaagtt |
| | | gaagagggcgacaacgccgtgctgcagtgtctgaagggcacatctgatggccccacacagca |
| | | gctgacctggtctagagagagccctctgaagcccttcctgaagctgtctctgggactgcctg |
| | | gactgggcatccatatgaggcctctggccatctggctgttcatcttcaacgtgtcccagcag |
| | | atgggcggcttctacctgtgtcaacctggacctccaagcgagaaggcttggcagcctggctg |
| | | gaccgtgaatgtggaaggatccggcgagctgttccggtggaatgtgtctgatctcggcggcc |
| | | tcggatgcggcctgaagaatagatctagcgagggccctagcagccccagcggaaaactgatg |
| | | agccccaagctgtacgtgtgggccaaagacagacccgagatttgggagggcgagcctccttg |
| | | tctgcctcctagagacagcctgaaccagagcctgagc |
| | | caggacctgacaatggcccctggatctacactgtggctgagctgtggcgtgccacctgacag |
| | | tgtgtctagaggccctctgtcttggacccacgtgcaccctaagggccctaagtctctgctga |
| | | gcctggaactgaaggacgacaggcccgccagagatatgtgggtcatggaaacaggc |
| | | ctgctgctgcctagagccacagcacaggatgccggcaagtactactgccacagaggcaacct |
| | | gaccatgagcttccacctggaaatcaccgccagacctgtcctgtggcactggctgcttagaa |
| | | ccggcggctggaaagtgtctgccgtgactctggcctacctgatcttctgcctgtgt |
| | | agcctcgtgggcatcctgcatctgcagagagcactggtcctgcggcggaagcggaagagaat |
| | | gaccgatcctaccagacggttctga |
| 29 | DAP10.6 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGRVFINMPGRGKRGRKKLLYIFKQP |
| | | FMRPVQTTQEEDGCSCRFPEEEEGGCELRVKFSRSADAPAYQ |
| | | QGQNQLYNELNLGRREEYDVLDKRRGRDPEMGGKPQRRKN |
| | | PQEGLYNELQKDKMAEAYSEIGMKGERRRGKGHDGLYQGL |
| | | STATKDTYDALHMQALPPRRAKRSGSGATNFSLLKQAGDVE |
| | | ENPGPMPPPRLLFFLLFLTPMEVRPEEPLVVKVEEGDNAVLQ |
| | | CLKGTSDGPTQQLTWSRESPLKPFLKLSLGLPGLGIHMRPLAI |
| | | WLFIFNVSQQMGGFYLCQPGPPSEKAWQPGWTVNVEGSGEL |
| | | FRWNVSDLGGLGCGLKNRSSEGPSSPSGKLMSPKLYVWAKD |
| | | RPEIWEGEPPCLPPRDSLNQSLSQDLTMAPGSTLWLSCGVPP |
| | | DSVSRGPLSWTHVHPKGPKSLLSLELKDDRPARDMWVMET |
| | | GLLLPRATAQDAGKYYCHRGNLTMSFHLEITARPVLWHWLL |
| | | RTGGWKVSAVTLAYLIFCLCSLVGILHLQRALVLRRKRKRM |
| | | TDPTRRF |
| 30 | DAP10.6 | atgtccgtgcctacccaggtgctgggcctgctgctgctgtggctgaccgacgccagatgcat |
| | Nucleic Acid | gatccacctgggccacatcctgttcctgctgctcctgcctgtggccgctgctcagaccaccc |
| | | ctggagaaagatctagcctgcccgctttctaccccgggaccagcggcagctgcagc |
| | | ggatgcggctctctgagcctgcctctgctggccggcctcgtggccgccgatgctgtggccag |
| | | cctgctgatcgtgggcgccgtgttcctgtgcgccagacccagaagaagccctgcccaggagg |
| | | atggcagagtgttcatcaacatgcctggcagaggcaaaagaggcagaaagaagctg |
| | | ctgtacatcttcaagcagcctttcatgcggcccgtgcagacaacccaggaggaggacggctg |
| | | tagctgtagattccccgaggaagaagaaggcggctgcgagcttagagtgaagttcagcagaa |
| | | gcgccgacgcccctgcttaccagcagggccagaaccagctgtataatgagctgaacctggga |
| | | agaagggaagagtacgacgtgctggacaagcggcggggcagagatcctgagatgggcggaaa |
| | | acctcaaagaaggaagaaccctcaggagggcctgtacaacgagctgcagaaagacaagatgg |
| | | ccgaggcctacagcgagatcggcatgaaaggcgagagacggcggggcaagggccacgacggc |
| | | ctgtaccaaggcctgtcaacagccaccaaggacacctacgacgccctgcacatgcaggccct |
| | | gccacctagacgcgcgaagcgatcaggcagcggggcgacaaatttcagccttctgaaacaag |
| | | caggcgacgtggaagaaaaccccggtccaatgcctcctcctcggctgctgttcttcctgctg |
| | | tttctgacccctatggaagtgcggcccgaggaacctctggtggtcaaagttgaagagggcga |
| | | caacgccgtgctgcagtgtctgaagggcacatctgatggccccacacagcagctgacctggt |
| | | ctagagagagccctctgaagcccttcctgaagctgtctctgggactgcctggactgggcatc |
| | | catatgaggcctctggccatctggctgttcatcttcaacgtgtcccagcagatgggcggctt |
| | | ctacctgtgtcaacctggacctccaagcgagaaggcttggcagcctggctggaccgtgaatg |
| | | tggaaggatccggcgagctgttccggtggaatgtgtctgatctcggcggcctcggatgcggc |
| | | ctgaagaatagatctagcgagggccctagcagccccagcggaaaactgatgagccccaagct |
| | | gtacgtgtgggccaaagacagacccgagatttgggagggcgagcctccttgtctgcctccta |
| | | gagacagcctgaaccagagcctgagccaggacctgacaatggcccctggatctacactgtgg |
| | | ctgagctgtggcgtgccacctgacagtgtgtctagaggccctctgtcttggacccacgtgca |
| | | ccctaagggccctaagtctctgctgagcctggaactgaaggacgacaggcccgccagagata |
| | | tgtgggtcatggaaacaggcctgctgctgcctagagccacagcacaggatgccggcaagtac |
| | | tactgccacagaggcaacctgaccatgagcttccacctggaaatcaccgccagacctgtcct |
| | | gtggcactggctgcttagaaccggcggctggaaagtgtctgccgtgactctggcctacctga |
| | | tcttctgcctgtgtagcctcgtgggcatcctgcatctgcagagagcactggtcctgcggcgg |
| | | aagcggaagagaatgaccgatcctaccagacggttctga |
| 31 | DAP10.13 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGRVYINMPGRGRAKRSGSGATNFS |
| | | LLKQAGDVEENPGPMPPPRLLFFLLFLTPMEVRPEEPLVVKV |
| | | EEGDNAVLQCLKGTSDGPTQQLTWSRESPLKPFLKLSLGLPG |
| | | LGIHMRPLAIWLFIFNVSQQMGGFYLCQPGPPSEKAWQPGW |
| | | TVNVEGSGELFRWNVSDLGGLGCGLKNRSSEGPSSPSGKLM |
| | | SPKLYVWAKDRPEIWEGEPPCLPPRDSLNQSLSQDLTMAPGS |
| | | TLWLSCGVPPDSVSRGPLSWTHVHPKGPKSLLSLELKDDRPA |
| | | RDMWVMETGLLLPRATAQDAGKYYCHRGNLTMSFHLEITA |
| | | RPVLWHWLLRTGGWKVSAVTLAYLIFCLCSLVGILHLQRAL |
| | | VLRRKRKRMTDPTRRF |
| 32 | DAP10.13 | atgtccgtgcctacccaggtgctgggcctgctgctgctgtggctgaccgacgccagatgcat |
| | Nucleic Acid | gatccacctgggccacatcctgtttctgctgctgcttcctgtggccgctgctcagacaacac |
| | | ctggcgagagatctagcctgcctgccttctatcctggcaccagcggctcttgttct |
| | | ggctgtggatctctgagcctgcctctgctggctggactggttgctgctgatgctgtggcctc |
| | | tctgctgattgtgggcgccgtgttcctgtgtgcccggcctagaagatctcccgctcaagagg |
| | | atggcagagtgtacatcaacatgcccggcagaggacgcgcgaagcgatcaggcagcggggcg |
| | | acaaatttcagccttctgaaacaagcaggcgacgtggaagaaaaccccggtccaatgcctcc |
| | | tcctcggctgctgttcttcctgctgtttctgacccctatggaagtgcggcccgaggaacctc |
| | | tggtggtcaaagttgaagagggcgacaacgccgtgctgcagtgtctgaagggcacatctgat |
| | | ggccccacacagcagctgacctggtctagagagagccctctgaagcccttcctgaagctgtc |
| | | tctgggactgcctggactgggcatccatatgaggcctctggccatctggctgttcatcttca |
| | | acgtgtcccagcagatgggcggcttctacctgtgtcaacctggacctccaagcgagaaggct |
| | | tggcagcctggctggaccgtgaatgtggaaggatccggcgagctgttccggtggaatgtgtc |
| | | tgatctcggcggcctcggatgcggcctgaagaatagatctagcgagggccctagcagcccca |
| | | gcggaaaactgatgagccccaagctgtacgtgtgggccaaagacagacccgagatttgggag |
| | | ggcgagcctccttgtctgcctcctagagacagcctg |
| | | aaccagagcctgagccaggacctgacaatggcccctggatctacactgtggctgagctgtgg |
| | | cgtgccacctgacagtgtgtctagaggccctctgtcttggacccacgtgcaccctaagggcc |
| | | ctaagtctctgctgagcctggaactgaaggacgacaggcccgccagagatatgtgggtcatg |
| | | gaaacaggcctgctgctgcctagagccacagcacaggatgccggcaagtactactgccacag |
| | | aggcaacctgaccatgagcttccacctggaaatcaccgccagacctgtcctgtggcactggc |
| | | tgcttagaaccggcggctggaaagtgtctgccgtgactctggcctacctg |
| | | atcttctgcctgtgtagcctcgtgggcatcctgcatctgcagagagcactggtcctgcggcg |
| | | gaagcggaagagaatgaccgatcctaccagacggttctga |
| 33 | DAP10.14 | MSVPTQVLGLLLLWLTDARCMIHLGHILFLLLLPVAAAQTTP |
| | Amino Acid | GERSSLPAFYPGTSGSCSGCGSLSLPLLAGLVAADAVASLLIV |
| | | GAVFLCARPRRSPAQEDGRVYINMPGRGRVKFSRSADAPAY |
| | | QQGQNQLYNELNLGRREEYDVLDKRRGRDPEMGGKPQRRK |
| | | NPQEGLYNELQKDKMAEAYSEIGMKGERRRGKGHDGLYQG |
| | | LSTATKDTYDALHMQALPPRRAKRSGSGATNFSLLKQAGDV |
| | | EENPGPMPPPRLLFFLLFLTPMEVRPEEPLVVKVEEGDNAVL |
| | | QCLKGTSDGPTQQLTWSRESPLKPFLKLSLGLPGLGIHMRPL |
| | | AIWLFIFNVSQQMGGFYLCQPGPPSEKAWQPGWTVNVEGSG |
| | | ELFRWNVSDLGGLGCGLKNRSSEGPSSPSGKLMSPKLYVWA |
| | | KDRPEIWEGEPPCLPPRDSLNQSLSQDLTMAPGSTLWLSCGV |
| | | PPDSVSRGPLSWTHVHPKGPKSLLSLELKDDRPARDMWVME |
| | | TGLLLPRATAQDAGKYYCHRGNLTMSFHLEITARPVLWHWL |
| | | LRTGGWKVSAVTLAYLIFCLCSLVGILHLQRALVLRRKRKR |
| | | MTDPTRRF |
| 34 | DAP10.14 | atgtccgtgcctacccaggtgctgggcctgctgctgctgtggctgaccgacgccagatgc |
| | Nucleic Acid | atgatccacctgggccacatcctgttcctgctgctgctgcctgtggccgctgcccagacc |
| | | acccctggagaaagatctagccttccagccttctaccccgggaccagcggaagctgcagc |
| | | ggctgcggcagcttgtctctgcctctgctggccggcctggtggccgccgacgctgttgca |
| | | tctctgctgatcgtgggcgccgtctttctgtgcgccagacccagacggagccctgctcag |
| | | gaagatggcagagtgtacatcaacatgcctggcagaggcagagtgaagttcagcagaagc |
| | | gccgatgcccctgcctatcagcagggccagaaccagctgtacaacgagctgaacctgggc |
| | | agacgcgaggaatacgacgtgctggacaagcggcggggacgggaccccgagatgggcggc |
| | | aagcctcaaagaaggaagaacccccaggagggcctgtataatgagctgcagaaagataag |
| | | atggccgaggcctacagcgagatcggcatgaaaggcgagagacggcggggcaagggccac |
| | | gacggcctgtaccagggcctgagcaccgccacaaaggacacctacgacgccctgcacatg |
| | | caggccctgccacctagacgcgcgaagcgatcaggcagcggggcgacaaatttcagcctt |
| | | ctgaaacaagcaggcgacgtggaagaaaaccccggtccaatgcctcctcctcggctgctg |
| | | ttcttcctgctgtttctgacccctatggaagtgcggcccgaggaacctctggtggtcaaa |
| | | gttgaagagggcgacaacgccgtgctgcagtgtctgaagggcacatctgatggccccaca |
| | | cagcagctgacctggtctagagagagccctctgaagcccttcctgaagctgtctctggga |
| | | ctgcctggactgggcatccatatgaggcctctggccatctggctgttcatcttcaacgtg |
| | | tcccagcagatgggcggcttctacctgtgtcaacctggacctccaagcgagaaggcttgg |
| | | cagcctggctggaccgtgaatgtggaaggatccggcgagctgttccggtggaatgtgtct |
| | | gatctcggcggcctcggatgcggcctgaagaatagatctagcgagggccctagcagcccc |
| | | agcggaaaactgatgagccccaagctgtacgtgtgggccaaagacagacccgagatttgg |
| | | gagggcgagcctccttgtctgcctcctagagacagcctgaaccagagcctgagccaggac |
| | | ctgacaatggcccctggatctacactgtggctgagctgtggcgtgccacctgacagtgtg |
| | | tctagaggccctctgtcttggacccacgtgcaccctaagggccctaagtctctgctgagc |
| | | ctggaactgaaggacgacaggcccgccagagatatgtgggtcatggaaacaggcctgctg |
| | | ctgcctagagccacagcacaggatgccggcaagtactactgccacagaggcaacctgacc |
| | | atgagcttccacctggaaatcaccgccagacctgtcctgtggcactggctgcttagaacc |
| | | ggcggctggaaagtgtctgccgtgactctggcctacctgatcttctgcctgtgtagcctc |
| | | gtgggcatcctgcatctgcagagagcactggtcctgcggcggaagcggaagagaatgacc |
| | | gatcctaccagacggttctga |
| 35 | DAP10.15 | MSVPTQVLGLLLLWLTDARCMIHLGHILFLLLLPVAAAQTTPGER |
| | Amino Acid | SSLPAFYPGTSGSCSGCGSLSLPLLAGLVAADAVASLLIVGAVFLC |
| | | ARPRRSPAQEDGRVYINMPGRGKRGRKKLLYIFKQPFMRPVQTT |
| | | QEEDGCSCRFPEEEEGGCELRVKFSRSADAPAYQQGQNQLYNEL |
| | | NLGRREEYDVLDKRRGRDPEMGGKPQRRKNPQEGLYNELQKDK |
| | | MAEAYSEIGMKGERRRGKGHDGLYQGLSTATKDTYDALHMQA |
| | | LPPRRAKRSGSGATNFSLLKQAGDVEENPGPMPPPRLLFFLLFLTP |
| | | MEVRPEEPLVVKVEEGDNAVLQCLKGTSDGPTQQLTWSRESPLK |
| | | PFLKLSLGLPGLGIHMRPLAIWLFIFNVSQQMGGFYLCQPGPPSEK |
| | | AWQPGWTVNVEGSGELFRWNVSDLGGLGCGLKNRSSEGPSSPS |
| | | GKLMSPKLYVWAKDRPEIWEGEPPCLPPRDSLNQSLSQDLTMAP |
| | | GSTLWLSCGVPPDSVSRGPLSWTHVHPKGPKSLLSLELKDDRPAR |
| | | DMWVMETGLLLPRATAQDAGKYYCHRGNLTMSFHLEITARPVL |
| | | WHWLLRTGGWKVSAVTLAYLIFCLCSLVGILHLQRALVLRRKRK |
| | | RMTDPTRRF |
| 36 | DAP10.15 | atgtccgtgcctacccaggtgctgggcctgctgctgctgtggctgaccgacgccagatgc |
| | Nucleic Acid | atgatccacctgggccacatcctgttcctgctgctgctgcctgtggccgccgctcaaaca |
| | | acccctggcgagagatctagcctgccagccttctaccccggtacaagcggatcttgcagc |
| | | ggctgcggcagcctgtctctgcctctgctggccggcctggtggctgctgatgctgtggcc |
| | | agcctgctgatcgtgggcgccgtgttcctgtgcgccagacctagaaggtcccctgcccag |
| | | gaggatggaagagtgtacatcaacatgcctggcagaggcaagagaggcagaaagaagctg |
| | | ctgtacatcttcaagcagcctttcatgagaccagtgcagaccacccaggaggaggacgga |
| | | tgtagctgcagattccccgaggaagaagaaggcggctgtgaactgagagtgaagttcagc |
| | | agaagcgccgacgcccctgcttatcagcagggccagaaccagctgtacaacgagctgaac |
| | | ctgggcagaagggaagagtacgacgtgctggacaagcggcggggacgggaccccgagatg |
| | | ggcggaaaacctcaaagacggaagaacccccaggagggcctttataatgagctgcagaaa |
| | | gataagatggccgaggcctacagcgagatcggcatgaaaggagaaagacggcggggcaaa |
| | | ggccacgacggcctgtaccagggcctgagcaccgccacaaaggacacctacgacgccctg |
| | | cacatgcaggccctgccccccagacgcgcgaagcgatcaggcagcggggcgacaaatttc |
| | | agccttctgaaacaagcaggcgacgtggaagaaaaccccggtccaatgcctcctcctcgg |
| | | ctgctgttcttcctgctgtttctgacccctatggaagtgcggcccgaggaacctctggtg |
| | | gtcaaagttgaagagggcgacaacgccgtgctgcagtgtctgaagggcacatctgatggc |
| | | cccacacagcagctgacctggtctagagagagccctctgaagcccttcctgaagctgtct |
| | | ctgggactgcctggactgggcatccatatgaggcctctggccatctggctgttcatcttc |
| | | aacgtgtcccagcagatgggcggcttctacctgtgtcaacctggacctccaagcgagaag |
| | | gcttggcagcctggctggaccgtgaatgtggaaggatccggcgagctgttccggtggaat |
| | | gtgtctgatctcggcggcctcggatgcggcctgaagaatagatctagcgagggccctagc |
| | | agccccagcggaaaactgatgagccccaagctgtacgtgtgggccaaagacagacccgag |
| | | atttgggagggcgagcctccttgtctgcctcctagagacagcctgaaccagagcctgagc |
| | | caggacctgacaatggcccctggatctacactgtggctgagctgtggcgtgccacctgac |
| | | agtgtgtctagaggccctctgtcttggacccacgtgcaccctaagggccctaagtctctg |
| | | ctgagcctggaactgaaggacgacaggcccgccagagatatgtgggtcatggaaacaggc |
| | | ctgctgctgcctagagccacagcacaggatgccggcaagtactactgccacagaggcaac |
| | | ctgaccatgagcttccacctggaaatcaccgccagacctgtcctgtggcactggctgctt |
| | | agaaccggcggctggaaagtgtctgccgtgactctggcctacctgatcttctgcctgtgt |
| | | agcctcgtgggcatcctgcatctgcagagagcactggtcctgcggcggaagcggaagaga |
| | | atgaccgatcctaccagacggttctga |
| 37 | DAP10-D57A | MIHLGHILFLLLLPVAAAQTTPGERSSLPAFYPGTSGSCSGCGSLSL |
| | | PLLAGLVAAAAVASLLIVGAVFLCARPRRSPAQEDGKVYINMPG |
| | | RG |
| 38 | DAP10-N88Q | MIHLGHILFLLLLPVAAAQTTPGERSSLPAFYPGTSGSCSGCGSLSL |
| | | PLLAGLVAADAVASLLIVGAVELCARPRRSPAQEDGKVYIQMPG |
| | | RG |
| 39 | DAP10-M89Q | MIHLGHILFLLLLPVAAAQTTPGERSSLPAFYPGTSGSCSGCGSLSL |
| | | PLLAGLVAADAVASLLIVGAVFLCARPRRSPAQEDGKVYINQPG |
| | | RG |
| 40 | CD28 | FWVLVVVGGVLACYSLLVTVAFIIFWVRSKRSRLLHSDYMN |
| | costimulatory | MTPRRPGPTRKHYQPYAPPRDFAAYRS |
| | domain | |
| 41 | CD28 | FWVRSKRSRLLHSDYMNMTPRRPGPTRKHYQPYAPPRDFAA |
| | costimulatory | YRS |
| | domain | |
| 42 | ICOS | TKKKYSSSVHDPNGEYMFMRAVNTAKKSRLTDVTL |
| | costimulatory | |
| | domain | |
| 43 | OX40 | RRDQRLPPDAHKPPGGGSFRTPIQEEQADAHSTLAKI |
| 44 | P2A cleavage | GSGATNFSLLKQAGDVEENPGP |
| | motif 2 | |
| | polypeptide | |
| 45 | Signal peptide | MSVPTQVLGLLLLWLTDARC |
| 46 | DAP10.3.1 | atgtccgtgcctacccaggtgctgggcctgctgctgctgtggctgaccgacgccagatgcat |
| | (+sig domain, | gatccacctgggccacatcctgtttctgctgctgcttcctgtggccgctgctcagacaacac |
| | −FP2A, −CD19t) | ctggcgagagatctagcctgcctgccttctatcctggcaccagcggctcttgttctggctgt |
| | Nucleic Acid | ggatctctgagcctgcctctgctggctggactggttgctgctgatgctgtggcctctctgct |
| | | gattgtgggcgccgtgttcctgtgtgcccggcctagaagatctcccgctcaagaggatggca |
| | | aggtgtacatcaacatgcccggcagaggaagagtgaagttcagcagaagcgccgatgcccct |
| | | gcctatcagcagggccagaaccagctgtacaacgagctgaacctgggcagacgcgaggaata |
| | | cgacgtgctggacaagcggcggggacgggaccccgagatgggcggcaagcctcaaagaagga |
| | | agaacccccaggagggcctgtataatgagctgcagaaagataagatggccgaggcctacagc |
| | | gagatcggcatgaaaggcgagagacggcggggcaagggccacgacggcctgtaccagggcct |
| | | gagcaccgccacaaaggacacctacgacgccctgcacatgcaggccctgccacctaga |
| 47 | DAP10.3.1 | MSVPTQVLGLLLLWLTDARCMIHLGHILFLLLLPVAAAQTTP |
| | (+sig domain, | GERSSLPAFYPGTSGSCSGCGSLSLPLLAGLVAADAVASLLIV |
| | −FP2A, −CD19t) | GAVFLCARPRRSPAQEDGKVYINMPGRGRVKFSRSADAPAY |
| | Amino Acid | QQGQNQLYNELNLGRREEYDVLDKRRGRDPEMGGKPQRRK |
| | | NPQEGLYNELQKDK |
| | | MAEAYSEIGMKGERRRGKGHDGLYQGLSTATKDTYDALHM |
| | | QALPPR |
| 48 | DAP10.4.1 | atgtccgtgcctacccaggtgctgggcctgctgctgctgtggctgaccgacgccagatgcat |
| | (+sig domain, | gatccacctgggccacatcctgttcctgctgctgctgcctgtggccgccgctcaaacaaccc |
| | −FP2A, −CD19t) | ctggcgagagatctagcctgccagccttctaccccggtacaagcggatcttgcagc |
| | Nucleic Acid | ggctgcggcagcctgtctctgcctctgctggccggcctggtggctgctgatgctgtggccag |
| | | cctgctgatcgtgggcgccgtgttcctgtgcgccagacctagaaggtcccctgcccaggagg |
| | | atggaaaggtgtacatcaacatgcctggcagaggcaagagaggcagaaagaagctg |
| | | ctgtacatcttcaagcagcctttcatgagaccagtgcagaccacccaggaggaggacggatg |
| | | tagctgcagattccccgaggaagaagaaggcggctgtgaactgagagtgaagttcagcagaa |
| | | gcgccgacgcccctgcttatcagcagggccagaaccagctgtacaacgagctgaac |
| | | ctgggcagaagggaagagtacgacgtgctggacaagcggcggggacgggaccccgagatg |
| | | ggcggaaaacctcaaagacggaagaacccccaggagggcctttataatgagctgcagaaaga |
| | | taagatggccgaggcctacagcgagatcggcatgaaaggagaaagacggcggggcaaa |
| | | ggccacgacggcctgtaccagggcctgagcaccgccacaaaggacacctacgacgccctgc |
| | | acatgcaggccctgccccccaga |
| 49 | DAP10.4.1 | MSVPTQVLGLLLLWLTDARCMIHLGHILFLLLLPVAAAQTTP |
| | (+sig domain, | GERSSLPAFYPGTSGSCSGCGSLSLPLLAGLVAADAVASLLIV |
| | −FP2A, −CD19t) | GAVFLCARPRRSPAQEDGKVYINMPGRGKRGRKKL |
| | Amino Acid | LYIFKQPFMRPVQTTQEEDGCSCRFPEEEEGGCELRVKFSRSA |
| | | DAPAYQQGQNQLYNELNLGRREEYDVLDKRRGRDPEMGGK |
| | | PQRRKNPQEGLYNELQKDKMAEAYSEIGMKGERRRGKGHD |
| | | GLYQGLSTATKDTYDALHMQALPPR |
| 50 | DAP10.5.1 | atgtccgtgcctacccaggtgctgggcctgctgctgctgtggctgaccgacgccagatgcat |
| | (+sig domain, | gatccacctgggacacatcctgttcctgctgctgcttccagtggccgccgctcaaacaaccc |
| | −FP2A, −CD19t) | ctggcgagagaagcagcctgcccgccttctaccccggtacatctggcagctgcagc |
| | Nucleic Acid | ggctgcggcagcttgtctctgcctctgctggccggactggtggctgctgatgccgtggccag |
| | | cctgctgatcgtgggcgccgtgttcctgtgcgccagacccagaaggtcccctgcccaggagg |
| | | acggcaaggtgttcatcaacatgcctggcagaggcaaaagaggcagaaagaagctg |
| | | ctgtacatcttcaagcagcctttcatgcggcctgtgcagaccacccaggaggaagatggctg |
| | | ctcttgtcgatttccagaagaggaagaaggcggctgtgaactgagagtgaagttcagcagat |
| | | ccgccgacgcccctgcttatcagcagggccagaaccagctgtataatgagctgaac |
| | | ctgggccggcgggaagagtacgacgtgctggacaagcggcggggcagagatcctgagatgg |
| | | gcggaaaacctcaaagaaggaagaacccccaggagggactgtacaacgagctgcagaagga |
| | | taagatggccgaggcctacagcgagatcggcatgaaaggagaaagaagaagaggaaaa |
| | | ggccacgacggcctgtaccagggcctgagcaccgccaccaaggacacctacgacgccctgc |
| | | acatgcaggccctgccacctaga |
| 51 | DAP10.5.1 | MSVPTQVLGLLLLWLTDARCMIHLGHILFLLLLPVAAAQTTP |
| | (+sig domain, | GERSSLPAFYPGTSGSCSGCGSLSLPLLAGLVAADAVASLLIV |
| | −FP2A, −CD19t) | GAVFLCARPRRSPAQEDGKVFINMPGRGKRGRKKL |
| | Amino Acid | LYIFKQPFMRPVQTTQEEDGCSCRFPEEEEGGCELRVKFSRSA |
| | | DAPAYQQGQNQLYNELNLGRREEYDVLDKRRGRDPEMGGK |
| | | PQRRKNPQEGLYNELQKDKMAEAYSEIGMKGERRRGK |
| | | GHDGLYQGLSTATKDTYDALHMQALPPR |
| 52 | DAP10.6.1 | atgtccgtgcctacccaggtgctgggcctgctgctgctgtggctgaccgacgccagatgcat |
| | (+sig domain, | gatccacctgggccacatcctgttcctgctgctcctgcctgtggccgctgctcagaccaccc |
| | −FP2A, −CD19t) | ctggagaaagatctagcctgcccgctttctaccccgggaccagcggcagctgcagc |
| | Nucleic Acid | ggatgcggctctctgagcctgcctctgctggccggcctcgtggccgccgatgctgtggccag |
| | | cctgctgatcgtgggcgccgtgttcctgtgcgccagacccagaagaagccctgcccaggagg |
| | | atggcagagtgttcatcaacatgcctggcagaggcaaaagaggcagaaagaagctg |
| | | ctgtacatcttcaagcagcctttcatgcggcccgtgcagacaacccaggaggaggacggctg |
| | | tagctgtagattccccgaggaagaagaaggcggctgcgagcttagagtgaagttcagcagaa |
| | | gcgccgacgcccctgcttaccagcagggccagaaccagctgtataatgagctgaac |
| | | ctgggaagaagggaagagtacgacgtgctggacaagcggcggggcagagatcctgagatgg |
| | | gcggaaaacctcaaagaaggaagaaccctcaggagggcctgtacaacgagctgcagaaaga |
| | | caagatggccgaggcctacagcgagatcggcatgaaaggcgagagacggggggcaag |
| | | ggccacgacggcctgtaccaaggcctgtcaacagccaccaaggacacctacgacgccctgca |
| | | catgcaggccctgccacctaga |
| 53 | DAP10.6.1 | MSVPTQVLGLLLLWLTDARCMIHLGHILFLLLLPVAAAQTTP |
| | (+sig domain, | GERSSLPAFYPGTSGSCSGCGSLSLPLLAGLVAADAVASLLIV |
| | −FP2A, −CD19t) | GAVFLCARPRRSPAQEDGRVFINMPGRGKRGRKKL |
| | Amino Acid | LYIFKQPFMRPVQTTQEEDGCSCRFPEEEEGGCELRVKFSRSA |
| | | DAPAYQQGQNQLYNELNLGRREEYDVLDKRRGRDPEMGGK |
| | | PQRRKNPQEGLYNELQKDKMAEAYSEIGMKGERRRGK |
| | | GHDGLYQGLSTATKDTYDALHMQALPPR |
| 54 | DAP10.13.1 | atgtccgtgcctacccaggtgctgggcctgctgctgctgtggctgaccgacgccagatgcat |
| | (+sig domain, | gatccacctgggccacatcctgtttctgctgctgcttcctgtggccgctgctcagacaacac |
| | −FP2A, −CD19t) | ctggcgagagatctagcctgcctgccttctatcctggcaccagcggctcttgttct |
| | Nucleic Acid | ggctgtggatctctgagcctgcctctgctggctggactggttgctgctgatgctgtggcctc |
| | | tctgctgattgtgggcgccgtgttcctgtgtgcccggcctagaagatctcccgctcaagagg |
| | | atggcagagtgtacatcaacatgcccggcagagga |
| 55 | DAP10.13.1 | MSVPTQVLGLLLLWLTDARCMIHLGHILFLLLLPVAAAQTTP |
| | (+sig domain, | GERSSLPAFYPGTSGSCSGCGSLSLPLLAGLVAADAVASLLIV |
| | −FP2A, −CD19t) | GAVFLCARPRRSPAQEDGRVYINMPGRG |
| | Amino Acid | |
| 56 | DAP10.14.1 | atgtccgtgcctacccaggtgctgggcctgctgctgctgtggctgaccgacgccagatgca |
| | (+sig domain, | tgatccacctgggccacatcctgttcctgctgctgctgcctgtggccgctgcccagaccacc |
| | −FP2A, −CD19t) | cctggagaaagatctagccttccagccttctaccccgggaccagcggaagctgcagc |
| | Nucleic Acid | ggctgcggcagcttgtctctgcctctgctggccggcctggtggccgccgacgctgttgcatc |
| | | tctgctgatcgtgggcgccgtctttctgtgcgccagacccagacggagccctgctcaggaag |
| | | atggcagagtgtacatcaacatgcctggcagaggcagagtgaagttcagcagaagc |
| | | gccgatgcccctgcctatcagcagggccagaaccagctgtacaacgagctgaacctgggcag |
| | | acgcgaggaatacgacgtgctggacaagcggcggggacgggaccccgagatgggcggcaa |
| | | gcctcaaagaaggaagaacccccaggagggcctgtataatgagctgcagaaagataag |
| | | atggccgaggcctacagcgagatcggcatgaaaggcgagagacggcggggcaagggccac |
| | | gacggcctgtaccagggcctgagcaccgccacaaaggacacctacgacgccctgcacatgca |
| | | ggccctgccacctaga |
| 57 | DAP10.14.1 | MSVPTQVLGLLLLWLTDARCMIHLGHILFLLLLPVAAAQTTP |
| | (+sig domain, | GERSSLPAFYPGTSGSCSGCGSLSLPLLAGLVAADAVASLLIV |
| | −FP2A, −CD19t) | GAVFLCARPRRSPAQEDGRVYINMPGRGRVKFSRS |
| | Amino Acid | ADAPAYQQGQNQLYNELNLGRREEYDVLDKRRGRDPEMGG |
| | | KPQRRKNPQEGLYNELQKDKMAEAYSEIGMKGERRRGKGH |
| | | DGLYQGLSTATKDTYDALHMQALPPR |
| 58 | DAP10.15.1 | atgtccgtgcctacccaggtgctgggcctgctgctgctgtggctgaccgacgccagatgcat |
| | (+sig domain, | gatccacctgggccacatcctgttcctgctgctgctgcctgtggccgccgctcaaacaaccc |
| | −FP2A, −CD19t) | ctggcgagagatctagcctgccagccttctaccccggtacaagcggatcttgcagcggctgc |
| | Nucleic Acid | ggcagcctgtctctgcctctgctggccggcctggtggctgctgatgctgtggccagcctgct |
| | | gatcgtgggcgccgtgttcctgtgcgccagacctagaaggtcccctgcccaggaggatggaa |
| | | gagtgtacatcaacatgcctggcagaggcaagagaggcagaaagaagctgctgtacatcttc |
| | | aagcagcctttcatgagaccagtgcagaccacccaggaggaggacggatgtagctgcagatt |
| | | ccccgaggaagaagaaggcggctgtgaactgagagtgaagttcagcagaagcgccgacgccc |
| | | ctgcttatcagcagggccagaaccagctgtacaacgagctgaacctgggcagaagggaagag |
| | | tacgacgtgctggacaagcggcggggacgggaccccgagatgggcggaaaacctcaaagacg |
| | | gaagaacccccaggagggcctttataatgagctgcagaaagataagatggccgaggcctaca |
| | | gcgagatcggcatgaaaggagaaagacggcggggcaaaggccacgacggcctgtaccagggc |
| | | ctgagcaccgccacaaaggacacctacgacgccctgcacatgcaggccctgccccccaga |
| 59 | DAP10.15.1 | MSVPTQVLGLLLLWLTDARCMIHLGHILFLLLLPVAAAQTTP |
| | (+sig domain, | GERSSLPAFYPGTSGSCS |
| | −FP2A, −CD19t) | GCGSLSLPLLAGLVAADAVASLLIVGAVFLCARPRRSPAQED |
| | Amino Acid | GRVYINMPGRGKRGRKKL |
| | | LYIFKQPFMRPVQTTQEEDGCSCRFPEEEEGGCELRVKFSRSA |
| | | DAPAYQQGQNQLYNELN |
| | | LGRREEYDVLDKRRGRDPEMGGKPQRRKNPQEGLYNELQK |
| | | DKMAEAYSEIGMKGERRRGK |
| | | GHDGLYQGLSTATKDTYDALHMQALPPR |
| 60 | DAP10.3.2 | atgatccacctgggccacatcctgtttctgctgctgcttcctgtggccgctgctcagacaac |
| | (−sig domain, | acctggcgagagatctagcctgcctgccttctatcctggcaccagcggctcttgttctggct |
| | −FP2A, −CD19t) | gtggatctctgagcctgcctctgctggctggactggttgctgctgatgctgtggcc |
| | Nucleic Acid | tctctgctgattgtgggcgccgtgttcctgtgtgcccggcctagaagatctcccgctcaaga |
| | | ggatggcaaggtgtacatcaacatgcccggcagaggaagagtgaagttcagcagaagcgccg |
| | | atgcccctgcctatcagcagggccagaaccagctgtacaacgagctgaacctgggc |
| | | agacgcgaggaatacgacgtgctggacaagcggcggggacgggaccccgagatgggcggc |
| | | aagcctcaaagaaggaagaacccccaggagggcctgtataatgagctgcagaaagataagat |
| | | ggccgaggcctacagcgagatcggcatgaaaggcgagagacggcggggcaagggccac |
| | | gacggcctgtaccagggcctgagcaccgccacaaaggacacctacgacgccctgcacatgca |
| | | ggccctgccacctaga |
| 61 | DAP10.3.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GKVYINMPGRGRVKFSRSADAPAYQQGQNQLYNELNLGRR |
| | Amino Acid | EEYDVLDKRRGRDPEMGGKPQRRKNPQEGLYNELQKDKMA |
| | | EAYSEIGMKGERRRGKGHDGLYQGLSTATKDTYDALHMQA |
| | | LPPR |
| 62 | DAP10.4.2 | atgatccacctgggccacatcctgttcctgctgctgctgcctgtggccgccgctcaaacaac |
| | (−sig domain, | ccctggcgagagatctagcctgccagccttctaccccggtacaagcggatcttgcagcggct |
| | −FP2A, −CD19t) | gcggcagcctgtctctgcctctgctggccggcctggtggctgctgatgctgtggcc |
| | Nucleic Acid | agcctgctgatcgtgggcgccgtgttcctgtgcgccagacctagaaggtcccctgcccagga |
| | | ggatggaaaggtgtacatcaacatgcctggcagaggcaagagaggcagaaagaagctgctgt |
| | | caatcttcaagcagcctttcatgagaccagtgcagaccacccaggaggaggacgga |
| | | tgtagctgcagattccccgaggaagaagaaggcggctgtgaactgagagtgaagttcagcag |
| | | aagcgccgacgcccctgcttatcagcagggccagaaccagctgtacaacgagctgaacctgg |
| | | gcagaagggaagagtacgacgtgctggacaagcggcggggacgggaccccgagatg |
| | | ggcggaaaacctcaaagacggaagaacccccaggagggcctttataatgagctgcagaaaga |
| | | taagatggccgaggcctacagcgagatcggcatgaaaggagaaagacggcggggcaaaggc |
| | | cacgacggcctgtaccagggcctgagcaccgccacaaaggacacctacgacgccctg |
| | | cacatgcaggccctgccccccaga |
| 63 | DAP10.4.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GKVYINMPGRGKRGRKKLLYIFKQPFMRPVQTTQEEDGCSC |
| | Amino Acid | RFPEEEEGGCELRVKFSRSADAPAYQQGQNQLYNELNLGRR |
| | | EEYDVLDKRRGRDPEMGGKPQRRKNPQEGLYNELQKDKMA |
| | | EAYSEIGMKGERRRGK |
| | | GHDGLYQGLSTATKDTYDALHMQALPPR |
| 64 | DAP10.5.2 | atgatccacctgggacacatcctgttcctgctgctgcttccagtggccgccgctcaaacaac |
| | (−sig domain, | ccctggcgagagaagcagcctgcccgccttctaccccggtacatctggcagctgcagcggct |
| | −FP2A, −CD19t) | gcggcagcttgtctctgcctctgctggccggactggtggctgctgatgccgtggcc |
| | Nucleic Acid | agcctgctgatcgtgggcgccgtgttcctgtgcgccagacccagaaggtcccctgcccagga |
| | | ggacggcaaggtgttcatcaacatgcctggcagaggcaaaagaggcagaaagaagctgctgt |
| | | acatcttcaagcagcctttcatgcggcctgtgcagaccacccaggaggaagatggc |
| | | tgctcttgtcgatttccagaagaggaagaaggcggctgtgaactgagagtgaagttcagcag |
| | | atccgccgacgcccctgcttatcagcagggccagaaccagctgtataatgagctgaacctgg |
| | | gccggcgggaagagtacgacgtgctggacaagcggcggggcagagatcctgagatg |
| | | ggcggaaaacctcaaagaaggaagaacccccaggagggactgtacaacgagctgcagaagg |
| | | ataagatggccgaggcctacagcgagatcggcatgaaaggagaaagaagaagaggaaaagg |
| | | ccacgacggcctgtaccagggcctgagcaccgccaccaaggacacctacgacgccctg |
| | | cacatgcaggccctgccacctaga |
| 65 | DAP10.5.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GKVFINMPGRGKRGRKKLLYIFKQPFMRPVQTTQEEDGCSC |
| | Amino Acid | RFPEEEEGGCELRVKFSRSADAPAYQQGQNQLYNELN |
| | | LGRREEYDVLDKRRGRDPEMGGKPQRRKNPQEGLYNELQK |
| | | DKMAEAYSEIGMKGERRRGKGHDGLYQGLSTATKDTYDAL |
| | | HMQALPPR |
| 66 | DAP10.6.2 | atgatccacctgggccacatcctgttcctgctgctcctgcctgtggccgctgctcagaccac |
| | (−sig domain, | ccctggagaaagatctagcctgcccgctttctaccccgggaccagcggcagctgcagcggat |
| | −FP2A, −CD19t) | gcggctctctgagcctgcctctgctggccggcctcgtggccgccgatgctgtggcc |
| | Nucleic Acid | agcctgctgatcgtgggcgccgtgttcctgtgcgccagacccagaagaagccctgcccagga |
| | | ggatggcagagtgttcatcaacatgcctggcagaggcaaaagaggcagaaagaagctgctgt |
| | | acatcttcaagcagcctttcatgcggcccgtgcagacaacccaggaggaggacggc |
| | | tgtagctgtagattccccgaggaagaagaaggcggctgcgagcttagagtgaagttcagcag |
| | | aagcgccgacgcccctgcttaccagcagggccagaaccagctgtataatgagctgaacctgg |
| | | gaagaagggaagagtacgacgtgctggacaagcggcggggcagagatcctgagatg |
| | | ggcggaaaacctcaaagaaggaagaaccctcaggagggcctgtacaacgagctgcagaaag |
| | | acaagatggccgaggcctacagcgagatcggcatgaaaggcgagagacggcggggcaagg |
| | | gccacgacggcctgtaccaaggcctgtcaacagccaccaaggacacctacgacgccctg |
| | | cacatgcaggccctgccacctaga |
| 67 | DAP10.6.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GRVFINMPGRGKRGRKKLLYIFKQPFMRPVQTTQEEDGCSCR |
| | Amino Acid | FPEEEEGGCELRVKFSRSADAPAYQQGQNQLYNELN |
| | | LGRREEYDVLDKRRGRDPEMGGKPQRRKNPQEGLYNELQK |
| | | DKMAEAYSEIGMKGERRRGKGHDGLYQGLSTATKDTYDAL |
| | | HMQALPPR |
| 68 | DAP10.13.2 | atgatccacctgggccacatcctgtttctgctgctgcttcctgtggccgctgctcagacaac |
| | (−sig domain, | acctggcgagagatctagcctgcctgccttctatcctggcaccagcggctcttgttctggct |
| | −FP2A, −CD19t) | gtggatctctgagcctgcctctgctggctggactggttgctgctgatgctgtggcc |
| | Nucleic Acid | tctctgctgattgtgggcgccgtgttcctgtgtgcccggcctagaagatctcccgctcaaga |
| | | ggatggcagagtgtacatcaacatgcccggcagagga |
| 69 | DAP10.13.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GRVYINMPGRG |
| | Amino Acid | |
| 70 | DAP10.14.2 | atgatccacctgggccacatcctgttcctgctgctgctgcctgtggccgctgcccagaccac |
| | (−sig domain, | ccctggagaaagatctagccttccagccttctaccccgggaccagcggaagctgcagcggct |
| | −FP2A, −CD19t) | gcggcagcttgtctctgcctctgctggccggcctggtggccgccgacgctgttgca |
| | Nucleic Acid | tctctgctgatcgtgggcgccgtctttctgtgcgccagacccagacggagccctgctcagga |
| | | agatggcagagtgtacatcaacatgcctggcagaggcagagtgaagttcagcagaagcgccg |
| | | atgcccctgcctatcagcagggccagaaccagctgtacaacgagctgaacctgggcagacgc |
| | | gaggaatacgacgtgctggacaagcggcggggacgggaccccgagatgggcggcaagcctca |
| | | aagaaggaagaacccccaggagggcctgtataatgagctgcagaaagataagatggccgagg |
| | | cctacagcgagatcggcatgaaaggcgagagacggcggggcaagggccacgacggcctgtac |
| | | cagggcctgagcaccgccacaaaggacacctacgacgccctgcacatgcaggccctgccacc |
| | | taga |
| 71 | DAP10.14.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GRVYINMPGRGRVKFSRSADAPAYQQGQNQLYNELNLGRR |
| | Amino Acid | EEYDVLDKRRGRDPEMGGKPQRRKNPQEGLYNELQKDK |
| | | MAEAYSEIGMKGERRRGKGHDGLYQGLSTATKDTYDALHM |
| | | QALPPR |
| 72 | DAP10.15.2 | atgatccacctgggccacatcctgttcctgctgctgctgcctgtggccgccgctcaaacaac |
| | (−sig domain, | ccctggcgagagatctagcctgccagccttctaccccggtacaagcggatcttgcagcggct |
| | −FP2A, −CD19t) | gcggcagcctgtctctgcctctgctggccggcctggtggctgctgatgctgtggcc |
| | Nucleic Acid | agcctgctgatcgtgggcgccgtgttcctgtgcgccagacctagaaggtcccctgcccagga |
| | | ggatggaagagtgtacatcaacatgcctggcagaggcaagagaggcagaaagaagctgctgt |
| | | acatcttcaagcagcctttcatgagaccagtgcagaccacccaggaggaggacgga |
| | | tgtagctgcagattccccgaggaagaagaaggcggctgtgaactgagagtgaagttcagcag |
| | | aagcgccgacgcccctgcttatcagcagggccagaaccagctgtacaacgagctgaacctgg |
| | | gcagaagggaagagtacgacgtgctggacaagcggcggggacgggaccccgagatg |
| | | ggcggaaaacctcaaagacggaagaacccccaggagggcctttataatgagctgcagaaaga |
| | | taagatggccgaggcctacagcgagatcggcatgaaaggagaaagacggcggggcaaaggc |
| | | cacgacggcctgtaccagggcctgagcaccgccacaaaggacacctacgacgccctg |
| | | cacatgcaggccctgccccccaga |
| 73 | DAP10.15.2 | MIHLGHILFLLLLPVAAAQTTPGERSSLPAFYPGTSGSCS |
| | (−sig domain, | GCGSLSLPLLAGLVAADAVASLLIVGAVFLCARPRRSPAQED |
| | −FP2A, −CD19t) | GRVYINMPGRGKRGRKKL |
| | Amino Acid | LYIFKQPFMRPVQTTQEEDGCSCRFPEEEEGGCELRVKFSRSA |
| | | DAPAYQQGQNQLYNELN |
| | | LGRREEYDVLDKRRGRDPEMGGKPQRRKNPQEGLYNELQK |
| | | DKMAEAYSEIGMKGERRRGK |
| | | GHDGLYQGLSTATKDTYDALHMQALPPR |
| 74 | NKG2D | MGWIRGRRSRHSWEMSEFHNYNLDLKKSDFSTRWQKQRCP |
| | | VVKSKCRENASPFFFCCFIAVAMGIRFIIMVAIWSAVFLNSLF |
| | | NQEVQIPLTESYCGPCPKNWICYKNNCYQFFDESKNWYESQ |
| | | ASCMSQNASLLKVYSKEDQDLLKLVKSYHWMGLVHIPTNG |
| | | SWQWEDGSILSPNLLTIIEMQKGDCALYASSFKGYIENCSTPN |
| | | TYICMQRTV |
| 75 | NKG2D | PFFFCCFIAVAMGIRFIIMVA |
| | transmembrane | |
| | domain | |
| 76 | NKG2D | PFFFCCFIAVAMGIRFIIMVAIWSAVELNSLFNQEVQIPLTESY |
| | ligand-binding | CGPCPKNWICYKNNCYQFFDESKNWYESQASCMSQNA |
| | domain and | SLLKVYSKEDQDLLKLVKSYHWMGLVHIPTNGSWQWEDGS |
| | transmembrane | ILSPNLLTIIEMQKGDCALYASSFKGYIENCSTPNTYICMQRT |
| | domain | V |
| 77 | Linker 1 | GSTSGSGKPGSGEGSTKG |
| | polypeptide | |
| 78 | DAP10 mature | QTTPGERSSLPAFYPGTSGSCSGCGSLSLPLLAGLVAADAVAS |
| | form | LLIVGAVFLCARPRRSPAQEDGKVYINMPGRG |
| 79 | DAP10 | LLAGLVAADAVASLLIVGAVF |
| | transmembrane | |
| | domain | |
| 80 | DAP10 | QTTPGERSSLPAFYPGTSGSCSGCGSLSLP |
| | extracellular | |
| | domain | |
| 81 | DAP10 | LLAGLVAADAVASLLIVGAVFLCARPRRSPAQEDGKVYINM |
| | transmembrane | PGRG |
| | and | |
| | cytoplasmic | |
| | domain | |
| 82 | CD32 signaling | RVKFSRSADAPAYQQGQNQLYNELNLGRREEYDVLDKRRG |
| | peptide 3 | RDPEMGGKPQRRKNPQEGLFNELQKDKMAEAFSEIGMKGER |
| | (1XX) | RRGKGHDGLFQGLSTATKDTFDALHMQALPPR |
| 83 | DAP10.21- | MIHLGHILFLLLLPVAAAQTTPGERSSLPAFYPGTSGSCS |
| | FP2A-EGFRt | GCGSLSLPLLAGLVAADAVASLLIVGAVFLCARPRRSPAQ |
| | Amino Acid | EDGRVFINMPGRGKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPQRRKNPQEGLYNELQK |
| | | DKMAEAYSEIGMKGERRRGKGHDGLYQGLSTATKDTYDAL |
| | | HMQALPPRRAKRSGSGATNFSLLKQAGDVEENPGPMLLLV |
| | | TSLLLCELPHPAFLLIPRKVCNGIGIGEFKDSLSINATNI |
| | | KHFKNCTSISGDLHILPVAFRGDSFTHTPPLDPQELDILK |
| | | TVKEITGFLLIQAWPENRTDLHAFENLEIIRGRTKQHGQF |
| | | SLAVVSLNITSLGLRSLKEISDGDVIISGNKNLCYANTIN |
| | | WKKLFGTSGQKTKIISNRGENSCKATGQVCHALCSPEGCW |
| | | GPEPRDCVSCRNVSRGRECVDKCNLLEGEPREFVENSECI |
| | | QCHPECLPQAMNITCTGRGPDNCIQCAHYIDGPHCVKTCP |
| | | AGVMGENNTLVWKYADAGHVCHLCHPNCTYGCTGPGLEG |
| | | CPTNGPKIPSIATGMVGALLLLLVVALGIGLFM |
| 84 | DAP10.21- | atgatccacctgggccacatcctgttcctgctgctcctgcctgtggccgctgctcagacc |
| | FP2A-EGFRt | acccctggagaaagatctagcctgcccgctttctaccccgggaccagcggcagctgcagc |
| | Nucleotide | ggatgcggctctctgagcctgcctctgctggccggcctcgtggccgccgatgctgtggcc |
| | | agcctgctgatcgtgggcgccgtgttcctgtgcgccagacccagaagaagccctgcccag |
| | | gaggatggcagagtgttcatcaacatgcctggcagaggcaaaagaggcagaaagaagctg |
| | | ctgtacatcttcaagcagcctttcatgcggcccgtgcagacaacccaggaggaggacggc |
| | | tgtagctgtagattccccgaggaagaagaaggcggctgcgagcttagagtgaagttcagc |
| | | agaagcgccgacgcccctgcttaccagcagggccagaaccagctgtataatgagctgaac |
| | | ctgggaagaagggaagagtacgacgtgctggacaagcggcggggcagagatcctgagatg |
| | | ggcggaaaacctcaaagaaggaagaaccctcaggagggcctgtacaacgagctgcagaaa |
| | | gacaagatggccgaggcctacagcgagatcggcatgaaaggcgagagacggcggggcaag |
| | | ggccacgacggcctgtaccaaggcctgtcaacagccaccaaggacacctacgacgccctg |
| | | cacatgcaggccctgccacctagacgcgcgaagcgatcaggcagcggggcgacaaatttc |
| | | agccttctgaaacaagcaggcgacgtggaagaaaaccccggtccaatgcttctcctggtg |
| | | acaagccttctgctctgtgagttaccacacccagcattcctcctgatcccacgcaaagtg |
| | | tgtaacggaataggtattggtgaatttaaagactcactctccataaatgctacgaatatt |
| | | aaacacttcaaaaactgcacctccatcagtggcgatctccacatcctgccggtggcattt |
| | | aggggtgactccttcacacatactcctcctctggatccacaggaactggatattctgaaa |
| | | accgtaaaggaaatcacagggtttttgctgattcaggcttggcctgaaaacaggacggac |
| | | ctccatgcctttgagaacctagaaatcatacgcggcaggaccaagcaacatggtcagttt |
| | | tctcttgcagtcgtcagcctgaacataacatccttgggattacgctccctcaaggagata |
| | | agtgatggagatgtgataatttcaggaaacaaaaatttgtgctatgcaaatacaataaac |
| | | tggaaaaaactgtttgggacctccggtcagaaaaccaaaattataagcaacagaggtgaa |
| | | aacagctgcaaggccacaggccaggtctgccatgccttgtgctcccccgagggctgctgg |
| | | ggcccggagcccagggactgcgtctcttgccggaatgtcagccgaggcagggaatgcgtg |
| | | gacaagtgcaaccttctggagggtgagccaagggagtttgtggagaactctgagtgcata |
| | | cagtgccacccagagtgcctgcctcaggccatgaacatcacctgcacaggacggggacca |
| | | gacaactgtatccagtgtgcccactacattgacggcccccactgcgtcaagacctgcccg |
| | | gcaggagtcatgggagaaaacaacaccctggtctggaagtacgcagacgccggccatgtg |
| | | tgccacctgtgccatccaaactgcacctacggatgcactgggccaggtcttgaaggctgt |
| | | ccaacgaatgggcctaagatcccgtccatcgccactgggatggtgggggccctcctcttg |
| | | ctgctggtggtggccctggggatcggcctcttcatgtgataa |
| 85 | DAP10.28- | MIHLGHILFLLLLPVAAAQTTPGERSSLPAFYPGTSGSCS |
| | FP2A-EGFRt | GCGSLSLPLLAGLVAADAVASLLIVGAVFLCARPRRSPAQ |
| | Amino Acid | EDGRVFINMPGRGKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPQRRKNPQEGLFNELQK |
| | | DKMAEAFSEIGMKGERRRGKGHDGLFQGLSTATKDTFDAL |
| | | HMQALPPRRAKRSGSGATNFSLLKQAGDVEENPGPMLLLV |
| | | TSLLLCELPHPAFLLIPRKVCNGIGIGEFKDSLSINATNI |
| | | KHFKNCTSISGDLHILPVAFRGDSFTHTPPLDPQELDILK |
| | | TVKEITGFLLIQAWPENRTDLHAFENLEIIRGRTKQHGQF |
| | | SLAVVSLNITSLGLRSLKEISDGDVIISGNKNLCYANTIN |
| | | WKKLFGTSGQKTKIISNRGENSCKATGQVCHALCSPEGCW |
| | | GPEPRDCVSCRNVSRGRECVDKCNLLEGEPREFVENSECI |
| | | QCHPECLPQAMNITCTGRGPDNCIQCAHYIDGPHCVKTCP |
| | | AGVMGENNTLVWKYADAGHVCHLCHPNCTYGCTGPGLEG |
| | | CPTNGPKIPSIATGMVGALLLLLVVALGIGLFM |
| 86 | DAP10.28- | atgatccacctgggccacatcctgttcctgctgctcctgcctgtggccgctgctcagacc |
| | FP2A-EGFRt | acccctggagaaagatctagcctgcccgctttctaccccgggaccagcggcagctgcagc |
| | Nucleotide | ggatgcggctctctgagcctgcctctgctggccggcctcgtggccgccgatgctgtggcc |
| | | agcctgctgatcgtgggcgccgtgttcctgtgcgccagacccagaagaagccctgcccag |
| | | gaggatggcagagtgttcatcaacatgcctggcagaggcaaaagaggcagaaagaagctg |
| | | ctgtacatcttcaagcagcctttcatgcggcccgtgcagacaacccaggaggaggacggc |
| | | tgtagctgtagattccccgaggaagaagaaggcggctgcgagcttcgagtgaagttcagt |
| | | agaagcgccgacgcccctgcctaccagcagggccagaaccagctgtacaacgagctgaac |
| | | ctgggcagacgcgaggaatacgacgtgctggacaagcggcggggcagagatcctgagatg |
| | | ggcggaaaacctcaaagaagaaagaacccccaggagggcctgttcaacgagctgcagaaa |
| | | gataagatggccgaggccttcagcgagatcggcatgaaaggcgagagacgacgtggaaag |
| | | ggccacgacggcttgtttcagggcctgagcaccgccacaaaggacaccttcgacgccctg |
| | | cacatgcaggccctcccccccagacgcgcgaagcgatcaggcagcggggcgacaaatttc |
| | | agccttctgaaacaagcaggcgacgtggaagaaaaccccggtccaatgcttctcctggtg |
| | | acaagccttctgctctgtgagttaccacacccagcattcctcctgatcccacgcaaagtg |
| | | tgtaacggaataggtattggtgaatttaaagactcactctccataaatgctacgaatatt |
| | | aaacacttcaaaaactgcacctccatcagtggcgatctccacatcctgccggtggcattt |
| | | aggggtgactccttcacacatactcctcctctggatccacaggaactggatattctgaaa |
| | | accgtaaaggaaatcacagggtttttgctgattcaggcttggcctgaaaacaggacggac |
| | | ctccatgcctttgagaacctagaaatcatacgcggcaggaccaagcaacatggtcagttt |
| | | tctcttgcagtcgtcagcctgaacataacatccttgggattacgctccctcaaggagata |
| | | agtgatggagatgtgataatttcaggaaacaaaaatttgtgctatgcaaatacaataaac |
| | | tggaaaaaactgtttgggacctccggtcagaaaaccaaaattataagcaacagaggtgaa |
| | | aacagctgcaaggccacaggccaggtctgccatgccttgtgctcccccgagggctgctgg |
| | | ggcccggagcccagggactgcgtctcttgccggaatgtcagccgaggcagggaatgcgtg |
| | | gacaagtgcaaccttctggagggtgagccaagggagtttgtggagaactctgagtgcata |
| | | cagtgccacccagagtgcctgcctcaggccatgaacatcacctgcacaggacggggacca |
| | | gacaactgtatccagtgtgcccactacattgacggcccccactgcgtcaagacctgcccg |
| | | gcaggagtcatgggagaaaacaacaccctggtctggaagtacgcagacgccggccatgtg |
| | | tgccacctgtgccatccaaactgcacctacggatgcactgggccaggtcttgaaggctgt |
| | | ccaacgaatgggcctaagatcccgtccatcgccactgggatggtgggggccctcctcttg |
| | | ctgctggtggtggccctggggatcggcctcttcatgtgataa |
| 87 | DAP10.29- | MIHLGHILFLLLLPVAAAQTTPGERSSLPAFYPGTSGSCS |
| | FP2A-EGFRt | GCGSLSLPLLAGLVAADAVASLLIVGAVFLCARPRRSPAQ |
| | Amino Acid | EDGRVFINMPGRGRVKFSRSADAPAYQQGQNQLYNELNLG |
| | | RREEYDVLDKRRGRDPEMGGKPQRRKNPQEGLFNELQKDK |
| | | MAEAFSEIGMKGERRRGKGHDGLFQGLSTATKDTFDALHM |
| | | QALPPRRAKRSGSGATNFSLLKQAGDVEENPGPMLLLVTS |
| | | LLLCELPHPAFLLIPRKVCNGIGIGEFKDSLSINATNIKH |
| | | FKNCTSISGDLHILPVAFRGDSFTHTPPLDPQELDILKTV |
| | | KEITGFLLIQAWPENRTDLHAFENLEIIRGRTKQHGQFSL |
| | | AVVSLNITSLGLRSLKEISDGDVIISGNKNLCYANTINWK |
| | | KLFGTSGQKTKIISNRGENSCKATGQVCHALCSPEGCWGP |
| | | EPRDCVSCRNVSRGRECVDKCNLLEGEPREFVENSECIQC |
| | | HPECLPQAMNITCTGRGPDNCIQCAHYIDGPHCVKTCPAG |
| | | VMGENNTLVWKYADAGHVCHLCHPNCTYGCTGPGLEGCPT |
| | | NGPKIPSIATGMVGALLLLLVVALGIGLFM |
| 88 | DAP10.29- | atgatccacctgggccacatcctgttcctgctgctcctgcctgtggccgctgctcagacc |
| | FP2A-EGFRt | acccctggagaaagatctagcctgcccgctttctaccccgggaccagcggcagctgcagc |
| | Nucleotide | ggatgcggctctctgagcctgcctctgctggccggcctcgtggccgccgatgctgtggcc |
| | | agcctgctgatcgtgggcgccgtgttcctgtgcgccagacccagaagaagccctgcccag |
| | | gaggatggcagagtgttcatcaacatgcctggcagaggccgagtgaagttcagtagaagc |
| | | gccgacgcccctgcctaccagcagggccagaaccagctgtacaacgagctgaacctgggc |
| | | agacgcgaggaatacgacgtgctggacaagcggcggggcagagatcctgagatgggcgga |
| | | aaacctcaaagaagaaagaacccccaggagggcctgttcaacgagctgcagaaagataag |
| | | atggccgaggccttcagcgagatcggcatgaaaggcgagagacgacgtggaaagggccac |
| | | gacggcttgtttcagggcctgagcaccgccacaaaggacaccttcgacgccctgcacatg |
| | | caggccctcccccccagacgcgcgaagcgatcaggcagcggggcgacaaatttcagcctt |
| | | ctgaaacaagcaggcgacgtggaagaaaaccccggtccaatgcttctcctggtgacaagc |
| | | cttctgctctgtgagttaccacacccagcattcctcctgatcccacgcaaagtgtgtaac |
| | | ggaataggtattggtgaatttaaagactcactctccataaatgctacgaatattaaacac |
| | | ttcaaaaactgcacctccatcagtggcgatctccacatcctgccggtggcatttaggggt |
| | | gactccttcacacatactcctcctctggatccacaggaactggatattctgaaaaccgta |
| | | aaggaaatcacagggtttttgctgattcaggcttggcctgaaaacaggacggacctccat |
| | | gcctttgagaacctagaaatcatacgcggcaggaccaagcaacatggtcagttttctctt |
| | | gcagtcgtcagcctgaacataacatccttgggattacgctccctcaaggagataagtgat |
| | | ggagatgtgataatttcaggaaacaaaaatttgtgctatgcaaatacaataaactggaaa |
| | | aaactgtttgggacctccggtcagaaaaccaaaattataagcaacagaggtgaaaacagc |
| | | tgcaaggccacaggccaggtctgccatgccttgtgctcccccgagggctgctggggcccg |
| | | gagcccagggactgcgtctcttgccggaatgtcagccgaggcagggaatgcgtggacaag |
| | | tgcaaccttctggagggtgagccaagggagtttgtggagaactctgagtgcatacagtgc |
| | | cacccagagtgcctgcctcaggccatgaacatcacctgcacaggacggggaccagacaac |
| | | tgtatccagtgtgcccactacattgacggcccccactgcgtcaagacctgcccggcagga |
| | | gtcatgggagaaaacaacaccctggtctggaagtacgcagacgccggccatgtgtgccac |
| | | ctgtgccatccaaactgcacctacggatgcactgggccaggtcttgaaggctgtccaacg |
| | | aatgggcctaagatcccgtccatcgccactgggatggtgggggccctcctcttgctgctg |
| | | gtggtggccctggggatcggcctcttcatgtgataa |
| 89 | Truncated | KEACPTGLYTHSGECCKACNLGEGVAQPCGANQTVCEPCLD |
| | LNGFR | SVTFSDVVSATEPCKPCTECVGLQSMSAPCVEADDAVCRCA |
| | Amino Acid | YGYYQDETTGRCEACRVCEAGSGLVFSCQDKQNTVCEECPD |
| | | GTYSDEANHVDPCLPCTVCEDTERQLRECTRWADAECEEIPG |
| | | RWITRSTPPEGSDSTAPSTQEPEAPPEQDLIASTVAGVVTTVM |
| | | GSSQPVVTRGTTDNLIPVYCSILAAVVVGLVAYIAFKR |
| 90 | Truncated | AAAGAGGCCTGTCCTACCGGCCTGTATACACACTCTGGCG |
| | LNGFR | AGTGCTGCAAGGCCTGCAATCTTGGAGAAGGCGTGGCACA |
| | Nucleic Acid | GCCTTGCGGCGCTAATCAGACAGTGTGCGAGCCTTGCCTG |
| | | GACAGCGTGACCTTTAGCGACGTGGTGTCTGCCACCGAGC |
| | | CATGCAAGCCTTGTACCGAGTGTGTGGGCCTGCAGAGCAT |
| | | GTCTGCCCCTTGTGTGGAAGCCGACGATGCCGTGTGTAGA |
| | | TGCGCCTACGGCTACTACCAGGACGAGACAACAGGCAGA |
| | | TGCGAGGCCTGTAGAGTGTGTGAAGCCGGCTCTGGACTGG |
| | | TGTTCAGCTGCCAAGACAAGCAGAACACCGTGTGCGAGG |
| | | AATGCCCCGATGGCACCTATAGCGACGAGGCCAACCATGT |
| | | GGATCCCTGCCTGCCTTGTACTGTGTGCGAAGATACCGAG |
| | | CGGCAGCTGCGCGAGTGTACAAGATGGGCTGATGCCGAGT |
| | | GCGAAGAGATCCCCGGCAGATGGATCACCAGAAGCACAC |
| | | CTCCAGAGGGCAGCGATAGCACAGCCCCTTCTACACAAGA |
| | | GCCCGAGGCTCCTCCTGAGCAGGATCTGATTGCCTCTACA |
| | | GTGGCCGGCGTGGTCACAACAGTGATGGGATCTTCTCAGC |
| | | CCGTGGTCACCAGAGGCACCACCGACAATCTGATCCCCGT |
| | | GTACTGTAGCATCCTGGCCGCCGTGGTTGTGGGACTCGTG |
| | | GCCTATATCGCCTTCAAGAGATGATAA |
| 91 | Truncated | RKVCNGIGIGEFKDSLSINATNIKHFKNCTSISGDLHILPVAFR |
| | EGFR | GDSFTHTPPLDPQELDILKTVKEITGFLLIQAWPENRTDLHAF |
| | (EGFRt) | ENLEIIRGRTKQHGQFSLAVVSLNITSLGLRSLKEISDGDVIISG |
| | Amino Acid | NKNLCYANTINWKKLFGTSGQKTKIISNRGENSCKATGQVC |
| | | HALCSPEGCWGPEPRDCVSCRNVSRGRECVDKCNLLEGEPR |
| | | EFVENSECIQCHPECLPQAMNITCTGRGPDNCIQCAHYIDGPH |
| | | CVKTCPAGVMGENNTLVWKYADAGHVCHLCHPNCTYGCT |
| | | GPGLEGCPTNGPKIPSIATGMVGALLLLLVVALGIGLFM |
| 92 | Truncated | cgcaaagtgtgtaacggaataggtattggtgaatttaaagactcactctccataaatgctac |
| | EGFR | gaatattaaacacttcaaaaactgcacctccatcagtggcgatctccacatcctgccggtgg |
| | (EGFRt) | catttaggggtgactccttcacacatactcctcctctggatccacaggaactggatattctg |
| | Nucleic acid | aaaaccgtaaaggaaatcacagggtttttgctgattcaggcttggcctgaaaacaggacgga |
| | | cctccatgcctttgagaacctagaaatcatacgcggcaggaccaagcaacatggtcagtttt |
| | | ctcttgcagtcgtcagcctgaacataacatccttgggattacgctccctcaaggagataagt |
| | | gatggagatgtgataatttcaggaaacaaaaatttgtgctatgcaaatacaataaactggaa |
| | | aaaactgtttgggacctccggtcagaaaaccaaaattataagcaacagaggtgaaaacagct |
| | | gcaaggccacaggccaggtctgccatgccttgtgctcccccgagggctgctggggcccggag |
| | | cccagggactgcgtctcttgccggaatgtcagccgaggcagggaatgcgtggacaagtgcaa |
| | | ccttctggagggtgagccaagggagtttgtggagaactctgagtgcatacagtgccacccag |
| | | agtgcctgcctcaggccatgaacatcacctgcacaggacggggaccagacaactgtatccag |
| | | tgtgcccactacattgacggcccccactgcgtcaagacctgcccggcaggagtcatgggaga |
| | | aaacaacaccctggtctggaagtacgcagacgccggccatgtgtgccacctgtgccatccaa |
| | | actgcacctacggatgcactgggccaggtcttgaaggctgtccaacgaatgggcctaagatc |
| | | ccgtccatcgccactgggatggtgggggccctcctcttgctgctggtggtggccctggggat |
| | | cggcctcttcatgTGATAA |
| 93 | Dap 10 signal | MIHLGHILFLLLLPVAAA |
| | peptide | |
| 94 | DAP10.6.3 | MIHLGHILFLLLLPVAAAQTTPGERSSLPAFYPGTSGSCSGCGSLSL |
| | (+sig domain, | PLLAGLVAADAVASLLIVGAVFLCARPRRSPAQEDGRVFINMPGR |
| | +FP2A, | GKRGRKKLLYIFKQPFMRPVQTTQEEDGCSCRFPEEEEGGCELRV |
| | +EGFRt) | KFSRSADAPAYQQGQNQLYNELNLGRREEYDVLDKRRGRDPEM |
| | Amino Acid | GGKPQRRKNPQEGLYNELQKDKMAEAYSEIGMKGERRRGKGH |
| | | DGLYQGLSTATKDTYDALHMQALPPRRAKRSGSGATNFSLLKQ |
| | | AGDVEENPGPMLLLVTSLLLCELPHPAFLLIPRKVCNGIGIGEFKD |
| | | SLSINATNIKHFKNCTSISGDLHILPVAFRGDSFTHTPPLDPQELDIL |
| | | KTVKEITGFLLIQAWPENRTDLHAFENLEIIRGRTKQHGQFSLAVV |
| | | SLNITSLGLRSLKEISDGDVIISGNKNLCYANTINWKKLFGTSGQK |
| | | TKIISNRGENSCKATGQVCHALCSPEGCWGPEPRDCVSCRNVSRG |
| | | RECVDKCNLLEGEPREFVENSECIQCHPECLPQAMNITCTGRGPD |
| | | NCIQCAHYIDGPHCVKTCPAGVMGENNTLVWKYADAGHVCHL |
| | | CHPNCTYGCTGPGLEGCPTNGPKIPSIATGMVGALLLLLVVALGI |
| | | GLFM |
| 95 | DAP10.6.3 | ATGATCCACCTGGGCCACATCCTGTTCCTGCTGCTCCTGCCTGT |
| | (+sig domain, | GGCCGCTGCTCAGACCACCCCTGGAGAAAGATCTAGCCTGCC |
| | +FP2A, | CGCTTTCTACCCCGGGACCAGCGGCAGCTGCAGCGGATGCGG |
| | +EGFRt) | CTCTCTGAGCCTGCCTCTGCTGGCCGGCCTCGTGGCCGCCGAT |
| | Nucleic Acid | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCAAAAGAGGCAGAAAGAAG |
| | | CTGCTGTACATCTTCAAGCAGCCTTTCATGCGGCCCGTGCAGA |
| | | CAACCCAGGAGGAGGACGGCTGTAGCTGTAGATTCCCCGAGG |
| | | AAGAAGAAGGCGGCTGCGAGCTTAGAGTGAAGTTCAGCAGAA |
| | | GCGCCGACGCCCCTGCTTACCAGCAGGGCCAGAACCAGCTGT |
| | | ATAATGAGCTGAACCTGGGAAGAAGGGAAGAGTACGACGTGC |
| | | TGGACAAGCGGCGGGGCAGAGATCCTGAGATGGGCGGAAAA |
| | | CCTCAAAGAAGGAAGAACCCTCAGGAGGGCCTGTACAACGAG |
| | | CTGCAGAAAGACAAGATGGCCGAGGCCTACAGCGAGATCGGC |
| | | ATGAAAGGCGAGAGACGGCGGGGCAAGGGCCACGACGGCCT |
| | | GTACCAAGGCCTGTCAACAGCCACCAAGGACACCTACGACGC |
| | | CCTGCACATGCAGGCCCTGCCACCTAGACGCGCGAAGCGATC |
| | | AGGCAGCGGGGCGACAAATTTCAGCCTTCTGAAACAAGCAGG |
| | | CGACGTGGAAGAAAACCCCGGTCCAatgcttctcctggtgacaagccttctgctct |
| | | gtgagttaccacacccagcattcctcctgatcccacgcaaagtgtgtaacggaataggtatt |
| | | ggtgaatttaaagactcactctccataaatgctacgaatattaaacacttcaaaaactgcac |
| | | ctccatcagtggcgatctccacatcctgccggtggcatttaggggtgactccttcacacata |
| | | ctcctcctctggatccacaggaactggatattctgaaaaccgtaaaggaaatcacagggttt |
| | | ttgctgattcaggcttggcctgaaaacaggacggacctccatgcctttgagaacctagaaat |
| | | catacgcggcaggaccaagcaacatggtcagttttctcttgcagtcgtcagcctgaacataa |
| | | catccttgggattacgctccctcaaggagataagtgatggagatgtgataatttcaggaaac |
| | | aaaaatttgtgctatgcaaatacaataaactggaaaaaactgtttgggacctccggtcagaa |
| | | aaccaaaattataagcaacagaggtgaaaacagctgcaaggccacaggccaggtctgccatg |
| | | ccttgtgctcccccgagggctgctggggcccggagcccagggactgcgtctcttgccggaat |
| | | gtcagccgaggcagggaatgcgtggacaagtgcaaccttctggagggtgagccaagggagtt |
| | | tgtggagaactctgagtgcatacagtgccacccagagtgcctgcctcaggccatgaacatca |
| | | cctgcacaggacggggaccagacaactgtatccagtgtgcccactacattgacggcccccac |
| | | tgcgtcaagacctgcccggcaggagtcatgggagaaaacaacaccctggtctggaagtacgc |
| | | agacgccggccatgtgtgccacctgtgccatccaaactgcacctacggatgcactgggccag |
| | | gtcttgaaggctgtccaacgaatgggcctaagatcccgtccatcgccactgggatggtgggg |
| | | gccctcctcttgctgctggtggtggccctggggatcggcctcttcatgTGATAA |
| 96 | DAP10.16 | MIHLGHILFLLLLPVAAAQTTPGERSSLPAFYPGTSGSCSGCGSLSL |
| | Amino acid | PLLAGLVAADAVASLLIVGAVFLCARPRRSPAQEDGRVFINMPGR |
| | | GKRGRKKLLYIFKQPFMRPVQTTQEEDGCSCRFPEEEEGGCELRV |
| | | KFSRSADAPAYQQGQNQLYNELNLGRREEYDVLDKRRGRDPEM |
| | | GGKPQRRKNPQEGLFNELQKDKMAEAFSEIGMKGERRRGKGHD |
| | | GLFQGLSTATKDTFDALHMQALPPRRAKRSGSGATNFSLLKQAG |
| | | DVEENPGPMPPPRLLFFLLFLTPMEVRPEEPLVVKVEEGDNAVLQ |
| | | CLKGTSDGPTQQLTWSRESPLKPFLKLSLGLPGLGIHMRPLAIWLF |
| | | IFNVSQQMGGFYLCQPGPPSEKAWQPGWTVNVEGSGELFRWNV |
| | | SDLGGLGCGLKNRSSEGPSSPSGKLMSPKLYVWAKDRPEIWEGE |
| | | PPCLPPRDSLNQSLSQDLTMAPGSTLWLSCGVPPDSVSRGPLSWT |
| | | HVHPKGPKSLLSLELKDDRPARDMWVMETGLLLPRATAQDAGK |
| | | YYCHRGNLTMSFHLEITARPVLWHWLLRTGGWKVSAVTLAYLIF |
| | | CLCSLVGILHLQRALVLRRKRKRMTDPTRRF |
| 97 | DAP10.16 | ATGATCCACCTGGGCCACATCCTGTTCCTGCTGCTCCTGCCTGT |
| | Nucleic acid | GGCCGCTGCTCAGACCACCCCTGGAGAAAGATCTAGCCTGCC |
| | | CGCTTTCTACCCCGGGACCAGCGGCAGCTGCAGCGGATGCGG |
| | | CTCTCTGAGCCTGCCTCTGCTGGCCGGCCTCGTGGCCGCCGAT |
| | | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCAAAAGAGGCAGAAAGAAG |
| | | CTGCTGTACATCTTCAAGCAGCCTTTCATGCGGCCCGTGCAGA |
| | | CAACCCAGGAGGAGGACGGCTGTAGCTGTAGATTCCCCGAGG |
| | | AAGAAGAAGGCGGCTGCGAGCTTCGAGTGAAGTTCAGTAGAA |
| | | GCGCCGACGCCCCTGCCTACCAGCAGGGCCAGAACCAGCTGT |
| | | ACAACGAGCTGAACCTGGGCAGACGCGAGGAATACGACGTGC |
| | | TGGACAAGCGGCGGGGCAGAGATCCTGAGATGGGCGGAAAA |
| | | CCTCAAAGAAGAAAGAACCCCCAGGAGGGCCTGTTCAACGAG |
| | | CTGCAGAAAGATAAGATGGCCGAGGCCTTCAGCGAGATCGGC |
| | | ATGAAAGGCGAGAGACGACGTGGAAAGGGCCACGACGGCTT |
| | | GTTTCAGGGCCTGAGCACCGCCACAAAGGACACCTTCGACGC |
| | | CCTGCACATGCAGGCCCTCCCCCCCAGACGCGCGAAGCGATC |
| | | AGGCAGCGGGGCGACAAATTTCAGCCTTCTGAAACAAGCAGG |
| | | CGACGTGGAAGAAAACCCCGGTCCAATGCCTCCTCCTCGGCTG |
| | | CTGTTCTTCCTGCTGTTTCTGACCCCTATGGAAGTGCGGCCCGA |
| | | GGAACCTCTGGTGGTCAAAGTTGAAGAGGGCGACAACGCCGT |
| | | GCTGCAGTGTCTGAAGGGCACATCTGATGGCCCCACACAGCA |
| | | GCTGACCTGGTCTAGAGAGAGCCCTCTGAAGCCCTTCCTGAAG |
| | | CTGTCTCTGGGACTGCCTGGACTGGGCATCCATATGAGGCCTC |
| | | TGGCCATCTGGCTGTTCATCTTCAACGTGTCCCAGCAGATGGG |
| | | CGGCTTCTACCTGTGTCAACCTGGACCTCCAAGCGAGAAGGCT |
| | | TGGCAGCCTGGCTGGACCGTGAATGTGGAAGGATCCGGCGAG |
| | | CTGTTCCGGTGGAATGTGTCTGATCTCGGCGGCCTCGGATGCG |
| | | GCCTGAAGAATAGATCTAGCGAGGGCCCTAGCAGCCCCAGCG |
| | | GAAAACTGATGAGCCCCAAGCTGTACGTGTGGGCCAAAGACA |
| | | GACCCGAGATTTGGGAGGGCGAGCCTCCTTGTCTGCCTCCTAG |
| | | AGACAGCCTGAACCAGAGCCTGAGCCAGGACCTGACAATGGC |
| | | CCCTGGATCTACACTGTGGCTGAGCTGTGGCGTGCCACCTGAC |
| | | AGTGTGTCTAGAGGCCCTCTGTCTTGGACCCACGTGCACCCTA |
| | | AGGGCCCTAAGTCTCTGCTGAGCCTGGAACTGAAGGACGACA |
| | | GGCCCGCCAGAGATATGTGGGTCATGGAAACAGGCCTGCTGC |
| | | TGCCTAGAGCCACAGCACAGGATGCCGGCAAGTACTACTGCC |
| | | ACAGAGGCAACCTGACCATGAGCTTCCACCTGGAAATCACCG |
| | | CCAGACCTGTCCTGTGGCACTGGCTGCTTAGAACCGGCGGCTG |
| | | GAAAGTGTCTGCCGTGACTCTGGCCTACCTGATCTTCTGCCTG |
| | | TGTAGCCTCGTGGGCATCCTGCATCTGCAGAGAGCACTGGTCC |
| | | TGCGGCGGAAGCGGAAGAGAATGACCGATCCTACCAGACGGT |
| | | TCTGATAA |
| 98 | DAP10.16.1 | MIHLGHILFLLLLPVAAAQTTPGERSSLPAFYPGTSGSCSGCGSLSL |
| | (+sig domain, | PLLAGLVAADAVASLLIVGAVFLCARPRRSPAQEDGRVFINMPGR |
| | −FP2A, | GKRGRKKLLYIFKQPFMRPVQTTQEEDGCSCRFPEEEEGGCELRV |
| | −CD19) | KFSRSADAPAYQQGQNQLYNELNLGRREEYDVLDKRRGRDPEM |
| | Amino acid | GGKPQRRKNPQEGLFNELQKDKMAEAFSEIGMKGERRRGKGHD |
| | | GLFQGLSTATKDTFDALHMQALPPR |
| 99 | DAP10.16.1 | ATGATCCACCTGGGCCACATCCTGTTCCTGCTGCTCCTGCCTGT |
| | (+sig domain, | GGCCGCTGCTCAGACCACCCCTGGAGAAAGATCTAGCCTGCC |
| | −FP2A, | CGCTTTCTACCCCGGGACCAGCGGCAGCTGCAGCGGATGCGG |
| | −CD19) | CTCTCTGAGCCTGCCTCTGCTGGCCGGCCTCGTGGCCGCCGAT |
| | Nucleic acid | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCAAAAGAGGCAGAAAGAAG |
| | | CTGCTGTACATCTTCAAGCAGCCTTTCATGCGGCCCGTGCAGA |
| | | CAACCCAGGAGGAGGACGGCTGTAGCTGTAGATTCCCCGAGG |
| | | AAGAAGAAGGCGGCTGCGAGCTTCGAGTGAAGTTCAGTAGAA |
| | | GCGCCGACGCCCCTGCCTACCAGCAGGGCCAGAACCAGCTGT |
| | | ACAACGAGCTGAACCTGGGCAGACGCGAGGAATACGACGTGC |
| | | TGGACAAGCGGCGGGGCAGAGATCCTGAGATGGGCGGAAAA |
| | | CCTCAAAGAAGAAAGAACCCCCAGGAGGGCCTGTTCAACGAG |
| | | CTGCAGAAAGATAAGATGGCCGAGGCCTTCAGCGAGATCGGC |
| | | ATGAAAGGCGAGAGACGACGTGGAAAGGGCCACGACGGCTT |
| | | GTTTCAGGGCCTGAGCACCGCCACAAAGGACACCTTCGACGC |
| | | CCTGCACATGCAGGCCCTCCCCCCCAGA |
| 100 | DAP10.16.2 | QTTPGERSSLPAFYPGTSGSCSGCGSLSLPLLAGLVAADAVASLLI |
| | (−sig domain, | VGAVFLCARPRRSPAQEDGRVFINMPGRGKRGRKKLLYIFKQPF |
| | −FP2A, | MRPVQTTQEEDGCSCRFPEEEEGGCELRVKFSRSADAPAYQQGQ |
| | −CD19) | NQLYNELNLGRREEYDVLDKRRGRDPEMGGKPQRRKNPQEGLF |
| | Amino acid | NELQKDKMAEAFSEIGMKGERRRGKGHDGLFQGLSTATKDTFD |
| | | ALHMQALPPR |
| 101 | DAP10.16.2 | CAGACCACCCCTGGAGAAAGATCTAGCCTGCCCGCTTTCTACC |
| | (−sig domain, | CCGGGACCAGCGGCAGCTGCAGCGGATGCGGCTCTCTGAGCC |
| | −FP2A, | TGCCTCTGCTGGCCGGCCTCGTGGCCGCCGATGCTGTGGCCAG |
| | −CD19) | CCTGCTGATCGTGGGCGCCGTGTTCCTGTGCGCCAGACCCAGA |
| | Nucleic acid | AGAAGCCCTGCCCAGGAGGATGGCAGAGTGTTCATCAACATG |
| | | CCTGGCAGAGGCAAAAGAGGCAGAAAGAAGCTGCTGTACATC |
| | | TTCAAGCAGCCTTTCATGCGGCCCGTGCAGACAACCCAGGAG |
| | | GAGGACGGCTGTAGCTGTAGATTCCCCGAGGAAGAAGAAGGC |
| | | GGCTGCGAGCTTCGAGTGAAGTTCAGTAGAAGCGCCGACGCC |
| | | CCTGCCTACCAGCAGGGCCAGAACCAGCTGTACAACGAGCTG |
| | | AACCTGGGCAGACGCGAGGAATACGACGTGCTGGACAAGCGG |
| | | CGGGGCAGAGATCCTGAGATGGGCGGAAAACCTCAAAGAAG |
| | | AAAGAACCCCCAGGAGGGCCTGTTCAACGAGCTGCAGAAAGA |
| | | TAAGATGGCCGAGGCCTTCAGCGAGATCGGCATGAAAGGCGA |
| | | GAGACGACGTGGAAAGGGCCACGACGGCTTGTTTCAGGGCCT |
| | | GAGCACCGCCACAAAGGACACCTTCGACGCCCTGCACATGCA |
| | | GGCCCTCCCCCCCAGA |
| 102 | DAP10.16.3 | MIHLGHILFLLLLPVAAAQTTPGERSSLPAFYPGTSGSCSGCGSLSL |
| | (+sig domain, | PLLAGLVAADAVASLLIVGAVFLCARPRRSPAQEDGRVFINMPGR |
| | +FP2A, | GKRGRKKLLYIFKQPFMRPVQTTQEEDGCCRFPEEEEGGCELRV |
| | +EGFRt) | KFSRSADAPAYQQGQNQLYNELNLGRREEYDVLDKRRGRDPEM |
| | Amino Acid | GGKPQRRKNPQEGLFNELQKDKMAEAFSEIGMKGERRRGKGHD |
| | | GLFQGLSTATKDTFDALHMQALPPRRAKRSGSGATNFSLLKQAG |
| | | DVEENPGPMLLLVTSLLLCELPHPAFLLIPRKVCNGIGIGEFKDSLS |
| | | INATNIKHFKNCTSISGDLHILPVAFRGDSFTHTPPLDPQELDILKT |
| | | VKEITGFLLIQAWPENRTDLHAFENLEIIRGRTKQHGQFSLAVVSL |
| | | NITSLGLRSLKEISDGDVIISGNKNLCYANTINWKKLFGTSGQKTK |
| | | IISNRGENSCKATGQVCHALCSPEGCWGPEPRDCVSCRNVSRGRE |
| | | CVDKCNLLEGEPREFVENSECIQCHPECLPQAMNITCTGRGPDNCI |
| | | QCAHYIDGPHCVKTCPAGVMGENNTLVWKYADAGHVCHLCHP |
| | | NCTYGCTGPGLEGCPTNGPKIPSIATGMVGALLLLLVVALGIGLF |
| | | M |
| 103 | DAP10.16.3 | ATGATCCACCTGGGCCACATCCTGTTCCTGCTGCTCCTGCCTGT |
| | (+sig domain, | GGCCGCTGCTCAGACCACCCCTGGAGAAAGATCTAGCCTGCC |
| | +FP2A, | CGCTTTCTACCCCGGGACCAGCGGCAGCTGCAGCGGATGCGG |
| | +EGFRt) | CTCTCTGAGCCTGCCTCTGCTGGCCGGCCTCGTGGCCGCCGAT |
| | Nucleic Acid | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCAAAAGAGGCAGAAAGAAG |
| | | CTGCTGTACATCTTCAAGCAGCCTTTCATGCGGCCCGTGCAGA |
| | | CAACCCAGGAGGAGGACGGCTGTAGCTGTAGATTCCCCGAGG |
| | | AAGAAGAAGGCGGCTGCGAGCTTCGAGTGAAGTTCAGTAGAA |
| | | GCGCCGACGCCCCTGCCTACCAGCAGGGCCAGAACCAGCTGT |
| | | ACAACGAGCTGAACCTGGGCAGACGCGAGGAATACGACGTGC |
| | | TGGACAAGCGGCGGGGCAGAGATCCTGAGATGGGCGGAAAA |
| | | CCTCAAAGAAGAAAGAACCCCCAGGAGGGCCTGTTCAACGAG |
| | | CTGCAGAAAGATAAGATGGCCGAGGCCTTCAGCGAGATCGGC |
| | | ATGAAAGGCGAGAGACGACGTGGAAAGGGCCACGACGGCTT |
| | | GTTTCAGGGCCTGAGCACCGCCACAAAGGACACCTTCGACGC |
| | | CCTGCACATGCAGGCCCTCCCCCCCAGACGCGCGAAGCGATC |
| | | AGGCAGCGGGGCGACAAATTTCAGCCTTCTGAAACAAGCAGG |
| | | CGACGTGGAAGAAAACCCCGGTCCAatgcttctcctggtgacaagccttctgctct |
| | | gtgagttaccacacccagcattcctcctgatcccacgcaaagtgtgtaacggaataggtatt |
| | | ggtgaatttaaagactcactctccataaatgctacgaatattaaacacttcaaaaactgcac |
| | | ctccatcagtggcgatctccacatcctgccggtggcatttaggggtgactccttcacacata |
| | | ctcctcctctggatccacaggaactggatattctgaaaaccgtaaaggaaatcacagggttt |
| | | ttgctgattcaggcttggcctgaaaacaggacggacctccatgcctttgagaacctagaaat |
| | | catacgcggcaggaccaagcaacatggtcagttttctcttgcagtcgtcagcctgaacataa |
| | | catccttgggattacgctccctcaaggagataagtgatggagatgtgataatttcaggaaac |
| | | aaaaatttgtgctatgcaaatacaataaactggaaaaaactgtttgggacctccggtcagaa |
| | | aaccaaaattataagcaacagaggtgaaaacagctgcaaggccacaggccaggtctgccatg |
| | | ccttgtgctcccccgagggctgctggggcccggagcccagggactgcgtctcttgccggaat |
| | | gtcagccgaggcagggaatgcgtggacaagtgcaaccttctggagggtgagccaagggagtt |
| | | tgtggagaactctgagtgcatacagtgccacccagagtgcctgcctcaggccatgaacatca |
| | | cctgcacaggacggggaccagacaactgtatccagtgtgcccactacattgacggcccccac |
| | | tgcgtcaagacctgcccggcaggagtcatgggagaaaacaacaccctggtctggaagtacgc |
| | | agacgccggccatgtgtgccacctgtgccatccaaactgcacctacggatgcactgggccag |
| | | gtcttgaaggctgtccaacgaatgggcctaagatcccgtccatcgccactgggatggtgggg |
| | | gccctcctcttgctgctggtggtggccctggggatcggcctcttcatgTGATAA |
| 104 | DAP10.17 | MIHLGHILFLLLLPVAAAQTTPGERSSLPAFYPGTSGSCSGCGSLSL |
| | Amino acid | PLLAGLVAADAVASLLIVGAVFLCARPRRSPAQEDGRVFINMPGR |
| | | GRVKFSRSADAPAYQQGQNQLYNELNLGRREEYDVLDKRRGRD |
| | | PEMGGKPQRRKNPQEGLFNELQKDKMAEAFSEIGMKGERRRGK |
| | | GHDGLFQGLSTATKDTFDALHMQALPPRRAKRSGSGATNFSLLK |
| | | QAGDVEENPGPMPPPRLLFFLLFLTPMEVRPEEPLVVKVEEGDNA |
| | | VLQCLKGTSDGPTQQLTWSRESPLKPFLKLSLGLPGLGIHMRPLAI |
| | | WLFIFNVSQQMGGFYLCQPGPPSEKAWQPGWTVNVEGSGELFR |
| | | WNVSDLGGLGCGLKNRSSEGPSSPSGKLMSPKLYVWAKDRPEIW |
| | | EGEPPCLPPRDSLNQSLSQDLTMAPGSTLWLSCGVPPDSVSRGPLS |
| | | WTHVHPKGPKSLLSLELKDDRPARDMWVMETGLLLPRATAQDA |
| | | GKYYCHRGNLTMSFHLEITARPVLWHWLLRTGGWKVSAVTLAY |
| | | LIFCLCSLVGILHLQRALVLRRKRKRMTDPTRRF |
| 105 | DAP10.17 | ATGATCCACCTGGGCCACATCCTGTTCCTGCTGCTCCTGCCTGT |
| | Nucleic acid | GGCCGCTGCTCAGACCACCCCTGGAGAAAGATCTAGCCTGCC |
| | | CGCTTTCTACCCCGGGACCAGCGGCAGCTGCAGCGGATGCGG |
| | | CTCTCTGAGCCTGCCTCTGCTGGCCGGCCTCGTGGCCGCCGAT |
| | | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCCGAGTGAAGTTCAGTAGAA |
| | | GCGCCGACGCCCCTGCCTACCAGCAGGGCCAGAACCAGCTGT |
| | | ACAACGAGCTGAACCTGGGCAGACGCGAGGAATACGACGTGC |
| | | TGGACAAGCGGCGGGGCAGAGATCCTGAGATGGGCGGAAAA |
| | | CCTCAAAGAAGAAAGAACCCCCAGGAGGGCCTGTTCAACGAG |
| | | CTGCAGAAAGATAAGATGGCCGAGGCCTTCAGCGAGATCGGC |
| | | ATGAAAGGCGAGAGACGACGTGGAAAGGGCCACGACGGCTT |
| | | GTTTCAGGGCCTGAGCACCGCCACAAAGGACACCTTCGACGC |
| | | CCTGCACATGCAGGCCCTCCCCCCCAGACGCGCGAAGCGATC |
| | | AGGCAGCGGGGCGACAAATTTCAGCCTTCTGAAACAAGCAGG |
| | | CGACGTGGAAGAAAACCCCGGTCCAATGCCTCCTCCTCGGCTG |
| | | CTGTTCTTCCTGCTGTTTCTGACCCCTATGGAAGTGCGGCCCGA |
| | | GGAACCTCTGGTGGTCAAAGTTGAAGAGGGCGACAACGCCGT |
| | | GCTGCAGTGTCTGAAGGGCACATCTGATGGCCCCACACAGCA |
| | | GCTGACCTGGTCTAGAGAGAGCCCTCTGAAGCCCTTCCTGAAG |
| | | CTGTCTCTGGGACTGCCTGGACTGGGCATCCATATGAGGCCTC |
| | | TGGCCATCTGGCTGTTCATCTTCAACGTGTCCCAGCAGATGGG |
| | | CGGCTTCTACCTGTGTCAACCTGGACCTCCAAGCGAGAAGGCT |
| | | TGGCAGCCTGGCTGGACCGTGAATGTGGAAGGATCCGGCGAG |
| | | CTGTTCCGGTGGAATGTGTCTGATCTCGGCGGCCTCGGATGCG |
| | | GCCTGAAGAATAGATCTAGCGAGGGCCCTAGCAGCCCCAGCG |
| | | GAAAACTGATGAGCCCCAAGCTGTACGTGTGGGCCAAAGACA |
| | | GACCCGAGATTTGGGAGGGCGAGCCTCCTTGTCTGCCTCCTAG |
| | | AGACAGCCTGAACCAGAGCCTGAGCCAGGACCTGACAATGGC |
| | | CCCTGGATCTACACTGTGGCTGAGCTGTGGCGTGCCACCTGAC |
| | | AGTGTGTCTAGAGGCCCTCTGTCTTGGACCCACGTGCACCCTA |
| | | AGGGCCCTAAGTCTCTGCTGAGCCTGGAACTGAAGGACGACA |
| | | GGCCCGCCAGAGATATGTGGGTCATGGAAACAGGCCTGCTGC |
| | | TGCCTAGAGCCACAGCACAGGATGCCGGCAAGTACTACTGCC |
| | | ACAGAGGCAACCTGACCATGAGCTTCCACCTGGAAATCACCG |
| | | CCAGACCTGTCCTGTGGCACTGGCTGCTTAGAACCGGCGGCTG |
| | | GAAAGTGTCTGCCGTGACTCTGGCCTACCTGATCTTCTGCCTG |
| | | TGTAGCCTCGTGGGCATCCTGCATCTGCAGAGAGCACTGGTCC |
| | | TGCGGCGGAAGCGGAAGAGAATGACCGATCCTACCAGACGGT |
| | | TCTGATAA |
| 106 | DAP10.17.1 | MIHLGHILFLLLLPVAAAQTTPGERSSLPAFYPGTSGSCSGCGSLSL |
| | (+sig domain, | PLLAGLVAADAVASLLIVGAVFLCARPRRSPAQEDGRVFINMPGR |
| | −FP2A, | GRVKFSRSADAPAYQQGQNQLYNELNLGRREEYDVLDKRRGRD |
| | −CD19) | PEMGGKPQRRKNPQEGLFNELQKDKMAEAFSEIGMKGERRRGK |
| | Amino acid | GHDGLFQGLSTATKDTFDALHMQALPPR |
| 107 | DAP10.17.1 | ATGATCCACCTGGGCCACATCCTGTTCCTGCTGCTCCTGCCTGT |
| | (+sig domain, | GGCCGCTGCTCAGACCACCCCTGGAGAAAGATCTAGCCTGCC |
| | −FP2A, | CGCTTTCTACCCCGGGACCAGCGGCAGCTGCAGCGGATGCGG |
| | −CD19) | CTCTCTGAGCCTGCCTCTGCTGGCCGGCCTCGTGGCCGCCGAT |
| | Nucleic acid | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCCGAGTGAAGTTCAGTAGAA |
| | | GCGCCGACGCCCCTGCCTACCAGCAGGGCCAGAACCAGCTGT |
| | | ACAACGAGCTGAACCTGGGCAGACGCGAGGAATACGACGTGC |
| | | TGGACAAGCGGCGGGGCAGAGATCCTGAGATGGGCGGAAAA |
| | | CCTCAAAGAAGAAAGAACCCCCAGGAGGGCCTGTTCAACGAG |
| | | CTGCAGAAAGATAAGATGGCCGAGGCCTTCAGCGAGATCGGC |
| | | ATGAAAGGCGAGAGACGACGTGGAAAGGGCCACGACGGCTT |
| | | GTTTCAGGGCCTGAGCACCGCCACAAAGGACACCTTCGACGC |
| | | CCTGCACATGCAGGCCCTCCCCCCCAGA |
| 108 | DAP10.17.2 | QTTPGERSSLPAFYPGTSGSCSGCGSLSLPLLAGLVAADAVASLLI |
| | (−sig domain, | VGAVFLCARPRRSPAQEDGRVFINMPGRGRVKFSRSADAPAYQQ |
| | −FP2A, | GQNQLYNELNLGRREEYDVLDKRRGRDPEMGGKPQRRKNPQEG |
| | −CD19) | LFNELQKDKMAEAFSEIGMKGERRRGKGHDGLFQGLSTATKDTF |
| | Amino acid | DALHMQALPPR |
| 109 | DAP10.17.2 | CAGACCACCCCTGGAGAAAGATCTAGCCTGCCCGCTTTCTACC |
| | (−sig domain, | CCGGGACCAGCGGCAGCTGCAGCGGATGCGGCTCTCTGAGCC |
| | −FP2A, | TGCCTCTGCTGGCCGGCCTCGTGGCCGCCGATGCTGTGGCCAG |
| | −CD19) | CCTGCTGATCGTGGGCGCCGTGTTCCTGTGCGCCAGACCCAGA |
| | Nucleic acid | AGAAGCCCTGCCCAGGAGGATGGCAGAGTGTTCATCAACATG |
| | | CCTGGCAGAGGCCGAGTGAAGTTCAGTAGAAGCGCCGACGCC |
| | | CCTGCCTACCAGCAGGGCCAGAACCAGCTGTACAACGAGCTG |
| | | AACCTGGGCAGACGCGAGGAATACGACGTGCTGGACAAGCGG |
| | | CGGGGCAGAGATCCTGAGATGGGCGGAAAACCTCAAAGAAG |
| | | AAAGAACCCCCAGGAGGGCCTGTTCAACGAGCTGCAGAAAGA |
| | | TAAGATGGCCGAGGCCTTCAGCGAGATCGGCATGAAAGGCGA |
| | | GAGACGACGTGGAAAGGGCCACGACGGCTTGTTTCAGGGCCT |
| | | GAGCACCGCCACAAAGGACACCTTCGACGCCCTGCACATGCA |
| | | GGCCCTCCCCCCCAGA |
| 110 | DAP10.17.3 | MIHLGHILFLLLLPVAAAQTTPGERSSLPAFYPGTSGSCSGCGSLSL |
| | (+sig domain, | PLLAGLVAADAVASLLIVGAVFLCARPRRSPAQEDGRVFINMPGR |
| | +FP2A, | GRVKFSRSADAPAYQQGQNQLYNELNLGRREEYDVLDKRRGRD |
| | +EGFRt) | PEMGGKPQRRKNPQEGLFNELQKDKMAEAFSEIGMKGERRRGK |
| | Amino Acid | GHDGLFQGLSTATKDTFDALHMQALPPRRAKRSGSGATNFSLLK |
| | | QAGDVEENPGPMLLLVTSLLLCELPHPAFLLIPRKVCNGIGIGEFK |
| | | DSLSINATNIKHFKNCTSISGDLHILPVAFRGDSFTHTPPLDPQELDI |
| | | LKTVKEITGFLLIQAWPENRTDLHAFENLEIIRGRTKQHGQFSLAV |
| | | VSLNITSLGLRSLKEISDGDVIISGNKNLCYANTINWKKLFGTSGQ |
| | | KTKIISNRGENSCKATGQVCHALCSPEGCWGPEPRDCVSCRNVSR |
| | | GRECVDKCNLLEGEPREFVENSECIQCHPECLPQAMNITCTGRGP |
| | | DNCIQCAHYIDGPHCVKTCPAGVMGENNTLVWKYADAGHVCH |
| | | LCHPNCTYGCTGPGLEGCPTNGPKIPSIATGMVGALLLLLVVALG |
| | | IGLFM |
| 111 | DAP10.17.3 | ATGATCCACCTGGGCCACATCCTGTTCCTGCTGCTCCTGCCTGT |
| | (+sig domain, | GGCCGCTGCTCAGACCACCCCTGGAGAAAGATCTAGCCTGCC |
| | +FP2A, | CGCTTTCTACCCCGGGACCAGCGGCAGCTGCAGCGGATGCGG |
| | +EGFRt) | CTCTCTGAGCCTGCCTCTGCTGGCCGGCCTCGTGGCCGCCGAT |
| | Nucleic Acid | GCTGTGGCCAGCCTGCTGATCGTGGGCGCCGTGTTCCTGTGCG |
| | | CCAGACCCAGAAGAAGCCCTGCCCAGGAGGATGGCAGAGTGT |
| | | TCATCAACATGCCTGGCAGAGGCCGAGTGAAGTTCAGTAGAA |
| | | GCGCCGACGCCCCTGCCTACCAGCAGGGCCAGAACCAGCTGT |
| | | ACAACGAGCTGAACCTGGGCAGACGCGAGGAATACGACGTGC |
| | | TGGACAAGCGGCGGGGCAGAGATCCTGAGATGGGCGGAAAA |
| | | CCTCAAAGAAGAAAGAACCCCCAGGAGGGCCTGTTCAACGAG |
| | | CTGCAGAAAGATAAGATGGCCGAGGCCTTCAGCGAGATCGGC |
| | | ATGAAAGGCGAGAGACGACGTGGAAAGGGCCACGACGGCTT |
| | | GTTTCAGGGCCTGAGCACCGCCACAAAGGACACCTTCGACGC |
| | | CCTGCACATGCAGGCCCTCCCCCCCAGACGCGCGAAGCGATC |
| | | AGGCAGCGGGGCGACAAATTTCAGCCTTCTGAAACAAGCAGG |
| | | CGACGTGGAAGAAAACCCCGGTCCAatgcttctcctggtgacaagccttctgctct |
| | | gtgagttaccacacccagcattcctcctgatcccacgcaaagtgtgtaacggaataggtatt |
| | | ggtgaatttaaagactcactctccataaatgctacgaatattaaacacttcaaaaactgcac |
| | | ctccatcagtggcgatctccacatcctgccggtggcatttaggggtgactccttcacacata |
| | | ctcctcctctggatccacaggaactggatattctgaaaaccgtaaaggaaatcacagggttt |
| | | ttgctgattcaggcttggcctgaaaacaggacggacctccatgcctttgagaacctagaaat |
| | | catacgcggcaggaccaagcaacatggtcagttttctcttgcagtcgtcagcctgaacataa |
| | | catccttgggattacgctccctcaaggagataagtgatggagatgtgataatttcaggaaac |
| | | aaaaatttgtgctatgcaaatacaataaactggaaaaaactgtttgggacctccggtcagaa |
| | | aaccaaaattataagcaacagaggtgaaaacagctgcaaggccacaggccaggtctgccatg |
| | | ccttgtgctcccccgagggctgctggggcccggagcccagggactgcgtctcttgccggaat |
| | | gtcagccgaggcagggaatgcgtggacaagtgcaaccttctggagggtgagccaagggagtt |
| | | tgtggagaactctgagtgcatacagtgccacccagagtgcctgcctcaggccatgaacatca |
| | | cctgcacaggacggggaccagacaactgtatccagtgtgcccactacattgacggcccccac |
| | | tgcgtcaagacctgcccggcaggagtcatgggagaaaacaacaccctggtctggaagtacgc |
| | | agacgccggccatgtgtgccacctgtgccatccaaactgcacctacggatgcactgggccag |
| | | gtcttgaaggctgtccaacgaatgggcctaagatcccgtccatcgccactgggatggtgggg |
| | | gccctcctcttgctgctggtggtggccctggggatcggcctcttcatgTGATAA |
| 112 | 3H7-5.1-P2A- | atgtccgtgcctacccaggtgctgggcctgctgctgctgtggctgaccgacgccagatgcga |
| | DAP10.1 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCT |
| | | GTTCCTGCTGCTGCTGCCTGTGGCCGCCGCTCAGACCACCCCTGGCGAGAGAAGCAGCCTGC |
| | | CCGCCTTCTACCCCGGGACCAGCGGATCTTGTAGCGGCTGCGGCAGCTTGTCTCTGCCTCTG |
| | | CTCGCCGGCCTGGTGGCTGCTGATGCCGTGGCCAGCCTGCTGATCGTGGGCGCCGTCTTTCT |
| | | GTGCGCCAGACCTAGAAGGTCCCCTGCCCAGGAGGACGGCAAGGTGTTCATCAACATGCCTG |
| | | GAAGAGGCTGA |
| 113 | 3H7-5.1-P2A- | MSVPTQVLGLLLLWLTDARCEIVMTQSPATLSVSPGERTTLSCRA |
| | DAP10.1 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVFINMPGRG* |
| 114 | 3H7-5.1-P2A- | atgtccgtgcctacccaggtgctgggcctgctgctgctgtggctgaccgacgccagatgcga |
| | DAP10.2 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCT |
| | | GTTCCTGCTGCTGCTCCCCGTGGCCGCCGCTCAGACCACCCCTGGAGAAAGAAGCAGCCTGC |
| | | CTGCCTTCTACCCCGGGACCAGCGGCTCATGCAGCGGTTGTGGCAGCCTGAGCCTGCCTCTG |
| | | CTGGCCGGCCTGGTGGCTGCTGATGCCGTGGCATCTCTGCTGATCGTGGGCGCCGTCTTTCT |
| | | GTGCGCCAGACCTAGACGGAGCCCCGCCCAGGAGGACGGCAGAGTGTTCATCAACATGCCTG |
| | | GCAGAGGCTGA |
| 115 | 3H7-5.1-P2A- | MSVPTQVLGLLLLWLTDARCEIVMTQSPATLSVSPGERTTLSCRA |
| | DAP10.2′ | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGRVFINMPGRG* |
| 116 | 3H7-5.1-P2A- | atgtccgtgcctacccaggtgctgggcctgctgctgctgtggctgaccgacgccagatgcga |
| | DAP10.3 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | Gccggaggggcaaggggcacgatggcctttaccagggtctcagtacagccaccaaggacacc |
| | | tacgacgcccttcacatgcaggccctgccccctcgcGGTAGCGGGGCTACGAACTTCTCCCT |
| | | TCTTAAACAAGCGGGAGACGTGAAGAAAATCCCGGACCTATGATCCACCTGGGCCACATCCT |
| | | GTTCCTGCTGCTGCTGCCTGTGGCCGCTGCCCAGACCACCCCTGGAGAAAGATCTAGCCTTC |
| | | CAGCCTTCTACCCCGGGACCAGCGGAAGCTGCAGCGGCTGCGGCAGCTTGTCTCTGCCTCTG |
| | | CTGGCCGGCCTGGTGGCCGCCGACGCTGTTGCATCTCTGCTGATCGTGGGCGCCGTCTTTCT |
| | | GTGCGCCAGACCCAGACGGAGCCCTGCTCAGGAAGATGGCAAAGTGTACATCAACATGCCTG |
| | | GCAGAGGCAGAGTGAAGTTCAGCAGAAGCGCCGATGCCCCTGCCTATCAGCA |
| | | GGGCCAGAACCAGCTGTACAACGAGCTGAACCTGGGCAGACG |
| | | CGAGGAATACGACGTGCTGGACAAGCGGCGGGGACGGGACC |
| | | CCGAGATGGGCGGCAAGCCTCAAAGAAGGAAGAACCCCCAG |
| | | GAGGGCCTGTATAATGAGCTGCAGAAAGATAAGATGGCCGAG |
| | | GCCTACAGCGAGATCGGCATGAAAGGCGAGAGACGGCGGGG |
| | | CAAGGGCCACGACGGCCTGTACCAGGGCCTGAGCACCGCCAC |
| | | AAAGGACACCTACGACGCCCTGCACATGCAGGCCCTGCCACC |
| | | TAGATGA |
| 117 | 3H7-5.1-P2A- | MSVPTQVLGLLLLWLTDARCEIVMTQSPATLSVSPGERTTLSCRA |
| | DAP10.3 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVYINMPGRGRVKFSRSADAPAYQQGQN |
| | | QLYNELNLGRREEYDVLDKRRGRDPEMGGKPQRRKNPQEGLYN |
| | | ELQKDKMAEAYSEIGMKGERRRGKGHDGLYQGLSTATKDTYDA |
| | | LHMQALPPR* |
| 118 | 3H7-5.1-P2A- | atgtccgtgcctacccaggtgctgggcctgctgctgctgtggctgaccgacgccagatgcga |
| | DAP10.4 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCT |
| | | GTTCCTGCTGCTGCTGCCTGTGGCCGCCGCTCAAACAACCCCTGGCGAGAGATCTAGCCTGC |
| | | CAGCCTTCTACCCCGGTACAAGCGGATCTTGCAGCGGCTGCGGCAGCCTGTCTCTGCCTCTG |
| | | CTGGCCGGCCTGGTGGCTGCTGATGCTGTGGCCAGCCTGCTGATCGTGGGCGCCGTGTTCCT |
| | | GTGCGCCAGACCTAGAAGGTCCCCTGCCCAGGAGGATGGAAAGGTGTACATCAACATGCCTG |
| | | GCAGAGGCAAGAGAGGCAGAAAGAAGCTGCTGTACATCTTCAAGCAGCCTTT |
| | | CATGAGACCAGTGCAGACCACCCAGGAGGAGGACGGATGTAG |
| | | CTGCAGATTCCCCGAGGAAGAAGAAGGCGGCTGTGAACTGAG |
| | | AGTGAAGTTCAGCAGAAGCGCCGACGCCCCTGCTTATCAGCA |
| | | GGGCCAGAACCAGCTGTACAACGAGCTGAACCTGGGCAGAAG |
| | | GGAAGAGTACGACGTGCTGGACAAGCGGCGGGGACGGGACC |
| | | CCGAGATGGGCGGAAAACCTCAAAGACGGAAGAACCCCCAG |
| | | GAGGGCCTTTATAATGAGCTGCAGAAAGATAAGATGGCCGAG |
| | | GCCTACAGCGAGATCGGCATGAAAGGAGAAAGACGGCGGGG |
| | | CAAAGGCCACGACGGCCTGTACCAGGGCCTGAGCACCGCCAC |
| | | AAAGGACACCTACGACGCCCTGCACATGCAGGCCCTGCCCCC |
| | | CAGATGA |
| 119 | 3H7-5.1-P2A- | MSVPTQVLGLLLLWLTDARCEIVMTQSPATLSVSPGERTTLSCRA |
| | DAP10.4 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVYINMPGRGKRGRKKLLYIFKQPFMRP |
| | | VQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDAL |
| | | HMQALPPR* |
| 120 | 3H7-5.1-P2A- | atgtccgtgcctacccaggtgctgggcctgctgctgctgtggctgaccgacgccagatgcga |
| | DAP10.5 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatcta |
| | | catctgggcgcccttggccgggacttgtggggtccttctcctgtcactggttatcacccttt |
| | | actgcaaacggggcagaaagaaactcctgtatatattcaaacaaccatttatgagaccagta |
| | | caaactactcaagaggaagatggctgtagctgccgatttccagaagaagaagaaggaggatg |
| | | tgaactgagagtgaagttcagcaggagcgcagacgcccccgcgtaccagcagggccagaacc |
| | | agctctataacgagctcaatctaggacgaagagaggagtacgatgttttggacaagagacgt |
| | | ggccgggaccctgagatggggggaaagccgcagagaaggaagaaccctcaggaaggcctgta |
| | | caatgaactgcagaaagataagatggcggaggcctacagtgagattgggatgaaaggcgagc |
| | | gccggaggggcaaggggcacgatggcctttaccagggtctcagtacagccaccaaggacacc |
| | | tacgacgcccttcacatgcaggccctgccccctcgcGGTAGCGGGGCTACGAACTTCTCCCT |
| | | TCTTAAACAAGCGGGAGACGTGGAAGAAAATCCCGGACCTATGATCCACCTGGGACACATCC |
| | | TGTTCCTGCTGCTGCTTCCAGTGGCCGCCGCTCAAACAACCCCTGGCGAGAGAAGCAGCCTG |
| | | CCCGCCTTCTACCCCGGTACATCTGGCAGCTGCAGCGGCTGCGGCAGCTTGTCTCTGCCTCT |
| | | GCTGGCCGGACTGGTGGCTGCTGATGCCGTGGCCAGCCTGCTGATCGTGGGCGCCGTGTTCC |
| | | TGTGCGCCAGACCCAGAAGGTCCCCTGCCCAGGAGGACGGCAAGGTGTTCATCAACATGCCT |
| | | GGCAGAGGCAAAAGAGGCAGAAAGAAGCTGCTGTACATCTTCAAGCAGCCTTT |
| | | CATGCGGCCTGTGCAGACCACCCAGGAGGAAGATGGCTGCTC |
| | | TTGTCGATTTCCAGAAGAGGAAGAAGGCGGCTGTGAACTGAG |
| | | AGTGAAGTTCAGCAGATCCGCCGACGCCCCTGCTTATCAGCAG |
| | | GGCCAGAACCAGCTGTATAATGAGCTGAACCTGGGCCGGCGG |
| | | GAAGAGTACGACGTGCTGGACAAGCGGCGGGGCAGAGATCCT |
| | | GAGATGGGCGGAAAACCTCAAAGAAGGAAGAACCCCCAGGA |
| | | GGGACTGTACAACGAGCTGCAGAAGGATAAGATGGCCGAGGC |
| | | CTACAGCGAGATCGGCATGAAAGGAGAAAGAAGAAGAGGAA |
| | | AAGGCCACGACGGCCTGTACCAGGGCCTGAGCACCGCCACCA |
| | | AGGACACCTACGACGCCCTGCACATGCAGGCCCTGCCACCTA |
| | | GATGA |
| 121 | 3H7-5.1-P2A- | MSVPTQVLGLLLLWLTDARCEIVMTQSPATLSVSPGERTTLSCRA |
| | DAP10.5 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVFINMPGRGKRGRKKLLYIFKQPFMRP |
| | | VQTTQEEDGCSCRFPEEEEGGCELRVKFSRSADAPAYQQGQNQL |
| | | YNELNLGRREEYDVLDKRRGRDPEMGGKPQRRKNPQEGLYNEL |
| | | QKDKMAEAYSEIGMKGERRRGKGHDGLYQGLSTATKDTYDAL |
| | | HMQALPPR* |
| 122 | 3H7-5.1-P2A- | atgtccgtgcctacccaggtgctgggcctgctgctgctgtggctgaccgacgccagatgcga |
| | DAP10.6 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCT |
| | | GTTCCTGCTGCTCCTGCCTGTGGCCGCTGCTCAGACCACCCCTGG |
| | | AGAAAGATCTAGCCTGCCCGCTTTCTACCCCGGGACCAGCGGC |
| | | AGCTGCAGCGGATGCGGCTCTCTGAGCCTGCCTCTGCTGGCCG |
| | | GCCTCGTGGCCGCCGATGCTGTGGCCAGCCTGCTGATCGTGGG |
| | | CGCCGTGTTCCTGTGCGCCAGACCCAGAAGAAGCCCTGCCCA |
| | | GGAGGATGGCAGAGTGTTCATCAACATGCCTGGCAGAGGCAA |
| | | AAGAGGCAGAAAGAAGCTGCTGTACATCTTCAAGCAGCCTTT |
| | | CATGCGGCCCGTGCAGACAACCCAGGAGGAGGACGGCTGTAG |
| | | CTGTAGATTCCCCGAGGAAGAAGAAGGCGGCTGCGAGCTTAG |
| | | AGTGAAGTTCAGCAGAAGCGCCGACGCCCCTGCTTACCAGCA |
| | | GGGCCAGAACCAGCTGTATAATGAGCTGAACCTGGGAAGAAG |
| | | GGAAGAGTACGACGTGCTGGACAAGCGGCGGGGCAGAGATC |
| | | CTGAGATGGGCGGAAAACCTCAAAGAAGGAAGAACCCTCAG |
| | | GAGGGCCTGTACAACGAGCTGCAGAAAGACAAGATGGCCGA |
| | | GGCCTACAGCGAGATCGGCATGAAAGGCGAGAGACGGCGGG |
| | | GCAAGGGCCACGACGGCCTGTACCAAGGCCTGTCAACAGCCA |
| | | CCAAGGACACCTACGACGCCCTGCACATGCAGGCCCTGCCAC |
| | | CTAGATGA |
| 123 | 3H7-5.1-P2A- | MSVPTQVLGLLLLWLTDARCEIVMTQSPATLSVSPGERTTLSCRA |
| | DAP10.6 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGRVFINMPGRGKRGRKKLLYIFKQPFMRPV |
| | | QTTQEEDGCSCRFPEEEEGGCELRVKFSRSADAPAYQQGQNQLY |
| | | NELNLGRREEYDVLDKRRGRDPEMGGKPQRRKNPQEGLYNELQ |
| | | KDKMAEAYSEIGMKGERRRGKGHDGLYQGLSTATKDTYDALH |
| | | MQALPPR* |
| 124 | 3H7-5.1-P2A- | atgtccgtgcctacccaggtgctgggcctgctgctgctgtggctgaccgacgccagatgcga |
| | DAP10.7 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCTGT |
| | | TCCTGCTGCTGCTGCCTGTGGCCGCTGCTCAGACCACACCTGG |
| | | CGAGAGAAGCAGCCTGCCCGCCTTCTACCCCGGTACAAGCGG |
| | | CTCTTGTTCTGGCTGCGGCAGCCTGAGCCTGCCTCTGCTCGCC |
| | | GGCCTGGTGGCCGCCGACGCCGTGGCATCTCTGCTGATCGTGG |
| | | GCGCCGTGTTCCTGTGCGCCAGACCTAGACGGAGCCCTGCCCA |
| | | GGAGGACGGCAAAGTGTACATCAACATGCCTGGAAGAGGCAA |
| | | GCGGGGCAGAAAGAAGCTGCTGTACATCTTCAAGCAGCCTTT |
| | | CATGAGACCCGTGCAGACCACCCAGGAGGAAGATGGCTGTAG |
| | | CTGCAGATTCCCCGAGGAAGAAGAGGGCGGATGCGAGCTGTG |
| | | A |
| 125 | 3H7-5.1-P2A- | MSVPTQVLGLLLLWLTDARCEIVMTQSPATLSVSPGERTTLSCRA |
| | DAP10.7 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVYINMPGRGKRGRKKLLYIFKQPFMRP |
| | | VQTTQEEDGCSCRFPEEEEGGCEL* |
| 126 | 3H7-5.1-P2A- | atgtccgtgcctacccaggtgctgggcctgctgctgctgtggctgaccgacgccagatgcga |
| | DAP10.8 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGACACATCCTG |
| | | TTCCTGCTGCTGCTGCCTGTGGCCGCTGCTCAGACCACCCCTG |
| | | GCGAGAGAAGCAGCCTGCCCGCCTTCTACCCCGGCACCAGCG |
| | | GATCTTGTAGCGGCTGCGGCAGCCTGAGCCTGCCTCTGCTCGC |
| | | CGGCCTGGTGGCCGCCGACGCCGTGGCATCTCTGCTGATCGTG |
| | | GGCGCCGTCTTTCTGTGCGCCAGACCTAGACGGAGCCCTGCCC |
| | | AGGAGGACGGCAAAGTGTTCATCAACATGCCTGGAAGAGGCA |
| | | AGCGGGGCAGAAAGAAGCTGCTGTACATCTTCAAGCAGCCTT |
| | | TCATGAGACCCGTGCAGACAACCCAGGAGGAAGATGGCTGCA |
| | | GCTGCAGATTCCCCGAGGAAGAGGAAGGCGGCTGTGAACTGT |
| | | GA |
| 127 | 3H7-5.1-P2A- | MSVPTQVLGLLLLWLTDARCEIVMTQSPATLSVSPGERTTLSCRA |
| | DAP10.8 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVFINMPGRGKRGRKKLLYIFKQPFMRP |
| | | VQTTQEEDGCSCRFPEEEEGGCEL* |
| 128 | 3H7-5.1-P2A- | atgtccgtgcctacccaggtgctgggcctgctgctgctgtggctgaccgacgccagatgcga |
| | DAP10.9 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCTGT |
| | | TCCTGCTGCTGCTGCCTGTGGCTGCCGCTCAGACAACCCCTGG |
| | | CGAGAGAAGCAGCCTGCCCGCCTTCTACCCCGGGACCAGCGG |
| | | CTCTTGTTCTGGCTGCGGCAGCCTGAGCCTGCCTCTGCTCGCC |
| | | GGCCTGGTGGCCGCCGACGCCGTGGCCAGCCTGCTGATCGTG |
| | | GGCGCCGTGTTCCTGTGCGCCAGACCTAGAAGGTCCCCTGCCC |
| | | AGGAGGACGGCAGAGTGTTCATCAACATGCCTGGCAGAGGCA |
| | | AGCGGGGCAGAAAGAAGCTGCTGTACATCTTCAAGCAGCCTT |
| | | TCATGCGGCCTGTGCAGACCACACAGGAGGAAGATGGCTGCA |
| | | GCTGCAGATTCCCCGAGGAAGAGGAAGGCGGATGTGAACTGT |
| | | GA |
| 129 | 3H7-5.1-P2A- | MSVPTQVLGLLLLWLTDARCEIVMTQSPATLSVSPGERTTLSCRA |
| | DAP10.9 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGRVFINMPGRGKRGRKKLLYIFKQPFMRPV |
| | | QTTQEEDGCSCRFPEEEEGGCEL* |
| 130 | 3H7-5.1-P2A- | atgtccgtgcctacccaggtgctgggcctgctgctgctgtggctgaccgacgccagatgcga |
| | DAP10.0 | aatagtgatgacgcagtctccagccaccctgtctgtgtctccaggggaaagaaccaccctct |
| | Nucleic Acid | cctgcagggccagtcagagtgttagcagcaacttagcctggtaccttcagaaacctggccag |
| | | gctcccaggctcctcatctatggtgcatccaccagggccactggtatcccagccaggttcag |
| | | tggcagtgggtctgggacagagttcattctcaccatcagcagcctgcagtctgaagattttg |
| | | cagtttattactgtcagcagtataataactggccgatcaccttcggccaagggacacggctg |
| | | gagattaaaggtggaggtggatctggaggaggaggatccggtggaggaggtgaagtgcaact |
| | | ggtggagtctgggggaggcttggtacagcctggcaggtccctgagactctcctgtgcagcct |
| | | ctggattcaccttttatgattatgccatgcactgggtccggcaagctccagggaagggcctg |
| | | gagtgggtctcaggtattagttggaatagtggttacataggctatgcggactctgtgaaggg |
| | | ccgattcaccatctccagagacaacgccaagaactccctgtatctgcaaatgaacagtctga |
| | | gagctgaggacacggccttgtattactgtgcaaaagataaCAGCTATGGAAAGTTCTACTAC |
| | | GGtttggacgtctggggccaagggaccacggtcaccgtctcctcaaccacgacgccagcgcc |
| | | gcgaccaccaacaccggcgcccaccatcgcgtcgcagcccctgtccctgcgcccagaggcgt |
| | | gccggccagcggcggggggcgcagtgcacacgagggggctggacttcgcctgtgatatctac |
| | | atctgggcgcccttggccgggacttgtggggtccttctcctgtcactggttatcacccttta |
| | | ctgcaaacggggcagaaagaaactcctgtatatattcaaacaaccatttatgagaccagtac |
| | | aaactactcaagaggaagatggctgtagctgccgatttccagaagaagaagaaggaggatgt |
| | | gaactgagagtgaagttcagcaggagcgcagacgcccccgcgtaccagcagggccagaacca |
| | | gctctataacgagctcaatctaggacgaagagaggagtacgatgttttggacaagagacgtg |
| | | gccgggaccctgagatggggggaaagccgcagagaaggaagaaccctcaggaaggcctgtac |
| | | aatgaactgcagaaagataagatggcggaggcctacagtgagattgggatgaaaggcgagcg |
| | | ccggaggggcaaggggcacgatggcctttaccagggtctcagtacagccaccaaggacacct |
| | | acgacgcccttcacatgcaggccctgccccctcgcGGTAGCGGGGCTACGAACTTCTCCCTT |
| | | CTTAAACAAGCGGGAGACGTGGAAGAAAATCCCGGACCTATGATCCACCTGGGCCACATCCT |
| | | GTTTCTGCTGCTGCTTCCTGTGGCCGCTGCTCAGACAACACCTGG |
| | | CGAGAGATCTAGCCTGCCTGCCTTCTATCCTGGCACCAGCGGC |
| | | TCTTGTTCTGGCTGTGGATCTCTGAGCCTGCCTCTGCTGGCTGG |
| | | ACTGGTTGCTGCTGATGCTGTGGCCTCTCTGCTGATTGTGGGC |
| | | GCCGTGTTCCTGTGTGCCCGGCCTAGAAGATCTCCCGCTCAAG |
| | | AGGATGGCAAGGTGTACATCAACATGCCCGGCAGAGGATGA |
| 131 | 3H7-5.1-P2A- | MSVPTQVLGLLLLWLTDARCEIVMTQSPATLSVSPGERTTLSCRA |
| | DAP10.0 | SQSVSSNLAWYLQKPGQAPRLLIYGASTRATGIPARFSGSGSGTEF |
| | Protein | ILTISSLQSEDFAVYYCQQYNNWPITFGQGTRLEIKGGGGSGGGG |
| | | SGGGGEVQLVESGGGLVQPGRSLRLSCAASGFTFYDYAMHWVR |
| | | QAPGKGLEWVSGISWNSGYIGYADSVKGRFTISRDNAKNSLYLQ |
| | | MNSLRAEDTALYYCAKDNSYGKFYYGLDVWGQGTTVTVSSTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYQQGQNQLYNELNLG |
| | | RREEYDVLDKRRGRDPEMGGKPQRRKNPQEGLYNELQKDKMA |
| | | EAYSEIGMKGERRRGKGHDGLYQGLSTATKDTYDALHMQALPP |
| | | RGSGATNFSLLKQAGDVEENPGPMIHLGHILFLLLLPVAAAQTTP |
| | | GERSSLPAFYPGTSGSCSGCGSLSLPLLAGLVAADAVASLLIVGA |
| | | VFLCARPRRSPAQEDGKVYINMPGRG* |

TABLE 4
SEQ ID NO: 22 Annotation

| Description | Nucleotide numbering |
|---|---|
| Signal Peptide | 1-60 |
| DAP10.0 | 61-339 |
| Furin P2A | 340-420 |
| Truncated CD19 | 421-1419 |

TABLE 5
SEQ ID NO: 24 Annotation

| Description | Nucleotide Numbering |
|---|---|
| Signal Peptide | 1-60 |
| DAP10.3 (DAP10.0 + CD3z) | 61-678 |
| DAP10.0 | 61-339 |
| CD3z Signaling domain | 340-678 |
| Furin P2A | 679-759 |
| Truncated CD19 | 760-1758 |

TABLE 6
SEQ ID NO: 26 Annotation

| Description | Nucleotide Numbering |
|---|---|
| Signal Peptide | 1-60 |
| DAP10.4 (DAP10.0 + 4-1BB + CD3z) | 61-804 |
| DAP10.0 | 61-339 |
| 4-1BB Co-stimulatory Domain | 340-465 |
| CD3z Signaling domain | 466-804 |
| Furin P2A | 805-885 |
| Truncated CD19 | 886-1884 |

TABLE 7
SEQ ID NO: 28 Annotation

| Description | Nucleotide Numbering |
|---|---|
| Signal Peptide | 1-60 |
| DAP10.5 (DAP10-Y86F + 4-1BB + CD3z) | 61-804 |
| DAP10-Y86F (Mutation in bold and underline) | 61-339 |
| 4-1BB Co-stimulatory Domain | 340-465 |
| CD3z Signaling Domain | 466-804 |
| Furin P2A | 805-885 |
| Truncated CD19 | 886-1884 |

TABLE 8
SEQ ID NO: 30 Annotation

| | Nucleotide |
|---|---|
| Description | Numbering |
| Signal Peptide | 1-60 |
| DAP10.6 (DAP10-K84R-Y86F + 4-1BB + CD3z) | 61-804 |
| DAP10-K84R-Y86F (Mutations in bold and | 61-339 |
| underline) | |
| 4-1BB Co-stimulatory Domain | 340-465 |
| CD3z Signaling Domain | 466-804 |
| Furin P2A | 805-885 |
| Truncated CD19 | 886-1884 |

TABLE 9
SEQ ID NO: 32 Annotation

| | Nucleotide |
|---|---|
| Description | Numbering |
| Signal Peptide | 1-60 |
| DAP10.13 (DAP10-K84R) (Mutation in bold and | 61-339 |
| underline) | |
| Furin P2A | 340-420 |
| Truncated CD19 | 421-1419 |

TABLE 10
SEQ ID NO: 34 Annotation

| Description | Numbering |
|---|---|
| Signal Peptide | 1-60 |
| DAP10.14 (DAP10-K84R + CD3z) | 61-678 |
| DAP10-K84R (Mutation in bold and underline) | 61-339 |
| CD3z Signaling domain | 340-678 |
| Furin P2A | 679-759 |
| Truncated CD19 | 760-1758 |

TABLE 11
SEQ ID NO: 36 Annotation

| Description | Numbering |
|---|---|
| Signal Peptide | 1-60 |
| DAP10.15 (DAP10-K84R + 4-1BB + CD3z) | 61-804 |
| DAP10-K84R (Mutation in bold and underline) | 61-339 |
| 4-1BB Co-stimulatory Domain | 340-465 |
| CD3z Signaling domain | 466-804 |
| Furin P2A | 805-885 |
| Truncated CD19 | 886-1884 |

TABLE 12
SEQ ID NO: 84 Annotation

| Description | Numbering |
|---|---|
| Signal Peptide | 1-54 |
| DAP10.6 (DAP10-K84R-Y86F-41BB-CD3z) | 1-744 |
| DAP10-K84R-Y86F | 1-279 |
| K84R | 250-252 |
| Y86F | 256-258 |
| 4-1BB Co-stimulatory Domain | 280-405 |
| CD3z Signaling domain | 406-744 |
| Furin P2A | 745-825 |
| GMCSFR alpha chain signal sequence | 826-891 |
| Truncated EGFR | 892-1902 |

TABLE 13
SEQ ID NO: 86 Annotation

| Description | Numbering |
|---|---|
| Signal Peptide | 1-54 |
| DAP10.16 (DAP10-K84R-Y86F-41BB-1XX | 1-744 |
| mutant with Y > F in ITAMS 2&3) | |
| DAP10-K84R-Y86F | 1-279 |
| K84R | 250-252 |
| Y86F | 256-258 |
| 4-1BB Co-stimulatory Domain | 280-405 |
| 1XX mutant with Y > F in ITAM 2&3 | 406-744 |
| Y > F in ITAM2 | 583-585 |
| Y > F in ITAM2 | 619-621 |
| Y > F in ITAM3 | 676-678 |
| Y > F in ITAM3 | 709-711 |
| Furin P2A | 745-825 |
| GMCSFR alpha chain signal sequence | 826-891 |
| Truncated EGFR | 892-1902 |

TABLE 14
SEQ ID NO: 88 Annotation

| Description | Numbering |
|---|---|
| Signal Peptide | 1-54 |
| DAP10.17 (DAP10-K84R-Y86F-1XX mutant with | 1-618 |
| Y > Fin ITAMS 2&3 without 41BB | |
| DAP10-K84R-Y86F | 1-279 |
| K84R | 250-252 |
| Y86F | 256-258 |
| 1XX mutant with Y > F in ITAM 2&3 | 280-618 |
| Y > F in ITAM2 | 457-459 |
| Y > F in ITAM2 | 493-495 |
| Y > F in ITAM3 | 550-552 |
| Y > F in ITAM3 | 583-585 |
| Furin P2A | 619-699 |
| GMCSFR alpha chain signal sequence | 700-765 |
| Truncated EGFR | 766-1776 |

TABLE 15
SEQ ID NO: 95 Annotation

| Description | Numbering |
|---|---|
| Signal Peptide | 1-54 |
| DAP10.6 (DAP10-K84R-Y86F-41BB-CD3z) | 1-744 |
| DAP10-K84R-Y86F | 1-279 |
| K84R | 250-252 |
| Y86F | 256-258 |
| 4-1BB Co-stimulatory Domain | 280-405 |
| CD3z Signaling domain | 406-744 |
| Furin P2A | 745-825 |
| GMCSFR alpha chain signal sequence | 826-891 |
| Truncated EGFR | 892-1896 |

TABLE 16
SEQ ID NO: 97 Annotation

| Description | Numbering |
|---|---|
| Signal Peptide | 1-54 |
| DAP10.16 (DAP10-K84R-Y86F-41BB-1XX | 1-744 |
| mutant with Y > F in ITAMS 2&3) | |
| DAP10-K84R-Y86F | 1-279 |
| K84R | 250-252 |
| Y86F | 256-258 |
| 4-1BB Co-stimulatory Domain | 280-405 |
| 1XX mutant with Y > F in ITAM 2&3 | 406-744 |
| Y > F in ITAM2 | 583-585 |
| Y > F in ITAM2 | 619-621 |
| Y > F in ITAM3 | 676-678 |
| Y > F in ITAM3 | 709-711 |
| Furin P2A | 745-825 |
| CD19 signal sequence | 826-885 |
| Truncated CD19 | 826-1824 |

TABLE 17
SEQ ID NO: 103 Annotation

| Description | Numbering |
|---|---|
| Signal Peptide | 1-54 |
| DAP10.16 (DAP10-K84R-Y86F-41BB-1XX | 1-744 |
| mutant with Y > F in ITAMS 2&3) | |
| DAP10-K84R-Y86F | 1-279 |
| K84R | 250-252 |
| Y86F | 256-258 |
| 4-1BB Co-stimulatory Domain | 280-405 |
| 1XX mutant with Y > F in ITAM 2&3 | 406-744 |
| Y > F in ITAM2 | 583-585 |
| Y > F in ITAM2 | 619-621 |
| Y > F in ITAM3 | 676-678 |
| Y > F in ITAM3 | 709-711 |
| Furin P2A | 745-825 |
| GMCSFR alpha chain signal sequence | 826-891 |
| Truncated EGFR | 892-1896 |

TABLE 18
SEQ ID NO: 105 Annotation

| Description | Numbering |
|---|---|
| Signal Peptide | 1-54 |
| DAP10.17 (DAP10-K84R-Y86F-1XX mutant | 1-618 |
| with Y > F in ITAMS 2&3 without 41BB) | |
| DAP10-K84R-Y86F | 1-279 |
| K84R | 250-252 |
| Y86F | 256-258 |
| 1XX mutant with Y > F in ITAM 2&3 | 280-618 |
| Y > F in ITAM2 | 457-459 |
| Y > F in ITAM2 | 493-495 |
| Y > F in ITAM3 | 550-552 |
| Y > F in ITAM3 | 583-585 |
| Furin P2A | 619-699 |
| CD19 Signal Sequence | 700-759 |
| Truncated CD19 | 766-1698 |

TABLE 19
SEQ ID NO: 111 Annotation

| Description | Numbering |
|---|---|
| Signal Peptide | 1-54 |
| DAP10.17 (DAP10-K84R-Y86F-1XX mutant | 1-618 |
| with Y > F in ITAMS 2&3 without 41BB | |
| DAP10-K84R-Y86F | 1-279 |
| K84R | 250-252 |
| Y86F | 256-258 |
| 1XX mutant with Y > F in ITAM 2&3 | 280-618 |
| Y > F in ITAM2 | 457-459 |
| Y > F in ITAM2 | 493-495 |
| Y > F in ITAM3 | 550-552 |
| Y > F in ITAM3 | 583-585 |
| Furin P2A | 619-699 |
| GMCSFR alpha chain signal sequence | 700-765 |
| Truncated EGFR | 766-1770 |

TABLE 20
SEQ ID NO: 112 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.1 (DAP10-Y86F) | 1528-1806 |
| Signal Peptide | 1528-1581 |
| DAP10-Y86F | 1582-1806 |
| Y86F | 1783-1785 |

TABLE 21
SEQ ID NO: 114 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.2 (DAP10-K84R-Y86F) | 1528-1806 |
| Signal Peptide | 1528-1581 |
| DAP10-K84R-Y86F | 1582-1806 |
| K84R | 1777-1779 |
| Y86F | 1783-1785 |

TABLE 22
SEQ ID NO: 116 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.3 (DAP10_WT-CD3z) | 1528-2145 |
| Signal Peptide | 1528-1581 |
| DAP10 WT | 1582-1806 |
| CD3z Signaling Domain | 1807-2145 |

TABLE 23
SEQ ID NO: 118 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.4 (DAP10_WT-41BB-CD3z) | 1528-2271 |
| Signal Peptide | 1528-1581 |
| DAP10 WT | 1582-1806 |
| 4-1BB Costimulatory Domain | 1807-1932 |
| CD3z Signaling Domain | 1933-2271 |

TABLE 24
SEQ ID NO: 120 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.5 (DAP10-Y86F-41BB-CD3z) | 1528-2271 |
| Signal Peptide | 1528-1581 |
| DAP10-Y86F | 1582-1806 |
| Y86F | 1783-1785 |
| 4-1BB Costimulatory Domain | 1807-1932 |
| CD3z Signaling Domain | 1933-2271 |

TABLE 25
SEQ ID NO: 122 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.6 (DAP10-K84R-Y86F-41BB-CD3z) | 1528-2271 |
| Signal Peptide | 1528-1581 |
| DAP10-K84R-Y86F | 1582-1806 |
| K84R | 1777-1779 |
| Y86F | 1783-1785 |
| 4-1BB Costimulatory Domain | 1807-1932 |
| CD3z Signaling Domain | 1933-2271 |

TABLE 26
SEQ ID NO: 124 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.7 (DAP10_WT-41BB) | 1528-1932 |
| Signal Peptide | 1528-1581 |
| DAP10 WT | 1582-1806 |
| 4-1BB Costimulatory Domain | 1807-1932 |

TABLE 27
SEQ ID NO: 126 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.8 (DAP10-Y86F-41BB) | 1528-1932 |
| Signal Peptide | 1528-1581 |
| DAP10-Y86F | 1582-1806 |
| Y86F | 1783-1785 |
| 4-1BB Costimulatory Domain | 1807-1932 |

TABLE 28
SEQ ID NO: 128 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.9 (DAP10-K84R-Y86F-41BB) | 1528-1932 |
| Signal Peptide | 1528-1581 |
| DAP10-K84R-Y86F | 1582-1806 |
| K84R | 1777-1779 |
| Y86F | 1783-1785 |
| 4-1BB Costimulatory Domain | 1807-1932 |

TABLE 29
SEQ ID NO: 130 Annotation

| Annotation | Numbering |
|---|---|
| Signal Peptide | 1-60 |
| 3H7 Light chain variable | 61-381 |
| GS Linker | 382-423 |
| 3H7 Heavy chain variable | 424-789 |
| CD8 Hinge and Transmembrane Domain | 790-996 |
| 4-1BB Costimulatory Domain | 997-1122 |
| CD3z Signaling Domain | 1123-1461 |
| P2A | 1462-1527 |
| DAP10.0 (DAP10_WT) | 1528-1806 |
| Signal Peptide | 1528-1581 |
| DAP10 WT | 1582-1806 |

1. A mammalian cell comprising a chimeric adapter (CAD) polypeptide comprising a DAP10 domain comprising a human DAP10 amino acid sequence, at least one of a costimulatory domain and/or an intracellular signaling domain, and specifically lacking an ectodomain comprising a functional extracellular receptor and/or ligand-binding domain, wherein the mammalian cell further comprises at least one chimeric receptor comprising an extracellular targeting domain that specifically binds to target antigens on a target cell.

2. The mammalian cell according to claim 1, wherein said chimeric receptor comprises at least one of an intracellular signaling domain and/or a costimulatory domain.

3. The mammalian cell according to claim 1 or claim 2, wherein said chimeric receptor comprises at least one DAP10-interacting domain.

4. The mammalian cell according to claim 3, wherein the DAP10-interacting domain comprises the amino acid sequence set forth in SEQ ID NO: 75, or an amino acid sequence comprising at least 80%, 90%, or 95% sequence identity to SEQ ID NO: 75.

5. The mammalian cell according to any one of claims 1-4, wherein a target antigen on a target cell is selected from the group consisting of CD20, BCMA, GPC3, TyrD, FcRL5, B7H6, CD70, PSMA, CD19 and FAP.

6. The mammalian cell according to any one of claims 1-5, wherein the CAD polypeptide comprises at least one costimulatory domain selected from TLR1, TLR2, TLR3, TLR4, TLR5, TLR6, TLR7, TLR8, TLR9, TLR10, CARD11, CD2, CD3C, CD7, CD27, CD28, CD30, CD40, CD54 (ICAM), CD70, CD80, CD83, CD86, CD134 (OX40), CD137 (4-1BB), CD278 (ICOS), FcR, LAT, NKD2C, SLP76, TRIM, and ZAP70, or combinations thereof.

7. The mammalian cell according to claim 6, wherein the at least one costimulatory domain of the CAD polypeptide is 4-1BB.

8. The mammalian cell according to claim 6, wherein the at least one costimulatory domain of the CAD polypeptide is CD28.

9. The mammalian cell according to any one of claims 1-8, wherein the CAD polypeptide comprises at least one intracellular signaling domain selected from CD3ζ, DAP12, LFA-1, and repeat (2-5) DAP10 YINM motifs.

10. The mammalian cell according to claim 9, wherein the at least one intracellular signaling domain of the CAD polypeptide is CD3ζ, optionally wherein CD3ζ has an amino acid sequence set forth as SEQ ID NO: 82.

11. The mammalian cell according to claim 9 or claim 10, wherein the at least one costimulatory domain of the CAD polypeptide is 4-1BB, and the at least one intracellular signaling domain of the CAD polypeptide is CD3ζ.

12. The mammalian cell according to claim 11, wherein the CAD polypeptide comprises, from N-terminus to C-terminus, the DAP10 domain, the 4-1BB costimulatory domain followed by the CD3ζ intracellular signaling domain.

13. The mammalian cell according to claim 6, wherein the CAD polypeptide comprises a 4-1BB costimulatory domain and a CD28 costimulatory domain.

14. The mammalian cell according to claim 13, wherein said CAD polypeptide comprises, from N-terminus to C-terminus, the DAP10 domain, the 4-1BB costimulatory domain followed by the CD28 costimulatory domain, followed in turn by a CD3ζ intracellular signaling domain, optionally wherein CD3ζ has an amino acid sequence set forth as SEQ ID NO: 82.

15. The mammalian cell according to claim 13, wherein said CAD polypeptide comprises, from N-terminus to C-terminus, the DAP10 domain, the CD28 costimulatory domain followed by the 4-1BB costimulatory domain, followed in turn by a CD3ζ signaling domain, optionally wherein CD3C has an amino acid sequence set forth as SEQ ID NO: 82.

16. The mammalian cell according to any one of claims 1-15, wherein the human DAP10 amino acid sequence comprises an amino acid sequence having:
(i) at least 90%, 95%, 97%, or 99% sequence identity to SEQ ID NO: 1;
(ii) at least 90%, 95%, 97%, or 99% sequence identity to SEQ ID NO: 78; or
(iii) at least 90%, 95%, 97%, or 99% sequence identity to SEQ ID NO: 81.

17. The mammalian cell according to any one of claims 1-16, wherein the human DAP10 amino acid sequence comprises a mutated human DAP10 amino acid sequence.

18. The mammalian cell according to claim 17, wherein the mutated human DAP10 amino acid sequence comprises amino acid substitutions at positions corresponding to K84 and/or Y86.

19. The mammalian cell according to claim 18, wherein the amino acid substitution at position K84 comprises a K84R substitution.

20. The mammalian cell according to claim 18 or claim 19, wherein the amino acid substitution at Y86 comprises a Y86F substitution.

21. The mammalian cell according to any one of claims 1-20, wherein the CAD polypeptide is encoded by an isolated nucleic acid that is operably linked to a regulatable promoter; and
wherein the chimeric receptor is encoded by an isolated nucleic acid that is operably linked to a regulatable promoter.

22. The mammalian cell according to claim 21, wherein the isolated nucleic acid that encodes the CAD polypeptide encodes for a cytokine and/or wherein the isolated nucleic acid that encodes the chimeric receptor encodes for a cytokine.

23. The mammalian cell according to claim 22 wherein the cytokine is selected from the group consisting of IL-2, IL-4, IL-7, IL-15, IL-21, and IL-23.

24. The mammalian cell according to claim 2, wherein when said chimeric receptor comprises an intracellular signaling domain, said CAD polypeptide comprises a costimulatory domain, and vice-versa.

25. The mammalian cell according to any one of claims 1-24, further comprising at least one receptor that associates with DAP10, wherein said at least one receptor is not the at least one chimeric receptor.

26. The mammalian cell according to claim 25, wherein said at least one receptor is exogenous.

27. The mammalian cell according to claim 26, wherein the exogenous receptor is over-expressed.

28. The mammalian cell according to any one of claims 26-27, wherein the at least one exogenous receptor is selected from NKG2D, Ly49H, Ly49D, Sirp-b1, Siglec-15, and Cd300lb.

29. The mammalian cell according to claim 28, wherein the at least one receptor is NKG2D.

30. The mammalian cell according to any one of claims 1-29, wherein the mammalian cell is an immune cell, preferably wherein said immune cell is a cytotoxic cell.

31. The mammalian cell according to any one of claims 1-30, wherein the mammalian cell exhibits in vitro and/or in vivo killing activity against the target cell that exhibits cell surface expression of the target antigens.

32. The mammalian cell according to claim 31, wherein the target cell is a hematological tumor cell.

33. The mammalian cell according to claim 31, wherein the target cell is a solid tumor cell.

34. The mammalian cell according to any one of claims 31-33, wherein said in vitro and/or in vivo killing activity is greater than an innate level of in vitro and/or in vivo killing activity in a control mammalian cell that lacks expression of one or both of the chimeric receptor and/or the CAD polypeptide.

35. The mammalian cell according to any one of claims 1-34, wherein the mammalian cell proliferates in response to contact with the target cell.

36. The mammalian cell according to any one of claims 1-35, wherein the mammalian cell exhibits increased proliferation in response to contact with the target cell as compared to a control mammalian cell that lacks expression of one or both of the chimeric receptor and/or the CAD polypeptide.

37. The mammalian cell according to any one of claims 35-36, wherein the mammalian cell proliferates in a host organism that comprises the target cell.

38. The mammalian cell according to any one of claims 1-37, wherein the mammalian cell expresses pro-inflammatory cytokines in response to contact with the target cell.

39. The mammalian cell according to claim 38, wherein the pro-inflammatory cytokines comprise tumor necrosis factor alpha or interferon gamma.

40. A plurality of the mammalian cells according to any one of claims 1-39.

41. The plurality of the mammalian cells according to claim 40, wherein the plurality of mammalian cells comprises at least about 10⁶ cells, at least 10⁷ cells, or at least 10⁸ cells, preferably from about 10⁸ to 10¹¹ cells.

42. A method of making the mammalian cell according to any one of claims 21-41, wherein the method comprises transfecting the mammalian cell(s) with a construct comprising the isolated nucleic acid that encodes for the CAD polypeptide and at least one construct that encodes for the at least one chimeric receptor.

43. The method of claim 42, wherein the method comprises retroviral transduction.

44. The method of claim 42 or claim 43, wherein the method comprises ex vivo expansion of the mammalian cell(s), wherein the ex vivo expansion is performed before transfection and/or after transfection of the isolated nucleic acid that encodes for the CAD polypeptide and at least one construct that encodes for the at least one chimeric receptor.

45. A pharmaceutical composition comprising a pharmaceutically acceptable excipient and the mammalian cell according to any one of claims 1-39, or the plurality of mammalian cells according to claim 40 or claim 41.

46. A method of activating the mammalian cell according to any one of claims 1-39, or the plurality of mammalian cells according to claim 40 or claim 41, comprising contacting the target cell with the mammalian cell.

47. The method of claim 46, wherein the mammalian cell, or the plurality of mammalian cells, are introduced into a subject in need thereof; and
wherein the activating occurs in the subject.

48. Use of the mammalian cell according to any one of claims 1-39, the plurality of mammalian cells according to claim 40 or claim 41, or the pharmaceutical composition of claim 45, in the preparation of a medicament for treating a subject with a condition for which the mammalian cell, or the plurality thereof, reduces at least one symptom or sign of said condition in the subject.

49. Use of a tumor cell killing effective amount of a mammalian cell according to any one of claims 31-34, or the plurality of mammalian cells according to claim 40 or claim 41, or the pharmaceutical composition of claim 45, in the preparation of a medicament for the treatment of cancer in a subject in need thereof.

50. A method of killing a tumor cell, the method comprising contacting the tumor cell with a tumor cell killing effective amount of the mammalian cell according to any one of claims 31-34, the plurality of mammalian cells according to claim 40 or claim 41, or the pharmaceutical composition according to claim 45.

51. The method according to claim 50, wherein the method comprises introducing a therapeutically effective amount of the mammalian cell(s) or the pharmaceutical composition into a host organism comprising the tumor cell.

52. The method of claim 51, wherein the method comprises introducing into the host organism comprising the tumor cell the therapeutically effective amount of the mammalian cell(s) or the pharmaceutical composition and simultaneously or sequentially administering one or more methods to elevate common chain gamma chain cytokine(s).

53. The method of claim 52, wherein the administering one or more methods to elevate common gamma chain cytokine(s) comprises administering simultaneously with introducing the mammalian cell(s) or sequentially an amount of common gamma chain cytokine(s) effective to increase proliferation, cytotoxic activity, persistence, or the combination thereof of the introduced mammalian cell(s), preferably wherein the method comprises administering IL-2, more preferably wherein the method comprises administering IL-15.

54. The method of claim 53, wherein the one or more methods to elevate common gamma chain cytokine(s) comprise administering an amount of common gamma chain cytokine(s) effective to increase proliferation, cytotoxic activity, persistence, or the combination thereof of the introduced mammalian cell(s) before and/or after introducing the mammalian cell(s) or the pharmaceutical composition.

55. The method of any one of claims 52-54, wherein the one or more methods to elevate common gamma chain cytokine(s) comprises lymphodepletion before introducing the mammalian cell(s).

56. The method according to any one of claims 52-55, wherein the one or more methods to elevate common gamma chain cytokine(s) comprises secretion of one or more common gamma chain cytokine(s) from the introduced mammalian cell(s).

57. The method of any one of claims 51 to 56, wherein the method reduces the in vivo tumor burden in the host organism, and/or increases the mean survival time of the host organism as compared to a control organism, wherein the control organism is not treated with the mammalian cell(s) or the pharmaceutical composition.

58. The method of any one of claims 50 to 57, wherein the method is a method of treating cancer in a subject in need thereof.
